(19) United States
(12) Patent Application Publication
(10) Pub. No.: US 2017/0304306 A1
(43) Pub. Date: Oct. 26, 2017

(54) SELECTIVE NaV1.7 INHIBITORS FOR THE TREATMENT OF DIABETES

(71) Applicant: CHROMOCELL CORPORATION, NORTH BRUNSWICK, NJ (US)

(72) Inventors: Yanlin WANG-FISCHER, Hillsborough, NJ (US); Olga BABICH, Cranford, NJ (US); Tina GARYANTES, Warren, NJ (US); Robert Z. LUO, New City, NY (US); Srinivasan P. Venkatachalan, Monmouth Junction, NJ (US)

(73) Assignee: CHROMOCELL CORPORATION, NORTH BRUNSWICK, NJ (US)

(21) Appl. No.: 15/510,047

(22) PCT Filed: Sep. 8, 2015
(86) PCT No.: PCT/US15/48927

§ 371 Date: Mar. 9, 2017

Related U.S. Application Data
(60) Provisional application No. 62/048,056, filed on Sep. 9, 2014.

(51) Int. Cl. A61K 31/519; A61K 31/4439; A61K 31/433; A61K 31/454; A61K 31/426

(52) U.S. Cl. CPC A61K 31/519; A61K 31/454; A61K 31/426; A61K 31/433; A61K 31/4439

(57) ABSTRACT

Provided herein are methods for treating or preventing prediabetes or diabetes, or maintaining or lowering blood or plasma glucose or maintaining or lowering blood or plasma glycated hemoglobin comprising administering to a subject in need thereof a therapeutically effective amount of a compound selectively inhibiting NaV1.7. In particular, provided herein are processes for the preparation of and intermediates used in the preparation of compounds selectively inhibiting NaV 1.7, such as the compounds of Formula I or compounds of Formula I′:

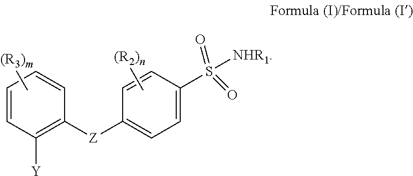

This application claims the benefit of U.S. provisional application No. 62/048,056 filed Sep. 9, 2014, which is incorporated by reference herein in its entirety.

### 1 FIELD

Provided herein are methods for treating or preventing prediabetes or diabetes, or maintaining or lowering blood or plasma glucose or maintaining or lowering blood or plasma glycated hemoglobin comprising administering to a subject in need thereof a therapeutically effective amount of a compound selectively inhibiting NaV1.7. In particular, provided herein are processes for the preparation of and intermediates used in the preparation of compounds selectively inhibiting NaV1.7.

### 2 BACKGROUND

Voltage-gated ion channels play a critical role in the electrical activity of neuronal and muscle cells. Large families of voltage-gated ion channels (e.g., sodium channels) have been identified. These ion channels have been the target of significant pharmacologic study, due to their potential role in a variety of pathological conditions.

Prediabetes and diabetes describe a group of metabolic diseases with high blood sugar levels over longer periods of time. Diabetes can result from insufficient production of the peptide hormone insulin. In other cases, diabetes can result from insulin resistance, i.e., an inability of cells to respond properly to insulin. If the blood sugar levels are higher than normal, but not high enough for a diagnosis of diabetes, the subject is prediabetic. There are three main types of diabetes: First, Type 1 results from the body's failure to produce sufficient levels of insulin. Second, Type 2 results from insulin resistance. Third, Gestational diabetes occurs when pregnant women without a previous history of diabetes develop a high blood glucose level. Another type of diabetes is latent autoimmune diabetes in adults (LADA). LADA is the most common term describing patients with a type 2 diabetic phenotype combined with islet antibodies and slowly progressive cell failure.

Type 2 diabetes, for example, is a serious and prevalent disease. Approximately 25.8 million people in the United States alone suffer from diabetes, whereby type 2 diabetes accounts for about 90-95% of all diagnosed diabetes cases. U.S. Patent Application Publication No. 2014/0228353 A1 at paragraph [0002]. The number of Americans with diabetes has more than tripled from 1980 to 2008. Id. Diabetes is also increasingly prevalent in other parts of the world, such as in certain Asian countries. Id. Rapid lifestyle and economic changes in, e.g., India and China, have led to a more sedentary lifestyle and poorer diet among the overall population, causing diabetes to become a major health concern. Id. There remains a continued need for novel and improved therapies that address this growing health concern.

### 3 SUMMARY

First, provided herein are methods for treating or preventing prediabetes comprising administering to a subject in need thereof a therapeutically effective amount of a compound selectively inhibiting NaV1.7.

Second, provided herein are methods for treating or preventing diabetes comprising administering to a subject in need thereof a therapeutically effective amount of a compound selectively inhibiting NaV1.7.

Third, provided herein are methods for maintaining or lowering blood or plasma glucose levels in a subject in need thereof comprising administering to the subject, a therapeutically effective amount of a compound selectively inhibiting NaV1.7.

Fourth, provided herein are methods for maintaining or lowering blood or plasma glycated hemoglobin levels in a subject in need thereof comprising administering to the subject, a therapeutically effective amount of a compound selectively inhibiting NaV1.7.

In one embodiment, provided herein are compounds of Formula (I′) for use in the methods disclosed:

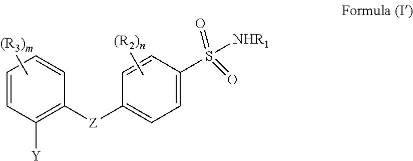

or a pharmaceutically acceptable salt, or a stereoisomeric or tautomeric form thereof,
wherein:
Z is —O— or —S—;
Y is —X—C(═O)NR₄R₅, —(CH₂)₃—NR₉R₁₀, or 4,5,6,7-tetrahydropyrazolo[1,5-a]pyrimidine-(2-yl or 3-yl);
X is (C₆-C₁₀)aryl or 5- or 6-membered heteroaryl;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
   R₄ and R₅ are not both H; and
   at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
      R₆ is (C₁-C₁₂)alkyl;
      R₇ and R₈ are each independently H, (C₁-C₁₂)alkyl, or R₇ and R₈ together form a 4- to 7-membered heterocycloalkyl ring;
R₉ is (C₁-C₆)alkyl, (C₃-C₈)cycloalkyl, pyrazolyl or pyridinyl; wherein R₉ is optionally further substituted with 1 or 2 substituents selected from the group consisting of —COOH, —COOR₁₁, —CONR₁₁R₁₂, —SO₂R₁₁, —SO₂NR₁₁R₁₂, —OH, —CN, —OR₁₁, and —NR₁₁R₁₂; wherein R₁₁ and R₁₂ may form a 6 membered heterocycloalkyl ring
R₁₀ is R₁₁, (C₃-C₆)alkynyl, (C₃-C₆)alkenyl, —COR₁₁, —COOR₁₁, —SO₂R₁₁, 5-methyl-2-oxo-1,3-dioxol-4-yl,

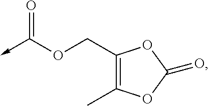

—COO—CH(CH₃)OCOCH(CH₃)₂; or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy; or R₉ and R₁₀ together form a unsubstituted 4- to 8-membered heterocycloalkyl ring, wherein said heterocycloalkyl ring is fused with a 5-membered heteroaryl; and
R₁₁ and R₁₂ are independently H or (C₁-C₆)alkyl, optionally substituted with 4- to 8-membered heterocycloalkyl ring; and
m and n are each independently 1, 2, 3, or 4.

In one embodiment, the compounds of Formula (I′) for use in the methods disclosed are those wherein Y is —(CH₂)₃—NR₉R₁₀.

In one embodiment, the compounds of Formula (I′) for use in the methods disclosed are those wherein R₁ is an aromatic 5- or 6-membered heterocycle, with 1-3 heteroatoms independently selected from the group consisting of N, O, and S. In one embodiment, the compounds of Formula (I′) for use in the methods disclosed are those wherein R₁ is pyridyl or pyrimidinyl. In one embodiment, the compounds of Formula (I′) for use in the methods disclosed are those wherein R₁ is an aromatic 5-membered heterocycle with 1 or 2 nitrogen atoms and optionally 1 or 2 sulphur atoms. In one embodiment, the compounds of Formula (I′) for use in the methods disclosed are those wherein R₁ is thiazolyl, isothiazolyl, or thiadiazolyl. In one embodiment, the compounds of Formula (I′) for use in the methods disclosed are those wherein R₁ is thiazolyl. In one embodiment, the compounds of Formula (I′) for use in the methods disclosed are those wherein R₁ is 1,2,4-thiadiazol-5-yl.

In one embodiment, the compounds of Formula (I′) for use in the methods disclosed are those wherein R₂ is independently at each occurrence —F or —Cl.

In one embodiment, the compounds of Formula (I′) for use in the methods disclosed are those wherein n is 1, 2, or 3. In one embodiment, the compounds of Formula (I′) for use in the methods disclosed are those wherein n is 2.

In one embodiment, the compounds of Formula (I′) for use in the methods disclosed are those wherein Z is —O.

In one embodiment, the compounds of Formula (I′) for use in the methods disclosed are those wherein R₃ is independently at each occurrence —H, —F, —Cl, or —Br. In one embodiment, the compounds of Formula (I′) for use in the methods disclosed are those wherein R₃ is —H or —Cl. In one embodiment, the compounds of Formula (I′) for use in the methods disclosed are those wherein R₃ is —Cl.

In one embodiment, the compounds of Formula (I′) for use in the methods disclosed are those wherein m is 1, 2, or 3. In one embodiment, the compounds of Formula (I′) for use in the methods disclosed are those wherein m is 1.

In one embodiment, the compounds of Formula (I′) for use in the methods disclosed are those wherein R₉ is (C₁-C₆)alkyl; wherein R₉ is optionally further substituted with 1 or 2 substituents selected from the group consisting of —COOH, —COOMe, —CONH₂, and —NH₂. In one embodiment, the compounds of Formula (I′) for use in the methods disclosed are those wherein R₉ is methyl or ethyl. In one embodiment, the compounds of Formula (I′) for use in the methods disclosed are those wherein R₉ is further substituted with —COOH.

In one embodiment, the compounds of Formula (I′) for use in the methods disclosed are those wherein R₁₀ is —H, —COMe, —COOEt. In one embodiment, the compounds of Formula (I′) for use in the methods disclosed are those wherein R₁₀ is —H or —COMe. In one embodiment, the compounds of Formula (I′) for use in the methods disclosed are those wherein R₁₀ is —H.

In one embodiment, the compounds of Formula (I′) for use in the methods disclosed are those R₁₀ is H and R₉ is (C₁-C₆)alkyl, wherein R₉ is further substituted with —COR₁₁R₁₂, and wherein R₁₁ and R₁₂ are independently H or (C₁-C₆)alkyl. In one embodiment, the compounds of Formula (I′) for use in the methods disclosed are those wherein the R₉ is methyl. In one embodiment, the compounds of Formula (I′) for use in the methods disclosed are those wherein the R₉ is further substituted with —CONH₂.

In one embodiment, the compounds of Formula (I′) for use in the methods disclosed are those wherein R₉ and R₁₀ together form a 4 to 8 membered heterocycloalkyl ring, wherein said heterocycloalkyl ring is substituted with 1 or 2 groups selected from the group consisting of —COOH, —COOMe, —COOEt, —CH₂—COOH, and —NH₂.

In one embodiment, the compounds of Formula (I′) for use in the methods disclosed are those wherein R₉ and R₁₀ together form a 4 to 8 membered heterocycloalkyl ring, wherein said heterocycloalkyl ring is substituted with 1 or 2 groups selected from the group consisting of —COOH, —CH₂—COOH, and —NH₂. In one embodiment, the compounds of Formula (I′) for use in the methods disclosed are those wherein R₉ and R₁₀ together form a piperidine substituted with 1 or 2 groups selected from the group consisting of —COOH, —COOMe, —COOEt, —CH₂—COOH, —CH₂—COOMe, —CH₂—COOEt, and —NH₂. In one embodiment, the compounds of Formula (I′) for use in the methods disclosed are those wherein R₉ and R₁₀ together form a piperidine substituted with 1 or 2 groups selected from the group consisting of —COOH, —CH₂—COOH, and —NH₂.

In one embodiment, the compounds of Formula (I′) for use in the methods disclosed are those wherein Y is —X—C(═O)NR₄R₅.

In one embodiment, the compounds of Formula (I′) for use in the methods disclosed are those wherein X is 5- or 6-membered heteroaryl. In one embodiment, the compounds of Formula (I′) for use in the methods disclosed are those wherein X is pyridyl or pyrimidinyl. In one embodiment, the compounds of Formula (I′) for use in the methods disclosed are those wherein X is pyridyl.

In one embodiment, the compounds of Formula (I′) for use in the methods disclosed are those wherein R₄ is H and R₅ is (C₁-C₉)alkyl. In one embodiment, the compounds of Formula (I′) for use in the methods disclosed are those wherein R₅ is methyl or ethyl, substituted with 1 or 2 substituents selected from the group consisting of —CO₂H, —CO₂R₆, and —CONR₇R₈. In one embodiment, the compounds of Formula (I′) for use in the methods disclosed are those wherein R₆ is (C₁-C₆)alkyl. In one embodiment, the compounds of Formula (I′) for use in the methods disclosed are those wherein R₅ is methyl or ethyl, substituted with —CO₂H.

In one embodiment, the compounds of Formula (I′) for use in the methods disclosed are those wherein Y is 4,5,6,7-tetrahydropyrazolo[1,5-a]pyrimidine-(2-yl or 3-yl). In one embodiment, the compounds of Formula (I′) for use in the methods disclosed are those wherein Y is 4,5,6,7-tetrahydropyrazolo[1,5-a]pyrimidine-3-yl.

In one embodiment, the compound for use in the methods disclosed is
3-(4-(2-(4-(N-(1,2,4-thiadiazol-5-yl)sulfamoyl)-2-chloro-5-fluorophenoxy)-5-chlorophenyl)picolinamido)propanoic acid,
2-(4-(2-(4-(N-(1,2,4-thiadiazol-5-yl)sulfamoyl)-2-chloro-5-fluorophenoxy)-5-chlorophenyl)picolinamido)acetic acid,
5-(4-(2-(4-(N-(1,2,4-thiadiazol-5-yl)sulfamoyl)-2-chloro-5-fluorophenoxy)-5-chlorophenyl)picolinamido)pentanoic acid,
4-(4-(2-(4-(N-(1,2,4-thiadiazol-5-yl)sulfamoyl)-2-chloro-5-fluorophenoxy)-5-chlorophenyl)picolinamido)butanoic acid,
2-(4-(2-(4-(N-(1,2,4-thiadiazol-5-yl)sulfamoyl)-2-chloro-5-fluorophenoxy)-5-chlorophenyl)picolinamido)propanoic acid,
(R)-2-(4-(2-(4-(N-(1,2,4-thiadiazol-5-yl)sulfamoyl)-2-chloro-5-fluorophenoxy)-5-chlorophenyl)picolinamido)propanoic acid,
2-(6-(2-(4-(N-(1,2,4-thiadiazol-5-yl)sulfamoyl)-2-chloro-5-fluorophenoxy)-5-chlorophenyl)picolinamido)acetic acid,
(S)-2-(4-(2-(4-(N-(1,2,4-thiadiazol-5-yl)sulfamoyl)-2-chloro-5-fluorophenoxy)-5-chlorophenyl)picolinamido)propanoic acid,
3-(4-(2-(4-(N-(1,2,4-thiadiazol-5-yl)sulfamoyl)-2-cyanophenoxy)-5-chlorophenyl)picolinamido)propanoic acid,
3-(4-(2-(4-(N-(1,2,4-thiadiazol-5-yl)sulfamoyl)-2,5-difluorophenoxy)-5-chlorophenyl)picolinamido)propanoic acid,
2-((3-(5-chloro-2-(2-chloro-5-fluoro-4-(N-(thiazol-4-yl)sulfamoyl)phenoxy)phenyl)propyl)amino)acetic acid,
3-((3-(2-(4-(N-(1,2,4-thiadiazol-5-yl)sulfamoyl)-2-chloro-5-fluorophenoxy)-5-chlorophenyl)propyl)amino)propanoic acid,
2-((3-(5-chloro-2-(2-chloro-5-fluoro-4-(N-(thiazol-2-yl)sulfamoyl)phenoxy)phenyl)propyl)amino)acetic acid,
1-(3-(5-chloro-2-(2-chloro-5-fluoro-4-(N-(thiazol-4-yl)sulfamoyl)phenoxy)phenyl)propyl)piperidine-4-carboxylic acid,
3-((3-(5-chloro-2-(2-chloro-5-fluoro-4-(N-(thiazol-4-yl)sulfamoyl)phenoxy)phenyl)propyl)amino)propanoic acid,
4-amino-1-(3-(5-chloro-2-(2-chloro-5-fluoro-4-(N-(thiazol-4-yl)sulfamoyl)phenoxy)phenyl)propyl)piperidine-4-carboxylic acid,
2-amino-4-((3-(5-chloro-2-(2-chloro-5-fluoro-4-(N-(thiazol-4-yl)sulfamoyl)phenoxy)phenyl)propyl)amino)butanoic acid,
2-((3-(5-chloro-2-(2,5-difluoro-4-(N-(thiazol-2-yl)sulfamoyl)phenoxy)phenyl)propyl)amino)acetic acid,
1-(3-(5-chloro-2-(2-chloro-5-fluoro-4-(N-(thiazol-4-yl)sulfamoyl)phenoxy)phenyl)propyl)piperidine-3-carboxylic acid,
2-((3-(2-(4-(N-(1,2,4-thiadiazol-5-yl)sulfamoyl)-2-chloro-5-fluorophenoxy)phenyl)propyl)amino)acetic acid,
2-((3-(5-chloro-2-(2,5-difluoro-4-(N-(thiazol-4-yl)sulfamoyl)phenoxy)phenyl)propyl)amino)acetic acid,
3-((3-(5-chloro-2-(2,5-difluoro-4-(N-(thiazol-4-yl)sulfamoyl)phenoxy)phenyl)propyl)amino)propanoic acid,
3-((3-(5-chloro-2-(2-cyano-4-(N-(thiazol-4-yl)sulfamoyl)phenoxy)phenyl)propyl)amino)propanoic acid,
methyl 2-((3-(5-chloro-2-(2-chloro-5-fluoro-4-(N-(thiazol-4-yl)sulfamoyl)phenoxy)phenyl)propyl)amino)acetate,
3-((3-(2-(2-chloro-5-fluoro-4-(N-(thiazol-4-yl)sulfamoyl)phenoxy)-5-fluorophenyl)propyl)amino)propanoic acid,
3-((3-(5-chloro-2-(2-chloro-5-fluoro-4-(N-(thiazol-4-yl)sulfamoyl)phenoxy)phenyl)propyl)amino)propanamide,
2-(N-(3-(5-chloro-2-(2-chloro-5-fluoro-4-(N-(thiazol-4-yl)sulfamoyl)phenoxy)phenyl)propyl)acetamido)acetic acid,
2-(1-(3-(2-(4-(N-(1,2,4-thiadiazol-5-yl)sulfamoyl)-2-chloro-5-fluorophenoxy)-5-chlorophenyl)propyl)piperidin-4-yl)acetic acid,
3-((3-(5-chloro-2-(2-chloro-5-fluoro-4-(N-(thiazol-2-yl)sulfamoyl)phenoxy)phenyl)propyl)amino)propanoic acid,
2-((3-(5-chloro-2-(2-chloro-5-fluoro-4-(N-(thiazol-4-yl)sulfamoyl)phenoxy)phenyl)propyl)amino)-N-methylacetamide,
5-chloro-4-(4-chloro-2-(3-((2-(methylsulfonyl)ethyl)amino)propyl)phenoxy)-2-fluoro-N-(thiazol-4-yl)benzenesulfonamide,
1-(3-(2-(4-(N-(1,2,4-thiadiazol-5-yl)sulfamoyl)-2-chloro-5-fluorophenoxy)-5-chlorophenyl)propyl)piperidine-4-carboxylic acid,
5-chloro-4-(4-chloro-2-(4,5,6,7-tetrahydropyrazolo[1,5-a]pyrimidin-3-yl)phenoxy)-2-fluoro-N-(thiazol-4-yl)benzenesulfonamide,
2-((3-(5-chloro-2-(2-chloro-5-fluoro-4-(N-(thiazol-2-yl)sulfamoyl)phenoxy)phenyl)propyl)(ethoxycarbonyl)amino)acetic acid,
ethyl 2-((3-(5-chloro-2-(2-chloro-5-fluoro-4-(N-(thiazol-2-yl)sulfamoyl)phenoxy)phenyl)propyl)amino)acetate,
4-(2-(3-((1H-pyrazol-4-yl)amino)propyl)-4-chlorophenoxy)-5-chloro-2-fluoro-N-(thiazol-2-yl)benzenesulfonamide,
3-((3-(5-chloro-2-(2,5-difluoro-4-(N-(thiazol-2-yl)sulfamoyl)phenoxy)phenyl)propyl)amino)propanoic acid,
5-chloro-4-(4-chloro-2-(3-((2-(methylsulfonyl)ethyl)amino)propyl)phenoxy)-2-fluoro-N-(thiazol-4-yl)benzenesulfonamide,
4-(2-(3-((1H-pyrazol-3-yl)amino)propyl)-4-chlorophenoxy)-5-chloro-2-fluoro-N-(thiazol-4-yl)benzenesulfonamide,
2-((3-(5-chloro-2-(2-chloro-5-fluoro-4-(N-(thiazol-4-yl)sulfamoyl)phenoxy)phenyl)propyl)amino)-N-methylacetamide,
2-((3-(5-chloro-2-(2-chloro-5-fluoro-4-(N-(thiazol-4-yl)sulfamoyl)phenoxy)phenyl)propyl)(methyl)amino)acetic acid,
5-chloro-4-(4-chloro-2-(3-(6,7-dihydro-1H-pyrazolo[4,3-c]pyridin-5(4H)-yl)propyl)phenoxy)-2-fluoro-N-(thiazol-4-yl)benzenesulfonamide,
2-((3-(5-chloro-2-(2-chloro-5-fluoro-4-(N-(thiazol-4-yl)sulfamoyl)phenoxy)phenyl)propyl)amino)acetamide,
isopentyl 2-((3-(5-chloro-2-(2-chloro-5-fluoro-4-(N-(thiazol-2-yl)sulfamoyl)phenoxy)phenyl)propyl)amino)acetate,
isopropyl 2-((3-(5-chloro-2-(2-chloro-5-fluoro-4-(N-(thiazol-2-yl)sulfamoyl)phenoxy)phenyl)propyl)amino)acetate,
methyl 2-((3-(5-chloro-2-(2-chloro-5-fluoro-4-(N-(thiazol-4-yl)sulfamoyl)phenoxy)phenyl)propyl)(methyl)amino)acetate,
2-((3-(5-chloro-2-(2-chloro-5-fluoro-4-(N-(thiazol-2-yl)sulfamoyl)phenoxy)phenyl)propyl)((pentyloxy)carbonyl)amino)acetic acid,
2-((3-(5-chloro-2-(2-chloro-5-fluoro-4-(N-(thiazol-2-yl)sulfamoyl)phenoxy)phenyl)propyl)(prop-2-yn-1-yl)amino)acetic acid,
5-chloro-4-(4-chloro-2-(3-(5,6-dihydroimidazo[1,2-a]pyrazin-7(8H)-yl)propyl)phenoxy)-2-fluoro-N-(thiazol-4-yl)benzenesulfonamide,
5-chloro-2-fluoro-4-(2-(4,5,6,7-tetrahydropyrazolo[1,5-a]pyrimidin-3-yl)phenoxy)-N-(thiazol-2-yl)benzenesulfonamide,
5-chloro-4-(4-chloro-2-(4,5,6,7-tetrahydropyrazolo[1,5-a]pyrimidin-3-yl)phenoxy)-2-fluoro-N-(thiazol-2-yl)benzenesulfonamide,
5-chloro-2-fluoro-4-(2-(4,5,6,7-tetrahydropyrazolo[1,5-a]pyrimidin-3-yl)phenoxy)-N-(thiazol-4-yl)benzenesulfonamide,
5-chloro-4-(4-chloro-2-(3-((2-(methylsulfonyl)ethyl)amino)propyl)phenoxy)-2-fluoro-N-(thiazol-2-yl)benzenesulfonamide,
2-((3-(2-(2-chloro-5-fluoro-4-(N-(thiazol-4-yl)sulfamoyl)phenoxy)phenyl)propyl)amino)acetamide,
2-((3-(5-chloro-2-(2-chloro-5-fluoro-4-(N-(thiazol-4-yl)sulfamoyl)phenoxy)phenyl)propyl)(prop-2-yn-1-yl)amino)acetic acid,
2-(allyl(3-(5-chloro-2-(2-chloro-5-fluoro-4-(N-(thiazol-2-yl)sulfamoyl)phenoxy)phenyl)propyl)amino)acetic acid,
2-((3-(5-chloro-2-(2-chloro-5-fluoro-4-(N-(thiazol-2-yl)sulfamoyl)phenoxy)phenyl)propyl)amino)acetamide,
2-(but-2-yn-1-yl(3-(5-chloro-2-(2-chloro-5-fluoro-4-(N-(thiazol-2-yl)sulfamoyl)phenoxy)phenyl)propyl)amino)acetic acid,
2-((3-(5-chloro-2-(2-chloro-5-fluoro-4-(N-(thiazol-2-yl)sulfamoyl)phenoxy)phenyl)propyl)(propyl)amino)acetic acid,
3-((3-(5-chloro-2-(2-chloro-5-fluoro-4-(N-(thiazol-2-yl)sulfamoyl)phenoxy)phenyl)propyl)(prop-2-yn-1-yl)amino)propanoic acid,
2-((3-(5-chloro-2-(2,5-difluoro-4-(N-(thiazol-2-yl)sulfamoyl)phenoxy)phenyl)propyl)(prop-2-yn-1-yl)amino)acetic acid,
ethyl 2-((3-(5-chloro-2-(2-chloro-5-fluoro-4-(N-(thiazol-2-yl)sulfamoyl)phenoxy)phenyl)propyl)(methyl)amino)acetate, or
2-((3-(5-chloro-2-(2,5-difluoro-4-(N-(thiazol-4-yl)sulfamoyl)phenoxy)phenyl)propyl)amino)acetamide;
   or a pharmaceutically acceptable salt, or a stereoisomeric or tautomeric form thereof

In one embodiment, the compound for use in the methods disclosed is
2-(4-(2-(4-(N-(1,2,4-thiadiazol-5-yl)sulfamoyl)-2-chloro-5-fluorophenoxy)-5-chlorophenyl)picolinamido)acetic acid,
3-(4-(2-(4-(N-(1,2,4-thiadiazol-5-yl)sulfamoyl)-2-chloro-5-fluorophenoxy)-5-chlorophenyl)picolinamido)propanoic acid,
2-(4-(2-(4-(N-(1,2,4-thiadiazol-5-yl)sulfamoyl)-2-chloro-5-fluorophenoxy)-5-chlorophenyl)picolinamido)propanoic acid,
3-((3-(2-(4-(N-(1,2,4-thiadiazol-5-yl)sulfamoyl)-2-chloro-5-fluorophenoxy)-5-chlorophenyl)propyl)amino)propanoic acid,
2-((3-(5-chloro-2-(2-chloro-5-fluoro-4-(N-(thiazol-4-yl)sulfamoyl)phenoxy)phenyl)propyl)amino)acetamide,
2-((3-(5-chloro-2-(2-chloro-5-fluoro-4-(N-(thiazol-2-yl)sulfamoyl)phenoxy)phenyl)propyl)((pentyloxy)carbonyl)amino)acetic acid, or
2-((3-(5-chloro-2-(2-chloro-5-fluoro-4-(N-(thiazol-2-yl)sulfamoyl)phenoxy)phenyl)propyl)(prop-2-yn-1-yl)amino)acetic acid;
   or a pharmaceutically acceptable salt, or a stereoisomeric or tautomeric form thereof.

In one embodiment, the compound for use in the methods disclosed is
3-((3-(5-chloro-2-(2-chloro-5-fluoro-4-(N-(thiazol-4-yl)sulfamoyl)phenoxy)phenyl)propyl)amino)propanoic acid,
2-((3-(5-chloro-2-(2-chloro-5-fluoro-4-(N-(thiazol-4-yl)sulfamoyl)phenoxy)phenyl)propyl)amino)acetamide,
2-((3-(5-chloro-2-(2-chloro-5-fluoro-4-(N-(thiazol-2-yl)sulfamoyl)phenoxy)phenyl)propyl)(prop-2-yn-1-yl)amino)acetic acid,
2-((3-(5-chloro-2-(2-chloro-5-fluoro-4-(N-(thiazol-4-yl)sulfamoyl)phenoxy)phenyl)propyl)(prop-2-yn-1-yl)amino)acetic acid,
2-((3-(5-chloro-2-(2-chloro-5-fluoro-4-(N-(thiazol-2-yl)sulfamoyl)phenoxy)phenyl)propyl)amino)acetamide,
2-((3-(5-chloro-2-(2-chloro-5-fluoro-4-(N-(thiazol-2-yl)sulfamoyl)phenoxy)phenyl)propyl)(propyl)amino)acetic acid,
2-((3-(5-chloro-2-(2,5-difluoro-4-(N-(thiazol-2-yl)sulfamoyl)phenoxy)phenyl)propyl)(prop-2-yn-1-yl)amino)acetic acid, or
2-((3-(5-chloro-2-(2,5-difluoro-4-(N-(thiazol-4-yl)sulfamoyl)phenoxy)phenyl)propyl)amino)acetamide;
   or a pharmaceutically acceptable salt, or a stereoisomeric or tautomeric form thereof.

In one embodiment, the subject has prediabetes. In one embodiment, the subject has diabetes. In one embodiment, diabetes is gestational diabetes, type-1 diabetes, type-2 diabetes, or latent autoimmune diabetes of adults. In one embodiment, type-2 diabetes is hyperinsulinemic Type 2 diabetes.

In one embodiment, the prediabetes or diabetes is caused by or accompanied by obesity. In one embodiment, the patient has not been previously treated for prediabetes.

In one embodiment, the compounds of Formula (I′) for use in the methods disclosed are those wherein the compound has an IC50 for NaV1.1, NaV1.2, NaV1.3, NaV 1.4, NaV1.5, NaV 1.6, NaV 1.8, and NaV1.9, that is each independently at least 10 fold, 20 fold, 50 fold, 100 fold, 200 fold, 500 fold, 1000 fold, 2000 fold, 5000 fold, or 10000 fold higher than the NaV 1.7 IC50 for said compound. In one embodiment, the compounds of Formula (I′) for use in the methods disclosed are those wherein the compound has a NaV1.3 IC50 of at least at least 10 fold, 20 fold, 50 fold, 100 fold, 200 fold, 500 fold, 1000 fold, 2000 fold, 5000 fold, or 10000 fold higher than the NaV 1.7 IC50 for said compound. In one embodiment, the IC50 is measured using an FDSS membrane potential assay or the patch-clamp method.

### 4 DETAILED DESCRIPTION

### 4.1 Definitions

A “Compound” or “Compounds” as used herein comprise a compound of Formula (I), a compound of Formula (I′), a compound of Formula (Ia), a compound of Formula (I′a), a compound of Formula (Ib), a compound of Formula (Ic), a compound of Formula (Id), a compound listed in Table 1, a compound listed in Table 2, a compound listed in Table 3, or a compound described in Section 4.3.

A “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the Compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Others are well known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

A “stereoisomer” or “stereoisomeric form” refers to one stereoisomer of a Compound that is substantially free of other stereoisomers of that Compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Compounds can have chiral centers and can occur as racemates, individual enantiomers or diastereomers, and mixtures thereof. All such isomeric forms are included within the embodiments disclosed herein, including mixtures thereof. The use of stereomerically pure forms of such Compounds, as well as the use of mixtures of those forms, are encompassed by the embodiments disclosed herein. For example, mixtures comprising equal or unequal amounts of the enantiomers of a particular Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N.Y., 1962); and Wilen, S. H., Tables of Resolving Agents and Optical Resolutions p. 268 (E. L. Eliel, Ed., Univ. of Notre Dame Press, Notre Dame, Ind., 1972).

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

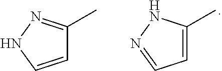

As readily understood by one skilled in the art, a wide variety of functional groups and other structures may exhibit tautomerism and all tautomers of the Compounds provided herein are within the scope of the present disclosure.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include, but are not limited to, phenyl, naphthyl and the like.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Examples include, but are not limited to, groups such as pyrrolyl, pyrazolyl, imidazolyl, triazolyl, tetrazolyl, oxazolyl, isoxazolyl, thiazolyl, isothiazolyl, thiadiazolyl (e.g., 1,2,4-thiadiazolyl), pyrrolyl, pyridyl, pyridazinyl, pyrimidinyl, pyrazinyl, thiophenyl, benzothiophenyl, furanyl, benzofuranyl, indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl, pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “partially unsaturated or aromatic heterocycle” is a partially unsaturated or aromatic ring system having one to four heteroatoms as ring atoms in a heteroaromatic ring system, wherein the remainder of the atoms are carbon atoms. If the “partially unsaturated or aromatic heterocycle” is an aromatic heterocycle, then the aromatic heterocycle is a “heteroaryl” as defined above. In one embodiment, the partially unsaturated or aromatic heterocycle is a partially unsaturated or aromatic 5- or 6-membered heterocycle. Examples of partially unsaturated heterocycles include, but are not limited to, groups such as 2,5-dihydro-1H-pyrrolyl, 2,5-dihydrofuranyl, 2,5-dihydrothiophenyl, 4,5-dihydrooxazolyl, 4,5-dihydrothiazolyl, 4,5-dihydro-1H-imidazolyl, 4,5-dihydro-1H-1,2,3-triazolyl, 1,2,5,6-tetrahydropyridinyl, and 1,4,5,6-tetrahydropyrimidinyl groups.

A “heterocycloalkyl” group is a non-aromatic cycloalkyl in which one to four of the ring carbon atoms are independently replaced with a heteroatom from the group consisting of O, S and N. Examples of a heterocycloalkyl group include, but are not limited to, morpholinyl, pyrrolidinyl, piperazinyl, (1,4)-dioxanyl, and (1,3)-dioxolanyl. Heterocycloalkyls can also be bonded at any ring atom (i.e., at any carbon atom or heteroatom of the heterocyclic ring). In one embodiment, the heterocycloalkyl is a 5- or 6-membered or 4- to 8-membered heterocycloalkyl.

An “alkyl” group is a saturated straight chain or branched non-cyclic hydrocarbon having, for example, from 1 to 12 carbon atoms, 1 to 9 carbon atoms, 1 to 6 carbon atoms, 1 to 4 carbon atoms, or 2 to 6 carbon atoms. Representative alkyl groups include -methyl, -ethyl, -n-propyl, -n-butyl, -n-pentyl and -n-hexyl; while branched alkyls include -isopropyl, -sec-butyl, -iso-butyl, -tert-butyl, -iso-pentyl, 2-methylpentyl, 3-methylpentyl, 4-methylpentyl, 2,3-dimethylbutyl and the like.

An “alkenyl” group is a partially unsaturated straight chain or branched non-cyclic hydrocarbon having, for example, from 3 to 6 carbon atoms, 3 to 4 carbon atoms, or 3 carbon atoms. Representative alkenyl groups include allyl, propenyl and the like.

An “alkynyl” group is a partially unsaturated straight chain or branched non-cyclic hydrocarbon having, for example, from 3 to 6 carbon atoms, 4 to 6 carbon atoms, or 3 carbon atoms. Representative alkynyl groups include propynyl, butynyl and the like.

A “cycloalkyl” group is a saturated cyclic alkyl group of from 3 to 12 carbon atoms having a single cyclic ring or multiple condensed or bridged rings. In some embodiments, the cycloalkyl group has 4 to 12 ring members, whereas in other embodiments the number of ring carbon atoms ranges, for example, from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, and the like, or multiple or bridged ring structures such as adamantyl and the like.

A “subject in need thereof” refers to a mammal (e.g., human, dog, horse, or cat) in need of treatment with any method provided herein. In one embodiment the subject is a patient.

An “adult” refers to a human over the age of 30.

### 4.2 Brief Description of the Drawings

FIG. 1 shows the change in food intake measured in the rat streptozotocin-induced model for diabetes. The change in food intake is shown for a diabetic vehicle group, a diabetic test compound treatment group, and a sham group. The diabetic test compound treatment group received a 60 mg/kg/day dose of compound 49 for 9 days. The beginning and the end of the treatment period is marked by a dotted line.

FIG. 2 shows the change in glucose level measured in the rat streptozotocin-induced model for diabetes. The change in glucose levels is shown for a diabetic vehicle group, a diabetic test compound treatment group, and a sham group. The diabetic test compound treatment group received a 60 mg/kg/day dose of compound 49 for 9 days. The beginning and the end of the treatment period is marked by a dotted line.

FIG. 3 shows the change in water intake measured in the rat streptozotocin-induced model for diabetes. The change in water intake is shown for a diabetic vehicle group, a diabetic test compound treatment group, and a sham group. The diabetic test compound treatment group received a 60 mg/kg/day dose of compound 49 for 9 days. The beginning and the end of the treatment period is marked by a dotted line.

### 4.3 Compounds

In one embodiment, the compounds provided herein are selective inhibitors of NaV 1.7. In a specific embodiment, the compound provided herein has an IC50 for NaV1.1, NaV1.2, NaV1.3, NaV 1.4, NaV1.5, NaV 1.6, NaV 1.8, and NaV1.9, that is each independently at least 10 fold, 20 fold, 50 fold, 100 fold, 200 fold, 500 fold, 1000 fold, 2000 fold, 5000 fold, or 10000 fold higher than the NaV 1.7 IC50 for said compound. In a particular embodiment, the IC50 at a given sodium channel is measured using an FDSS membrane potential assay or the patch-clamp method or any other method known in the art, such as the methods described in WO2007/109324 to Fraser et al.

In one embodiment, the compounds provided herein are any of the compounds disclosed or discussed in Bagal et al., 2014, “Recent progress in sodium channel modulators for pain,” Bioorganic & Medicinal Chemistry Letters 24(16), Pages 3690-3699.

In one embodiment, the compounds provided herein are aryloxysulfonamides, sulfonated amines, aryloxysulfonylated amides, acylsulfonyl ureas, arylindazole sulfonylated amides, bicyclic core sulfonamides, substituted piperazine or piperazine methylenoxy arylsulfonamides, benzo-oxazolone core sulfonamides, cycloalkyloxyaryl-sulfonamides, aryloxybiaryls, biaryls, cyclopropyl-spiro-piperidines, pyridinyl morpholinones, or oxazolotriazoles, heteroarylamides, or pyrrolopyridinones, biaryl spiro-pyrrolidine-lactams, or spiro-piperidines.

In one embodiment, the compounds provided herein are aryloxysulfonamides or sulfonated amines. In a specific embodiment, the compounds provided herein are, for example, those disclosed in US2013/0005706 to Corkey et al., WO2013/114250 to Bagal et al., and WO2012/007868 to Brown et al.

In one embodiment, the compounds provided herein are aryloxysulfonylated amides, acylsulfonyl ureas, or arylindazole sulfonylated amides. In a specific embodiment, the compounds provided herein are, for example, those disclosed in WO2013/093688 to Storer et al., WO2013/088315 to Rawson et al., WO2012/095781 to Bell et al., WO2014008458 to Dehnhardt et al., WO2013177224 to Andrez et al.

In one embodiment, the compounds provided herein are bicyclic core sulfonamides. In a specific embodiment, the compounds provided herein are, for example, those disclosed in WO2013/025883 to Dineen et al., WO2013/086229 to Boezio et al., WO2013/122897 to Boezio et al., WO2013/134518 to Dineen et al., WO2014/066490 to Pero et al., WO2014066491 to Pero et al., and WO2014/061970 to Kim et al.

In one embodiment, the compounds provided herein are substituted piperazine or piperazine methylenoxy arylsulfonamides or aryloxysulfonamides. In a specific embodiment, the compounds provided herein are, for example, those disclosed in WO2013/064983 to Sun et al. and WO2013/064984 to Liu et al.

In one embodiment, the compounds provided herein are benzo-oxazolone core sulfonamides. In a specific embodiment, the compounds provided herein are, for example, those disclosed in WO2013/063459 to Layton et al.

In one embodiment, the compounds provided herein are cycloalkyloxyaryl-sulfonamides. In a specific embodiment, the compounds provided herein are, for example, those disclosed in WO2013/118854 to Shinozuka et al.

In one embodiment, the compounds provided herein are aryloxybiaryls. In a specific embodiment, the compounds provided herein are, for example, those disclosed in WO2013/136170 to Tafesse et al., WO2013/072758 to Shao, WO2013064884 to Engel et al., WO2013/064884 to Yao, WO2013/064883 to Yao, WO2013030665 to Ni et al., and WO2012085650 to Ni et al.

In one embodiment, the compounds provided herein are biaryls, cyclopropyl-spiro-piperidines, pyridinyl morpholinones, or oxazolotriazoles. In a specific embodiment, the compounds provided herein are, for example, those disclosed in WO2013/131018 to Pajouhesh et al., WO2012/047703 to Ho et al., WO2013/161929 to Hattori et al., and WO2013/161928 to Hattori et al.

In one embodiment, the compounds provided herein are heteroarylamides or pyrrolopyridinones. In a specific embodiment, the compounds provided herein are, for example, those disclosed in WO2012/053186 to Yamagishi et al., WO2013/161312 to Kawamura et al., and WO2013/161308 to Yamagishi et al.

In one embodiment, the compounds provided herein are biaryl Spiro-pyrrolidine-lactams. In a specific embodiment, the compounds provided herein are, for example, those disclosed in WO2013179049 to Giblin et al., WO2013175206 to Giblin et al., WO2013175205 to Giblin et al., WO2013093496 to Witty et al., and WO2013093497 to Witty et al.

In one embodiment, the compounds provided herein are spiro-piperidines. In a specific embodiment, the compounds provided herein are, for example, those disclosed in US20120196869 to Hadida-Ruah et al., WO2014022639 to Littler et al., WO2012125613 to Hadida-Ruah et al., WO2013109521 to Hadida-Ruah et al.

In one embodiment the compounds provided herein are AZD3161, PF-04856264, CNV1014802, DSP-2230, PF-05089771, XEN402, and XEN403.

Provided herein are compounds of Formula (I),

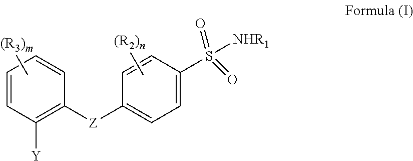

or a pharmaceutically acceptable salt, or a stereoisomeric or tautomeric form thereof, wherein:
Z is —O— or —S—;
Y is —X—C(═O)NR₄R₅, —(CH₂)₃—NR₉R₁₀, or 4,5,6,7-tetrahydropyrazolo[1,5-a]pyrimidine-(2-yl or 3-yl);
X is (C₆-C₁₀)aryl or 5- or 6-membered heteroaryl;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
   R₄ and R₅ are not both H; and
   at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
      R₆ is (C₁-C₁₂)alkyl;
      R₇ and R₈ are each independently H, (C₁-C₁₂)alkyl, or R₇ and R₈ together form a 4- to 7-membered heterocycloalkyl ring;
R₉ is (C₁-C₆)alkyl, (C₃-C₈)cycloalkyl, pyrazolyl or pyridinyl; wherein R₉ is optionally further substituted with 1 or 2 substituents selected from the group consisting of —COOH, —COOR₁₁, —CONR₁₁R₁₂, —SO₂R₁₁, —SO₂NR₁₁R₁₂, —OH, —CN, —OR₁₁, and —NR₁₁R₁₂; wherein R₁₁ and R₁₂ may form a 6 membered heterocycloalkyl ring
R₁₀ is R₁₁, —COR₁₁, —COOR₁₁, —SO₂R₁₁, 5-methyl-2-oxo-1,3-dioxol-4-yl,

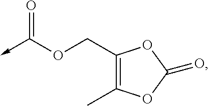

—COO—CH(CH₃)OCOCH(CH₃)₂; or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy;
   R₁₁ and R₁₂ are independently H or (C₁-C₆)alkyl, optionally substituted with 4- to 8-membered heterocycloalkyl ring; and
   m and n are each independently 1, 2, 3, or 4.

In a certain embodiment, the compounds of Formula (I′)

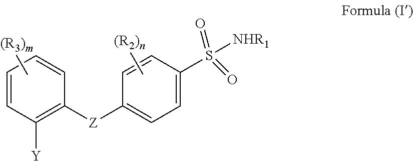

are those wherein
R₁₀ is R₁₁, (C₃-C₆)alkynyl, (C₃-C₆)alkenyl, —COR₁₁, —COOR₁₁, —SO₂R₁₁, 5-methyl-2-oxo-1,3-dioxol-4-yl,

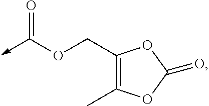

—COO—CH(CH₃)OCOCH(CH₃)₂; or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy; or R₉ and R₁₀ together form a unsubstituted 4- to 8-membered heterocycloalkyl ring, wherein said heterocycloalkyl ring is fused with a 5-membered heteroaryl; and
   wherein all other substituents are defined as in paragraph [0051] above.

In a certain embodiment, the compounds of Formula (I) or Formula (I′) are those wherein Y is —(CH₂)₃—NR₉R₁₀.

In a particular embodiment, the compounds of Formula (I) or Formula (I′) are those wherein R₁ is an aromatic 5- or 6-membered heterocycle, with 1-3 heteroatoms independently selected from the group consisting of N, O, and S.

In a particular embodiment, the compounds of Formula (I) or Formula (I′) are those wherein R₁ is pyridyl or pyrimidinyl.

In a particular embodiment, the compounds of Formula (I) or Formula (I′) are those wherein R₁ is an aromatic 5-membered heterocycle with 1 or 2 nitrogen atoms and optionally 1 or 2 sulphur atoms. In a particular embodiment, the compounds of Formula (I) or Formula (I′) are those wherein R₁ is thiazolyl, isothiazolyl, or thiadiazolyl. In a particular embodiment, the compounds of Formula (I) or Formula (I′) are those wherein R₁ is thiazolyl. In a particular embodiment, the compounds of Formula (I) or Formula (I′) are those wherein R₁ is 1,2,4-thiadiazol-5-yl. In a particular embodiment, the compounds of Formula (I) or Formula (I′) are those wherein R₁ is thiadiazol-4-yl.

In a particular embodiment, the compounds of Formula (I) or Formula (I′) are those wherein R₂ is independently at each occurrence —F or —Cl.

In a particular embodiment, the compounds of Formula (I) or Formula (I′) are those wherein n is 1, 2, or 3. In a particular embodiment, the compounds of Formula (I) or Formula (I′) are those wherein n is 2.

In a particular embodiment, the compounds of Formula (I) or Formula (I′) are those wherein Z is —O—.

In a particular embodiment, the compounds of Formula (I) or Formula (I′) are those wherein R₃ is independently at each occurrence —H, —F, —Cl, or —Br. In a particular embodiment, the compounds of Formula (I) or Formula (I′) are those wherein R₃ is —H or —Cl. In a particular embodiment, the compounds of Formula (I) or Formula (I′) are those wherein R₃ is —Cl.

In a particular embodiment, the compounds of Formula (I) or Formula (I′) are those wherein m is 1, 2, or 3. In a particular embodiment, the compounds of Formula (I) or Formula (I′) are those wherein m is 1.

In a particular embodiment, the compounds of Formula (I) or Formula (I′) are those wherein R₉ is (C₁-C₆)alkyl; wherein R₉ is optionally further substituted with 1 or 2 substituents selected from the group consisting of —COOH, —COOMe, —CONH₂, and —NH₂. In a particular embodiment, the compounds of Formula (I) or Formula (I′) are those wherein R₉ is methyl or ethyl. In a particular embodiment, the compounds of Formula (I) or Formula (I′) are those wherein R₉ is further substituted with —COOH.

In a particular embodiment, the compounds of Formula (I) or Formula (I′) are those wherein R₁₀ is H and R₉ is (C₁-C₆)alkyl; wherein R₉ is further substituted with —CONR₁₁R₁₂, and wherein R₁₁ and R₁₂ are independently H or (C₁-C₆)alkyl. In a particular embodiment, the compounds of Formula (I) or Formula (I′) are those wherein R₉ is further substituted with —CONH₂. In a particular embodiment, the compounds of Formula (I) or Formula (I′) are those wherein R₉ is methyl and wherein R₉ is further substituted with —CONH₂.

In a particular embodiment, the compounds of Formula (I) or Formula (I′) are those wherein R₁₀ is —H, —COMe, —COOEt. In a particular embodiment, the compounds of Formula (I) or Formula (I′) are those wherein R₁₀ is —H or —COMe. In a particular embodiment, the compounds of Formula (I) or Formula (I′) are those wherein R₁₀ is —H.

In a particular embodiment, the compounds of Formula (I) or Formula (I′) are those wherein R₉ and R₁₀ together form a 4 to 8 membered heterocycloalkyl ring, wherein said heterocycloalkyl ring is substituted with 1 or 2 groups selected from the group consisting of —COOH, —COOMe, —COOEt, —CH₂—COOH, and —NH₂. In a particular embodiment, the compounds of Formula (I) are those wherein R₉ and R₁₀ together form a 4 to 8 membered heterocycloalkyl ring, wherein said heterocycloalkyl ring is substituted with 1 or 2 groups selected from the group consisting of —COOH, —CH₂—COOH, and —NH₂.

In a particular embodiment, the compounds of Formula (I) or Formula (I′) are those wherein R₉ and R₁₀ together form a piperidine substituted with 1 or 2 groups selected from the group consisting of —COOH, —COOMe, —COOEt, —CH₂—COOH, —CH₂—COOMe, —CH₂—COOEt, and —NH₂. In a particular embodiment, the compounds of Formula (I) or Formula (I′) are those wherein R₉ and R₁₀ together form a piperidine substituted with 1 or 2 groups selected from the group consisting of —COOH, —CH₂—COOH, and —NH₂.

In a certain embodiment, the compounds of Formula (I) or Formula (I′) are those wherein Y is —X—C(═O)NR₄R₅.

In a particular embodiment, the compounds of Formula (I) or Formula (I′) are those wherein R₁ is an aromatic 5- or 6-membered heterocycle, with 1-3 heteroatoms independently selected from the group consisting of N, O, and S.

In a particular embodiment, the compounds of Formula (I) or Formula (I′) are those wherein R₁ is pyridyl or pyrimidinyl.

In a particular embodiment, the compounds of Formula (I) or Formula (I′) are those wherein R₁ is an aromatic 5-membered heterocycle with 1 or 2 nitrogen atoms and optionally 1 or 2 sulphur atoms. In a particular embodiment, the compounds of Formula (I) or Formula (I′) are those wherein R₁ is thiazolyl, isothiazolyl, or thiadiazolyl. In a particular embodiment, the compounds of Formula (I) or Formula (I′) are those wherein R₁ is thiazolyl. In a particular embodiment, the compounds of Formula (I) or Formula (I′) are those wherein R₁ is 1,2,4-thiadiazol-5-yl.

In a particular embodiment, the compounds of Formula (I) or Formula (I′) are those wherein R₂ is independently at each occurrence —F or —Cl.

In a particular embodiment, the compounds of Formula (I) or Formula (I′) are those wherein n is 1, 2, or 3. In a particular embodiment, the compounds of Formula (I) or Formula (I′) are those wherein n is 2.

In a particular embodiment, the compounds of Formula (I) or Formula (I′) are those wherein Z is —O—.

In a particular embodiment, the compounds of Formula (I) or Formula (I′) are those wherein R₃ is independently at each occurrence —H, —F, —Cl, or —Br. In a particular embodiment, the compounds of Formula (I) or Formula (I′) are those wherein R₃ is —H or —Cl. In a particular embodiment, the compounds of Formula (I) or Formula (I′) are those wherein R₃ is —Cl.

In a particular embodiment, the compounds of Formula (I) or Formula (I′) are those wherein m is 1, 2, or 3. In a particular embodiment, the compounds of Formula (I) or Formula (I′) are those wherein m is 1.

In a particular embodiment, the compounds of Formula (I) or Formula (I′) are those wherein X is 5- or 6-membered heteroaryl. In a particular embodiment, the compounds of Formula (I) or Formula (I′) are those wherein X is pyridyl or pyrimidinyl. In a particular embodiment, the compounds of Formula (I) or Formula (I′) are those wherein X is pyridyl.

In a particular embodiment, the compounds of Formula (I) or Formula (I′) are those wherein R₄ is H and R₅ is (C₁-C₉)alkyl.

In a particular embodiment, the compounds of Formula (I) or Formula (I′) are those wherein R₅ is methyl or ethyl, substituted with 1 or 2 substituents selected from the group consisting of —CO₂H, —CO₂R₆, and —CONR₇R₈.

In a particular embodiment, the compounds of Formula (I) or Formula (I′) are those wherein R₆ is (C₁-C₆)alkyl.

In a particular embodiment, the compounds of Formula (I) or Formula (I′) are those wherein R₅ is methyl or ethyl, substituted with —CO₂H.

In a certain embodiment, the compounds of Formula (I) or Formula (I′) are those wherein Y is 4,5,6,7-tetrahydropyrazolo[1,5-a]pyrimidine-(2-yl or 3-yl). In a particular embodiment, the compounds of Formula (I) or Formula (I′) are those wherein Y is 4,5,6,7-tetrahydropyrazolo[1,5-a]pyrimidine-3-yl.

In a particular embodiment, the compounds of Formula (I) or Formula (I′) are those wherein R₁ is an aromatic 5- or 6-membered heterocycle, with 1-3 heteroatoms independently selected from the group consisting of N, O, and S.

In a particular embodiment, the compounds of Formula (I) or Formula (I′) are those wherein R₁ is pyridyl or pyrimidinyl.

In a particular embodiment, the compounds of Formula (I) or Formula (I′) are those wherein R₁ is an aromatic 5-membered heterocycle with 1 or 2 nitrogen atoms and optionally 1 or 2 sulphur atoms. In a particular embodiment, the compounds of Formula (I) or Formula (I′) are those wherein R₁ is thiazolyl, isothiazolyl, or thiadiazolyl. In a particular embodiment, the compounds of Formula (I) or Formula (I′) are those wherein R₁ is thiazolyl. In a particular embodiment, the compounds of Formula (I) or Formula (I′) are those wherein R₁ is 1,2,4-thiadiazol-5-yl.

In a particular embodiment, the compounds of Formula (I) or Formula (I′) are those wherein R₂ is independently at each occurrence —F or —Cl.

In a particular embodiment, the compounds of Formula (I) or Formula (I′) are those wherein n is 1, 2, or 3. In a particular embodiment, the compounds of Formula (I) or Formula (I′) are those wherein n is 2.

In a particular embodiment, the compounds of Formula (I) or Formula (I′) are those wherein Z is —O—.

In a particular embodiment, the compounds of Formula (I) or Formula (I′) are those wherein R₃ is independently at each occurrence —H, —F, —Cl, or —Br. In a particular embodiment, the compounds of Formula (I) or Formula (I′) are those wherein R₃ is —H or —Cl. In a particular embodiment, the compounds of Formula (I) or Formula (I′) are those wherein R₃ is —Cl.

In a particular embodiment, the compounds of Formula (I) or Formula (I′) are those wherein m is 1, 2, or 3. In a particular embodiment, the compounds of Formula (I) or Formula (I′) are those wherein m is 1.

In a certain embodiment, the compounds of Formula (I) or Formula (I′) are those wherein the compound is selected from the group consisting of the compounds in Table 1 or a pharmaceutically acceptable salt, or a stereoisomeric or tautomeric form thereof.

TABLE 1

| Compound | Compound structure | Chemical name* |
|---|---|---|
| 1 | 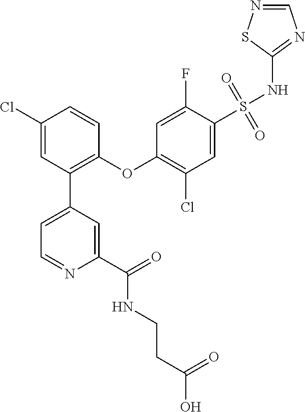 [SMILES: O=C(O)CCNC(=O)c1cc(-c2cc(Cl)ccc2Oc2cc(F)c(S(=O)(=O)Nc3ncns3)cc2Cl)ccn1] | 3-(4-(2-(4-(N-(1,2,4-thiadiazol-5-yl) sulfamoyl)-2-chloro-5-fluorophenoxy)-5- chlorophenyl)picolinamido)propanoic acid |
| 2 | 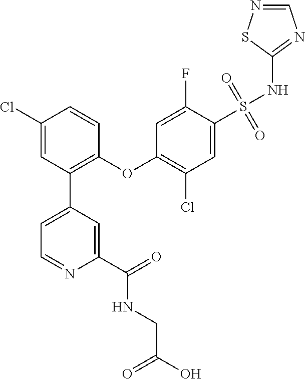 [SMILES: O=C(O)CNC(=O)c1cc(-c2cc(Cl)ccc2Oc2cc(F)c(S(=O)(=O)Nc3ncns3)cc2Cl)ccn1] | 2-(4-(2-(4-(N-(1,2,4-thiadiazol-5-yl) sulfamoyl)-2-chloro-5-fluorophenoxy)-5- chlorophenyl)picolinamido)acetic acid |
| 3 | 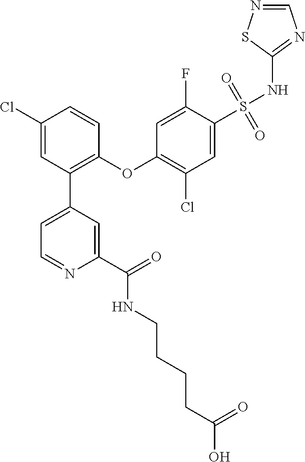 [SMILES: O=C(O)CCCCNC(=O)c1cc(-c2cc(Cl)ccc2Oc2cc(F)c(S(=O)(=O)Nc3ncns3)cc2Cl)ccn1] | 5-(4-(2-(4-(N-(1,2,4-thiadiazol-5-yl) sulfamoyl)-2-chloro-5-fluorophenoxy)-5- chlorophenyl)picolinamido)pentanoic acid |
| 4 | 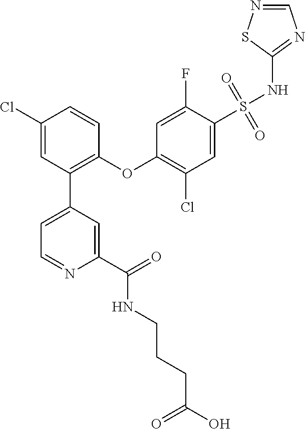 [SMILES: O=C(O)CCCNC(=O)c1cc(-c2cc(Cl)ccc2Oc2cc(F)c(S(=O)(=O)Nc3ncns3)cc2Cl)ccn1] | 4-(4-(2-(4-(N-(1,2,4-thiadiazol-5-yl) sulfamoyl)-2-chloro-5-fluorophenoxy)-5- chlorophenyl)picolinamido)butanoic acid |
| 5 | 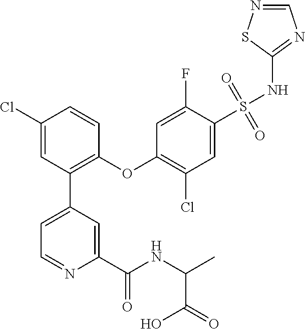 [SMILES: CC(NC(=O)c1cc(-c2cc(Cl)ccc2Oc2cc(F)c(S(=O)(=O)Nc3ncns3)cc2Cl)ccn1)C(=O)O] | 2-(4-(2-(4-(N-(1,2,4-thiadiazol-5-yl) sulfamoyl)-2-chloro-5-fluorophenoxy)-5- chlorophenyl)picolinamido)propanoic acid |
| 6 | 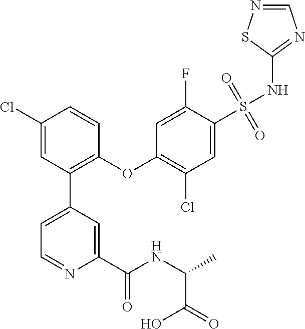 [SMILES: C[C@@H](NC(=O)c1cc(-c2cc(Cl)ccc2Oc2cc(F)c(S(=O)(=O)Nc3ncns3)cc2Cl)ccn1)C(=O)O] | (R)-2-(4-(2-(4-(N-(1,2,4-thiadiazol-5-yl) sulfamoyl)-2-chloro-5-fluorophenoxy)-5- chlorophenyl)picolinamido)propanoic acid |
| 7 | 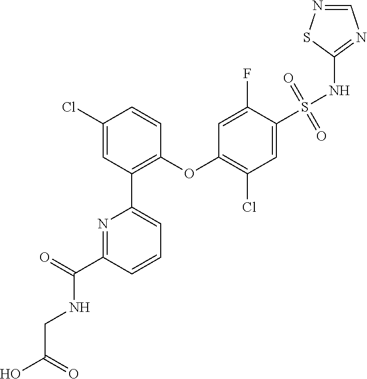 [SMILES: O=C(O)CNC(=O)c1cccc(-c2cc(Cl)ccc2Oc2cc(F)c(S(=O)(=O)Nc3ncns3)cc2Cl)n1] | 2-(6-(2-(4-(N-(1,2,4-thiadiazol-5-yl) sulfamoyl)-2-chloro-5-fluorophenoxy)-5- chlorophcnyl)picolinamido)acetic acid |
| 8 | 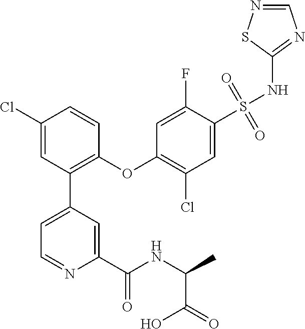 [SMILES: C[C@H](NC(=O)c1cc(-c2cc(Cl)ccc2Oc2cc(F)c(S(=O)(=O)Nc3ncns3)cc2Cl)ccn1)C(=O)O] | (S)-2-(4-(2-(4-(N-(1,2,4-thiadiazol-5-yl) sulfamoyl)-2-chloro-5-fluorophenoxy)-5- chlorophenyl)picolinamido)propanoic acid |
| 9 | 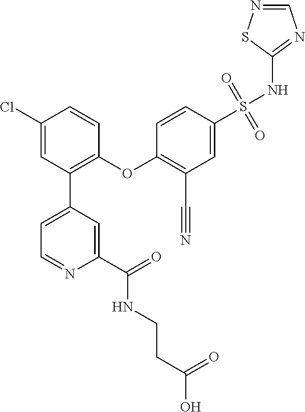 [SMILES: N#Cc1cc(S(=O)(=O)Nc2ncns2)ccc1Oc1ccc(Cl)cc1-c1ccnc(C(=O)NCCC(=O)O)c1] | 3-(4-(2-(4-(N-(1,2,4-thiadiazol-5-yl) sulfamoyl)-2-cyanophenoxy)-5- chlorophenyl)picolinamido)propanoic acid |
| 10 | 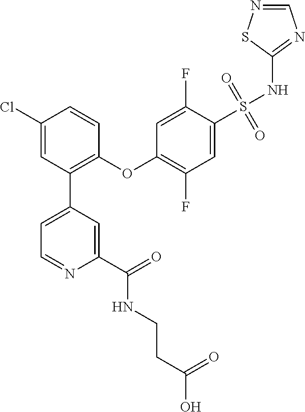 [SMILES: O=C(O)CCNC(=O)c1cc(-c2cc(Cl)ccc2Oc2cc(F)c(S(=O)(=O)Nc3ncns3)cc2F)ccn1] | 3-(4-(2-(4-(N-(1,2,4-thiadiazol-5-yl) sulfamoyl)-2,5-difluorophenoxy)-5- chlorophenyl)picolinamido)propanoic acid |
| 11 | 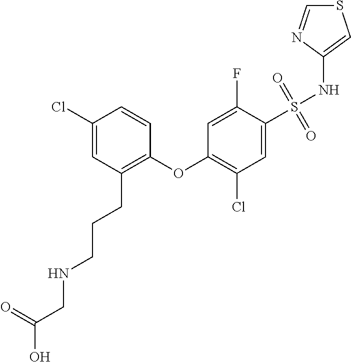 [SMILES: O=C(O)CNCCCc1cc(Cl)ccc1Oc1cc(F)c(S(=O)(=O)Nc2cscn2)cc1Cl] | 2-((3-(5-chloro-2-(2-chloro-5-fluoro- 4-(N-(thiazol-4-yl)sulfamoyl)phenoxy) phenyl)propyl)amino)acetic acid |
| 12 | 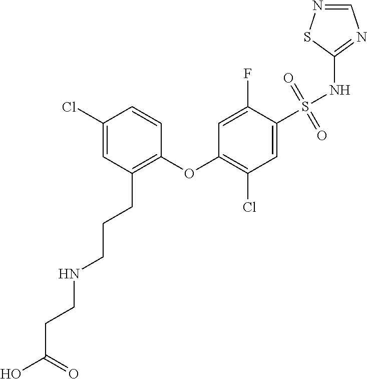 [SMILES: O=C(O)CCNCCCc1cc(Cl)ccc1Oc1cc(F)c(S(=O)(=O)Nc2ncns2)cc1Cl] | 3-((3-(2-(4-(N-(1,2,4-thiadiazol-5-yl) sulfamoyl)-2-chloro-5-fluorophenoxy)-5- chlorophenyl)propyl)amino)propanoic acid |
| 13 | 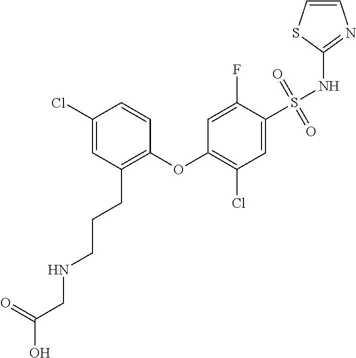 [SMILES: O=C(O)CNCCCc1cc(Cl)ccc1Oc1cc(F)c(S(=O)(=O)Nc2nccs2)cc1Cl] | 2-((3-(5-chloro-2-(2-chloro-5-fluoro-4- (N-(thiazol-2-yl)sulfamoyl)phenoxy)phenyl) propyl)amino)acetic acid |
| 14 | 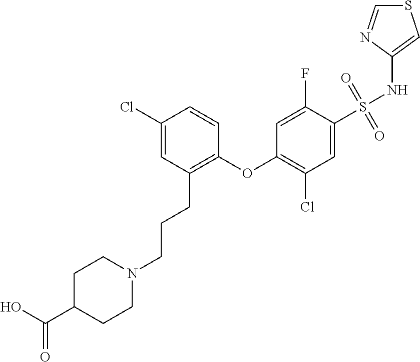 [SMILES: O=C(O)C1CCN(CCCc2cc(Cl)ccc2Oc2cc(F)c(S(=O)(=O)Nc3cscn3)cc2Cl)CC1] | 1-(3-(5-chloro-2-(2-chloro-5-fluoro-4- (N-(thiazol-4-yl)sulfamoyl)phenoxy)phenyl) propyl)piperidine-4-carboxylic acid |
| 15 | 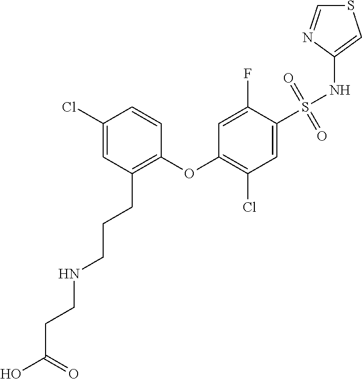 [SMILES: O=C(O)CCNCCCc1cc(Cl)ccc1Oc1cc(F)c(S(=O)(=O)Nc2cscn2)cc1Cl] | 3-((3-(5-chloro-2-(2-chloro-5- fluoro-4-(N-(thiazol-4-yl)sulfamoyl) phenoxy)phenyl)propyl)amino)propanoic acid |
| 16 | 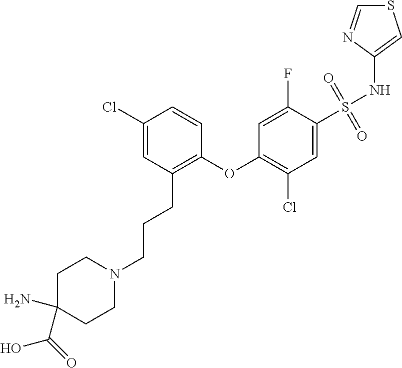 [SMILES: NC1(C(=O)O)CCN(CCCc2cc(Cl)ccc2Oc2cc(F)c(S(=O)(=O)Nc3cscn3)cc2Cl)CC1] | 4-amino-1-(3-(5-chloro-2-(2-chloro- 5-fluoro-4-(N-(thiazol-4-yl)sulfamoyl) phenoxy)phenyl)propyl)piperidine-4- carboxylic acid |
| 17 | 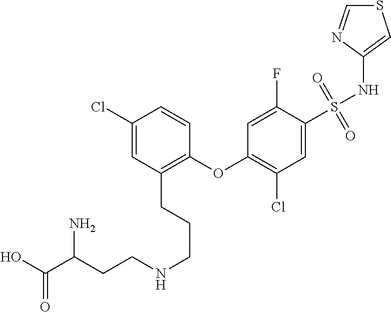 [SMILES: NC(CCNCCCc1cc(Cl)ccc1Oc1cc(F)c(S(=O)(=O)Nc2cscn2)cc1Cl)C(=O)O] | 2-amino-4-((3-(5-chloro-2-(2-chloro- 5-fluoro-4-(N-(thiazol-4-yl)sulfamoyl) phenoxy)phenyl)propyl)amino)butanoic acid |
| 18 | 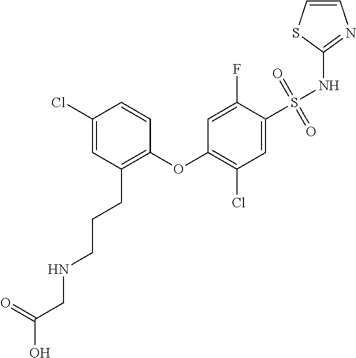 [SMILES: O=C(O)CNCCCc1cc(Cl)ccc1Oc1cc(F)c(S(=O)(=O)Nc2nccs2)cc1Cl] | 2-((3-(5-chloro-2-(2,5-difluoro-4-(N- (thiazol-2-yl)sulfamoyl)phenoxy)phenyl) propyl)amino)acetic acid |
| 19 | 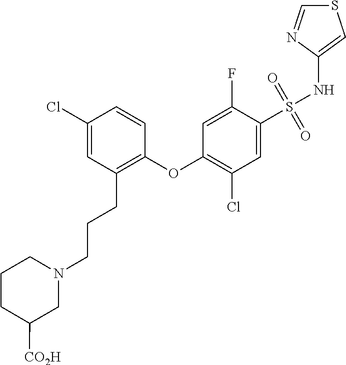 [SMILES: O=C(O)C1CCCN(CCCc2cc(Cl)ccc2Oc2cc(F)c(S(=O)(=O)Nc3cscn3)cc2Cl)C1] | 1-(3-(5-chloro-2-(2-chloro-5-fluoro-4-(N- (thiazol-4-yl)sulfamoyl)phenoxy)phenyl) propyl)piperidine-3-carboxylic acid |
| 20 | 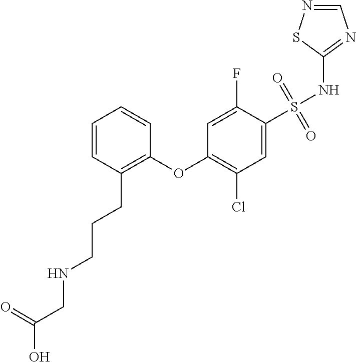 [SMILES: O=C(O)CNCCCc1ccccc1Oc1cc(F)c(S(=O)(=O)Nc2ncns2)cc1Cl] | 2-((3-(2-(4-(N-(1,2,4-thiadiazol-5-yl) sulfamoyl)-2-chloro-5-fluorophenoxy) phenyl)propyl)amino)acetic acid |
| 21 | 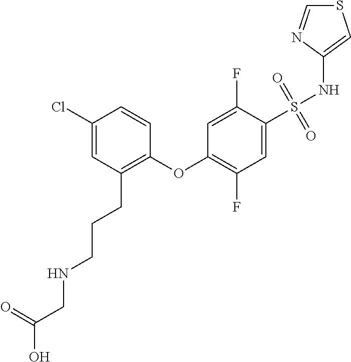 [SMILES: O=C(O)CNCCCc1cc(Cl)ccc1Oc1cc(F)c(S(=O)(=O)Nc2cscn2)cc1F] | 2-((3-(5-chloro-2-(2,5-difluoro-4- (N-(thiazol-4-yl)sulfamoyl)phenoxy) phenyl)propyl)amino)acetic acid |
| 22 | 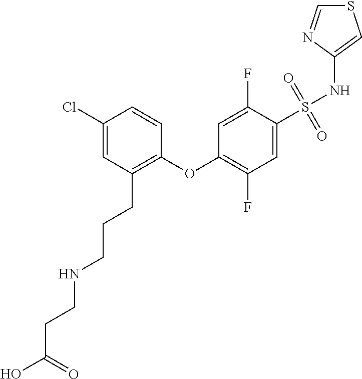 [SMILES: O=C(O)CCNCCCc1cc(Cl)ccc1Oc1cc(F)c(S(=O)(=O)Nc2cscn2)cc1F] | 3-((3-(5-chloro-2-(2,5-difluoro- 4-(N-(thiazol-4-yl)sulfamoyl) phenoxy)phenyl)propyl)amino acid |
| 23 | 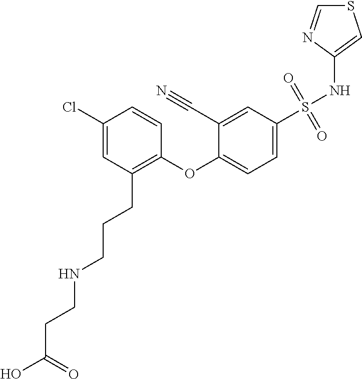 [SMILES: N#Cc1cc(S(=O)(=O)Nc2cscn2)ccc1Oc1ccc(Cl)cc1CCCNCCC(=O)O] | 3-((3-(5-chloro-2-(2-cyano-4-(N- (thiazol-4-yl)sulfamoyl)phenoxy) phenyl)propyl)amino)propanoic acid |
| 24 | 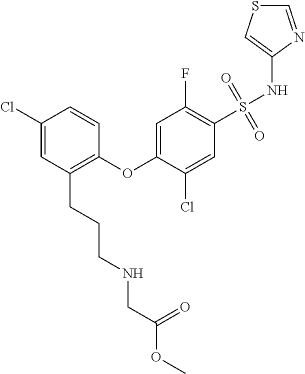 [SMILES: COC(=O)CNCCCc1cc(Cl)ccc1Oc1cc(F)c(S(=O)(=O)Nc2cscn2)cc1Cl] | methyl 2-((3-(5-chloro-2-(2- chloro-5-fluoro-4-(N- (thiazol-4-yl)sulfamoyl)phenoxy) phenyl)propyl)amino)acetate |
| 25 | 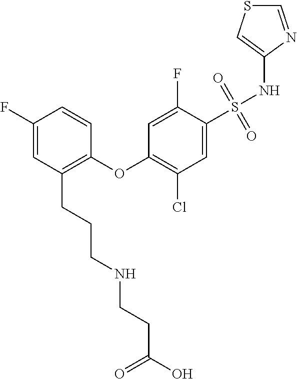 [SMILES: O=C(O)CCNCCCc1cc(F)ccc1Oc1cc(F)c(S(=O)(=O)Nc2cscn2)cc1Cl] | 3-((3-(2-(2-chloro-5-fluoro- 4-(N-(thiazol-4- yl)sulfamoyl)phenoxy)-5- fluorophenyl)propyl)amino) propanoic acid |
| 26 | 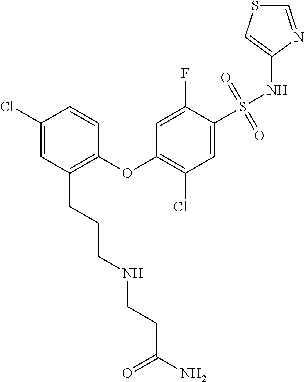 [SMILES: NC(=O)CCNCCCc1cc(Cl)ccc1Oc1cc(F)c(S(=O)(=O)Nc2cscn2)cc1Cl] | 3-((3-(5-chloro-2-(2-chloro-5- fluoro-4-(N-(thiazol-4- yl)sulfamoyl)phenoxy)phenyl) propyl)amino)propanamide |
| 27 | 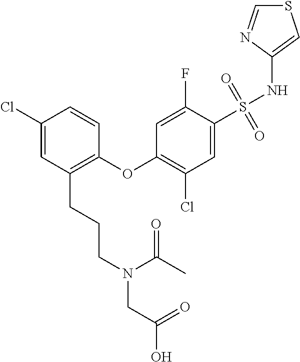 [SMILES: CC(=O)N(CCCc1cc(Cl)ccc1Oc1cc(F)c(S(=O)(=O)Nc2cscn2)cc1Cl)CC(=O)O] | 2-(N-(3-(5-chloro-2-(2-chloro-5- fluoro-4-(N-(thiazol-4- yl)sulfamoyl)phenoxy)phenyl) propyl)acetamido)acetic acid |
| 28 | 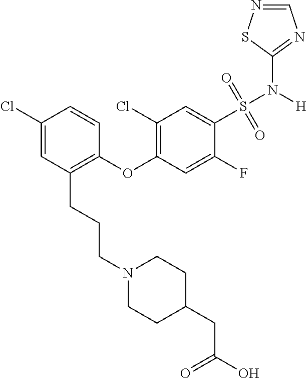 [SMILES: O=C(O)CC1CCN(CCCc2cc(Cl)ccc2Oc2cc(F)c(S(=O)(=O)Nc3ncns3)cc2Cl)CC1] | 2-(1-(3-(2-(4-(N-(1,2,4- thiadiazol-5-yl)sulfamoyl)-2- chloro-5-fluorophenoxy)-5- chlorophenyl)propyl) piperidin-4-yl)acetic acid |
| 29 | 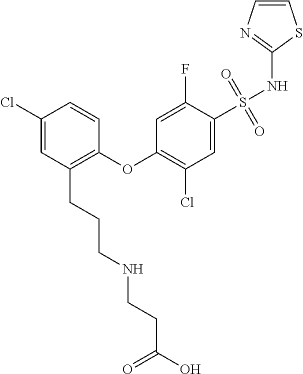 [SMILES: O=C(O)CCNCCCc1cc(Cl)ccc1Oc1cc(F)c(S(=O)(=O)Nc2nccs2)cc1Cl] | 3-((3-(5-chloro-2-(2-chloro-5- fluoro-4-(N-(thiazol-2- yl)sulfamoyl)phenoxy)phenyl) propyl)amino)propanoic acid |
| 30 | 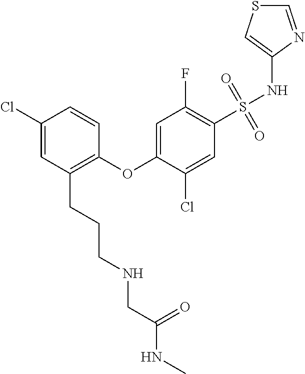 [SMILES: CNC(=O)CNCCCc1cc(Cl)ccc1Oc1cc(F)c(S(=O)(=O)Nc2cscn2)cc1Cl] | 2-((3-(5-chloro-2-(2-chloro-5- fluoro-4-(N-(thiazol-4- yl)sulfamoyl)phenoxy)phenyl) propyl)amino)-N- methylacetamide |
| 31 | 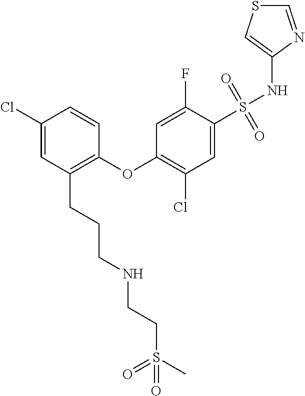 [SMILES: CS(=O)(=O)CCNCCCc1cc(Cl)ccc1Oc1cc(F)c(S(=O)(=O)Nc2cscn2)cc1Cl] | 5-chloro-4-(4-chloro-2-(3-((2- (methylsulfonyl)ethyl)amino) propyl)phenoxy)-2- fluoro-N-(thiazol-4-yl) benzenesulfonamide |
| 32 | 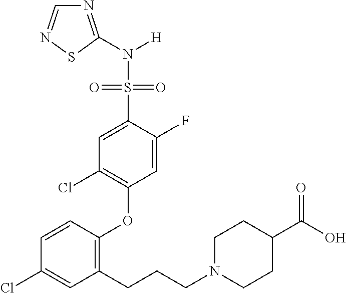 [SMILES: O=C(O)C1CCN(CCCc2cc(Cl)ccc2Oc2cc(F)c(S(=O)(=O)Nc3ncns3)cc2Cl)CC1] | 1-(3-(2-(4-(N-(1,2,4-thiadiazol- 5-yl)sulfamoyl)-2- chloro-5-fluorophenoxy)-5- chlorophenyl)propyl)piperidine- 4-carboxylic acid |
| 33 | 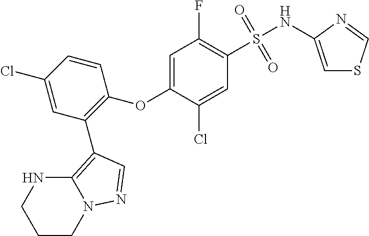 [SMILES: O=S(=O)(Nc1cscn1)c1cc(Cl)c(Oc2ccc(Cl)cc2-c2cnn3c2NCCC3)cc1F] | 5-chloro-4-(4-chloro-2- (4,5,6,7-tetrahydropyrazolo [1,5-a]pyrimidin-3-yl)phenoxy)-2- fluoro-N-(thiazol-4-yl) benzenesulfonamide |

*Chemical Names automatically generated with ChemDraw Ultra, Version 12.0.

In a certain embodiment, the compounds of Formula (I) or Formula (I′) are those wherein the compound is selected from the group consisting of the compounds in Table 2 or a pharmaceutically acceptable salt, or a stereoisomeric or tautomeric form thereof.

TABLE 2

| Prophetic | | |
|---|---|---|
| Compound | Compound structure | Chemical name* |
| 36 | 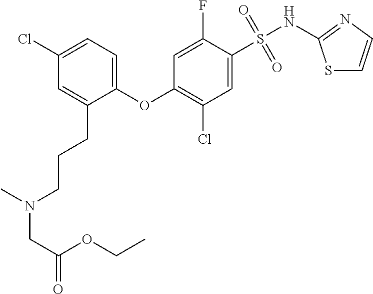 [SMILES: CCOC(=O)CN(C)CCCc1cc(Cl)ccc1Oc1cc(F)c(S(=O)(=O)Nc2nccs2)cc1Cl] | ethyl 2-((3-(5-chloro-2- (2-chloro-5-fluoro-4-(N- (thiazol-2-yl)sulfamoyl) phenoxy)phenyl)propyl) (methyl)amino)acetate |
| 37 | 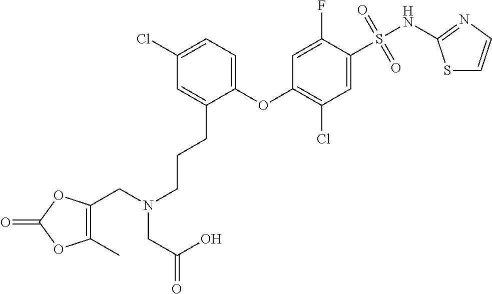 [SMILES: Cc1oc(=O)oc1CN(CCCc1cc(Cl)ccc1Oc1cc(F)c(S(=O)(=O)Nc2nccs2)cc1Cl)CC(=O)O] | 2-((3-(5-chloro-2-(2-chloro- 5-fluoro-4-(N-(thiazol-2-yl) sulfamoyl)phenoxy)phenyl) propyl)((5-methyl-2-oxo-1,3- dioxol-4-yl)methyl)amino) acetic acid |
| 38 | 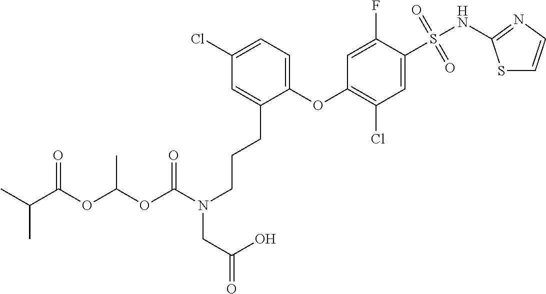 [SMILES: CC(OC(=O)C(C)C)OC(=O)N(CCCc1cc(Cl)ccc1Oc1cc(F)c(S(=O)(=O)Nc2nccs2)cc1Cl)CC(=O)O] | 2-((3-(5-chloro-2-(2-chloro- 5-fluoro-4-(N-(thiazol-2-yl) sulfamoyl)phenoxy)phenyl) propyl)((1-(isobutyryloxy) ethoxy)carbonyl)amino) acetic acid |
| 39 | 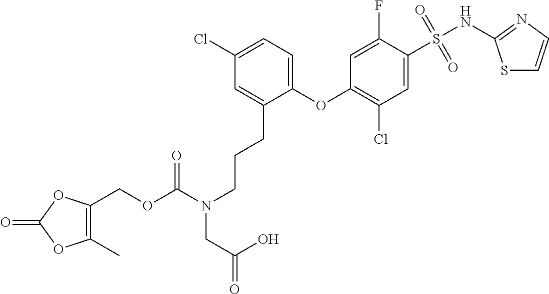 [SMILES: Cc1oc(=O)oc1COC(=O)N(CCCc1cc(Cl)ccc1Oc1cc(F)c(S(=O)(=O)Nc2nccs2)cc1Cl)CC(=O)O] | 2-((3-(5-chloro-2-(2-chloro- 5-fluoro-4-(N-(thiazol-2-yl) sulfamoyl)phenoxy)phenyl) propyl)(((5-methyl-2-oxo-1,3- dioxol-4-yl)methoxy)carbonyl) amino)acetic acid |
| 40 | 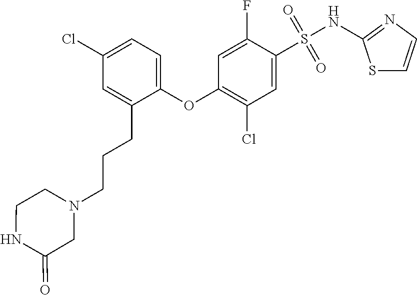 [SMILES: O=C1CN(CCCc2cc(Cl)ccc2Oc2cc(F)c(S(=O)(=O)Nc3nccs3)cc2Cl)CCN1] | 5-chloro-4-(4-chloro-2-(3- (3-oxopiperazin-1-yl)propyl) phenoxy)-2-fluoro-N-(thiazol- 2-yl)benzenesulfonamide |
| 41 | 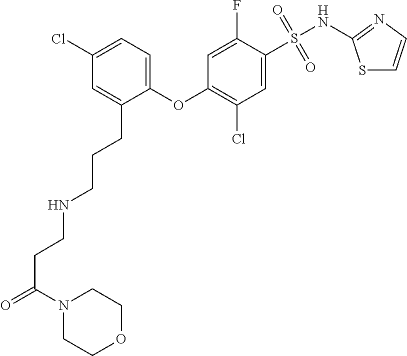 [SMILES: O=C(CCNCCCc1cc(Cl)ccc1Oc1cc(F)c(S(=O)(=O)Nc2nccs2)cc1Cl)N1CCOCC1] | 5-chloro-4-(4-chloro-2-(3- ((3-morpholino-3-oxopropyl) amino)propyl)phenoxy)-2- fluoro-N-(thiazol-2-yl) benzenesulfonamide |

*Chemical Names automatically generated with ChemDraw Ultra, Version 12.0.

In a certain embodiment, the compounds of Formula (I) or Formula (I′) are those wherein the compound is selected from the group consisting of the compounds in Table 3 or a pharmaceutically acceptable salt, or a stereoisomeric or tautomeric form thereof.

TABLE 3

| Compound | Compound structure | Chemical name* |
|---|---|---|
| 34 | 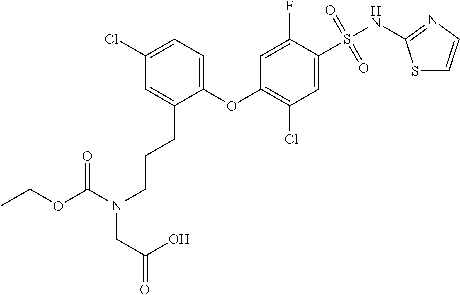 [SMILES: CCOC(=O)N(CCCc1cc(Cl)ccc1Oc1cc(F)c(S(=O)(=O)Nc2nccs2)cc1Cl)CC(=O)O] | 2-((3-(5-chloro-2-(2-chloro- 5-fluoro-4-(N-(thiazol-2- yl)sulfamoyl)phenoxy)phenyl) propyl)(ethoxycarbonyl) amino)acetic acid |
| 35 | 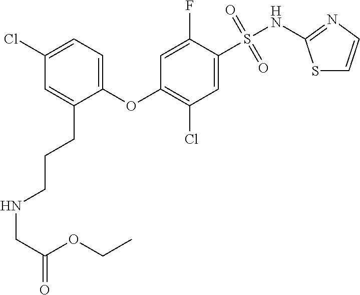 [SMILES: CCOC(=O)CNCCCc1cc(Cl)ccc1Oc1cc(F)c(S(=O)(=O)Nc2nccs2)cc1Cl] | ethyl 2-((3-(5-chloro-2- (2-chloro-5-fluoro-4-(N- (thiazol-2-yl)sulfamoyl) phenoxy)phenyl)propyl) amino)acetate |
| 42 | 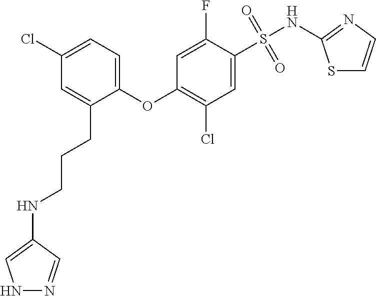 [SMILES: O=S(=O)(Nc1nccs1)c1cc(Cl)c(Oc2ccc(Cl)cc2CCCNc2cn[nH]c2)cc1F] | 4-(2-(3-((1H-pyrazol-4-yl) amino)propyl)-4- chlorophenoxy)-5-chloro- 2-fluoro-N-(thiazol-2- yl)benzenesulfonamide |
| 43 | 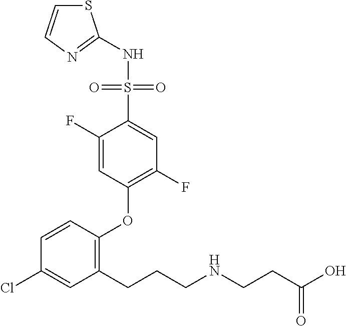 [SMILES: O=C(O)CCNCCCc1cc(Cl)ccc1Oc1cc(F)c(S(=O)(=O)Nc2nccs2)cc1F] | 3-((3-(5-chloro-2-(2,5- difluoro-4-(N-(thiazol-2- yl)sulfamoyl)phenoxy) phenyl)propyl)amino) propanoic acid |
| 44 | 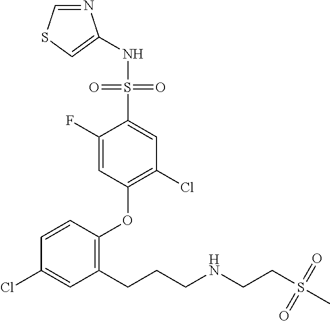 [SMILES: CS(=O)(=O)CCNCCCc1cc(Cl)ccc1Oc1cc(F)c(S(=O)(=O)Nc2cscn2)cc1Cl] | 5-chloro-4-(4-chloro-2- (3-((2-(methylsulfonyl) ethyl)amino)propyl) phenoxy)-2-fluoro-N- (thiazol-4-yl)benzenesulfonamide |
| 45 | 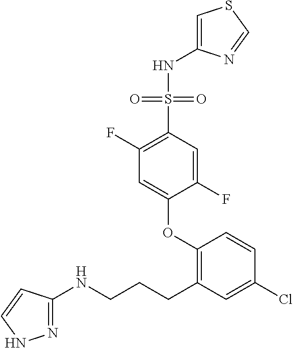 [SMILES: O=S(=O)(Nc1cscn1)c1cc(F)c(Oc2ccc(Cl)cc2CCCNc2cc[nH]n2)cc1F] | 4-(2-(3-((1H-pyrazol-3-yl) amino)propyl)-4-chlorophenoxy)- 5-chloro-2-fluoro-N-(thiazol-4- yl)benzenesulfonamide |
| 46 | 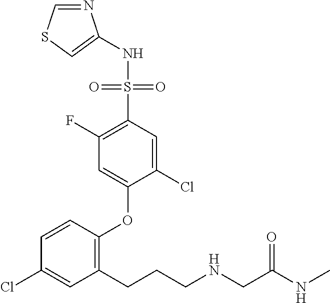 [SMILES: CNC(=O)CNCCCc1cc(Cl)ccc1Oc1cc(F)c(S(=O)(=O)Nc2cscn2)cc1Cl] | 2-((3-(5-chloro-2- (2-chloro-5-fluoro-4-(N- (thiazol-4-yl)sulfamoyl) phenoxy)phenyl)propyl) amino)-N-methylacetamide |
| 47 | 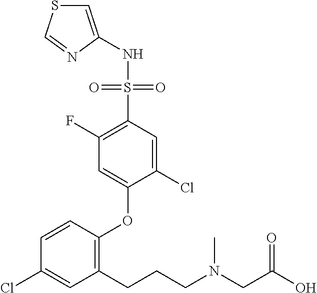 [SMILES: CN(CCCc1cc(Cl)ccc1Oc1cc(F)c(S(=O)(=O)Nc2cscn2)cc1Cl)CC(=O)O] | 2-((3-(5-chloro-2- (2-chloro-5-fluoro-4-(N- (thiazol-4-yl)sulfamoyl) phenoxy)phcnyl)propyl) (methyl)amino)acetic acid |
| 48 | 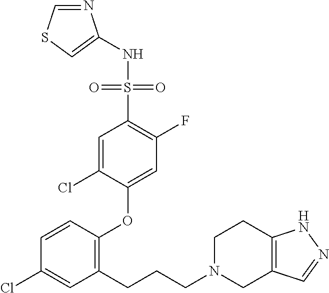 [SMILES: O=S(=O)(Nc1cscn1)c1cc(Cl)c(Oc2ccc(Cl)cc2CCCN2CCc3[nH]ncc3C2)cc1F] | 5-chloro-4-(4-chloro- 2-(3-(6,7-dihydro-1H- pyrazolo[4,3-c]pyridin- 5(4H)-yl)propyl)phenoxy)- 2-fluoro-N-(thiazol-4- yl)benzenesulfonamide |
| 49 | 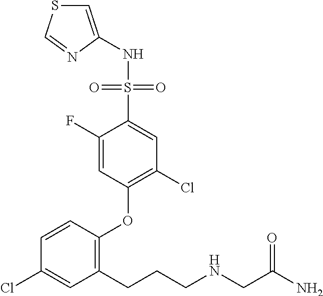 [SMILES: NC(=O)CNCCCc1cc(Cl)ccc1Oc1cc(F)c(S(=O)(=O)Nc2cscn2)cc1Cl] | 2-((3-(5-chloro-2- (2-chloro-5-fluoro-4-(N- (thiazol-4-yl)sulfamoyl) phenoxy)phenyl)propyl) amino)acetamide |
| 50 | 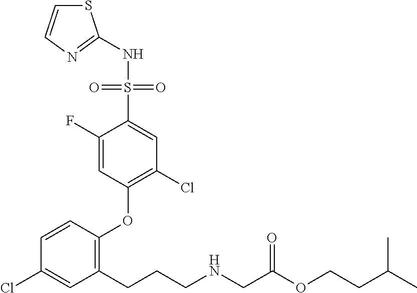 [SMILES: CC(C)CCOC(=O)CNCCCc1cc(Cl)ccc1Oc1cc(F)c(S(=O)(=O)Nc2nccs2)cc1Cl] | isopentyl 2-((3-(5-chloro- 2-(2-chloro-5-fluoro-4- (N-(thiazol-2-yl)sulfamoyl) phenoxy)phenyl)propyl) amino)acetate |
| 51 | 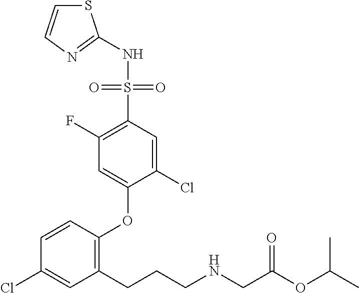 [SMILES: CC(C)OC(=O)CNCCCc1cc(Cl)ccc1Oc1cc(F)c(S(=O)(=O)Nc2nccs2)cc1Cl] | isopropyl 2-((3-(5-chloro- 2-(2-chloro-5-fluoro-4- (N-(thiazol-2-yl)sulfamoyl) phenoxy)phenyl)propyl) amino)acetate |
| 52 | 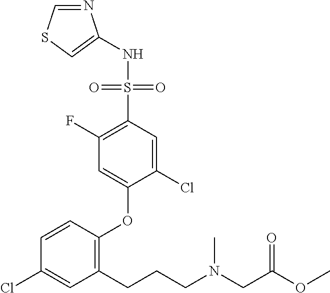 [SMILES: COC(=O)CN(C)CCCc1cc(Cl)ccc1Oc1cc(F)c(S(=O)(=O)Nc2cscn2)cc1Cl] | methyl 2-((3-(5-chloro-2- (2-chloro-5-fluoro-4- (N-(thiazol-4-yl)sulfamoyl) phenoxy)phenyl)propyl) (methyl)amino)acetate |
| 53 | 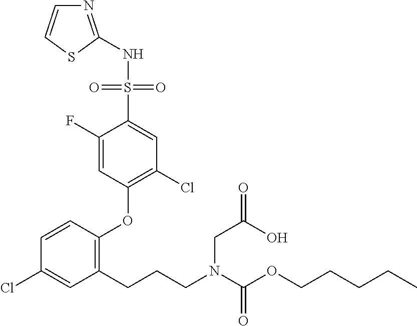 [SMILES: CCCCCOC(=O)N(CCCc1cc(Cl)ccc1Oc1cc(F)c(S(=O)(=O)Nc2nccs2)cc1Cl)CC(=O)O] | 2-((3-(5-chloro-2- (2-chloro-5-fluoro-4-(N- (thiazol-2-yl)sulfamoyl) phenoxy)phenyl)propyl) ((pentyloxy)carbonyl) amino)acetic acid |
| 54 | 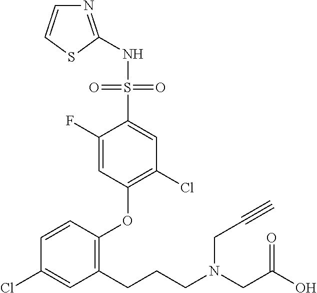 [SMILES: C#CCN(CCCc1cc(Cl)ccc1Oc1cc(F)c(S(=O)(=O)Nc2nccs2)cc1Cl)CC(=O)O] | 2-((3-(5-chloro-2- (2-chloro-5-fluoro-4-(N- (thiazol-2-yl)sulfamoyl) phenoxy)phenyl)propyl) (prop-2-yn-1-yl)amino) acetic acid |
| 55 | 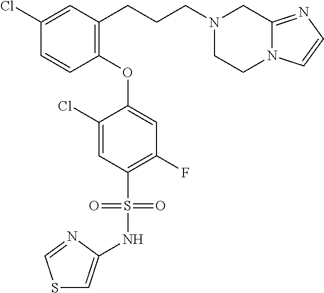 [SMILES: O=S(=O)(Nc1cscn1)c1cc(Cl)c(Oc2ccc(Cl)cc2CCCN2CCn3ccnc3C2)cc1F] | 5-chloro-4-(4-chloro- 2-(3-(5,6-dihydroimidazo [1,2-a]pyrazin-7(8H)- yl)propyl)phenoxy)-2- fluoro-N-(thiazol-4- yl)benzenesulfonamide |
| 56 | 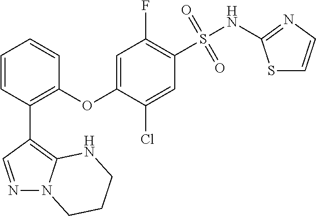 [SMILES: O=S(=O)(Nc1nccs1)c1cc(Cl)c(Oc2ccccc2-c2cnn3c2NCCC3)cc1F] | 5-chloro-2-fluoro-4-(2- (4,5,6,7-tetrahydropyrazolo [1,5-a]pyrimidin-3-yl) phenoxy)-N-(thiazol-2- yl)benzenesulfonamide |
| 57 | 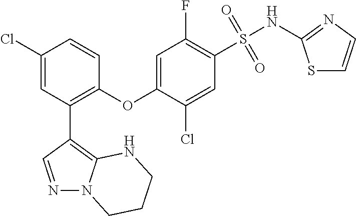 [SMILES: O=S(=O)(Nc1nccs1)c1cc(Cl)c(Oc2ccc(Cl)cc2-c2cnn3c2NCCC3)cc1F] | 5-chloro-4-(4-chloro-2- (4,5,6,7-tetrahydropyrazolo [1,5-a]pyrimidin-3-yl) phenoxy)-2-fluoro-N- (thiazol-2-yl) benzenesulfonamide |
| 58 | 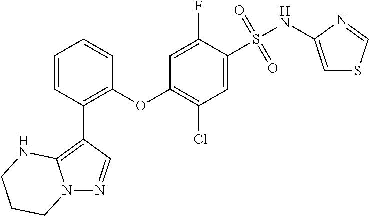 [SMILES: O=S(=O)(Nc1cscn1)c1cc(Cl)c(Oc2ccccc2-c2cnn3c2NCCC3)cc1F] | 5-chloro-2-fluoro-4-(2- (4,5,6,7-tetrahydropyrazolo [1,5-a]pyrimidin-3- yl)phenoxy)-N-(thiazol-4- yl)benzenesulfonamide |
| 59 | 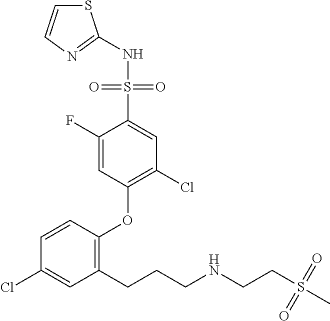 [SMILES: CS(=O)(=O)CCNCCCc1cc(Cl)ccc1Oc1cc(F)c(S(=O)(=O)Nc2nccs2)cc1Cl] | 5-chloro-4-(4-chloro- 2-(3-((2-(methylsulfonyl) ethyl)amino)propyl) phenoxy)-2-fluoro-N- (thiazol-2-yl) benzenesulfonamide |
| 60 | 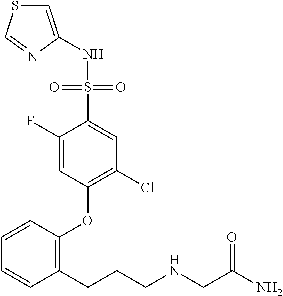 [SMILES: NC(=O)CNCCCc1ccccc1Oc1cc(F)c(S(=O)(=O)Nc2cscn2)cc1Cl] | 2-((3-(2-(2-chloro-5- fluoro-4-(N-(thiazol-4- yl)sulfamoyl)phenoxy) phenyl)propyl)amino) acetamide |
| 61 | 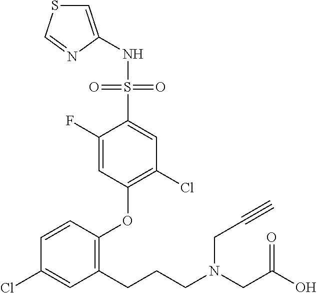 [SMILES: C#CCN(CCCc1cc(Cl)ccc1Oc1cc(F)c(S(=O)(=O)Nc2cscn2)cc1Cl)CC(=O)O] | 2-((3-(5-chloro-2- (2-chloro-5-fluoro-4-(N- (thiazol-4-yl)sulfamoyl) phenoxy)phenyl)propyl) (prop-2-yn-1-yl)amino) acetic acid |
| 62 | 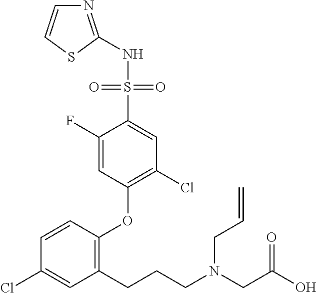 [SMILES: C=CCN(CCCc1cc(Cl)ccc1Oc1cc(F)c(S(=O)(=O)Nc2nccs2)cc1Cl)CC(=O)O] | 2-(allyl(3-(5-chloro-2- (2-chloro-5-fluoro-4-(N- (thiazol-2-yl)sulfamoyl) phenoxy)phenyl)propyl) amino)acetic acid |
| 63 | 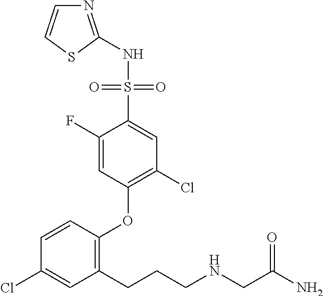 [SMILES: NC(=O)CNCCCc1cc(Cl)ccc1Oc1cc(F)c(S(=O)(=O)Nc2nccs2)cc1Cl] | 2-((3-(5-chloro-2- (2-chloro-5-fluoro-4-(N- (thiazol-2-yl)sulfamoyl) phenoxy)phenyl)propyl) amino)acetamide |
| 64 | 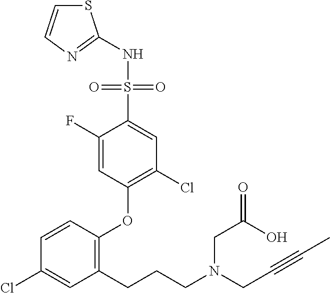 [SMILES: CC#CCN(CCCc1cc(Cl)ccc1Oc1cc(F)c(S(=O)(=O)Nc2nccs2)cc1Cl)CC(=O)O] | 2-(but-2-yn-1-yl(3- (5-chloro-2-(2-chloro-5- fluoro-4-(N-(thiazol-2- yl)sulfamoyl)phenoxy) phenyl)propyl)amino) acetic acid |
| 65 | 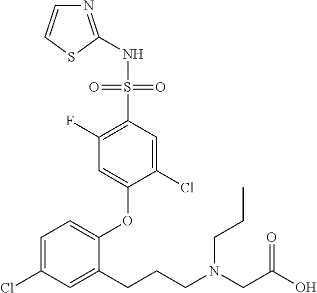 [SMILES: CCCN(CCCc1cc(Cl)ccc1Oc1cc(F)c(S(=O)(=O)Nc2nccs2)cc1Cl)CC(=O)O] | 2-((3-(5-chloro-2- (2-chloro-5-fluoro-4-(N- (thiazol-2-yl)sulfamoyl) phenoxy)phenyl)propyl) (propyl)amino)acetic acid |
| 66 | 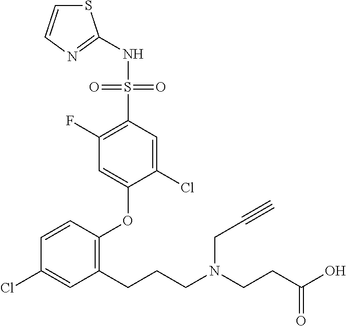 [SMILES: C#CCN(CCCc1cc(Cl)ccc1Oc1cc(F)c(S(=O)(=O)Nc2nccs2)cc1Cl)CCC(=O)O] | 3-((3-(5-chloro-2- (2-chloro-5-fluoro-4-(N- (thiazol-2-yl)sulfamoyl) phenoxy)phenyl)propyl) (prop-2-yn-1-yl)amino) propanoic acid |
| 67 | 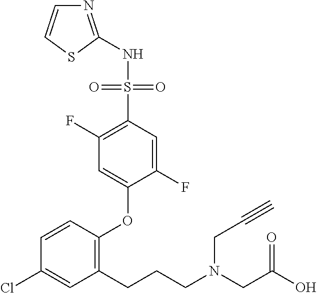 [SMILES: C#CCN(CCCc1cc(Cl)ccc1Oc1cc(F)c(S(=O)(=O)Nc2nccs2)cc1F)CC(=O)O] | 2-((3-(5-chloro-2- (2,5-difluoro-4-(N- (thiazol-2-yl)sulfamoyl) phenoxy)phenyl)propyl) (prop-2-yn-1-yl)amino) acetic acid |
| 68 | 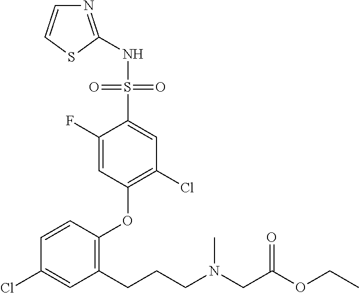 [SMILES: CCOC(=O)CN(C)CCCc1cc(Cl)ccc1Oc1cc(F)c(S(=O)(=O)Nc2nccs2)cc1Cl] | ethyl 2-((3-(5-chloro-2- (2-chloro-5-fluoro-4-(N- (thiazol-2-yl)sulfamoyl) phenoxy)phenyl)propyl) (methyl)amino)acetate |
| 69 | 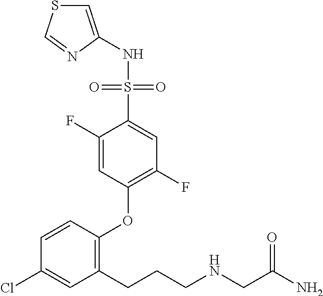 [SMILES: NC(=O)CNCCCc1cc(Cl)ccc1Oc1cc(F)c(S(=O)(=O)Nc2cscn2)cc1F] | 2-((3-(5-chloro-2- (2,5-difluoro-4-(N- (thiazol-4-yl)sulfamoyl) phenoxy)phenyl)propyl) amino)acetamide |

*Chemical Names automatically generated with ChemDraw Ultra, Version 12.0.

For the proposes of this disclosure, Table 1, Table 2, and Table 3 serve to define that a particular structure is associated with a particular name. Whenever a particular name is recited in this disclosure or the claims, the chemical structure associated with that particular name shall be the structure identified in Table 1, Table 2, or Table 3.

In a particular embodiment, the compounds of Formula (I) or Formula (I′) are those wherein the compound is
2-(4-(2-(4-(N-(1,2,4-thiadiazol-5-yl)sulfamoyl)-2-chloro-5-fluorophenoxy)-5-chlorophenyl)picolinamido)acetic acid,
3-(4-(2-(4-(N-(1,2,4-thiadiazol-5-yl)sulfamoyl)-2-chloro-5-fluorophenoxy)-5-chlorophenyl)picolinamido)propanoic acid,
2-(4-(2-(4-(N-(1,2,4-thiadiazol-5-yl)sulfamoyl)-2-chloro-5-fluorophenoxy)-5-chlorophenyl)picolinamido)propanoic acid, or
3-((3-(2-(4-(N-(1,2,4-thiadiazol-5-yl)sulfamoyl)-2-chloro-5-fluorophenoxy)-5-chlorophenyl)propyl)amino)propanoic acid;
   or a pharmaceutically acceptable salt, or a stereoisomeric or tautomeric form thereof.

In a particular embodiment, the compounds of Formula (I) or Formula (I′) are those wherein the compound is
2-((3-(5-chloro-2-(2-chloro-5-fluoro-4-(N-(thiazol-4-yl)sulfamoyl)phenoxy)phenyl)propyl)amino)acetamide,
2-((3-(5-chloro-2-(2-chloro-5-fluoro-4-(N-(thiazol-2-yl)sulfamoyl)phenoxy)phenyl)propyl)((pentyloxy)carbonyl)amino)acetic acid, or
2-((3-(5-chloro-2-(2-chloro-5-fluoro-4-(N-(thiazol-2-yl)sulfamoyl)phenoxy)phenyl)propyl)(prop-2-yn-1-yl)amino)acetic acid;
   or a pharmaceutically acceptable salt, or a stereoisomeric or tautomeric form thereof.

In a particular embodiment, the compounds of Formula (I) or Formula (I′) are those wherein the compound is
3-((3-(5-chloro-2-(2-chloro-5-fluoro-4-(N-(thiazol-4-yl)sulfamoyl)phenoxy)phenyl)propyl)amino)propanoic acid,
2-((3-(5-chloro-2-(2-chloro-5-fluoro-4-(N-(thiazol-4-yl)sulfamoyl)phenoxy)phenyl)propyl)amino)acetamide,
2-((3-(5-chloro-2-(2-chloro-5-fluoro-4-(N-(thiazol-2-yl)sulfamoyl)phenoxy)phenyl)propyl)(prop-2-yn-1-yl)amino)acetic acid,
2-((3-(5-chloro-2-(2-chloro-5-fluoro-4-(N-(thiazol-4-yl)sulfamoyl)phenoxy)phenyl)propyl)(prop-2-yn-1-yl)amino)acetic acid,
2-((3-(5-chloro-2-(2-chloro-5-fluoro-4-(N-(thiazol-2-yl)sulfamoyl)phenoxy)phenyl)propyl)amino)acetamide,
2-((3-(5-chloro-2-(2-chloro-5-fluoro-4-(N-(thiazol-2-yl)sulfamoyl)phenoxy)phenyl)propyl)(propyl)amino)acetic acid,
2-((3-(5-chloro-2-(2,5-difluoro-4-(N-(thiazol-2-yl)sulfamoyl)phenoxy)phenyl)propyl)(prop-2-yn-1-yl)amino)acetic acid, or
2-((3-(5-chloro-2-(2,5-difluoro-4-(N-(thiazol-4-yl)sulfamoyl)phenoxy)phenyl)propyl)amino)acetamide;
   or a pharmaceutically acceptable salt, or a stereoisomeric or tautomeric form thereof.

Further provided herein are compounds of Formula (Ia),

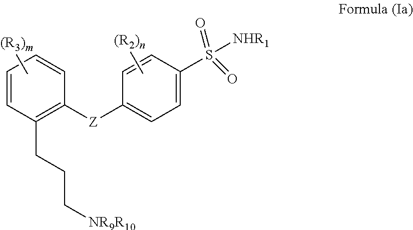

or a pharmaceutically acceptable salt, or a stereoisomeric or tautomeric form thereof, wherein:
Z is —O— or —S—;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₉ is (C₁-C₆)alkyl, (C₃-C₈)cycloalkyl, pyrazolyl or pyridinyl; wherein R₉ is optionally further substituted with 1 or 2 substituents selected from the group consisting of —COOH, —COOR₁₁, —CONR₁₁R₁₂, —SO₂R₁₁, —SO₂NR₁₁R₁₂, —OH, —CN, —OR₁₁, and —NR₁₁R₁₂; wherein R₁₁ and R₁₂ may form a 6 membered heterocycloalkyl ring
R₁₀ is R₁₁, —COR₁₁, —COOR₁₁, —SO₂R₁₁, 5-methyl-2-oxo-1,3-dioxol-4-yl,

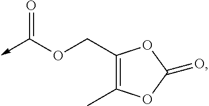

—COO—CH(CH₃)OCOCH(CH₃)₂; or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy;
R₁₁ and R₁₂ are independently H or (C₁-C₆)alkyl, optionally substituted with 4- to 8-membered heterocycloalkyl ring; and
m and n are each independently 1, 2, 3, or 4.

In a certain embodiment, the compounds of Formula (I′a)

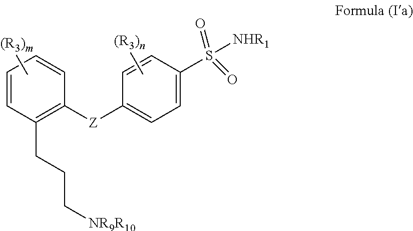

are those wherein
R₁₀ is R₁₁, (C₃-C₆)alkynyl, (C₃-C₆)alkenyl, —COR₁₁, —COOR₁₁, —SO₂R₁₁, 5-methyl-2-oxo-1,3-dioxol-4-yl,

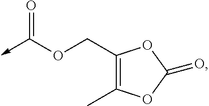

—COO—CH(CH₃)OCOCH(CH₃)₂; or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy; or R₉ and R₁₀ together form a unsubstituted 4- to 8-membered heterocycloalkyl ring, wherein said heterocycloalkyl ring is fused with a 5-membered heteroaryl; and
   wherein all other substituents are defined as in paragraph [00115] above.

In a particular embodiment, the compounds of Formula (Ia) or Formula (I′a) are those wherein R₁ is an aromatic 5- or 6-membered heterocycle, with 1-3 heteroatoms independently selected from the group consisting of N, O, and S.

In a particular embodiment, the compounds of Formula (Ia) or Formula (I′a) are those wherein R₁ is pyridyl or pyrimidinyl.

In a particular embodiment, the compounds of Formula (Ia) or Formula (I′a) are those wherein R₁ is an aromatic 5-membered heterocycle with 1 or 2 nitrogen atoms and optionally 1 or 2 sulphur atoms. In a particular embodiment, the compounds of Formula (Ia) or Formula (I′a) are those wherein R₁ is thiazolyl, isothiazolyl, or thiadiazolyl. In a particular embodiment, the compounds of Formula (Ia) or Formula (I′a) are those wherein R₁ is thiazolyl. In a particular embodiment, the compounds of Formula (Ia) or Formula (I′a) are those wherein R₁ is 1,2,4-thiadiazol-5-yl. In a particular embodiment, the compounds of Formula (Ia) or Formula (I′a) are those wherein R₁ is thiadiazol-4-yl.

In a particular embodiment, the compounds of Formula (Ia) or Formula (I′a) are those wherein R₂ is independently at each occurrence —F or —Cl.

In a particular embodiment, the compounds of Formula (Ia) or Formula (I′a) are those wherein n is 1, 2, or 3. In a particular embodiment, the compounds of Formula (Ia) or Formula (I′a) are those wherein n is 2.

In a particular embodiment, the compounds of Formula (Ia) or Formula (I′a) are those wherein Z is —O—.

In a particular embodiment, the compounds of Formula (Ia) or Formula (I′a) are those wherein R₃ is independently at each occurrence —H, —F, —Cl, or —Br. In a particular embodiment, the compounds of Formula (Ia) or Formula (I′a) are those wherein R₃ is —H or —Cl. In a particular embodiment, the compounds of Formula (Ia) or Formula (I′a) are those wherein R₃ is —Cl.

In a particular embodiment, the compounds of Formula (Ia) or Formula (I′a) are those wherein m is 1, 2, or 3. In a particular embodiment, the compounds of Formula (Ia) or Formula (I′a) are those wherein m is 1.

In a particular embodiment, the compounds of Formula (Ia) or Formula (I′a) are those wherein R₉ is (C₁-C₆)alkyl; wherein R₉ is optionally further substituted with 1 or 2 substituents selected from the group consisting of —COOH, —COOMe, —CONH₂, and —NH₂. In a particular embodiment, the compounds of Formula (Ia) or Formula (I′a) are those wherein R₉ is methyl or ethyl. In a particular embodiment, the compounds of Formula (Ia) or Formula (I′a) are those wherein R₉ is further substituted with —COOH.

In a particular embodiment, the compounds of Formula (Ia) or Formula (I′a) are those wherein R₁₀ is H and R₉ is (C₁-C₆)alkyl; wherein R₉ is further substituted with —CONR₁₁R₁₂, and wherein R₁₁ and R₁₂ are independently H or (C₁-C₆)alkyl. In a particular embodiment, the compounds of Formula (Ia) or Formula (I′a) are those wherein R₉ is further substituted with —CONH₂. In a particular embodiment, the compounds of Formula (Ia) or Formula (I′a) are those wherein R₉ is methyl and wherein R₉ is further substituted with —CONH₂.

In a particular embodiment, the compounds of Formula (Ia) or Formula (I′a) are those wherein R₁₀ is —H, —COMe, —COOEt. In a particular embodiment, the compounds of Formula (Ia) or Formula (I′a) are those wherein R₁₀ is —H or —COMe. In a particular embodiment, the compounds of Formula (Ia) or Formula (I′a) are those wherein R₁₀ is —H.

In a particular embodiment, the compounds of Formula (Ia) or Formula (I′a) are those wherein R₉ and R₁₀ together form a 4 to 8 membered heterocycloalkyl ring, wherein said heterocycloalkyl ring is substituted with 1 or 2 groups selected from the group consisting of —COOH, —COOMe, —COOEt, —CH₂—COOH, and —NH₂. In a particular embodiment, the compounds of Formula (I) are those wherein R₉ and R₁₀ together form a 4 to 8 membered heterocycloalkyl ring, wherein said heterocycloalkyl ring is substituted with 1 or 2 groups selected from the group consisting of —COOH, —CH₂—COOH, and —NH₂.

In a particular embodiment, the compounds of Formula (Ia) or Formula (I′a) are those wherein R₉ and R₁₀ together form a piperidine substituted with 1 or 2 groups selected from the group consisting of —COOH, —COOMe, —COOEt, —CH₂—COOH, —CH₂—COOMe, —CH₂—COOEt, and —NH₂. In a particular embodiment, the compounds of Formula (Ia) or Formula (I′a) are those wherein R₉ and R₁₀ together form a piperidine substituted with 1 or 2 groups selected from the group consisting of —COOH, —CH₂—COOH, and —NH₂.

In a particular embodiment, the compounds of Formula (Ia) or Formula (I′a) are selected from the group consisting of
2-((3-(5-chloro-2-(2-chloro-5-fluoro-4-(N-(thiazol-4-yl)sulfamoyl)phenoxy)phenyl)propyl)amino)acetic acid,
3-((3-(2-(4-(N-(1,2,4-thiadiazol-5-yl)sulfamoyl)-2-chloro-5-fluorophenoxy)-5-chlorophenyl)propyl)amino)propanoic acid,
2-((3-(5-chloro-2-(2-chloro-5-fluoro-4-(N-(thiazol-2-yl)sulfamoyl)phenoxy)phenyl)propyl)amino)acetic acid,
1-(3-(5-chloro-2-(2-chloro-5-fluoro-4-(N-(thiazol-4-yl)sulfamoyl)phenoxy)phenyl)propyl)piperidine-4-carboxylic acid,
3-((3-(5-chloro-2-(2-chloro-5-fluoro-4-(N-(thiazol-4-yl)sulfamoyl)phenoxy)phenyl)propyl)amino)propanoic acid,
4-amino-1-(3-(5-chloro-2-(2-chloro-5-fluoro-4-(N-(thiazol-4-yl)sulfamoyl)phenoxy)phenyl)propyl)piperidine-4-carboxylic acid,
2-amino-4-((3-(5-chloro-2-(2-chloro-5-fluoro-4-(N-(thiazol-4-yl)sulfamoyl)phenoxy)phenyl)propyl)amino)butanoic acid,
2-((3-(5-chloro-2-(2,5-difluoro-4-(N-(thiazol-2-yl)sulfamoyl)phenoxy)phenyl)propyl)amino)acetic acid,
1-(3-(5-chloro-2-(2-chloro-5-fluoro-4-(N-(thiazol-4-yl)sulfamoyl)phenoxy)phenyl)propyl)piperidine-3-carboxylic acid,
2-((3-(2-(4-(N-(1,2,4-thiadiazol-5-yl)sulfamoyl)-2-chloro-5-fluorophenoxy)phenyl)propyl)amino)acetic acid,
2-((3-(5-chloro-2-(2,5-difluoro-4-(N-(thiazol-4-yl)sulfamoyl)phenoxy)phenyl)propyl)amino)acetic acid,
3-((3-(5-chloro-2-(2,5-difluoro-4-(N-(thiazol-4-yl)sulfamoyl)phenoxy)phenyl)propyl)amino)propanoic acid,
3-((3-(5-chloro-2-(2-cyano-4-(N-(thiazol-4-yl)sulfamoyl)phenoxy)phenyl)propyl)amino)propanoic acid,
methyl 2-((3-(5-chloro-2-(2-chloro-5-fluoro-4-(N-(thiazol-4-yl)sulfamoyl)phenoxy)phenyl)propyl)amino)acetate,
3-((3-(2-(2-chloro-5-fluoro-4-(N-(thiazol-4-yl)sulfamoyl)phenoxy)-5-fluorophenyl)propyl)amino)propanoic acid,
3-((3-(5-chloro-2-(2-chloro-5-fluoro-4-(N-(thiazol-4-yl)sulfamoyl)phenoxy)phenyl)propyl)amino)propanamide,
2-(N-(3-(5-chloro-2-(2-chloro-5-fluoro-4-(N-(thiazol-4-yl)sulfamoyl)phenoxy)phenyl)propyl)acetamido)acetic acid,
2-(1-(3-(2-(4-(N-(1,2,4-thiadiazol-5-yl)sulfamoyl)-2-chloro-5-fluorophenoxy)-5-chlorophenyl)propyl)piperidin-4-yl)acetic acid,
3-((3-(5-chloro-2-(2-chloro-5-fluoro-4-(N-(thiazol-2-yl)sulfamoyl)phenoxy)phenyl)propyl)amino)propanoic acid,
2-((3-(5-chloro-2-(2-chloro-5-fluoro-4-(N-(thiazol-4-yl)sulfamoyl)phenoxy)phenyl)propyl)amino)-N-methylacetamide,
5-chloro-4-(4-chloro-2-(3-((2-(methylsulfonyl)ethyl)amino)propyl)phenoxy)-2-fluoro-N-(thiazol-4-yl)benzenesulfonamide,
1-(3-(2-(4-(N-(1,2,4-thiadiazol-5-yl)sulfamoyl)-2-chloro-5-fluorophenoxy)-5-chlorophenyl)propyl)piperidine-4-carboxylic acid,
2-((3-(5-chloro-2-(2-chloro-5-fluoro-4-(N-(thiazol-2-yl)sulfamoyl)phenoxy)phenyl)propyl)(ethoxycarbonyl)amino)acetic acid,
ethyl 2-((3-(5-chloro-2-(2-chloro-5-fluoro-4-(N-(thiazol-2-yl)sulfamoyl)phenoxy)phenyl)propyl)amino)acetate, and
4-(2-(3-((1H-pyrazol-4-yl)amino)propyl)-4-chlorophenoxy)-5-chloro-2-fluoro-N-(thiazol-2-yl)benzenesulfonamide;
   or a pharmaceutically acceptable salt, or a stereoisomeric or tautomeric form thereof.

In a particular embodiment, the compounds of Formula (Ia) or Formula (I′a) are selected from the group comprising
ethyl 2-((3-(5-chloro-2-(2-chloro-5-fluoro-4-(N-(thiazol-2-yl)sulfamoyl)phenoxy)phenyl)propyl)(methyl)amino)acetate,
2-((3-(5-chloro-2-(2-chloro-5-fluoro-4-(N-(thiazol-2-yl)sulfamoyl)phenoxy)phenyl)propyl)((5-methyl-2-oxo-1,3-dioxol-4-yl)methyl)amino)acetic acid,
2-((3-(5-chloro-2-(2-chloro-5-fluoro-4-(N-(thiazol-2-yl)sulfamoyl)phenoxy)phenyl)propyl)-((1-(isobutyryloxy)ethoxy)carbonyl)amino)acetic acid,
2-((3-(5-chloro-2-(2-chloro-5-fluoro-4-(N-(thiazol-2-yl)sulfamoyl)phenoxy)phenyl)propyl)(((5-methyl-2-oxo-1,3-dioxol-4-yl)methoxy)carbonyl)amino)acetic acid,
5-chloro-4-(4-chloro-2-(3-(3-oxopiperazin-1-yl)propyl)phenoxy)-2-fluoro-N-(thiazol-2-yl)benzenesulfonamide, and
5-chloro-4-(4-chloro-2-(3-((3-morpholino-3-oxopropyl)amino)propyl)phenoxy)-2-fluoro-N-(thiazol-2-yl)benzenesulfonamide;
   or a pharmaceutically acceptable salt, or a stereoisomeric or tautomeric form thereof.

In a particular embodiment, the compounds of Formula (Ia) or Formula (I′a) are selected from the group comprising
3-((3-(5-chloro-2-(2,5-difluoro-4-(N-(thiazol-2-yl)sulfamoyl)phenoxy)phenyl)propyl)amino)propanoic acid,
5-chloro-4-(4-chloro-2-(3-((2-(methylsulfonyl)ethyl)amino)propyl)phenoxy)-2-fluoro-N-(thiazol-4-yl)benzenesulfonamide,
4-(2-(3-((1H-pyrazol-3-yl)amino)propyl)-4-chlorophenoxy)-5-chloro-2-fluoro-N-(thiazol-4-yl)benzenesulfonamide,
2-((3-(5-chloro-2-(2-chloro-5-fluoro-4-(N-(thiazol-4-yl)sulfamoyl)phenoxy)phenyl)propyl)amino)-N-methylacetamide,
2-((3-(5-chloro-2-(2-chloro-5-fluoro-4-(N-(thiazol-4-yl)sulfamoyl)phenoxy)phenyl)propyl)(methyl)amino)acetic acid,
5-chloro-4-(4-chloro-2-(3-(6,7-dihydro-1H-pyrazolo[4,3-c]pyridin-5(4H)-yl)propyl)phenoxy)-2-fluoro-N-(thiazol-4-yl)benzenesulfonamide,
2-((3-(5-chloro-2-(2-chloro-5-fluoro-4-(N-(thiazol-4-yl)sulfamoyl)phenoxy)phenyl)propyl)amino)acetamide,
isopentyl 2-((3-(5-chloro-2-(2-chloro-5-fluoro-4-(N-(thiazol-2-yl)sulfamoyl)phenoxy)phenyl)propyl)amino)acetate,
isopropyl 2-((3-(5-chloro-2-(2-chloro-5-fluoro-4-(N-(thiazol-2-yl)sulfamoyl)phenoxy)phenyl)propyl)amino)acetate,
methyl 2-((3-(5-chloro-2-(2-chloro-5-fluoro-4-(N-(thiazol-4-yl)sulfamoyl)phenoxy)phenyl)propyl)(methyl)amino)acetate,
2-((3-(5-chloro-2-(2-chloro-5-fluoro-4-(N-(thiazol-2-yl)sulfamoyl)phenoxy)phenyl)propyl)((pentyloxy)carbonyl)amino)acetic acid,
2-((3-(5-chloro-2-(2-chloro-5-fluoro-4-(N-(thiazol-2-yl)sulfamoyl)phenoxy)phenyl)propyl)(prop-2-yn-1-yl)amino)acetic acid,
5-chloro-4-(4-chloro-2-(3-(5,6-dihydroimidazo[1,2-a]pyrazin-7(8H)-yl)propyl)phenoxy)-2-fluoro-N-(thiazol-4-yl)benzenesulfonamide,
5-chloro-4-(4-chloro-2-(3-((2-(methylsulfonyl)ethyl)amino)propyl)phenoxy)-2-fluoro-N-(thiazol-2-yl)benzenesulfonamide,
2-((3-(2-(2-chloro-5-fluoro-4-(N-(thiazol-4-yl)sulfamoyl)phenoxy)phenyl)propyl)amino)acetamide,
2-((3-(5-chloro-2-(2-chloro-5-fluoro-4-(N-(thiazol-4-yl)sulfamoyl)phenoxy)phenyl)propyl)(prop-2-yn-1-yl)amino)acetic acid, 2-(allyl(3-(5-chloro-2-(2-chloro-5-fluoro-4-(N-(thiazol-2-yl)sulfamoyl)phenoxy)phenyl)propyl)amino)acetic acid,
2-((3-(5-chloro-2-(2-chloro-5-fluoro-4-(N-(thiazol-2-yl)sulfamoyl)phenoxy)phenyl)propyl)amino)acetamide,
2-(but-2-yn-1-yl(3-(5-chloro-2-(2-chloro-5-fluoro-4-(N-(thiazol-2-yl)sulfamoyl)phenoxy)phenyl)propyl)amino)acetic acid,
2-((3-(5-chloro-2-(2-chloro-5-fluoro-4-(N-(thiazol-2-yl)sulfamoyl)phenoxy)phenyl)propyl)(propyl)amino)acetic acid,
3-((3-(5-chloro-2-(2-chloro-5-fluoro-4-(N-(thiazol-2-yl)sulfamoyl)phenoxy)phenyl)propyl)(prop-2-yn-1-yl)amino)propanoic acid,
2-((3-(5-chloro-2-(2,5-difluoro-4-(N-(thiazol-2-yl)sulfamoyl)phenoxy)phenyl)propyl)(prop-2-yn-1-yl)amino)acetic acid,
ethyl 2-((3-(5-chloro-2-(2-chloro-5-fluoro-4-(N-(thiazol-2-yl)sulfamoyl)phenoxy)phenyl)propyl)(methyl)amino)acetate, and
2-((3-(5-chloro-2-(2,5-difluoro-4-(N-(thiazol-4-yl)sulfamoyl)phenoxy)phenyl)propyl)amino)acetamide;
   or a pharmaceutically acceptable salt, or a stereoisomeric or tautomeric form thereof

Provided herein are compounds of Formula (Ib),

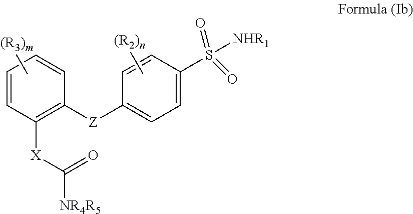

or a pharmaceutically acceptable salt, or a stereoisomeric or tautomeric form thereof, wherein:
Z is —O— or —S—;
X is (C₆-C₁₀)aryl or 5- or 6-membered heteroaryl;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
   R₄ and R₅ are not both H; and
   at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
      R₆ is (C₁-C₁₂)alkyl;
      R₇ and R₈ are each independently H, (C₁-C₁₂)alkyl, or R₇ and R₈ together form a 4- to 7-membered heterocycloalkyl ring; and
m and n are each independently 1, 2, 3, or 4.

In a particular embodiment, the compounds of Formula (Ib) are those wherein R₁ is an aromatic 5- or 6-membered heterocycle, with 1-3 heteroatoms independently selected from the group consisting of N, O, and S.

In a particular embodiment, the compounds of Formula (Ib) are those wherein R₁ is pyridyl or pyrimidinyl.

In a particular embodiment, the compounds of Formula (Ib) are those wherein R₁ is an aromatic 5-membered heterocycle with 1 or 2 nitrogen atoms and optionally 1 or 2 sulphur atoms. In a particular embodiment, the compounds of Formula (Ib) are those wherein R₁ is thiazolyl, isothiazolyl, or thiadiazolyl. In a particular embodiment, the compounds of Formula (Ib) are those wherein R₁ is thiazolyl. In a particular embodiment, the compounds of Formula (Ib) are those wherein R₁ is 1,2,4-thiadiazol-5-yl.

In a particular embodiment, the compounds of Formula (Ib) are those wherein R₂ is independently at each occurrence —F or —Cl.

In a particular embodiment, the compounds of Formula (Ib) are those wherein n is 1, 2, or 3. In a particular embodiment, the compounds of Formula (Ib) are those wherein n is 2.

In a particular embodiment, the compounds of Formula (Ib) are those wherein Z is —O—.

In a particular embodiment, the compounds of Formula (Ib) are those wherein R₃ is independently at each occurrence —H, —F, —Cl, or —Br. In a particular embodiment, the compounds of Formula (Ib) are those wherein R₃ is —H or —Cl. In a particular embodiment, the compounds of Formula (Ib) are those wherein R₃ is —Cl.

In a particular embodiment, the compounds of Formula (Ib) are those wherein m is 1, 2, or 3. In a particular embodiment, the compounds of Formula (Ib) are those wherein m is 1.

In a particular embodiment, the compounds of Formula (Ib) are those wherein X is 5- or 6-membered heteroaryl. In a particular embodiment, the compounds of Formula (Ib) are those wherein X is pyridyl or pyrimidinyl. In a particular embodiment, the compounds of Formula (Ib) are those wherein X is pyridyl.

In a particular embodiment, the compounds of Formula (Ib) are those wherein R₄ is H and R₅ is (C₁-C₉)alkyl.

In a particular embodiment, the compounds of Formula (Ib) are those wherein R₅ is methyl or ethyl, substituted with 1 or 2 substituents selected from the group consisting of —CO₂H, —CO₂R₆, and —CONR₇R₈.

In a particular embodiment, the compounds of Formula (Ib) are those wherein R₆ is (C₁-C₆)alkyl.

In a particular embodiment, the compounds of Formula (Ib) are those wherein R₅ is methyl or ethyl, substituted with —CO₂H.

Provided herein are compounds of Formula (Ic),

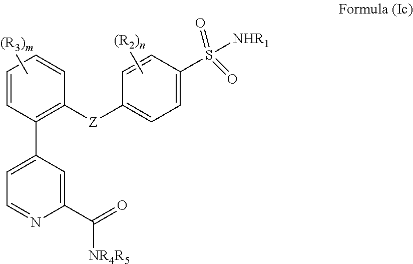

or a pharmaceutically acceptable salt, or a stereoisomeric or tautomeric form thereof, wherein:
Z is —O— or —S—;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
   R₄ and R₅ are not both H; and
   at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
      R₆ is (C₁-C₁₂)alkyl;
      R₇ and R₈ are each independently H, (C₁-C₁₂)alkyl, or R₇ and R₈ together form a 4- to 7-membered heterocycloalkyl ring; and
m and n are each independently 1, 2, 3, or 4.

In a particular embodiment, the compounds of Formula (Ic) are those wherein R₁ is an aromatic 5- or 6-membered heterocycle, with 1-3 heteroatoms independently selected from the group consisting of N, O, and S.

In a particular embodiment, the compounds of Formula (Ic) are those wherein R₁ is pyridyl or pyrimidinyl.

In a particular embodiment, the compounds of Formula (Ic) are those wherein R₁ is an aromatic 5-membered heterocycle with 1 or 2 nitrogen atoms and optionally 1 or 2 sulphur atoms. In a particular embodiment, the compounds of Formula (Ic) are those wherein R₁ is thiazolyl, isothiazolyl, or thiadiazolyl. In a particular embodiment, the compounds of Formula (Ic) are those wherein R₁ is thiazolyl. In a particular embodiment, the compounds of Formula (Ic) are those wherein R₁ is 1,2,4-thiadiazol-5-yl.

In a particular embodiment, the compounds of Formula (Ic) are those wherein R₂ is independently at each occurrence —F or —Cl.

In a particular embodiment, the compounds of Formula (Ic) are those wherein n is 1, 2, or 3. In a particular embodiment, the compounds of Formula (Ic) are those wherein n is 2.

In a particular embodiment, the compounds of Formula (Ic) are those wherein Z is —O—.

In a particular embodiment, the compounds of Formula (Ic) are those wherein R₃ is independently at each occurrence —H, —F, —Cl, or —Br. In a particular embodiment, the compounds of Formula (I) are those wherein R₃ is —H or —Cl. In a particular embodiment, the compounds of Formula (Ic) are those wherein R₃ is —Cl.

In a particular embodiment, the compounds of Formula (Ic) are those wherein m is 1, 2, or 3. In a particular embodiment, the compounds of Formula (Ic) are those wherein m is 1.

In a particular embodiment, the compounds of Formula (Ic) are those wherein X is 5- or 6-membered heteroaryl. In a particular embodiment, the compounds of Formula (Ic) are those wherein X is pyridyl or pyrimidinyl. In a particular embodiment, the compounds of Formula (Ic) are those wherein X is pyridyl.

In a particular embodiment, the compounds of Formula (Ic) are those wherein R₄ is H and R₅ is (C₁-C₉)alkyl.

In a particular embodiment, the compounds of Formula (Ic) are those wherein R₅ is methyl or ethyl, substituted with 1 or 2 substituents selected from the group consisting of —CO₂H, —CO₂R₆, and —CONR₇R₈.

In a particular embodiment, the compounds of Formula (Ic) are those wherein R₆ is (C₁-C₆)alkyl.

In a particular embodiment, the compounds of Formula (Ic) are those wherein R₅ is methyl or ethyl, substituted with —CO₂H.

In a particular embodiment, the compounds of Formula (Ic) are selected from the group consisting of
3-(4-(2-(4-(N-(1,2,4-thiadiazol-5-yl)sulfamoyl)-2-chloro-5-fluorophenoxy)-5-chlorophenyl)picolinamido)propanoic acid,
2-(4-(2-(4-(N-(1,2,4-thiadiazol-5-yl)sulfamoyl)-2-chloro-5-fluorophenoxy)-5-chlorophenyl)picolinamido)acetic acid,
5-(4-(2-(4-(N-(1,2,4-thiadiazol-5-yl)sulfamoyl)-2-chloro-5-fluorophenoxy)-5-chlorophenyl)picolinamido)pentanoic acid,
4-(4-(2-(4-(N-(1,2,4-thiadiazol-5-yl)sulfamoyl)-2-chloro-5-fluorophenoxy)-5-chlorophenyl)picolinamido)butanoic acid,
2-(4-(2-(4-(N-(1,2,4-thiadiazol-5-yl)sulfamoyl)-2-chloro-5-fluorophenoxy)-5-chlorophenyl)picolinamido)propanoic acid,
(R)-2-(4-(2-(4-(N-(1,2,4-thiadiazol-5-yl)sulfamoyl)-2-chloro-5-fluorophenoxy)-5-chlorophenyl)picolinamido)propanoic acid,
(S)-2-(4-(2-(4-(N-(1,2,4-thiadiazol-5-yl)sulfamoyl)-2-chloro-5-fluorophenoxy)-5-chlorophenyl)picolinamido)propanoic acid,
3-(4-(2-(4-(N-(1,2,4-thiadiazol-5-yl)sulfamoyl)-2-cyanophenoxy)-5-chlorophenyl)picolinamido)propanoic acid, and
3-(4-(2-(4-(N-(1,2,4-thiadiazol-5-yl)sulfamoyl)-2,5-difluorophenoxy)-5-chlorophenyl)picolinamido)propanoic acid; or
   a pharmaceutically acceptable salt, or a stereoisomeric or tautomeric form thereof.

Provided herein are compounds of Formula (Id),

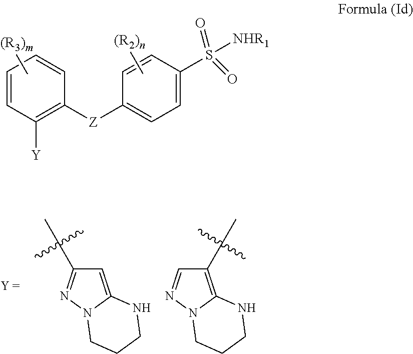

or a pharmaceutically acceptable salt, or a stereoisomeric or tautomeric form thereof, wherein:
Y is 4,5,6,7-tetrahydropyrazolo[1,5-a]pyrimidine-(2-yl or 3-yl);
Z is —O— or —S—;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy; and
m and n are each independently 1, 2, 3, or 4.

In a certain embodiment, the compounds of Formula (Id) are those wherein Y is 4,5,6,7-tetrahydropyrazolo[1,5-a]pyrimidine-(2-yl or 3-yl). In a particular embodiment, the compounds of Formula (Id) are those wherein Y is 4,5,6,7-tetrahydropyrazolo[1,5-a]pyrimidine-3-yl.

In a particular embodiment, the compounds of Formula (Id) are those wherein R₁ is an aromatic 5- or 6-membered heterocycle, with 1-3 heteroatoms independently selected from the group consisting of N, O, and S.

In a particular embodiment, the compounds of Formula (Id) are those wherein R₁ is pyridyl or pyrimidinyl.

In a particular embodiment, the compounds of Formula (Id) are those wherein R₁ is an aromatic 5-membered heterocycle with 1 or 2 nitrogen atoms and optionally 1 or 2 sulphur atoms. In a particular embodiment, the compounds of Formula (Id) are those wherein R₁ is thiazolyl, isothiazolyl, or thiadiazolyl. In a particular embodiment, the compounds of Formula (Id) are those wherein R₁ is thiazolyl. In a particular embodiment, the compounds of Formula (Id) are those wherein R₁ is 1,2,4-thiadiazol-5-yl.

In a particular embodiment, the compounds of Formula (Id) are those wherein R₂ is independently at each occurrence —F or —Cl.

In a particular embodiment, the compounds of Formula (Id) are those wherein n is 1, 2, or 3. In a particular embodiment, the compounds of Formula (Id) are those wherein n is 2.

In a particular embodiment, the compounds of Formula (Id) are those wherein Z is —O—.

In a particular embodiment, the compounds of Formula (Id) are those wherein R₃ is independently at each occurrence —H, —F, —Cl, or —Br. In a particular embodiment, the compounds of Formula (Id) are those wherein R₃ is —H or —Cl. In a particular embodiment, the compounds of Formula (Id) are those wherein R₃ is —Cl.

In a particular embodiment, the compounds of Formula (Id) are those wherein m is 1, 2, or 3. In a particular embodiment, the compounds of Formula (Id) are those wherein m is 1.

In a particular embodiment, the compound of Formula (Id) is
5-chloro-4-(4-chloro-2-(4,5,6,7-tetrahydropyrazolo[1,5-a]pyrimidin-3-yl)phenoxy)-2-fluoro-N-(thiazol-4-yl)benzenesulfonamide, or a pharmaceutically acceptable salt, or a stereoisomeric or tautomeric form thereof.

In a particular embodiment, the compound of Formula (Id) is
5-chloro-2-fluoro-4-(2-(4,5,6,7-tetrahydropyrazolo[1,5-a]pyrimidin-3-yl)phenoxy)-N-(thiazol-2-yl)benzenesulfonamide,
5-chloro-4-(4-chloro-2-(4,5,6,7-tetrahydropyrazolo[1,5-a]pyrimidin-3-yl)phenoxy)-2-fluoro-N-(thiazol-2-yl)benzenesulfonamide, or
5-chloro-2-fluoro-4-(2-(4,5,6,7-tetrahydropyrazolo[1,5-a]pyrimidin-3-yl)phenoxy)-N-(thiazol-4-yl)benzenesulfonamide;
   or a pharmaceutically acceptable salt, or a stereoisomeric or tautomeric form thereof.

It should also be noted the Compounds provided herein can contain unnatural proportions of atomic isotopes at one or more of the atoms. For example, the Compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched Compound. The term “isotopically enriched” refers to an atom having an isotopic composition other than the natural isotopic composition of that atom. “Isotopically enriched” may also refer to a Compound containing at least one atom having an isotopic composition other than the natural isotopic composition of that atom. The term “isotopic composition” refers to the amount of each isotope present for a given atom. Radiolabeled and isotopically enriched Compounds are useful as therapeutic agents, e.g., cancer and inflammation therapeutic agents; research reagents, e.g., binding assay reagents; and diagnostic agents, e.g., in vivo imaging agents. All isotopic variations of the Compounds as described herein, whether radioactive or not, are intended to be encompassed within the scope of the embodiments provided herein. In some embodiments, there are provided isotopologues of the Compounds, for example, the isotopologues are deuterium, carbon-13, or nitrogen-15 enriched Compounds.

In certain embodiments, a Compound provided herein inhibits the activity of a sodium ion channel, such as a voltage-gated sodium ion channel. In more specific embodiments, such a voltage-gated sodium ion channel is NaV1.7 (whose alpha subunit is encoded by the human gene SCN9A).

In certain embodiments, a Compound provided herein reduces the sodium ion flux through NaV1.7 by at least 10%, 20%, 30%, 40%, 50%, 60%, 70%, 80%, 90%, 95%, 98%, 99%, or 100%, or by ranges between any of the recited percentages (e.g., 10-20%, 10-30%, 10-40%, 20-30%, or 20-40%) relative to the activated channel in the absence of the Compound.

In certain embodiments, a Compound provided herein, desensitizes the response of NaV1.7 to the change in membrane potential such that the channel requires at least 10%, 20%, 30%, 40%, 50%, 60%, 70%, 80%, 90%, or 100%, or ranges between any of the recited percentages (e.g., 10-20%, 10-30%, 10-40%, 20-30%, or 20-40%) higher change in membrane potential to be activated relative to the channel in the absence of the Compound.

In certain embodiments, a Compound provided herein, affects a voltage-gated sodium ion channel, e.g., NaV1.7, in one or more of the following states: resting (closed), activated (open), or inactivated (closed). In certain embodiments, a Compound provided herein, affects activation, inactivation, or deinactivation of a voltage-gated sodium ion channel, e.g., NaV1.7.

In certain embodiments, a Compound provided herein, inhibits NaV1.7 specifically, i.e., the compound inhibits NaV1.7 to at least 10%, 20%, 30%, 40%, 50%, 60%, 70%, 80%, 90%, 100%, 250%, 500%, 750%, or 1000% higher degree than another voltage-gated sodium ion channel (such as NaV1.1, NaV1.2, NaV1.3, NaV1.4, NaV1.5, NaV1.6, NaV1.8, and/or NaV1.9), or to a higher degree between any of the recited percentages (e.g., 10-20%, 10-30%, 10-40%, 20-30%, or 20-40%) than another voltage-gated sodium channel. In certain embodiments, a Compound provided herein, inhibits NaV1.7 specifically, i.e., the compound inhibits NaV1.7 to at least 10%, 20%, 30%, 40%, 50%, 60%, 70%, 80%, 90%, 100%, 250%, 500%, 750%, or 1000% higher degree than one or more voltage-gated sodium ion channel selected from NaV1.1, NaV1.2, NaV1.3, NaV1.4, NaV1.5, NaV1.6, NaV1.8, and NaV1.9, or to a higher degree between any of the recited percentages (e.g., 10-20%, 10-30%, 10-40%, 20-30%, or 20-40%) than one ore more of NaV1.1, NaV1.2, NaV1.3, NaV1.4, NaV1.5, NaV1.6, NaV1.8, and NaV1.9.

In certain embodiments, a Compound provided herein binds to NaV1.7 with at least 5-fold, 10-fold, 50-fold, 100-fold, 500-fold, or 1000-fold higher affinity than it binds to either one of or all of NaV1.1, NaV1.2, NaV1.3, NaV1.4, NaV1.5, NaV1.6, NaV1.8, and NaV1.9. In certain embodiments, a Compound provided herein binds to NaV1.7 with at least 5-fold, 10-fold, 50-fold, 100-fold, 500-fold, or 1000-fold higher affinity than it binds to one or more sodium channels selected from NaV1.1, NaV1.2, NaV1.3, NaV1.4, NaV1.5, NaV1.6, NaV1.8, and NaV1.9.

In certain embodiments, a Compound provided herein binds to the inactivated (closed) state of NaV1.7 with at least 5-fold, 10-fold, 50-fold, 100-fold, 500-fold, or 1000-fold higher affinity than to any other state of NaV1.7, i.e., deactivated (closed) and activated (open).

In certain embodiments, a Compound provided herein binds to NaV1.7 with at least 5-fold, 10-fold, 50-fold, 100-fold, 500-fold, or 1000-fold higher affinity than it binds to one or more sodium channels selected from NaV1.1, NaV1.2, NaV1.3, NaV1.4, NaV1.5, NaV1.6, NaV1.8, and NaV1.9.

In one embodiment, Compound provided herein has an IC50 for NaV1.1, NaV1.2, NaV1.3, NaV 1.4, NaV1.5, NaV 1.6, NaV 1.8, and NaV1.9, that is each independently at least 10 fold, 20 fold, 50 fold, 100 fold, 200 fold, 500 fold, 1000 fold, 2000 fold, 5000 fold, or 10000 fold higher than the NaV 1.7 IC50 for said Compound. In one embodiment, a Compound provided herein has an IC50 for one or more of NaV1.1, NaV1.2, NaV1.3, NaV 1.4, NaV1.5, NaV 1.6, NaV 1.8, and NaV1.9, that is each independently at least 10 fold, 20 fold, 50 fold, 100 fold, 200 fold, 500 fold, 1000 fold, 2000 fold, 5000 fold, or 10000 fold higher than the NaV 1.7 IC50 for said Compound. In one embodiment, the compound has a NaV1.3 IC50 of at least at least 10 fold, 20 fold, 50 fold, 100 fold, 200 fold, 500 fold, 1000 fold, 2000 fold, 5000 fold, or 10000 fold higher than the NaV 1.7 IC50 for said compound.

In one embodiment, the IC50 is measured using an FDSS membrane potential assay or the patch-clamp method.

Any assay known to the skilled artisan can be used to test the effect of a compound provided herein on a voltage-gated sodium ion channel. A wide variety of assay methods are known in the art to profile Compounds provided herein against human sodium channels stably expressed in human embryonic kidney (HEK293) cells. Such assays are disclosed, for example, in WO2007/109324 to Fraser et al., which is incorporated herewith in its entirety. In particular, such assays are disclosed in Example 3, pages 94-99 of WO2007/109324, which is herewith incorporated in its entirety.

In certain embodiments, a cell culture assay is used, wherein the voltage-gated sodium ion channel is recombinantly expressed in the cultured cells. In certain more specific embodiments, the alpha subunit of the voltage-gated sodium ion channel is expressed but no accessory proteins are recombinantly expressed in the same cell. In a specific embodiment, SCN9A and SCN9B1 and SCN9B2 are co-expressed in the same cell. In other embodiments, the alpha subunit of the voltage-gated sodium ion channel is expressed and at least one accessory protein (e.g., a beta-subunit) is co-expressed in the same cell.

In certain embodiments, an FDSS membrane potential assay can be used to test the activity of the voltage-gated sodium ion channel (see the Section entitled “FDSS Membrane Potential In-Vitro Assay” below). In other embodiments, the current through a voltage-gated sodium ion channel is tested using the patch clamp method (see the Section entitled “Patchliner Electrophysiological In-Vitro Assay” below)

### 4.4 Methods for Making Compounds

A compound of Formula (Ia) or a compound of Formula (I′a) can be synthesized according to synthetic Scheme 1. An R₃ substituted 2-hydroxybenzaldehyde or 2-mercaptobenzaldehyde is reacted under Horner-Wadsworth-Emmons (“HWE”) conditions with formylmethylene-triphenylphosphorane to give an α,β-unsaturated aldehyde, Intermediate A. Intermediate A is reacted with HNR₉R₁₀ under reductive amination conditions using, for example, sodium borohydride, to give Intermediate B. Intermediate B is then reduced to give Intermediate C using, for example, hydrogen in the presence of metal catalyst, such as palladium on carbon. Intermediate C is reacted with a fluoro-substituted phenylsulfonamide, wherein the sulfonamide nitrogen is optionally protected by a group (“PG”), such as tert-butoxycarbonyl (“BOC”) or 2,4-dimethoxybenzyl, in presence of a base, such as potassium carbonate, to give Intermediate D. Deprotection of the sulfonamide group of Intermediate D by using, for example, hydrochloric acid, gives a compound of Formula (Ia) or a compound of Formula (I′a).

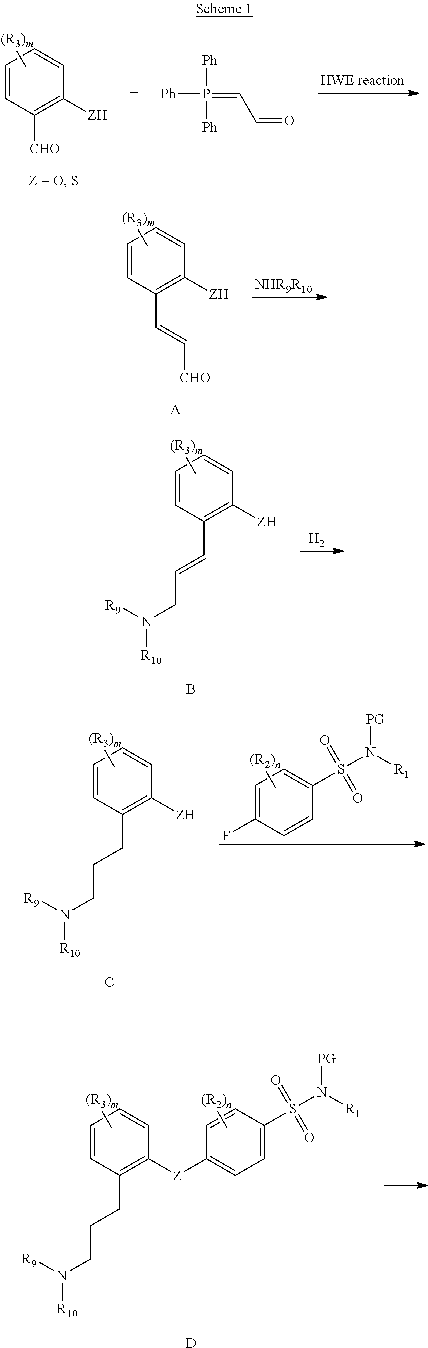

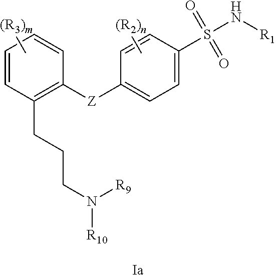

A compound of Formula (Ib) can be prepared according to synthetic Scheme 2. A Suzuki coupling between an R₃ substituted 2-hydroxy-boronic acid or 2-mercapto-boronic acid and derivative of X, wherein X is, for example, a (C₆-C₁₀)aryl or 5- or 6-membered heteroaryl, such as a 4-halo-picolinonitrile or a 4-halo-picolinic ester (e.g., a methyl picolinate), wherein the halo substituent is, for example, a chloro or bromo substituent, provides Intermediate E. Intermediate E is reacted with a base, such as potassium hydroxide, to give Intermediate F. Intermediate F is reacted with NHR₄R₅ to form the amide Intermediate G using, for example, 1-ethyl-3-(3-dimethylaminopropyl)carbodiimide (“EDC”) and 1-hydroxy-1H-benzotriazole (“HOBt”). Intermediate G is reacted with a fluoro-substituted phenylsulfonamide, wherein the sulfonamide nitrogen is optionally protected by a group, such as BOC or 2,4-dimethoxybenzyl, in presence of a base, such as potassium carbonate, to give Intermediate H. Deprotection of the sulfonamide group of Intermediate H by using, for example, hydrochloric acid, gives a compound of Formula (Ib).

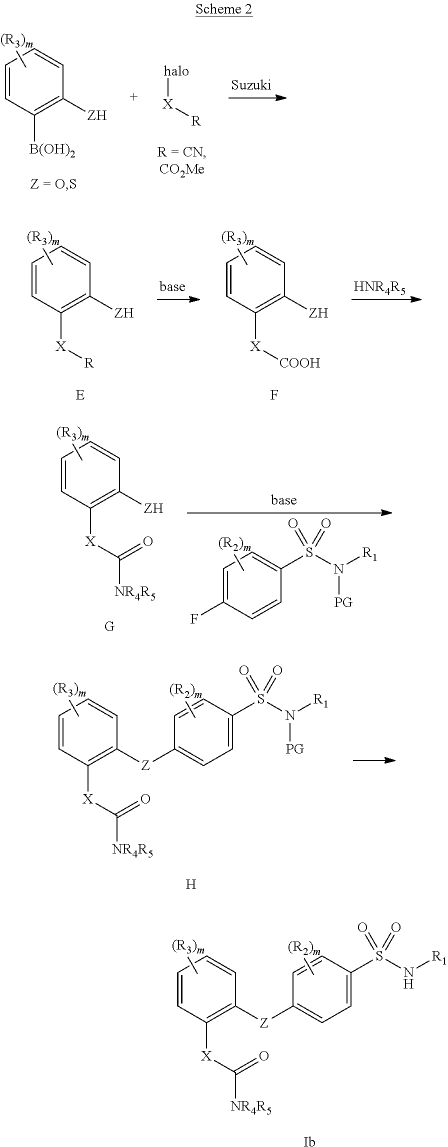

A compound of Formula (Ic) can be prepared according to synthetic Scheme 3. A Suzuki coupling between an R₃ substituted 2-hydroxy-boronic acid or 2-mercapto-boronic acid and pyridine derivative, such as a 4-halo-picolinonitrile or a 4-halo-picolinic ester (e.g., a methyl picolinate), wherein the halo substituent is, for example, a chloro or bromo substituent, provides Intermediate I. Intermediate I is reacted with a base, such as potassium hydroxide, to give Intermediate J. Intermediate J is reacted with NHR₄R₅ to form the amide Intermediate K using, for example, EDC and HOBt. Intermediate K is reacted with a fluoro-substituted phenylsulfonamide, wherein the sulfonamide nitrogen is optionally protected by a group, such as BOC or 2,4-dimethoxybenzyl, in presence of a base, such as potassium carbonate, to give Intermediate L. Deprotection of the sulfonamide group of Intermediate L by using, for example, hydrochloric acid, gives a compound of Formula (Ic).

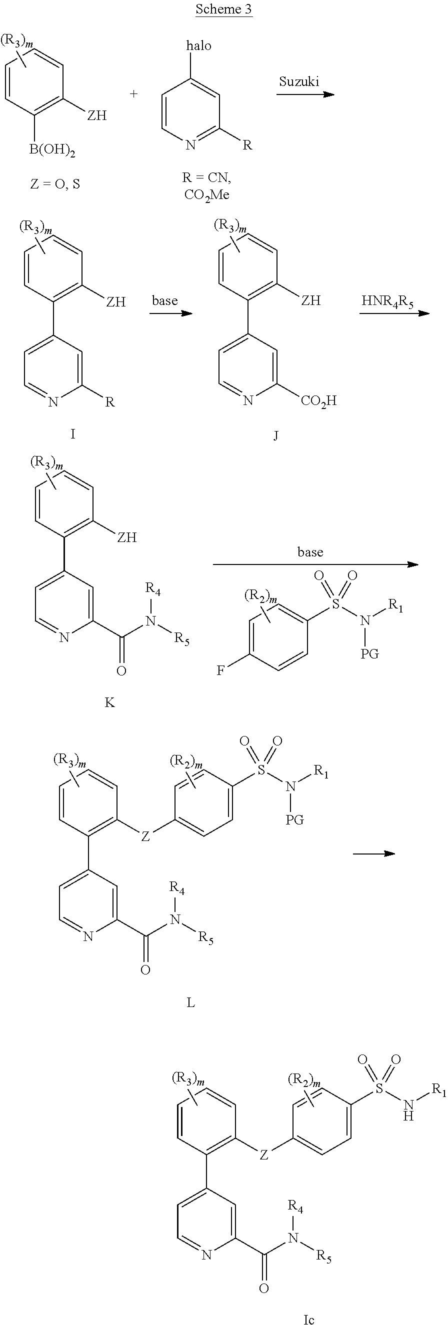

A compound of Formula (Id) can be prepared according to synthetic Scheme 4. Phenylacetonitrile derivative M with a protected hydroxy or thiol group, such as a methyl protected hydroxy group, i.e., a —OMe group, is formylated by using, for example, Na/ethyl formate or NaOEt/ethyl formate to give Intermediate N. Intermediate N is reacted with hydrazine to provide Intermediate O. Intermediate O is reacted with dihaloalkanes, such as 1,3-dibromopropane, under basic conditions, for example, in presence of NaH or Cs₂CO₃, to give Intermediate P. Intermediate P, after deprotection of the phenol or thiol, for example, by reacting a methyl protected hydroxy group with BBr₃, can undergo same synthetic sequence as described Scheme 1, Scheme 2, or Scheme 3 to give compound S, which is a compound of Formula (Id). Furthermore, Intermediate W, which is deprotected and subjected to the procedures described and referred to in this paragraph to give compounds of Formula (Id), can be obtained as follows: Intermediate T is reacted under Suzuki conditions in presence of a base and a palladium catalyst with Intermediate U or U′, wherein R of Intermediate U or U′ is a nitro group or a suitably protected amino group, to give Intermediate V. Intermediate V is subjected to conditions, which reduce the nitro group to an amino group or deprotect the nitrogen to release an amino group, such as zinc in acetic acid or hydrogen and Raney-Nickel, to give Intermediate W.

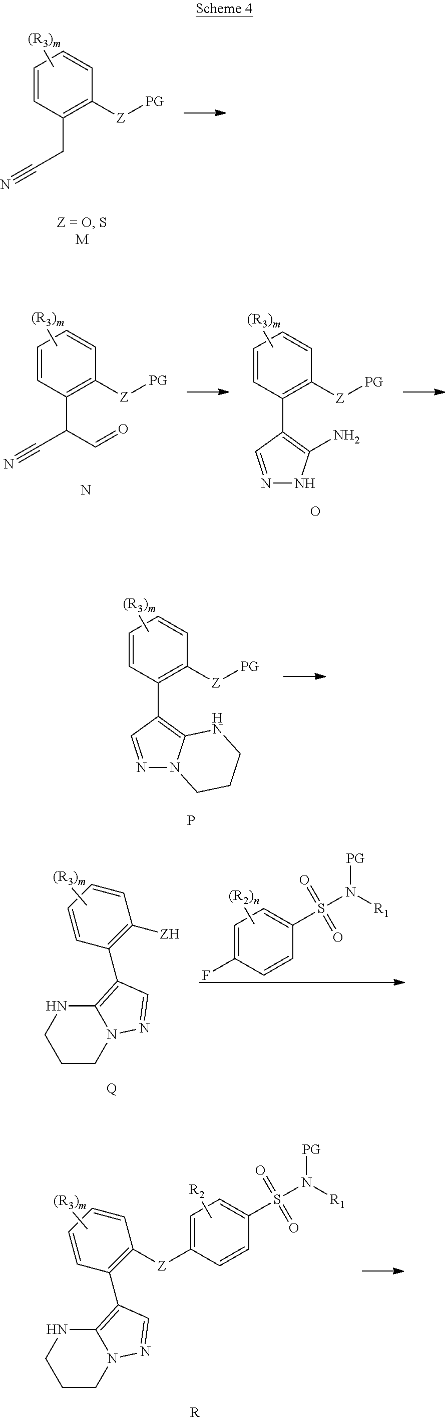

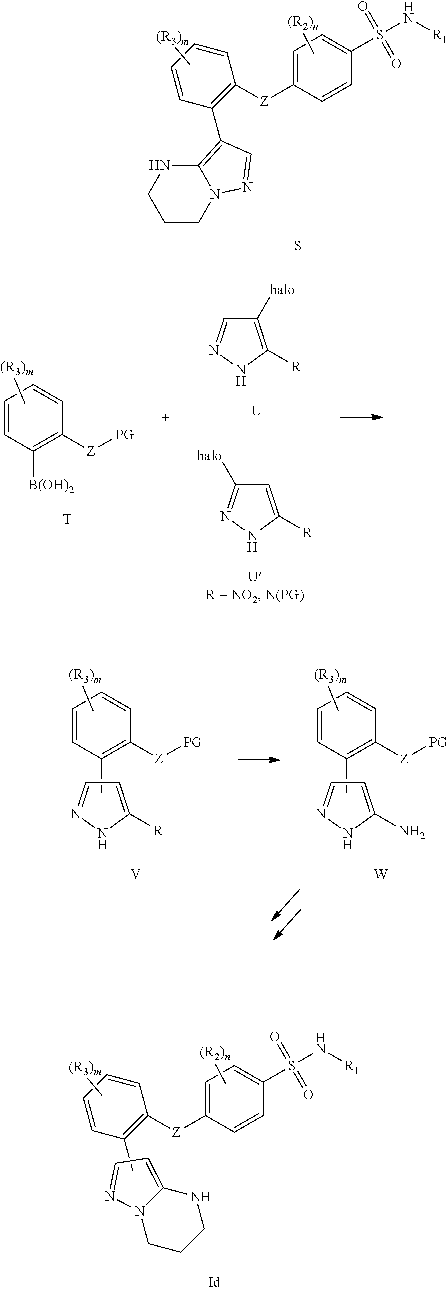

### 4.5 Methods of Use

First, provided herein are methods for treating or preventing prediabetes comprising administering to a subject in need thereof a therapeutically effective amount of a compound selectively inhibiting NaV1.7.

Second, provided herein are methods for treating or preventing diabetes comprising administering to a subject in need thereof a therapeutically effective amount of a compound selectively inhibiting NaV1.7.

Third, provided herein are methods for maintaining or lowering blood or plasma glucose levels in a subject in need thereof comprising administering to the subject, a therapeutically effective amount of a compound selectively inhibiting NaV1.7.

Fourth, provided herein are methods for maintaining or lowering blood or plasma glycated hemoglobin levels in a subject in need thereof comprising administering to the subject, a therapeutically effective amount of a compound selectively inhibiting NaV1.7.

In one embodiment the compound selectively inhibiting NaV1.7 used in the methods disclosed is a Compound provided herein (i.e., a compound of Formula (I), a compound of Formula (I′), a compound of Formula (Ia), a compound of Formula (I′a), a compound of Formula (Ib), a compound of Formula (Ic), a compound of Formula (Id), a compound listed in Table 1, Table 2, or Table 3, or a compound described in Section 4.3), or a pharmaceutically acceptable salt, solvate or tautomeric form thereof. In one embodiment, the compound selectively inhibiting NaV1.7 used in the methods disclosed is not a compound of Formula (I), a compound of Formula (I′), a compound of Formula (Ia), a compound of Formula (I′a), a compound of Formula (Ib), a compound of Formula (Ic), a compound of Formula (Id), or a compound listed in Table 1, Table 2, or Table 3. In one embodiment, the compound selectively inhibiting NaV1.7 used in the methods disclosed is not compound 49.

In one embodiment, the subject has prediabetes.

In another embodiment, the subject has diabetes. In certain embodiments, diabetes is gestational diabetes, type-1 diabetes, type-2 diabetes, or latent autoimmune diabetes of adults. In one embodiment, diabetes is gestational diabetes. In one embodiment, diabetes is type-1 diabetes. In one embodiment, diabetes is type-2 diabetes. In one embodiment, type-2 diabetes is hyperinsulinemic Type 2 diabetes. In one embodiment, wherein diabetes is latent autoimmune diabetes of adults.

Blood or plasma glucose may be determined by any method known in the art, such as a commercially available blood glucose meter, a lancet device with lancets, or commercially available test strips.

Blood or plasma glycated hemoglobin may be determined by any method known in the art, such as the A1C test using, for example, the methods provided by the NGSP (“National Glycohemoglobin Standardization Program”). See http://www.ngsp.org/index.asp (last accessed Aug. 27, 2014) for further details.

In one embodiment, the methods of treating prediabetes or treating diabetes or lowering blood or plasma glucose lower the blood or plasma glucose in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the blood or plasma glucose prior to the administration of a Compound provided herein. In one embodiment, the blood or plasma glucose continues to lower or remains at a reduced level relative to the blood or plasma glucose prior to the administration of a Compound provided herein after administration of the Compound has stopped. In a specific embodiment, the blood or plasma glycated hemoglobin continues to lower or remains at the reduced level for at least about 5 days, 10 days, 15 days, 20 days, 1 month, 3 months, 6 months, 1 year, 2 years, 3 years, or 5 years, after an administration period of at least about 1 day, 5 days, 10 days, 15 days, 20 days, 1 month, 3 months, 6 months, or 1 year.

In one embodiment, the methods of treating prediabetes or treating diabetes or lowering blood or plasma glycated hemoglobin lower the blood or plasma glycated hemoglobin in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the blood or plasma glycated hemoglobin prior to the administration of a Compound provided herein. In one embodiment, the blood or plasma glycated hemoglobin continues to lower or remains at a reduced level relative to the blood or plasma glycated hemoglobin prior to the administration of a Compound provided herein after administration of the Compound has stopped. In a specific embodiment, the blood or plasma glycated hemoglobin continues to lower or remains at the reduced level for at least about 5 days, 10 days, 15 days, 20 days, 1 month, 3 months, 6 months, 1 year, 2 years, 3 years, or 5 years, after an administration period of at least about 1 day, 5 days, 10 days, 15 days, 20 days, 1 month, 3 months, 6 months, or 1 year.

The diagnosis and classification of diabetes mellitus is described by the American Diabetes Association in Diabetes Care 37, Supplement 1, S67-S90 (2014) (“ADA 2014”). Erratum to “Diagnosis and Classification of Diabetes Mellitus,” Diabetes Care 37, Supplement 1: S81-S90 (2014) published in Diabetes Care 37, 887 (2014).

### 4.5.1.1 Diagnosis of Diabetes

In one embodiment, a subject is in need of: treatment for diabetes; or maintaining or lowering blood or plasma glucose; or maintaining or lowering blood or plasma glycated hemoglobin, if the subject shows:

| | |
|---|---|
| (1) | A1C equal or greater than about 6.5%. The test should be performed |
| | in a laboratory using a method that is NGSP (“National Glyco- |
| | hemoglobin Standardization Program”) certified and standardized |
| | to the DCCT (“Diabetes Control and Complications Trial”)assay.* |

OR

| | |
|---|---|
| (2) | FPG (“fasting plasma glucose”) equal or greater than about 126 |
| | mg/dL (7.0 mmol/L). Fasting is defined as no substantial caloric |
| | intake for about at least 8 hours.* |

OR

| | |
|---|---|
| (3) | Two-hour plasma glucose equal or greater than about 200 mg/dL |
| | (11.1 mmol/L) during an OGTT (“oral glucose tolerance test”). The |
| | test should be performed as described by the World Health |
| | Organization, using a glucose load containing the equivalent |
| | of about 75 g anhydrous glucose dissolved in water.* |

OR

| | |
|---|---|
| (4) | In a patient with classic symptoms of hyperglycemia or hyper- |
| | glycemic crisis, a random plasma glucose equal or greater than |
| | about 200 mg/dL (11.1 mmol/L). |

*In the absence of unequivocal hyperglycemia, criteria 1-3 should be confirmed by repeat testing.

A1C (also known as, inter alia, hemoglobin A1c, HbA1c, glycohemoglobin, glycated hemoglobin, or glycosylated hemoglobin) is a widely used marker of chronic glycemia, reflecting average blood glucose levels over a 2- to 3-month period of time. The test plays a critical role in the management of subjects with diabetes, since it correlates well with both microvascular and, to a lesser extent, macrovascular complications and is widely used as the standard biomarker for the adequacy of glycemic management. ADA 2014 at S87, left column.

FPG test checks the subject's fasting blood glucose levels. Fasting means not eating or drinking (except water) for at least 8 hours before the test. In one embodiment, the FPG test is run in the morning, before the subject had breakfast.

In the OGTT, which is the most commonly performed version of the glucose tolerance test, a standard dose of glucose is orally administered to a subject and blood samples taken afterward (about 2 hours later) to determine how quickly glucose is cleared from the blood. A random plasma glucose test is a measure of how much glucose a subject has circulating in the blood. “Random” means that the subject has blood drawn at any time. Whether the subject has fasted or recently eaten will not affect the test.

Further, in connection with Item (4) in paragraph [00204] above, the symptoms of hyperglycemia or hyperglycemic crisis include, but are not limited to: frequent urination, increased thirst, blurred vision, fatigue, headache, fruity-smelling breath, nausea and vomiting, shortness of breath, dry mouth, weakness, confusion, coma, and abdominal pain.

In one embodiment, the methods of treating diabetes, or maintaining or lowering blood or plasma glucose, or maintaining or lowering blood or plasma glycated hemoglobin, maintain the A1C level in a subject in need thereof or lower the A1C level in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the A1C level prior to the administration of a Compound provided herein. In a particular embodiment, the methods of treating diabetes, or lowering blood or plasma glucose, or lowering blood or plasma glycated hemoglobin lower the A1C level to at least about 14%, 13%, 12%, 11%, 10%, 9%, 8%, 7%, 6.5%, 6.2%, 6.0%, 5.7%, 5.5%, 5.2%, 5.0%, 4.7%, 4.5%, 4.2%, 4.0%, 3.7%, 3.5%, 3.2%, or 3.0%, or to at least a range formed by any two of the foregoing percentages, for example, to at least about 4.5% to about 6%, or to at least about 5.7% to 6.4%.

In one embodiment, the methods of treating diabetes, or maintaining or lowering blood or plasma glucose, or maintaining or lowering blood or plasma glycated hemoglobin maintain the FPG level in a subject in need thereof or lower the FPG level in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the FPG level prior to the administration of a Compound provided herein. In a particular embodiment, the methods of treating diabetes, or lowering blood or plasma glucose, or lowering blood or plasma glycated hemoglobin lower the FPG level to at least about 150 mg/dL, 145 mg/dL, 140 mg/dL, 135 mg/dL, 130 mg/dL, 126 mg/dL, 125 mg/dL, 120 mg/dL, 115 mg/dL, 110 mg/dL, 105 mg/dL, 100 mg/dL, 99 mg/dL, 95 mg/dL, 90 mg/dL, 85 mg/dL, 80 mg/dL, 75 mg/dL, 70 mg/dL, or 60 mg/dL, or to at least a range formed by any two of the foregoing percentages, for example, to at least about 70 mg/dL to about 99 mg/dL, or to at least about 100 mg/dL to about 125 mg/dL.

In one embodiment, the methods of treating diabetes, or maintaining or lowering blood or plasma glucose, or maintaining or lowering blood or plasma glycated hemoglobin maintain the two-hour plasma glucose during an OGTT in a subject in need thereof or lower the two-hour plasma glucose during an OGTT in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the two-hour plasma glucose during an OGTT prior to the administration of a Compound provided herein. In a particular embodiment, the methods of treating diabetes or lowering blood or plasma glucose or lowering blood or plasma glycated hemoglobin lower the two-hour plasma glucose during an OGTT using a glucose load of, for example, 75 g of anhydrous glucose dissolved in water, to at least about 300 mg/dL, 270 mg/dL, 250 mg/dL, 220 mg/dL, 200 mg/dL, 199 mg/dL, 190 mg/dL, 180 mg/dL, 170 mg/dL, 160 mg/dL, 150 mg/dL, 140 mg/dL, 139 mg/dL, 130 mg/dL, 120 mg/dL, 110 mg/dL, or 100 mg/dL, or to at least a range formed by any two of the foregoing percentages, for example, to at least about 199 mg/dL to about 140 mg/dL.

In one embodiment, the methods of treating diabetes, or lowering blood or plasma glucose, or lowering blood or plasma glycated hemoglobin lower the A1C level or the FPG level or the A1C, or any combination thereof, in a subject in need thereof such that the subject is no longer diagnosed as having diabetes in view of the criteria discussed in this Section.

### 4.5.1.2 Diagnosis of Prediabetes

In one embodiment, a subject is in need of: treatment for prediabetes; or maintaining or lowering blood or plasma glucose; or maintaining or lowering blood or plasma glycated hemoglobin, if the subject shows:

| | |
|---|---|
| (1) | FPG (“fasting plasma glucose”) of about 100 mg/dL (5.6 mmol/L) |
| | to about 125 mg/dL (6.9 mmol/L) (IFG (“impaired fasting glucose”) |

OR

| | |
|---|---|
| (2) | 2-h PG (“plasma glucose”) in the 75-g OGTT (“oral glucose |
| | tolerance test”) of about 140 mg/dL (7.8 mmol/L) to about 199 |
| | mg/dL (11.0 mmol/L) (IGT (“impaired glucose tolerance”) |

OR

| | |
|---|---|
| (3) | A1C of about 5.7 to about 6.4% |

For all three tests, risk is continuous, extending below the lower limit of the range and becoming disproportionately greater at higher ends of the range.

FPG test checks the subject's fasting blood glucose levels. Fasting means not eating or drinking (except water) for at least 8 hours before the test. In the OGTT, which is the most commonly performed version of the glucose tolerance test, a standard dose of glucose is orally administered to a subject and blood samples taken afterward (about 2 hours later) to determine how quickly glucose is cleared from the blood. A1C (also known as, inter alia, hemoglobin A1c, HbA1c, glycohemoglobin, glycated hemoglobin, or glycosylated hemoglobin) is a widely used marker of chronic glycemia, reflecting average blood glucose levels over a 2- to 3-month period of time. ADA 2014 at S87, left column.

In one embodiment, the methods of treating prediabetes, or maintaining or lowering blood or plasma glucose, or maintaining or lowering blood or plasma glycated hemoglobin maintain the FPG level in a subject in need thereof or lower the FPG level in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the FPG level prior to the administration of a Compound provided herein. In a particular embodiment, the methods of treating prediabetes or lowering blood or plasma glucose or lowering blood or plasma glycated hemoglobin lower the FPG level to at least about 125 mg/dL, 120 mg/dL, 115 mg/dL, 110 mg/dL, 105 mg/dL, 100 mg/dL, 99 mg/dL, 95 mg/dL, 90 mg/dL, 85 mg/dL, 80 mg/dL, 75 mg/dL, 70 mg/dL, or 60 mg/dL, or to at least a range formed by any two of the foregoing percentages, for example, to at least about 99 mg/dL to about 70 mg/dL.

In one embodiment, the methods of treating prediabetes, or maintaining or lowering blood or plasma glucose, or maintaining or lowering blood or plasma glycated hemoglobin maintain the two-hour plasma glucose during an OGTT in a subject in need thereof or lower the two-hour plasma glucose during an OGTT in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the two-hour plasma glucose during an OGTT prior to the administration of a Compound provided herein. In a particular embodiment, the methods of treating prediabetes or lowering blood or plasma glucose or lowering blood or plasma glycated hemoglobin lower the two-hour plasma glucose during an OGTT using a glucose load of, for example, 75 g of anhydrous glucose dissolved in water, to at least about 199 mg/dL, 190 mg/dL, 180 mg/dL, 170 mg/dL, 160 mg/dL, 150 mg/dL, 140 mg/dL, 139 mg/dL, 130 mg/dL, 120 mg/dL, 110 mg/dL, or 100 mg/dL, or to at least a range formed by any two of the foregoing percentages, for example, to at least about 139 mg/dL to about 100 mg/dL.

In one embodiment, the methods of treating prediabetes, or maintaining or lowering blood or plasma glucose, or maintaining or lowering blood or plasma glycated hemoglobin maintain the A1C level in a subject in need thereof or lower the A1C level in a subject in need thereof by at least about 5%, 10%, 15%, 20%, 25%, 30%, 35%, 40%, 45%, 50%, 55%, 60%, 65%, 70%, 75%, 80%, 85%, or 90%, or any range resulting from a combination of any two of the foregoing percentages, for example, at least about 5% to about 10% or at least about 15% to about 50%, relative to the A1C level prior to the administration of a Compound provided herein. In a particular embodiment, the methods of treating prediabetes or lowering blood or plasma glucose or lowering blood or plasma glycated hemoglobin lower the A1C level to at least about 5.7%, 5.6%, 5.5%, 5.2%, 5.0%, 4.7%, 4.5%, 4.2%, 4.0%, 3.7%, 3.5%, 3.2%, or 3.0%, or to at least a range formed by any two of the foregoing percentages, for example, to at least about 5.6% to about 3.0%.

In one embodiment, the methods of treating prediabetes or lowering blood or plasma glucose or lowering blood or plasma glycated hemoglobin lower the FPG level or the plasma glucose level or the A1C, or any combination thereof, in a subject in need thereof such that the subject is no longer diagnosed as having prediabetes in view of the criteria discussed in this Section.

### 4.5.1.3 Diagnosis of Gestational Diabetes

In one embodiment, a subject is in need of: treatment for diabetes, wherein diabetes is gestational diabetes; or maintaining or lowering blood or plasma glucose; or maintaining or lowering blood or plasma glycated hemoglobin, if the subject shows:

One Step Method (IADPSG (“International Association of the Diabetes and Pregnancy Study Groups” Consensus):

Perform a 75 g OGTT, with plasma glucose measurement fasting and at about 1 h and at about 2 h, at about 24-28 weeks of gestation in women not previously diagnosed with overt diabetes. The OGTT should be performed in the morning after an overnight fast of at least about 8 h. The diagnosis of gestational diabetes is made when any of the following plasma glucose values are met:

(1) Fasting: equal or greater than about 92 mg/dL (5.1 mmol/L);

(2) 1 h: equal or greater than about 180 mg/dL (10.0 mmol/L); and

(3) 2 h: equal or greater than about 153 mg/dL (8.5 mmol/L).

Two Step Method (NIH (“National Institutes of Health”) Consensus):

Perform an about 50 g GLT (“glucose load test,” nonfasting), with plasma glucose measurement at about 1 h, at about 24-28 weeks of gestation in women not previously diagnosed with overt diabetes. If the plasma glucose level measured about 1 h after the load is equal or greater than 140 mg/dL (7.8 mmol/L), proceed to about 100 g OGTT (Step 2). The American College of Obstetricians and Gynecologists (“ACOG”) recommends a lower threshold of about 135 mg/dL (7.5 mmol/L) in high-risk ethnic minorities with higher prevalence of gestational diabetes and some experts also recommend about 130 mg/dL (7.2 mmol/L). The about 100 g OGTT should be performed when the patient is fasting.

The diagnosis of gestational diabetes is made when at least two of the following four plasma glucose levels (measured fasting, about 1 h, about 2h, about 3h after the OGTT) are met or exceeded:

| | | NDDG (“National Diabetes |
|---|---|---|
| | Carpenter/Coustan* or | Data Group”)^{#} |
| (1) Fasting | 95 mg/dL (5.3 mmol/L) | 105 mg/dL (5.8 mmol/L) |
| (2) 1 h | 180 mg/dL (10.0 mmol/L | 190 mg/dL (10.6 mmol/L) |
| (3) 2 h | 155 mg/dL (8.6 mmol/L) | 165 mg/dL (9.2 mmol/L) |
| (4) 3 h | 140 mg/dL (7.8 mmol/L) | 145 mg/dL (8.0 mmol/L) |

*Carpenter and Coustan, “Criteria for screening tests for gestational diabetes,” Am. J. Obstet. Gynecol. 44: 768-773 (1982).
^{#}National Diabetes Data Group, “Classification and diagnosis of diabetes mellitus and other categories of glucose intolerance.” Diabetes 28: 1039-1057 (1979).

In one embodiment, the methods of treating diabetes, wherein diabetes is gestational diabetes, or lowering blood or plasma glucose or lowering blood or plasma glycated hemoglobin lower the plasma glucose level such that the subject is no longer diagnosed as having gestational diabetes using the One Step Test or the Two Step Test, or both.

### 4.5.1.4 Diagnosis of Latent Autoimmune Diabetes in Adults

In one embodiment, a subject is in need of: treatment for diabetes, wherein diabetes is Latent Autoimmune Diabetes in Adults; or maintaining or lowering blood or plasma glucose; or maintaining or lowering blood or plasma glycated hemoglobin, if the subject shows at least two of the following characteristics:
younger than age 50 at diabetes diagnosis
normal weight (a body mass index less than 25)
acute symptoms (such as extreme thirst, frequent urination, or unintentional weight loss) when diagnosed with diabetes
a personal history of another autoimmune disease, such as autoimmune thyroid disease, rheumatoid arthritis, or celiac disease
a family history of type 1 diabetes or other autoimmune diseases

In another embodiment, a subject is in need of: treatment for diabetes, wherein diabetes is Latent Autoimmune Diabetes in Adults; or maintaining or lowering blood or plasma glucose; or maintaining or lowering blood or plasma glycated hemoglobin, if the subject shows elevated levels of pancreatic autoantibodies and has recently been diagnosed with diabetes, but does not require insulin. In a specific embodiment, the presence of antibodies is measured using a GAD (“Glutamic Acid Decarboxylase”) Antibody Test. A GAD Antibody Test is a blood test, which measures whether the body of the subject is producing a type of antibody, which destroys its own GAD cells.

In one embodiment, the methods of treating diabetes, wherein diabetes is Latent Autoimmune Diabetes in Adults, or lowering blood or plasma glucose or lowering blood or plasma glycated hemoglobin lower the plasma glucose level such that the subject is no longer diagnosed as having Latent Autoimmune Diabetes.

### 4.5.1.5 Patient Populations

In one embodiment, the prediabetes or diabetes is caused by or accompanied by obesity. In a certain embodiment, an obese subject has a body mass index (“BMI”) of at least about 30 kg/m². Diagnosis and Management of Obesity, American Academy of Family Physicians, 2013, available at http://www.aafp.org/dam/AAFP/documents/patient_care/fitness/obesity-diagnosis-management.pdf (last accessed Aug. 28, 2014). The BMI is calculated as follows:

BMI=(weight in kg)/(height of subject in meters)².

In one embodiment, the subject in need for treatment of prediabetes or diabetes or in need for maintaining or lowering blood or plasma glucose, or in need for maintaining or lowering glycated hemoglobin has not been previously treated for prediabetes or diabetes.

In one embodiment, the subject in need for treatment of prediabetes or diabetes or in need for maintaining or lowering blood or plasma glucose, or in need for maintaining or lowering glycated hemoglobin shows hypersensitivity and allergic reactions, including, but not limited to, anaphylaxis, to insulin medications, such as HUMALOG®.

In one embodiment, the subject in need for treatment of prediabetes or diabetes or in need for maintaining or lowering blood or plasma glucose, or in need for maintaining or lowering glycated hemoglobin is at risk for hypokalemia. All insulin products, such as HUMALOG®, cause a shift in potassium from the extracellular to intracellular space, possibly leading to hypokalemia. Untreated hypokalemia may cause, e.g., respiratory paralysis, ventricular arrhythmia, and death. Subjects at risk for hypokalemia are, e.g., subjects using potassium-lowering medications, subjects taking medications sensitive to serum potassium concentrations, and subjects receiving intravenously administered insulin.

In one embodiment, the subject in need for treatment of prediabetes or diabetes or in need for maintaining or lowering blood or plasma glucose, or in need for maintaining or lowering glycated hemoglobin is female. In one embodiment, the subject in need for treatment of prediabetes or diabetes or in need for maintaining or lowering blood or plasma glucose, or in need for maintaining or lowering glycated hemoglobin is pregnant. In one embodiment, the subject in need for treatment of prediabetes or diabetes or in need for maintaining or lowering blood or plasma glucose, or in need for maintaining or lowering glycated hemoglobin is male.

In one embodiment, the subject in need for treatment of prediabetes or diabetes or in need for maintaining or lowering blood or plasma glucose, or in need for maintaining or lowering glycated hemoglobin is at least about 10, 15, 20, 25, 30, 35, 40, 45, 50, 55, 60, 65, 70, 75, 80, 85, or 90 years old. In one embodiment, the subject in need for treatment of prediabetes or diabetes or in need for maintaining or lowering blood or plasma glucose, or in need for maintaining or lowering glycated hemoglobin is less than about 10, 15, 20, 25, 30, 35, 40, 45, 50, 55, 60, 65, 70, 75, 80, 85, or 90 years old. In a specific embodiment, the age of the subject described in this paragraph ranges from about 10, 15, 20, 25, 30, 35, 40, 45, 50, 55, 60, 65, 70, 75, 80, or 85, 90 years (the “First List”) to about 10, 15, 20, 25, 30, 35, 40, 45, 50, 55, 60, 65, 70, 75, 80, 85, or 90 years (the “Second List”), e.g., 10-45 year, 30-90 years, or any age range resulting from a combination of a number of the First List with a number of the Second List, wherein the number of the Second List is greater than the number of the First List.

In one embodiment, the subject in need for treatment of prediabetes or diabetes or in need for maintaining or lowering blood or plasma glucose, or in need for maintaining or lowering glycated hemoglobin is a nursing subject. Sulfonylurea drugs stimulate the beta cells of the pancreas to release insulin. Some sulfonylurea drugs are known to be excreted in human milk. Because of the potential for hypoglycemia in nursing infants may exist, the use of sulfonylureas in nursing subjects should be avoided.

In one embodiment, the subject in need for treatment of prediabetes or diabetes or in need for maintaining or lowering blood or plasma glucose, or in need for maintaining or lowering glycated hemoglobin is a subject with New York Heart Association (“NYHA”) Class III or IV heart failure. Doctors usually classify heart failure according to the severity of a subject's symptoms. The table below describes the most commonly used classification system, the NYHA Functional Classification. The system places patients in one of four categories based on how much they are limited during physical activity. Some diabetes medications, such as rosiglitazone (AVANDIA®) are contraindicated for subjects described in this paragraph.

| | Functional Capacity: How a patient with cardiac disease |
|---|---|
| Class | feels during physical activity |
| I | Patients with cardiac disease but resulting in no limitation |
| | of physical activity. Ordinary physical activity does not cause |
| | undue fatigue, palpitation, dyspnea or anginal pain. |
| II | Patients with cardiac disease resulting in slight limitation |
| | of physical activity. They are comfortable at rest. Ordinary |
| | physical activity results in fatigue, palpitation, dyspnea or |
| | anginal pain. |
| III | Patients with cardiac disease resulting in marked limitation |
| | of physical activity. They are comfortable at rest. Less than |
| | ordinary activity causes fatigue, palpitation, dyspnea or |
| | anginal pain. |
| IV | Patients with cardiac disease resulting in inability to carry |
| | on any physical activity without discomfort. Symptoms of heart |
| | failure or the anginal syndrome may be present even at rest. If |
| | any physical activity is undertaken, discomfort increases. |

http://www.heart.org/HEARTORG/Conditions/HeartFailure/AboutHeartFailure/Classes-of-Heart-Failure_UCM_306328_Article.jsp (last accessed Aug. 28, 2014).

In one embodiment, the subject in need for treatment of prediabetes or diabetes or in need for maintaining or lowering blood or plasma glucose, or in need for maintaining or lowering glycated hemoglobin is a subject with a hypersensitivity reaction to dipeptidyl peptidase-4 (“DPP-4”) inhibitors, such as sitagliptin (JANUVIA®). These reactions include, but are not limited to, anaphylaxis, angioedema, and exfoliative skin conditions, such as Stevens-Johnson syndrome.

In one embodiment, the subject in need for treatment of prediabetes or diabetes or in need for maintaining or lowering blood or plasma glucose, or in need for maintaining or lowering glycated hemoglobin is a subject with normal kidney function (glomerular filtration rate (“GFR”) above about 90 mL/min/1.73 m² and no proteinuria), with chronic kidney disease (Stage 1) (GFR above about 90 mL/min/1.73 m² with evidence of kidney damage), with chronic kidney disease (Stage 2) (mild, GFR of about 60 to about 89 mL/min/1.73 m² with evidence of kidney damage), with chronic kidney disease (Stage 3) (moderate, GFR of about 30 to about 59 mL/min/1.73 m²), with chronic kidney disease (Stage 4) (severe, GFR of about 15 to about 29 mL/min/1.73 m²), or with chronic kidney disease (Stage 5) (kidney failure, GFR less than about 15 mL/min/1.73 m², wherein the subject may or may not require dialysis).

### 4.6 Pharmaceutical Compositions and Routes of Administration

Provided herein are pharmaceutical compositions comprising a Compound provided herein and a pharmaceutically acceptable carrier. In a particular embodiment, the pharmaceutical compositions are those, wherein the composition is suitable for topical, oral, subcutaneous, or intravenous administration.

Provided herein are compositions comprising an effective amount of a Compound and compositions comprising an effective amount of a Compound and a pharmaceutically acceptable carrier or vehicle. In some embodiments, the pharmaceutical composition described herein are suitable for oral, parenteral, mucosal, transdermal or topical administration.

The Compounds can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the Compound in the pharmaceutical composition may be at a level that will exercise the desired effect; for example, about 0.1 mg/kg to about 1000 mg/kg or about 0.5 mg/kg to about 100 mg/kg of a patient's body weight in unit dosage for both oral and parenteral administration.

The dose of a Compound to be administered to a patient is rather widely variable and can be the judgment of a health-care practitioner. In general, the Compounds can be administered one to four times a day in a dose of about 0.1 mg/kg of a patient's body weight to about 1000 mg/kg of a patient's body weight in a patient, but the above dosage may be properly varied depending on the age, body weight and medical condition of the patient and the type of administration. In one embodiment, the dose is about 0.05 mg/kg of a patient's body weight to about 500 mg/kg of a patient's body weight, 0.05 mg/kg of a patient's body weight to about 100 mg/kg of a patient's body weight, about 0.5 mg/kg of a patient's body weight to about 100 mg/kg of a patient's body weight, about 0.1 mg/kg of a patient's body weight to about 50 mg/kg of a patient's body weight or about 0.1 mg/kg of a patient's body weight to about 25 mg/kg of a patient's body weight. In one embodiment, one dose is given per day. In another embodiment, two doses are given per day. In any given case, the amount of the Compound administered will depend on such factors as the solubility of the active component, the formulation used and the route of administration.

In another embodiment, provided herein are methods for the treatment of prediabetes, and diabetes; and methods of maintaining or lowering blood or plasma glucose, and maintaining or lowering glycated hemoglobin; comprising the administration of about 7.5 mg/day to about 75 g/day, about 3.75 mg/day to about 37.5 g/day, about 3.75 mg/day to about 7.5 g/day, about 37.5 mg/day to about 7.5 g/day, about 7.5 mg/day to about 3.75 g/day, about 3.75 mg/day to about 1.875 g/day, about 3.75 mg/day to about 1,000 mg/day, about 3.75 mg/day to about 800 mg/day, about 3.75 mg/day to about 500 mg/day, about 3.75 mg/day to about 300 mg/day, or about 3.75 mg/day to about 150 mg/day of a Compound to a patient in need thereof. In a particular embodiment, the methods disclosed herein comprise the administration of 1 mg/day, 5 mg/day, 10 mg/day, 15 mg/day, 20 mg/day, 30 mg/day, 40 mg/day, 45 mg/day, 50 mg/day, 60 mg/day, 75 mg/day, 100 mg/day, 125 mg/day, 150 mg/day, 200 mg/day, 250 mg/day, 300 mg/day, 400 mg/day, 600 mg/day, 800 mg/day, 1,000 mg/day, 1,500 mg/day, 2,000 mg/day, 2,500 mg/day, 5,000 mg/day, or 7,500 mg/day of a Compound to a in need thereof.

In another embodiment, provided herein are unit dosage formulations that comprise between about 7.5 mg to about 75 g, about 3.75 mg to about 37.5 g, about 3.75 mg to about 7.5 g, about 37.5 mg to about 7.5 g, about 7.5 mg to about 3.75 g, about 3.75 mg to about 1.875 g, about 3.75 mg to about 1,000 mg, about 3.75 mg to about 800 mg, about 3.75 mg to about 500 mg, about 3.75 mg to about 300 mg, or about 3.75 mg to about 150 mg of a Compound.

In a particular embodiment, provided herein are unit dosage formulation comprising about 1 mg, 5 mg, 10 mg, 15 mg, 20 mg, 30 mg, 40 mg, 45 mg, 50 mg, 60 mg, 75 mg, 100 mg, 125 mg, 150 mg, 200 mg, 250 mg, 300 mg, 400 mg, 600 mg, 800 mg 1,000 mg, 1,500 mg, 2,000 mg, 2,500 mg, 5,000 mg, or 7,500 mg of a Compound.

In another embodiment, provided herein are unit dosage formulations that comprise a Compound dosage that achieves a target plasma concentration of the Compound in a patient or an animal model. In a particular embodiment, provided herein are unit dosage formulations that achieves a plasma concentration of the Compound ranging from approximately 0.001 μg/mL to approximately 100 mg/mL, approximately 0.01 μg/mL to approximately 100 mg/mL, approximately 0.01 μg/mL to approximately 10 mg/mL, approximately 0.1 μg/mL to approximately 10 mg/mL, approximately 0.1 μg/mL to approximately 500 μg/mL, approximately 0.1 μg/mL to approximately 500 μg/mL, approximately 0.1 μg/mL to approximately 100 μg/mL, or approximately 0.5 μg/mL to approximately 10 μg/mL in a patient or an animal model. To achieve such plasma concentrations, a Compound or a pharmaceutical composition thereof may be administered at doses that vary from 0.001 μg to 100,000 mg, depending upon the route of administration. In certain embodiments, subsequent doses of a Compound may be adjusted accordingly based on the plasma concentrations of the Compound achieved with initial doses of the Compound or pharmaceutical composition thereof administered to the subject.

A Compound can be administered once, twice, three, four or more times daily.

A Compound can be administered orally for reasons of convenience. In one embodiment, when administered orally, a Compound is administered with a meal and water. In another embodiment, the Compound is dispersed in water or juice (e.g., apple juice or orange juice) and administered orally as a suspension. In another embodiment, when administered orally, a Compound is administered in a fasted state.

The Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a Compound without an additional carrier, excipient or vehicle.

In another embodiment, provided herein are compositions comprising an effective amount of a Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts. In general, all of the compositions are prepared according to known methods in pharmaceutical chemistry. Capsules can be prepared by mixing a Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Compound can be delayed or prolonged by proper formulation. For example, a slowly soluble pellet of the Compound can be prepared and incorporated in a tablet or capsule, or as a slow-release implantable device. The technique also includes making pellets of several different dissolution rates and filling capsules with a mixture of the pellets. Tablets, capsules, or pellets can be coated with a film that resists dissolution for a predictable period of time (the coating may comprise, for example, polymethylacrylates or ethyl cellulose). Even the parenteral preparations can be made long-acting, by dissolving or suspending the Compound in oily or emulsified vehicles that allow it to disperse slowly in the serum.

### 5 EXAMPLES

### 5.1 Biological Examples

### 5.1.1 In Vitro Assays

A wide variety of assay methods are known in the art to profile Compounds provided herein against human sodium channels stably expressed in human embryonic kidney (HEK293) cells. Such assays are disclosed, for example, in WO2007/109324 to Fraser et al., which is incorporated herewith in its entirety. In particular, such assays are disclosed in Example 3, pages 94-99 of WO2007/109324, which is herewith incorporated in its entirety.

Recombinant NaV Cell Lines

In vitro assays were performed in recombinant cell line that stably express a heterotrimeric protein of interest from an introduced nucleic acid encoding the alpha subunit (hNav1.7, SCN9A), the beta subunit (SCNB1) and the beta subunit (SCNB2). The cell line was engineered using Human Embryonic Kidney 293 cells (HEK293) as host background. Additional cell lines stably expressing recombinant human NaVs (e.g., NaV1.1, NaV1.2, NaV1.3, NaV1.4, NaV1.5, NaV1.6, NaV1.7 or NaV1.8) alpha subunit alone or in combination with various beta subunits in Chinese Hamster Ovary (CHO) or HEK293 cells as a host background can also be used in in vitro assays.

To generate cells and cell lines provided herein, one can use, for example, the technology described in U.S. Pat. No. 6,692,965 and WO/2005/079462. Both of these documents are incorporated herein by reference in their entirety. This technology provides real-time assessment of millions of cells such that any desired number of clones (from hundreds to thousands of clones) expressing the desired gene(s) can be selected. Using cell sorting techniques, such as flow cytometric cell sorting (e.g., with a FACS machine) or magnetic cell sorting (e.g., with a MACS machine), one cell per well is automatically deposited with high statistical confidence in a culture vessel (such as a 96 well culture plate). The speed and automation of the technology allows multigene recombinant cell lines to be readily isolated.

FDSS Membrane Potential In-Vitro Assay

Cells stably expressing hNaV1.7 α, β1 and β2 subunits were maintained under standard cell culture conditions in Dulbecco's Modified Eagles medium supplemented with 10% fetal bovine serum, glutamine and HEPES. On the day before assay, the cells were harvested from stock plates using cell dissociation reagent, e.g., trypsin, CDB (GIBCO) or cell-stripper (Mediatech), and plated at 10,000-25,000 cells per well in 384 well plates in growth media. The assay plates were maintained in a 37° C. cell culture incubator under 5% CO₂ for 22-48 hours. The media was then removed from the assay plates and membrane potential fluorescent dye diluted in load buffer (137 mM NaCl, 5 mM KCl, 1.25 mM CaCl₂, 25 mM HEPES, 10 mM glucose) was added.

Membrane potential dye(s): Blue membrane potential dye (Molecular Devices Inc.), or membrane potential-sensitive dye, HLB021-152 (AnaSpec) were combined with a fluorescence quencher, e.g., Dipicrylamine (DPA), Acid Violet 17 (AV 17), Diazine Black (DB), HLB30818, FD and C Black Shade, Trypan Blue, Bromophenol Blue, HLB30701, HLB30702, HLB30703, Nitrazine Yellow, Nitro Red, DABCYL (Molecular Probes), FD and C Red NO. 40, QSY (Molecular Probes), metal ion quenchers (e.g., Co², Ni², Cu²), and iodide ions.

The cells were incubated with the membrane potential dye for 45-60 mins at 37° C. The dye-loaded assay plates were then placed in the high-throughput fluorescent plate reader (Hamamatsu FDSS). The kinetic read was started with assay plate imaging every second. After 10 s, the assay buffer alone, or test compound diluted in the assay buffer, were added to the cells (1^{st} addition step) and the kinetic read continued every 2 s for 2 mins total after which cells were stimulated with veratridine and scorpion venom (2^{nd} addition step) diluted in assay buffer to evaluate the effects of the test compounds.

Veratridine and scorpion venom proteins modulate the activity of voltage-gated sodium channels through a combination of mechanisms, including an alteration of the inactivation kinetics. The resulting activation of sodium channels in stable NaV1.7-expressing cells changes cell membrane potential and the fluorescent signal increases as a result of depolarization.

Control response elicited by veratridine and scorpion venom with buffer only (without test compounds added) was taken as the maximal response. Assay results are expressed in relative fluorescence units (RFU) and can be determined by using the maximum signal during the 2^{nd} addition/stimulation step or by computing the difference of maximum and minimum signal during the 2^{nd} addition/stimulation step. The signal inhibition was estimated for each test compound concentration in triplicate. The data were analyzed using GraphPad Prism software to determine the IC50 value for the test compound.

Veratridine and scorpion venom from Leiurus quinquestriatus quinquestriatus can be purchased from Sigma-Aldrich (St. Louis, Mo.). Stock solutions were prepared as 10 mM (veratridine) in DMSO and as 1 mg/ml (scorpion venom) in de-ionised water. The sodium channels agonists were diluted in assay buffer to a 4× concentration with final concentration being 2-25 μM for veratridine and 2-20 μg/ml for scorpion venom.

Test compounds were prepared as 2-10 mM stock in DMSO. The stock solutions were further diluted in DMSO in serial dilution steps and then transferred to assay buffer as 4× of the final assay concentrations. Test compounds were added during the first addition (pre-stimulation) step in the kinetic read. All test compound concentrations were evaluated in triplicate.

Compounds 1, 2, 3, 12, 13, 16, 26, and 32 showed NaV1.7 IC50 values less than 0.13 μM; Compounds 4, 5, 6, 7, 8, 9, 10, 15, 18, 20, and 28 showed NaV1.7 IC50 values between 0.13 and 1.0 μM; Compounds 14, 17, 19, 21, 22, and 23 showed NaV1.7 IC50 values greater than 1.0 μM and less than 20.0 μM.

Compound 54 showed NaV1.7 IC50 value of less than 0.1 μM. Compounds 35, 43, 46, 55, 57, and 59 showed NaV1.7 IC50 values between 0.1 μM and 0.5 μM. Compounds 34, 48, 49, 50, 51, 56 and 68 showed NaV1.7 IC50 values of greater than 0.5 μM and equal or less than 1.0 μM. Compounds 42, 45, 47, 52, and 58 showed NaV1.7 IC50 values of greater than 1.0 μM and less than 20.0 μM.

Patchliner Electrophysiological In-Vitro Assay

The recording of sodium current from stable HEK293 cell lines expressing NaV1.7 or NaV1.5 or CHO cell lines expressing NaV1.1, NaV1.2, NaV1.3, NaV1.4, NaV1.6 or NaV1.8 was done on a Patchliner® instrument, Nanion Technologies. The Patchliner® is a fully automated bench-top patch clamp platform and can record simultaneously from up to eight single cells with GΩ seals.

For patch-clamp experiments, cells were grown under standard culturing conditions in Dulbecco's Modified Eagles medium supplemented with 10% fetal bovine serum, glutamine and HEPES. Cells were harvested and kept in suspension for up to 4 hours with no significant change in quality or ability to patch. Whole cell patch clamp recordings were conducted according to Nanion's standard procedure for the Patchliner®. Experiments were conducted at room temperature.

Voltage protocols were designed to establish: 1) peak current amplitude (Iₘₐₓ), 2) test potential (Vₘₐₓ) and 3) half-inactivation potential (V_{1/2}) for each of the eight individual cells. To determine V_{1/2}, a standard steady-state inactivation protocol was executed using a series of fifteen 500 ms depolarizing pre-pulses in 10 mV increments (starting at −130 mV) and immediately followed by a 10 ms test pulse to Vₘₐₓ. To estimate test compound affinity to the inactivated state of sodium channel (Kᵢ), the holding potential for each cell was set automatically to the V_{1/2} calculated from a steady-state inactivation data. The current was activated with the following voltage protocol: holding at V_{1/2} for 2-5 seconds, return to the −120 mV for 5-10 ms to relieve fast inactivation, stepping to test potential (Vₘₐₓ) for 10-20 ms. This voltage protocol was repeated every 10 seconds to establish the baseline with 2-3 buffer additions followed by the test compound addition. The dose-dependent inhibition was analyzed using Nanion's Data Analysis Package.

Compounds 1, 2, 5, 6, 8, 11, 12, 13, 15, 16, 20, 24, 26, 28, 29 and 32 showed NaV1.7 IC50 values less than 0.1 μM; Compounds 14, 17, 18, 19, 21, 22, 23, 25 and 33 showed NaV1.7 IC50 value between 0.1 and 1.0 μM.

Compounds 44, 49, 53, 54, 60, 61, 62, 63, 64, 65, 66, 67, and 69 showed NaV1.7 IC50 values of less than 0.1 μM. Compound 34 and 52 showed an NaV1.7 IC50 value of greater than 0.1 μM and equal or less than 0.5 μM. Compounds 47 and 58 showed NaV1.7 IC50 values of greater than 1.0 μM and less than 10.0 μM. Compounds 43, 44, 47, 49, 54, 56, 58, 59, 60, 61, 62, 63, 65 and 66 showed a NaV1.5 IC50 greater than 10.0 μM. Compounds 1 and 49 showed NaV1.1 and NaV1.8 IC50 values greater than 10 μM and NaV1.4 IC50 values greater than 3 μM. Compound 1 showed an NaV 1.3 IC50 of greater than 10 μM. Compound 1 and 49 showed at least 10-fold selectivity for NaV1.7 compared to NaV1.2 and NaV1.6. The results described in paragraphs [00275] and [00276] were measured in the Patchliner Electrophysiological Assay as described in this section starting at paragraph [00271]).

In an assay similar to the one described in this section starting at paragraph [00271] using CHO or HEK293 cell lines expressing NaV1.3, compound 49 showed an IC50 of greater than 10 μM.

In-vitro Cytochrome P450 (CYP450) Inhibition Assay

We evaluated interaction of drug candidates with cytochrome P450 enzymes which are a major determinant of drug clearance via oxidative metabolism using a high throughput compatible, fluorescence based CYP450 screening assay (Vivid® CYP450, Invitrogen) according to manufacturer's directions.

In brief, test compounds at four different concentrations (μM—6.0, 2.0, 0.7, 0.2), a positive control (Ketoconazole) and a solvent control were incubated at room temperature in unique wells of a 96-well microtiter plate with CYP3A4 enzyme complex for 20 minutes. A pre-read fluorescence (Ex—485 nm/Em—530 nm) was measured at the start of the incubation using a Tecan Safire² microplate reader-monochromator to determine background fluorescence. At the end of the incubation period, enzyme substrate and co-enzyme were added and the reaction was kinetically monitored for 1 hour by measuring fluorescence every minute. Effect of test compounds on inhibition of CYP3A4 metabolism of provided substrate was determined by calculating the ratio of the effective reaction rate in presence of test compound to that in the absence of inhibitor.

Compounds 9, 11, 13, 14, 15, 17, 18, 19, 21, and 22 showed 0-25% CYP3A4 inhibition at 6 μM test concentration; Compounds 5, 6, 8, 10 and 16 showed 25-50% CYP3A4 inhibition at 6 μM test concentration; Compounds 1, 2, 3, 4, 12, 20 and 32 showed 50-100% CYP3A4 inhibition at 6 μM test concentration.

### 5.1.2 In Vivo Assay

Evaluation of Anti-Diabetic Effect in In Vivo Model of Diabetes

250-350 g male Sprague-Dawley rats from appropriate animal resources were used. Type I diabetes was induced by a single injection, (intraperitoneally, intravenously or intramuscularly) of 50-100 mg/kg of streptozotocin (STZ, Sigma Chemicals, St. Louis, Mo. or VWR) freshly dissolved in sodium citrate (0.01 M, pH 4.5). Sham animals were given either saline or same vehicle injection. Following a wait time of about two days, inducement of diabetes was confirmed in STZ-injected rats by measuring the plasma glucose concentrations in blood samples from the tail vein after a fast of 6 hours. The glucose level was assayed using a mini glucose monitor (kit for AlphaTRAK 2 meter, available from Abbott Laboratories). Screening for hyperglycemia in STZ-injected animals was done, with only animals with a final blood glucose level ≧300 mg/dl being selected for the study. Glucose levels in the sham animals remained normal. Other parameters (water intake, food intake, and bodyweight) were monitored before the treatment with a test compound and after the cessation of the treatment.

The animals selected for the study showed stable signs of diabetic condition, e.g., hyperglycemia, increased water and food intake with no gain in bodyweight or loss of body weight. Only animals with a final (fasted 6 hrs from 8 am-2 pm) blood glucose level ≧300 mg/dl were included in the study, animals that showed no hyperglycemia (blood glucose level <300 mg/dl) were excluded from study. The baseline glucose levels, daily food and water intake and behavioral tests (von Frey, paw pressure and plantar tests) were measured for each animal once weekly for 28 weeks, and once every 2-4 weeks for an additional 28-30 weeks. At a selected week (started from week 6) for a compound test, diabetic rats were divided into two test groups: a vehicle control group and a test compound treated group (n=10 each group). A sham group (saline only without STZ i.p. injection, n=10) was added as a normal, non-diabetic control group. To minimize animal stress associated with repeated daily handling, the compound treated diabetic group of animals received the test compound (compound 49) at a daily dose of 60 mg/kg in their drinking water containing 2% PEG600 and 1% glycofurol (the test compound concentration in the drinking water was based on the individual animal's average daily water consumption as determined the pre-dosing period, which averaged 0.1-0.11 mg/ml). The vehicle control group received drinking water containing 2% PEG600 and 1% glycofurol without the test compound. The treatment continued for 9 days. On day 10 drinking water with test compound or vehicle (2% PEG600 and 1% glycofurol) was replaced with plain drinking water in all groups. The monitoring of glucose levels, food and water intake as well as behavior tests continued for additional 28-30 weeks.

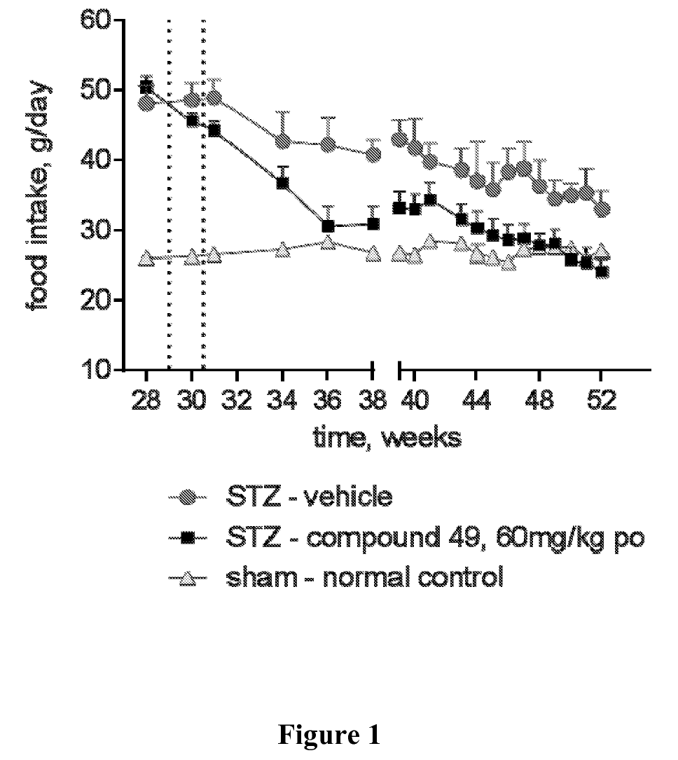

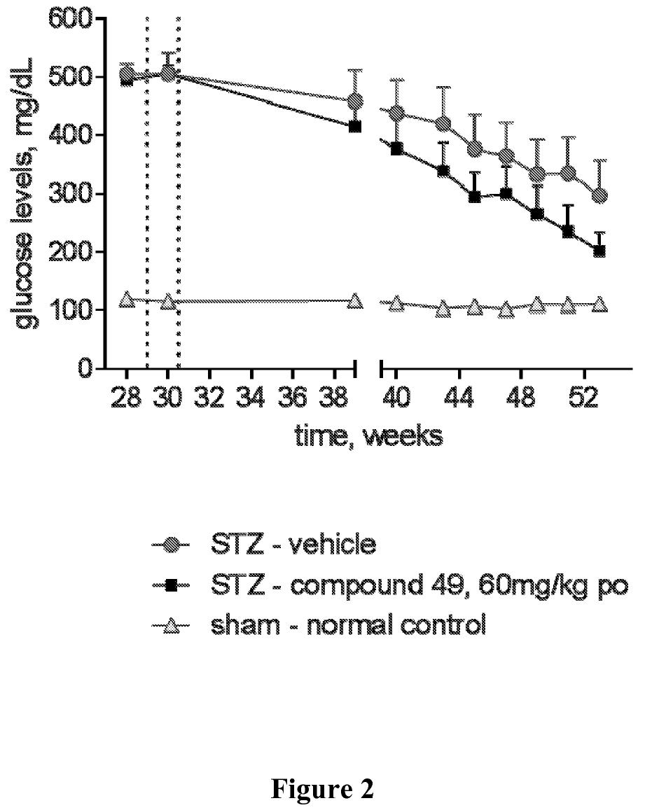

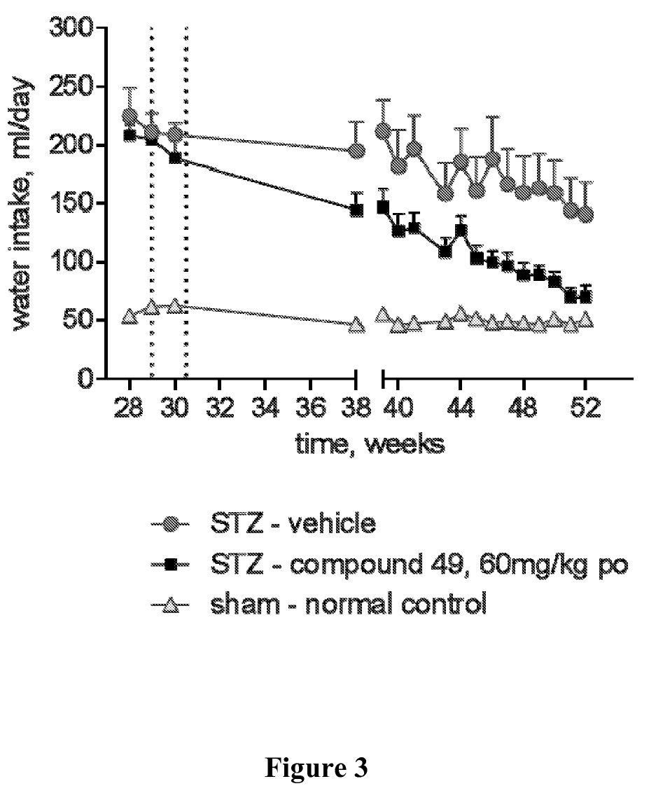

FIGS. 1-3 show food intake, glucose level, and water intake, respectively, for the vehicle control group, the test compound treated group (compound 49, 60 mg/kg/day), and the sham group. After the treatment with compound 49 stopped on day 9 (treatment period marked by dotted lines in each of FIGS. 1-3), the test compound treated group showed improvements in general appearance and/or health. In particular, FIGS. 1, 2, and 3 show that the food intake, glucose level, and water intake, all of which are manifestations of diabetes in the animal model, were significantly reduced compared to the vehicle control group. The significant reduction of food intake, glucose level, and water intake of the compound treated group compared to the vehicle control group continues through week 52. The sham group showed no significant change in food intake, glucose level, or water intake over the course of the experiment. Since the standard deviation in the sham groups are less than 5%, no error bars are shown.

### 5.2 Examples of NaV Inhibitors

### 5.2.1 General Methods

### 5.2.1.1 LCMS Method

Method-A

LC-MS was carried out on Acquity H-Class UPLC, PDA and SQ Detector. The column used was BEH C18 50×2.1 mm, 1.7 micron and column flow was 0.55 ml/min. Mobile phase were used (A) 0.1% Formic acid+5 mM Ammonium Acetate in water and (B) 0.1% Formic acid in Acetonitrile. The UV spectra were recorded at its lambda Max and Mass spectra were recorded using ESI technique. The following gradient is used to monitor reaction progress and analyze final products.

| Time (min) | % A | % B |
|---|---|---|
| 0.01 | 95 | 05 |
| 0.40 | 95 | 05 |
| 0.80 | 65 | 35 |
| 1.20 | 45 | 55 |
| 2.50 | 00 | 100 |
| 3.30 | 00 | 100 |
| 3.31 | 95 | 05 |
| 4.00 | 95 | 05 |

Method-B

LC-MS was carried out on Waters LC alliance 2995, PDA 2996 and SQ Detector. The column used was X-BRIDGE C18 150×4.6 mm×5 micron and column flow was 1.0 ml/min. Mobile phase were used (A) 0.1% Ammonia in water and (B) 0.1% Ammonia in Acetonitrile. The UV spectra were recorded at its lambda Max and Mass spectra were recorded using ESI technique. The following gradient is used to monitor reaction progress and analyze final products.

| Time (min) | % A | % B |
|---|---|---|
| 0.01 | 90 | 10 |
| 5.00 | 10 | 90 |
| 7.00 | 00 | 100 |
| 11.00 | 00 | 100 |
| 11.01 | 90 | 10 |
| 12.00 | 90 | 10 |

Method-C

LC-MS was carried out on Waters LC alliance 2995, PDA 2996 and SQ Detector. The column used was X-BRIDGE C18 150×4.6 mm×5 micron and column flow was 1.0 ml/min. Mobile phase were used (A) 0.1% Ammonia in water and (B) 0.1% Ammonia in Acetonitrile. The UV spectra were recorded at its lambda Max and Mass spectra were recorded using ESI technique. The following gradient is used to monitor reaction progress and analyze final products.

| Time (mm) | % A | % B |
|---|---|---|
| 0.01 | 100 | 00 |
| 7.00 | 50 | 50 |
| 9.00 | 00 | 100 |
| 11.00 | 00 | 100 |
| 11.01 | 100 | 00 |
| 12.00 | 100 | 00 |

Method-D

LC-MS was carried out on Waters LC alliance 2995, PDA 2996 and SQ Detector. The column used was X-BRIDGE C18 150×4.6 mm×5 micron and column flow was 1.0 ml/min. Mobile phase were used (A) 20 mM Ammonium Acetate in water and (B) 100% Methanol. The UV spectra were recorded at its lambda Max and Mass spectra were recorded using ESI technique. The following gradient is used to monitor reaction progress and analyze final products.

| Time (min) | % A | % B |
|---|---|---|
| 0.01 | 90 | 10 |
| 5.00 | 10 | 90 |
| 7.00 | 00 | 100 |
| 11.00 | 00 | 100 |
| 11.01 | 90 | 10 |
| 12.00 | 90 | 10 |

5.2.1.2 HPLC Method

Method-A

HPLC was carried out on Waters e2695, PDA Detector. The column used was Phenomenex Gemini, C18 150×4.6 mm, 5 micron and column flow was 1.00 ml/min. Mobile phase were used (A) 0.1% Formic acid in water and (B) 0.1% Formic acid in Acetonitrile. The UV spectra were recorded at its lambda Max. The following gradient is used.

| Time (min) | % A | % B |
|---|---|---|
| 0.01 | 90 | 10 |
| 7.00 | 10 | 90 |
| 9.00 | 00 | 100 |
| 13.00 | 00 | 100 |
| 13.01 | 90 | 10 |
| 17.00 | 90 | 10 |

Method-B

HPLC was carried out on Waters e2695, PDA Detector. The column used was Phenomenex Gemini, C18 150×4.6 mm, 5 micron and column flow was 1.00 ml/min. Mobile phase were used (A) 0.1% Formic acid in water and (B) 0.1% Formic acid in Acetonitrile. The UV spectra were recorded at its lambda Max. The following gradient is used.

| Time (mm) | % A | % B |
|---|---|---|
| 0.01 | 100 | 00 |
| 7.00 | 50 | 50 |
| 9.00 | 00 | 100 |
| 13.00 | 00 | 100 |
| 13.01 | 100 | 00 |
| 17.00 | 100 | 00 |

Method-C

HPLC was carried out on Waters e2695, PDA Detector. The column used was X-BRIDGE, C18 150×4.6 mm, 5 micron and column flow was 1.00 ml/min. Mobile phase were used (A) 0.1% Ammonia in water and (B) 0.1% Ammonia in Acetonitrile. The UV spectra were recorded at its lambda Max. The following gradient is used.

| Time (min) | % A | % B |
|---|---|---|
| 0.01 | 90 | 10 |
| 7.00 | 10 | 90 |
| 9.00 | 00 | 100 |
| 13.00 | 00 | 100 |
| 13.01 | 90 | 10 |
| 17.00 | 90 | 10 |

Method-D

HPLC was carried out on Waters e2695, PDA Detector. The column used was X-BRIDGE, C18 150×4.6 mm, 5 micron and column flow was 1.00 ml/min. Mobile phase were used (A) 0.1% Ammonia in water and (B) 0.1% Ammonia in Acetonitrile. The UV spectra were recorded at its lambda Max. The following gradient is used.

| Time (min) | % A | % B |
|---|---|---|
| 0.01 | 100 | 00 |
| 7.00 | 50 | 50 |
| 9.00 | 00 | 100 |
| 13.00 | 00 | 100 |
| 13.01 | 100 | 00 |
| 17.00 | 100 | 00 |

### 5.2.1.3 PREP HPLC Method

Method-A

PREP HPLC was carried out on Shimadzu UFLC, LC-20 AP, and UV Detector.

The column used was Sunfire OBD, C18 250×19 mm, 5 micron and column flow was 18.00 ml/min. Mobile phase were used (A) 0.1% HCL in water and (B) 100% Acetonitrile. The UV spectra were recorded at its lambda Max. The following gradient was used.

| Time (min) | % A | % B |
|---|---|---|
| 0.01 | 90 | 10 |
| 7.00 | 10 | 90 |
| 9.00 | 00 | 100 |
| 13.00 | 00 | 100 |
| 13.01 | 90 | 10 |
| 17.00 | 90 | 10 |

Method-B

PREP HPLC was carried out on Shimadzu UFLC, LC-20 AP, and UV Detector. The column used was Sunfire OBD, C18 250×19 mm, 5 micron and column flow was 18.00 ml/min. Mobile phase were used (A) 0.1% Formic acid in water and (B) 0.1% Formic acid in Acetonitrile. The UV spectra were recorded at its lambda Max. The following gradient was used.

| Time (min) | % A | % B |
|---|---|---|
| 0.01 | 90 | 10 |
| 7.00 | 10 | 90 |
| 9.00 | 00 | 100 |
| 13.00 | 00 | 100 |
| 13.01 | 90 | 10 |
| 17.00 | 90 | 10 |

Method-C

PREP HPLC was carried out on Shimadzu UFLC, LC-20 AP, and UV Detector. The column used was X-BRIDGE, C18 250×19 mm, 5 micron and column flow was 18.00 ml/min. Mobile phase were used (A) 0.1% Ammonia in water and (B) 0.1% Ammonia in Acetonitrile. The UV spectra were recorded at its lambda Max. The following gradient was used.

| Time (min) | % A | % B |
|---|---|---|
| 0.01 | 90 | 10 |
| 7.00 | 10 | 90 |
| 9.00 | 00 | 100 |
| 13.00 | 00 | 100 |
| 13.01 | 90 | 10 |
| 17.00 | 90 | 10 |

### 5.2.1.4 List of Abbreviations

Ac=Acetyl

EtOAc=ethyl acetate

Bn=Benzyl

Boc=tert-Butoxycarbonyl

Bzl=Benzyl

DBU=1,8-Diazabyciclo[5.4.0]undec-7-ene

DCC=1,3-Dicyclohexylcarbodiimide

DCM=Dichloromethane

DEAD=Diethyl azodicarboxylate

DIC=Diisopropylcarbodiimide

DIPEA=Diisopropylethylamine

D. M. water=demineralized water

DME=1,2-Dimethoxyethane

DMF=N,N-Dimethylformamide

DMSO=Dimethylsulphoxide

EDC=1-Ethyl-3-(3-dimethylaminopropy)carbodiimide hydrochloride

Et₂O=Diethyl ether

HOBt=1-Hydroxybenzotriazole

IPA=Isopropyl alcohol

KHMDS=Potassium bis(trimethylsilyl)amide

LAH=Lithium aluminium hydride

LDA=Lithium diisopropylamide

LHMDS=Lithium bis(trimethylsilyl)amide

MOM=Methoxymethyl

NaHMDS=Sodium bis(trimethylsilyl)amide

NBS=N-Bromosuccinimide

Ph=Phenyl

PMB=p-Methoxybenzyl

Py=Pyridine

TEA=Triethylamine

TFA=Trifluoroacetic acid

THF=Tetrahydrofurane

Tol=p-Toluoyl

### 5.2.2 Examples

### Example 1: Synthesis of 3-(4-(2-(4-(N-1,2,4-thiadiazol-5-ylsulfamoyl)-2-chloro-5-fluorophenoxy)-5-chlorophenyl)picolinamido)propanoic acid

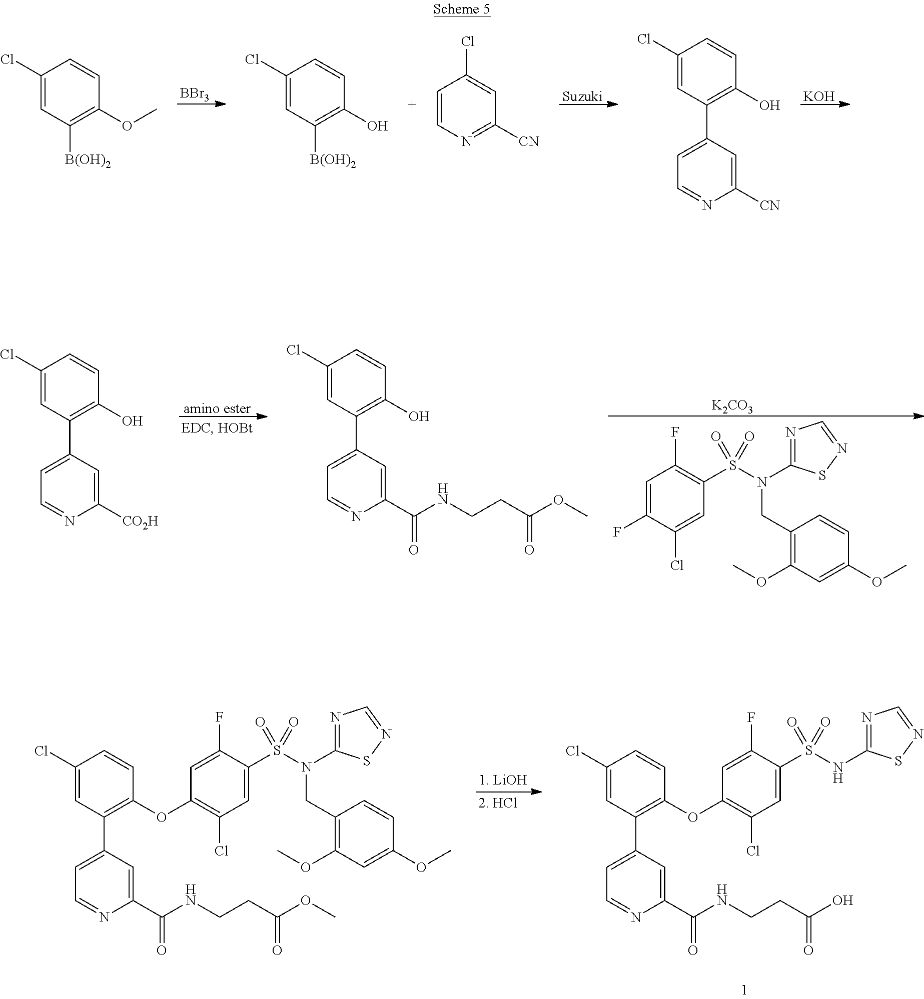

### Step 1: Preparation of (5-chloro-2-hydroxyphenyl)boronic acid

A solution of 5-chloro-2-methoxyphenylboronic acid (10.0 g, 53.6 mmol) in dichloromethan (100 ml) was cooled to temperature between 5-10° C. To the above mixture, 100 ml 1M solution of borontribromide in DCM was added drop wise using a pressure equalizing dropping funnel, over a period of 30 minutes. The resulting reaction mixture was then stirred at room temperature for 30 minutes. After completion of reaction, the mixture was poured drop wise on to an ice cold saturated sodium bicarbonate solution (600 ml). The resulting mixture was allowed to stir at room temperature for 1 hr. The DCM layer was separated out and the aqueous layer thus collected was cooled to temperature between 10-15° C. 1N solution of dilute hydrochloric acid was then added to the above cooled aqueous layer and this resulted in precipitate formation. The solid was filtered off under vacuo and dried to afford 9 g (yield: 97%) of product. LC-MS: m/z=170.9 (M+H).

### Step 2: Preparation of 4-(5-chloro-2-hydroxyphenyl)picolinonitrile

To a solution of 4-Chloropicolinonitrile (1.0 g, 7.2 mmol) in IPA:toluene (7 ml:7 ml) were sequentially added (5-chloro-2-hydroxyphenyl)boronic acid (1.49 g, 8.65 mmol) and potassium carbonate (3.99 g, 21.64 mmol) at room temperature. The resulting reaction mixture was degassed for 15 minutes by purging with nitrogen. Thereafter calculated quantity of Tetrakis (0.416 g, 0.36 mmol) was added to the reaction mixture and nitrogen purging was further continued for next 20 minutes. The resulting reaction mixture was then refluxed at 100° C. for 20 hours. After completion of the reaction, the mixture was concentrated under vacuo. To the resulting crude mass water (50 ml) was added and the mixture was extracted with ethyl acetate (3×25 ml). The combined organic extract was washed with water (20 ml), brine (20 ml), dried over sodium sulfate and concentrated under vacuo to get the desired crude product. The crude product was purified by column chromatography using normal phase silica gel. The desired product eluted at around 20-30% ethyl acetate in hexane. Evaporation of the product fractions gave 0.8 g (yield, 48%) of desired product as a solid. LC-MS: m/z=231.1 (M+H).

### Step 3: Preparation of 4-(5-chloro-2-hydroxyphenyl)picolinic acid)

To a solution of 4-(5-chloro-2-hydroxyphenyl)picolinonitrile (0.5 g, 2.17 mmol) in THF (20 ml) was added a solution of potassium hydroxide (4.276 g, 14 mmol) in water (10 ml) at room temperature. The resulting reaction mixture was then refluxed at 100° C. for 5 hours. After completion of the reaction, the mixture was concentrated under vacuo. Ice cold water was added in to the reaction mixture, the resulting mixture was then acidified between pH 3-6 with 1N HCl. The resulting solid precipitate was filtered and dried to afford 0.5 g (yield, 93%) of product as a solid. LC-MS: m/z=249.8 (M+H).

### Step 4: Preparation of methyl 3-(4-(5-chloro-2-hydroxyphenyl)-picolinamido)propanoate)

To a solution of 4-(5-chloro-2-hydroxyphenyl)picolinic acid (0.6 g, 2.40 mmol) in THF (20 ml) was sequentially added EDC (0.69 g, 3.61 mmol) and HOBT (0.49 g, 3.61 mmol) at 0° C. The reaction mixture was stirred at 0° C. for 30 minutes. Beta-alanine methyl ester (0.40 g, 2.88 mol) was added at 0° C. The reaction mixture temperature was then allowed to rise to room temperature and stirred for 20 hours. After completion of reaction, water (50 ml) was added in to the reaction mixture. The resulting mixture was then extracted with ethyl acetate (3×25 ml). The combined organic extract was washed with water (20 ml), brine (20 ml), dried over sodium sulfate and concentrated under vacuo to get the desired crude product. The crude product was purified by column chromatography using normal phase silica gel. The desired product eluted at around 0-5% Methanol in dichloromethane. Evaporation of the product fractions gave 0.72 g (yield: 89%) of desired product. LC-MS: m/z=335.6 (M+H).

### Step 5: Synthesis of methyl-3-(4-(5-chloro-2-(2-chloro-4-(N-(2,4-dimethoxybenzyl)-N-(1,2,4-thiadiazol-5-yl)sulfamoyl)-5-fluorophenoxy)phenyl)picolinamido)propanoate)

To a solution of methyl 3-(4-(5-chloro-2-hydroxyphenyl)picolinamido)propanoate) (0.72 g, 2.15 mmol) in DMF (10 ml) was added K₂CO₃ (0.59 g, 4.3 mol) in one portion under nitrogen atmosphere at room temperature. The resulting reaction mixture was then allowed to stir at room temperature for 15 minutes. To the above reaction mixture was then added calculated quantity of 5-chloro-N-(2,4-dimethoxybenzyl)-2,4-difluoro-N-(1,2,4-thiadiazol-5-yl)benzenesulfonamide (1.0 g, 2.15 mol). The resulting reaction mixture was further allowed to stir at room temperature for 3 hours. After completion of reaction, water (10 ml) was added and the resulting mixture was extracted with ethyl acetate (3×25 ml). The combined organic extract was washed with water (20 ml), brine (20 ml), dried over sodium sulfate and concentrated under vacuo. The crude product was purified by column chromatography using normal phase silica gel. The desired product eluted at around 20 to 25% ethyl acetate in hexane. Evaporation of the product fractions gave 1.0 g (yield: 60%) of desired product. LC-MS: m/z=776.3 (M+H).

### Step 6: Preparation of 3-(4-(5-chloro-2-(2-chloro-4-(N-(2,4-dimethoxybenzyl)-N-(1,2,4-thiadiazol-5-yl)sulfamoyl)-5-fluorophenoxy)phenyl)picolinamido)propanoic acid)

To the solution of methyl-3-(4-(5-chloro-2-(2-chloro-4-(N-(2,4-dimethoxybenzyl)-N-(1,2,4-thiadiazol-5-yl)sulfamoyl)-5-fluorophenoxy)phenyl)picolinamido)propanoate) (1.0 g, 1.28 mmol) in THF (10 mL) was added a solution of Lithium hydroxide monohydrate (0.27 g, 6.43 mmol) in water (5 ml). The resulting reaction mixture was then allowed to stir at room temperature for 3 hours. After completion of reaction, ice cold water was added in to the reaction mixture, the resulting mixture was acidified between pH 4-6 with 1N HCl. The resulting acidic aqueous was extracted with Ethyl acetate (3×25 ml). The combined organic extract was washed with water (20 ml), brine (20 ml), dried over sodium sulphate and concentrated under vacuo. The crude product was purified by column chromatography using normal phase silica gel. The desired product eluted at around 0 to 5% methanol in dichloromethane. Evaporation of the product fractions gave 1 g (yield: 99%) of desired product. LC-MS: m/z=762.8 (M+H).

### Step 7: Preparation of 3-(4-(2-(4-(N-1,2,4-thiadiazol-5-ylsulfamoyl)-2-chloro-5-fluorophenoxy)-5-chlorophenyl)picolinamido)propanoic acid

To the solution of 3-(4-(5-chloro-2-(2-chloro-4-(N-(2,4-dimethoxybenzyl)-N-(1,2,4-thiadiazol-5-yl)sulfamoyl)-5-fluorophenoxy)phenyl)picolinamido)propanoic acid) (1.0 g, 1.3 mmol) in DCM (10 ml) was added drop wise 4N solution of hydrochloric acid in ethyl acetate (0.5 ml) at room temperature. The resulting reaction mixture was further stirred at room temperature for 2 hour. After completion of reaction, pentane (20 ml) was added in to the reaction mixture which resulted in precipitation of solid. The solid thus obtained was washed twice with pentane (15 ml) and dried under vacuo. The resulting crude material was further purified by Prep HPLC using 0.1% HCl in water:acetonitrile mobile phase. Evaporation of the pure Prep fractions gave 0.29 g (yield: 34%) of desired product as HCl salt. LC-MS: m/z=612.9 (M+H). 1H NMR (DMSO-d6), δ 9.03 (br, 1H), 8.71 (d, J=4.8 Hz, 1H), 8.51 (s, 1H), 8.20 (s, 1H), 7.88 (d, J=7.2 Hz, 1H), 7.80 (br, 2H), 7.60 (d, J=8.4 Hz, 1H), 7.28 (d, J=8.4 Hz, 1H), 7.22 (d, J=10.8 Hz, 1H), 4.01 (br, 2H).

The following nine compounds were synthesized according to the synthetic scheme described for Example 1.

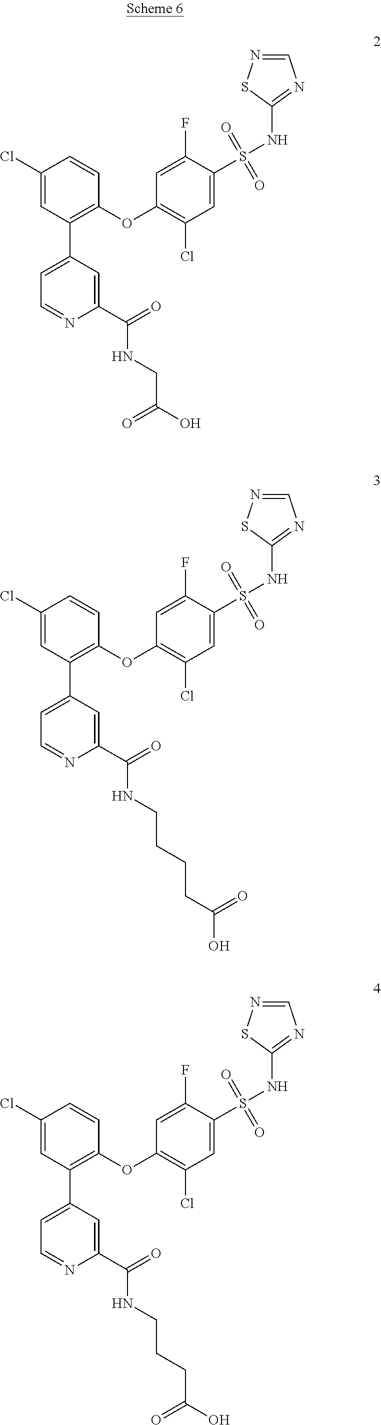 [SMILES: O=C(O)CNC(=O)c1cc(-c2cc(Cl)ccc2Oc2cc(F)c(S(=O)(=O)Nc3ncns3)cc2Cl)ccn1 ; O=C(O)CCCCNC(=O)c1cc(-c2cc(Cl)ccc2Oc2cc(F)c(S(=O)(=O)Nc3ncns3)cc2Cl)ccn1 ; O=C(O)CCCNC(=O)c1cc(-c2cc(Cl)ccc2Oc2cc(F)c(S(=O)(=O)Nc3ncns3)cc2Cl)ccn1]

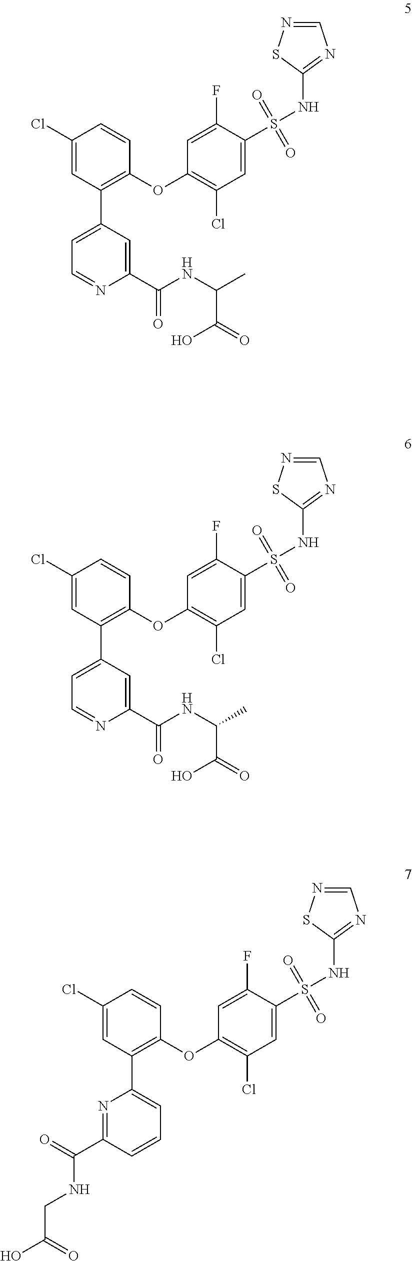 [SMILES: CC(NC(=O)c1cc(-c2cc(Cl)ccc2Oc2cc(F)c(S(=O)(=O)Nc3ncns3)cc2Cl)ccn1)C(=O)O ; C[C@@H](NC(=O)c1cc(-c2cc(Cl)ccc2Oc2cc(F)c(S(=O)(=O)Nc3ncns3)cc2Cl)ccn1)C(=O)O ; O=C(O)CNC(=O)c1cccc(-c2cc(Cl)ccc2Oc2cc(F)c(S(=O)(=O)Nc3ncns3)cc2Cl)n1]

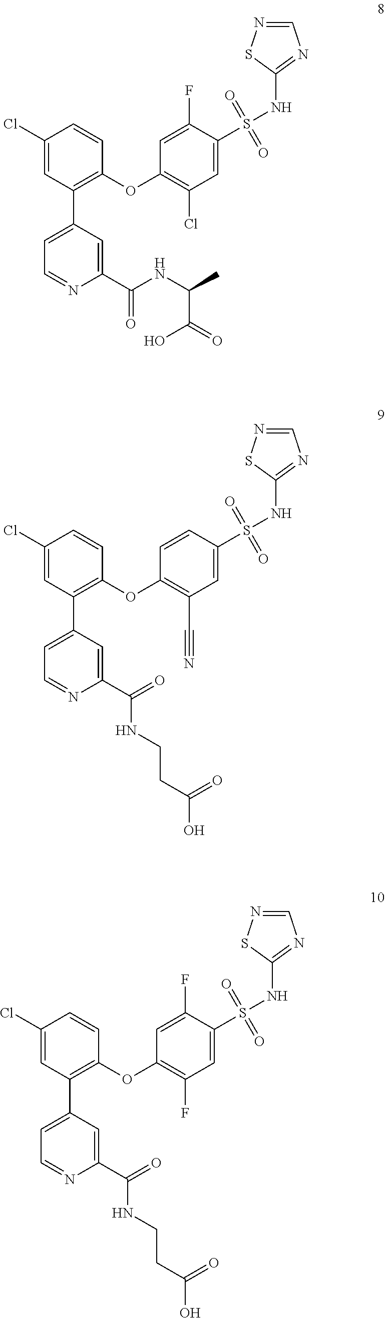 [SMILES: C[C@H](NC(=O)c1cc(-c2cc(Cl)ccc2Oc2cc(F)c(S(=O)(=O)Nc3ncns3)cc2Cl)ccn1)C(=O)O ; N#Cc1cc(S(=O)(=O)Nc2ncns2)ccc1Oc1ccc(Cl)cc1-c1ccnc(C(=O)NCCC(=O)O)c1 ; O=C(O)CCNC(=O)c1cc(-c2cc(Cl)ccc2Oc2cc(F)c(S(=O)(=O)Nc3ncns3)cc2F)ccn1]

### Example 2: 2-(4-(2-(4-(N-(1,2,4-thiadiazol-5-yl)sulfamoyl)-2-chloro-5-fluorophenoxy)-5-chlorophenyl)picolinamido)acetic acid

Compound 2 was synthesized according to the procedure described for the synthesis of example 1 by replacing beta-alanine methyl ester with glycine methyl ester hydrochloride in step 4. LC-MS: m/z=598.5 (M+H). 1H NMR (DMSO-d6), δ 9.03 (t, J=6.0 Hz, 1H), 8.71 (d, J=4.8 Hz, 1H), 8.53 (s, 1H), 8.19 (s, 1H), 7.88 (d, J=7.2 Hz, 1H), 7.78-7.81 (m, 2H), 7.60 (dd, J=2.4, 8.8 Hz, 1H), 7.29 (d, J=8.8 Hz, 1H), 7.22 (d, J=10.8 Hz, 1H), 4.00 (br, 2H).

### Example 3: 5-(4-(2-(4-(N-(1,2,4-thiadiazol-5-yl)sulfamoyl)-2-chloro-5-fluorophenoxy)-5-chlorophenyl)picolinamido)pentanoic acid

Compound 3 was synthesized according to the procedure described for the synthesis of compound 1 by replacing beta-alanine methyl ester methyl 5-aminopentanoate in step 4. LC-MS: m/z=640.2 (M+H).

### Example 4: 4-(4-(2-(4-(N-(1,2,4-thiadiazol-5-yl)sulfamoyl)-2-chloro-5-fluorophenoxy)-5-chlorophenyl)picolinamido)butanoic acid

Compound 4 was synthesized according to the procedure described for the synthesis of compound 1 by replacing beta-alanine methyl ester with methyl 4-aminobutanoate in step 4. LC-MS: m/z=626.6 (M+H). 1H NMR (MeOH-d4), δ 8.65 (d, J=4.8 Hz, 1H), 8.27 (s, 1H), 8.26 (s, 1H), 7.91 (d, J=6.8 Hz, 1H), 7.74 (d, J=4.4 Hz, 1H), 7.71 (d, J=2.4 Hz, 1H), 7.60 (dd, J=2.8, 8.8 Hz, 1H), 7.24 (d, J=8.8 Hz, 1H), 6.94 (s, 1H), 6.78 (d, J=10.8 Hz, 1H), 3.75 (br, 2H), 2.41 (t, J=7.2 Hz, 2H), 1.97 (t, J=7.2 Hz, 2H).

### Example 5: (Rac)-2-(4-(2-(4-(N-1,2,4-thiadiazol-5-ylsulfamoyl)-2-chloro-5-fluorophenoxy)-5-chlorophenyl)picolinamido)propanoic acid

Compound 5 was synthesized according to the procedure described for the synthesis of compound 1 by replacing beta-alanine methyl ester with DL-alanine methyl ester hydrochloride in step 4. LC-MS: m/z=613.8 (M+H). 1H NMR (MeOH-d4), δ 8.65 (d, J=5.6 Hz, 1H), 8.27 (s, 1H), 8.25 (s, 1H), 7.90 (d, J=6.8 Hz, 1H), 7.74 (dd, J=1.6, 4.8 Hz, 1H), 7.70 (d, J=2.4 Hz, 1H), 7.59 (dd, J=2.8, 8.8 Hz, 1H), 7.23 (d, J=8.8 Hz, 1H), 6.78 (d, J=10.8 Hz, 1H), 4.63 (q, J=7.2 Hz, 1H), 1.56 (d, J=7.6 Hz, 3H).

### Example 6: (R)-2-(4-(2-(4-(N-1,2,4-thiadiazol-5-ylsulfamoyl)-2-chloro-5-fluorophenoxy)-5-chlorophenyl)picolinamido)propanoic acid

Compound 6 was synthesized according to the procedure described for the synthesis of compound 1 by replacing beta-alanine methyl ester with D-alanine methyl ester hydrochloride in step 4. LC-MS: m/z=613.8 (M+H). 1H NMR (MeOH-d4), δ 8.67 (d, J=5.2 Hz, 1H), 8.27 (s, 1H), 8.25 (s, 1H), 7.91 (d, J=7.2 Hz, 1H), 7.75 (dd, J=2.0, 5.2 Hz, 1H), 7.71 (d, J=2.8 Hz, 1H), 7.60 (dd, J=2.4, 8.4 Hz, 1H), 7.24 (d, J=8.8 Hz, 1H), 6.78 (d, J=10.8 Hz, 1H), 4.63 (q, J=7.2 Hz, 1H), 1.56 (d, J=7.6 Hz, 3H).

### Example 7: 2-(6-(2-(4-(N-(1,2,4-thiadiazol-5-yl)sulfamoyl)-2-chloro-5-fluorophenoxy)-5-chlorophenyl)picolinamido)acetic acid

Compound 7 was synthesized according to the procedure described for the synthesis of compound 1 by replacing 4-Chloropicolinonitrile with 6-chloropicolinonitrile in step 2. LC-MS: m/z=597.7 (M+H). 1H-NMR (MeOD), δ 8.19 (s, 1H), 8.00-8.07 (m, 4H), 7.9s (d, J=6.8 Hz, 1H), 7.59 (dd, J=2.4, 8.8 Hz, 1H), 7.25 (d, J=8.8 Hz, 1H), 6.72 (d, J=10.4 Hz, 1H), 4.09 (s, 2H).

### Example 8: (S)-2-(4-(2-(4-(N-1,2,4-thiadiazol-5-ylsulfamoyl)-2-chloro-5-fluorophenoxy)-5-chlorophenyl)picolinamido)propanoic acid

Compound 8 was synthesized according to the procedure described for the synthesis of compound 1 by replacing beta-alanine methyl ester with L-alanine methyl ester hydrochloride in step 4. LC-MS: m/z=612.6 (M+H). 1H NMR (DMSO-d6), δ 8.85 (d, J=7.6 Hz, 1H), 8.71 (d, J=5.6 Hz, 1H), 8.52 (s, 1H), 8.19 (s, 1H), 7.88 (d, J=7.2 Hz, 1H), 7.78-7.80 (m, 2H), 7.60 (dd, J=2.4, 8.8 Hz, 1H), 7.28 (d, J=8.8 Hz, 1H), 7.22 (d, J=10.8 Hz, 1H), 4.47 (q, J=7.2 Hz, 1H), 1.42 (d, J=7.2 Hz, 3H).

### Example 9: 3-(4-(2-(4-(N-(1,2,4-thiadiazol-5-yl)sulfamoyl)-2-cyanophenoxy)-5-chlorophenyl)picolinamido)propanoic acid

Compound 9 was synthesized according to the procedure described for the synthesis of compound 1 by replacing 5-chloro-N-(2,4-dimethoxybenzyl)-2,4-difluoro-N-(1,2,4-thiadiazol-5-yl)benzenesulfonamide with 3-cyano-N-(2,4-dimethoxybenzyl)-4-fluoro-N-(1,2,4-thiadiazol-5-yl)benzenesulfonamide in step 5. LC-MS: m/z=584.8 (M+H). 1H-NMR (MeOD), δ 8.63 (d, J=4.8 1H), 8.23 (s, 1H), 8.19 (s, 1H), 8.14 (d, J=2.0 Hz, 1H), 7.95 (dd, J=2.4, 8.8 Hz, 1H), 7.74-7.76 (m, 2H), 7.63 (dd, J=2.4, 8.8 Hz, 1H), 6.97 (d, J=10.0 Hz, 1H), 3.68 (t, J=6.8 Hz, 2H), 2.65 (t, J=6.8 Hz, 2H).

### Example 10: 3-(4-(2-(4-(N-(1,2,4-thiadiazol-5-yl)sulfamoyl)-2,5-difluorophenoxy)-5-chlorophenyl)picolinamido)propanoic acid

Compound 10 was synthesized according to the procedure described for the synthesis of compound 1 by replacing 5-chloro-N-(2,4-dimethoxybenzyl)-2,4-difluoro-N-(1,2,4-thiadiazol-5-yl)benzenesulfonamide with N-(2,4-dimethoxybenzyl)-2,4,5-trifluoro-N-(1,2,4-thiadiazol-5-yl)benzenesulfonamide in step 5. LC-MS: m/z=595.8 (M+H). 1H-NMR (MeOD), δ 8.66 (d, J=4.8 1H), 8.28 (s, 1H), 8.26 (s, 1H), 7.69-7.77 (m, 3H), 7.56 (dd, J=2.8, 8.8 Hz, 1H), 6.94 (dd, J=6.4, 10.0 Hz, 1H), 3.70 (t, J=6.4 Hz, 2H), 2.67 (t, J=6.8 Hz, 2H).

### Example 11: Preparation of 2-(3-(5-chloro-2-(2-chloro-5-fluoro-4-(N-thiazol-4-ylsulfamoyl)phenoxy)phenyl)propylamino) acetic acid

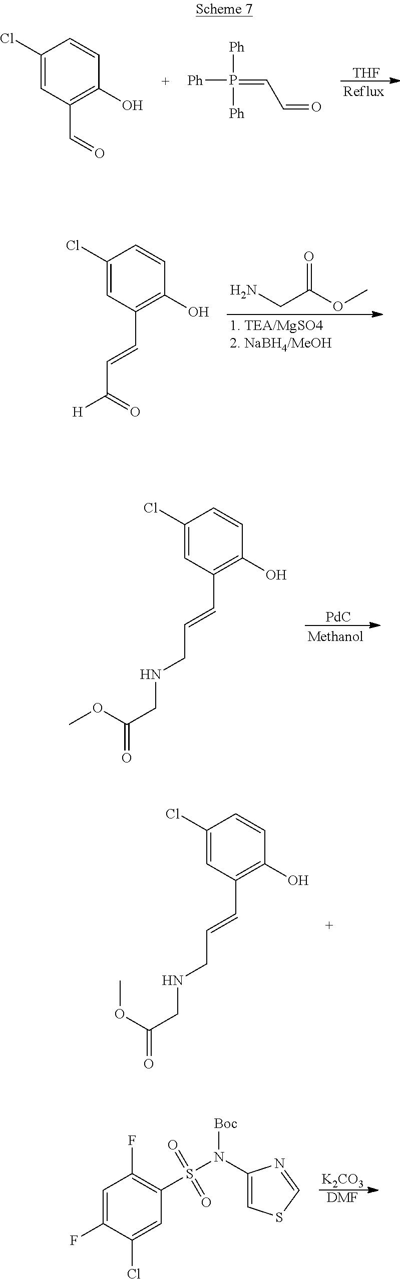

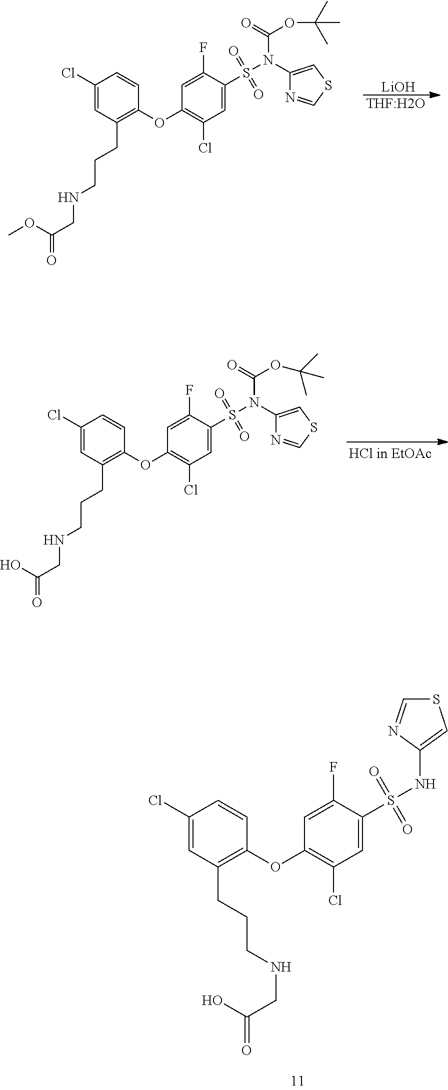 [SMILES: COC(=O)CNCCCc1cc(Cl)ccc1Oc1cc(F)c(S(=O)(=O)N(C(=O)OC(C)(C)C)c2cscn2)cc1Cl ; "LiOH" ; "THF:H2O" ; CC(C)(C)OC(=O)N(c1cscn1)S(=O)(=O)c1cc(Cl)c(Oc2ccc(Cl)cc2CCCNCC(=O)O)cc1F ; O=C(O)CNCCCc1cc(Cl)ccc1Oc1cc(F)c(S(=O)(=O)Nc2cscn2)cc1Cl]

### Step 1: Preparation of 3-(5-chloro-2-hydroxyphenyl)acrylaldehyde

To a solution of 5-chloro-2-hydroxybenzaldehyde (20 g, 127 mmol) in THF (300 ml) was added (formylmethylene)triphenylphosphorane (43 g, 140 mmol) at room temperature. The resulting reaction mixture was refluxed at 100° C. for 20 hours. The reaction mixture was cooled to room temperature, and extracted with water (200 ml) and ethyl acetate (3×250 ml). The combined organic phase was washed with water (200 ml), brine (200 ml), dried over sodium sulphate and concentrated under vacuo to give the desired crude product. The crude product was purified by column chromatography using normal phase silica gel. The desired product eluted at around 20-30% ethyl acetate in hexane. Evaporation of the product fractions gave 20 g (yield, 87%) of desired compound as yellow solid. LC-MS: m/z=183.4 (M+H).

### Step 2: Preparation of methyl 2-(3-(5-chloro-2-hydroxyphenyl) allylamino) acetate

To a solution of 3-(5-chloro-2-hydroxyphenyl)acrylaldehyde (5 g, 27 mmol) and glycine methyl ester hydrochloride (4.1 g, 32 mmol) in dichloromethane (80 ml) was added magnesium sulphate (6 g, 50 mmol) and triethylamine (12 ml, 82 mmol) at room temperature. The above reaction mixture was stirred at room temperature for 18 hours. The resulting reaction mixture was then concentrated under vacuo. The concentrated mass thus obtained was dissolved in methanol (50 ml) and cooled to a temperature between 5-10° C. To the above mixture, sodium borohydride (3.0 g, 82 mmol) was added in small portions over a period of 20 minutes; during addition temperature of the reaction mixture was maintained between 10-20° C. The reaction mixture was allowed to stir at room temperature for 2 hours and concentrated under vacuo. Water (100 ml) was added to the above crude mass and the resulting mixture was extracted with ethyl acetate (3×100 ml). The combined organic extract was washed with water (50 ml), brine (50 ml), dried over sodium sulphate and concentrated under vacuo to get the desired crude product. The crude product was purified by column chromatography using normal phase silica gel. The desired product eluted at around 1-5% methanol in dichloromethane. Evaporation of the product fractions gave 4 g (yield, 58%) of desired compound as yellow solid. LC-MS: m/z=256.43 (M+H).

### Step 3: Preparation of methyl 2-(3-(5-chloro-2-hydroxyphenyl) propylamino) acetate

To a solution of methyl 2-(3-(5-chloro-2-hydroxyphenyl) allylamino) acetate (3.5 g, 13.6 mmol) in methanol (80 ml) was carefully added 10% Palladium on carbon with 50% moisture (0.145 g, 1.3 mmol). Hydrogen gas was then bubbled into the reaction mixture at room temperature for a period of 30 minutes. After completion of the reaction, the reaction mixture was filtered through celite. The celite bed was carefully washed with some amount of methanol. The filtrate thus obtained was concentrated under vacuo to afford 3 g (yield, 85%) of compound as colorless liquid and used as is in the next step. LC-MS: m/z=258.5 (M+H).

### Step 4: Preparation of methyl 2-(3-(2-(4-(N-(tert-butoxycarbonyl)-N-(thiazol-4-yl) sulfamoyl)-2-chloro-5-fluorophenoxy)-5-chlorophenyl) propylamino) acetate

To a solution methyl 2-(3-(5-chloro-2-hydroxyphenyl) propylamino) acetate (0.7 g, 2.7 mmol) in DMF (8 ml) was added K₂CO₃ (1.2 g, 8.1 mmol) in one portion under nitrogen atmosphere at room temperature. The resulting reaction mixture was then stirred at room temperature for 15 minutes. To the above mixture was added tert-butyl 5-chloro-2,4-difluorophenylsulfonyl(thiazol-4-yl)carbamate (1.22 g, 2.9 mmol) at room temperature and the resulting reaction mixture was stirred at room temperature for 3 hrs. After completion of reaction, water (10 ml) was added and the resulting mixture was extracted with ethyl acetate (3×25 ml). The combined organic extract was washed with water (20 ml), brine (20 ml), dried over sodium sulphate and concentrated under vacuo. The crude product was purified by column chromatography using normal phase silica gel. The desired product eluted at around 20 to 25% Ethyl acetate in Hexane. Evaporation of the product fractions gave 0.6 g (yield, 36%) of desired compound as a solid. LC-MS: m/z=648.4 (M+H).

### Step 5: Preparation of 2-(3-(2-(4-(N-(tert-butoxycarbonyl)-N-(thiazol-4-yl)sulfamoyl)-2-chloro-5-fluorophenoxy)-5-chlorophenyl)propylamino)acetic acid

To the solution of methyl 2-(3-(2-(4-(N-(tert-butoxycarbonyl)-N-(thiazol-4-yl) sulfamoyl)-2-chloro-5-fluorophenoxy)-5-chlorophenyl) propylamino) acetate (0.6 g, 0.9 mmol) in THF (10 mL) was added a solution of lithium hydroxide monohydrate (0.0529, 4.6 mmol) in water (6 ml) at room temperature. The resulting reaction mixture was stirred at room temperature for 3 hours. After completion of reaction ice cold water (15 ml) was added in to the reaction mixture, the resulting mixture was then acidified between 4-6 pH with aqueous 1N hydrochloric acid. The resulting acidic aqueous was extracted with ethyl acetate (3×25 ml). The combined organic extract was washed with water (20 ml), brine (20 ml), dried over sodium sulphate and concentrated under vacuo to afford 0.5 g (yield, 85%) of compound as white solid. This material was used in the next step as is.

### Step 6: Preparation of 2-(3-(5-chloro-2-(2-chloro-5-fluoro-4-(N-thiazol-4-ylsulfamoyl) phenoxy) phenyl) propylamino) acetic acid

To the solution of 2-(3-(2-(4-(N-(tert-butoxycarbonyl)-N-(thiazol-4-yl)sulfamoyl)-2-chloro-5-fluorophenoxy)-5-chlorophenyl)propylamino)acetic acid (0.5 g, 0.78 mmol) in dichloromethane (15 ml) was added drop-wise a 4N solution of hydrochloric acid in ethyl acetate (0.5 ml) at room temperature. The resulting reaction mixture was stirred room temperature for 2 hours. After completion of reaction, pentane (20 ml) was added in to the reaction mixture which resulted in precipitation of solid. The solvent layer was decanted off; the solid thus obtained was washed twice with pentane (15 ml) and dried under vacuo. The resulting crude material was further purified by Prep HPLC using 0.1% hydrochloric acid in Water:Acetonitrile mobile phase. Evaporation of the pure product fractions obtained from Prep HPLC provided HCl salt of the desired product (0.16 g, 38% yield). LC-MS: m/z=533.9 (M+H). 1H-NMR (MeOD), δ 8.77 (d, J=2.4 Hz, 1H), 8.03 (d, J=6.8 Hz, 1H), 7.49 (d, J=2.4 Hz, 1H), 7.37 (dd, J=2.8, 8.8 Hz, 1H), 7.12 (d, J=2.4 Hz, 1H), 7.03 (d, J=8.8 Hz, 1H), 6.76 (d, J=10.8 Hz, 1H), 3.8 (s, 2H), 3.09-3.05 (m, 2H), 2.68 (t, J=7.6 Hz, 2H), 2.04-2.01 (m, 2H).

The compounds 12 to 32 were synthesized according to the synthetic scheme described for example 11.

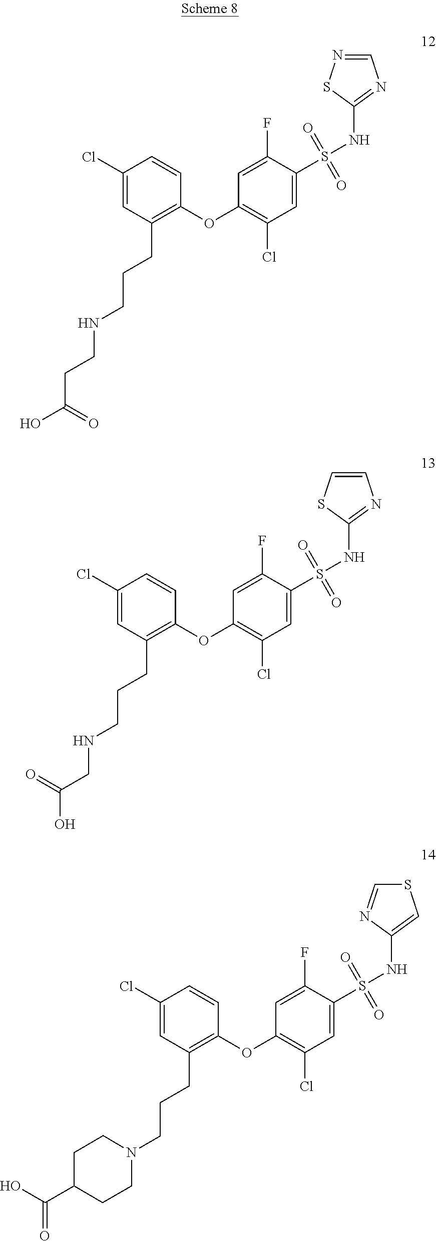 [SMILES: O=C(O)CCNCCCc1cc(Cl)ccc1Oc1cc(F)c(S(=O)(=O)Nc2ncns2)cc1Cl ; O=C(O)CNCCCc1cc(Cl)ccc1Oc1cc(F)c(S(=O)(=O)Nc2nccs2)cc1Cl ; O=C(O)C1CCN(CCCc2cc(Cl)ccc2Oc2cc(F)c(S(=O)(=O)Nc3cscn3)cc2Cl)CC1]

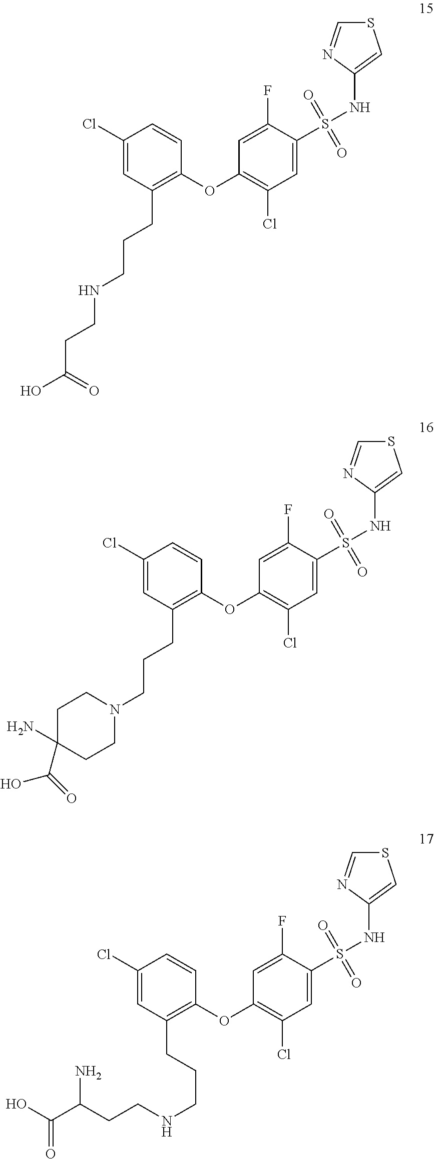 [SMILES: O=C(O)CCNCCCc1cc(Cl)ccc1Oc1cc(F)c(S(=O)(=O)Nc2cscn2)cc1Cl ; NC1(C(=O)O)CCN(CCCc2cc(Cl)ccc2Oc2cc(F)c(S(=O)(=O)Nc3cscn3)cc2Cl)CC1 ; NC(CCNCCCc1cc(Cl)ccc1Oc1cc(F)c(S(=O)(=O)Nc2cscn2)cc1Cl)C(=O)O]

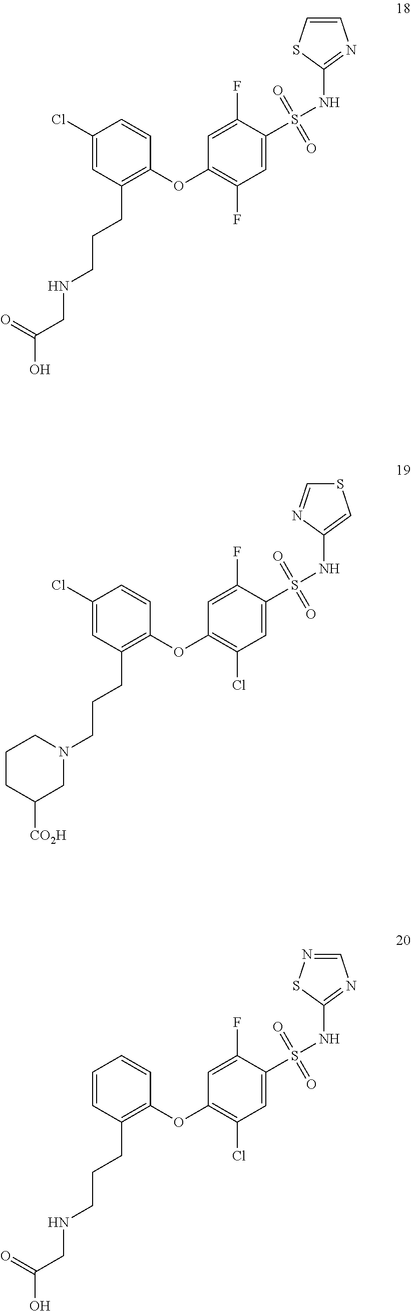 [SMILES: O=C(O)CNCCCc1cc(Cl)ccc1Oc1cc(F)c(S(=O)(=O)Nc2nccs2)cc1F ; O=C(O)C1CCCN(CCCc2cc(Cl)ccc2Oc2cc(F)c(S(=O)(=O)Nc3cscn3)cc2Cl)C1 ; O=C(O)CNCCCc1ccccc1Oc1cc(F)c(S(=O)(=O)Nc2ncns2)cc1Cl]

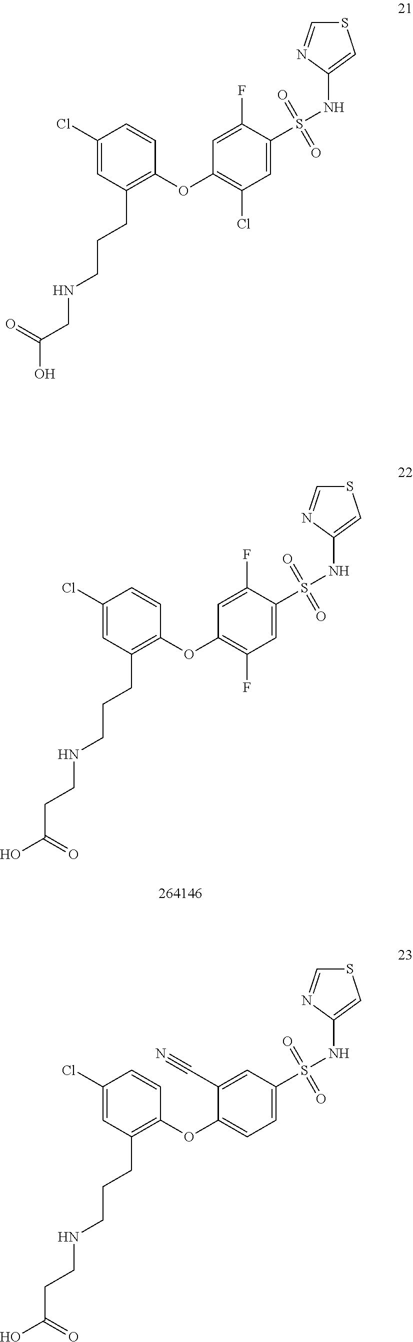 [SMILES: O=C(O)CNCCCc1cc(Cl)ccc1Oc1cc(F)c(S(=O)(=O)Nc2cscn2)cc1Cl ; O=C(O)CCNCCCc1cc(Cl)ccc1Oc1cc(F)c(S(=O)(=O)Nc2cscn2)cc1F ; N#Cc1cc(S(=O)(=O)Nc2cscn2)ccc1Oc1ccc(Cl)cc1CCCNCCC(=O)O]

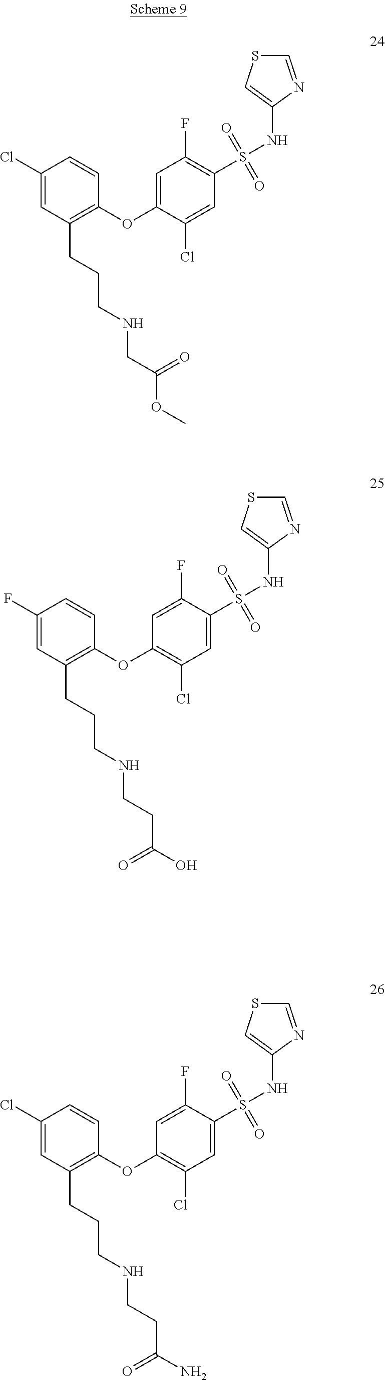 [SMILES: COC(=O)CNCCCc1cc(Cl)ccc1Oc1cc(F)c(S(=O)(=O)Nc2cscn2)cc1Cl ; O=C(O)CCNCCCc1cc(F)ccc1Oc1cc(F)c(S(=O)(=O)Nc2cscn2)cc1Cl ; NC(=O)CCNCCCc1cc(Cl)ccc1Oc1cc(F)c(S(=O)(=O)Nc2cscn2)cc1Cl]

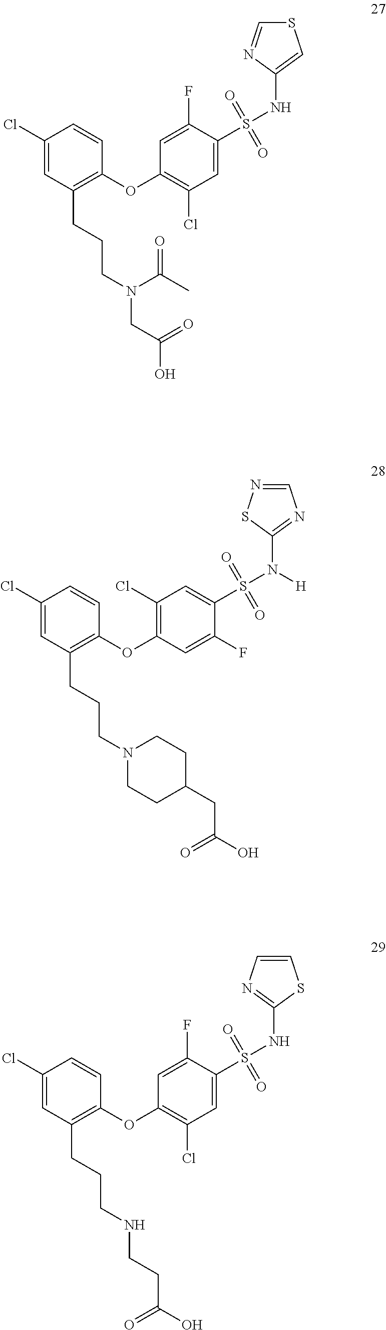 [SMILES: CC(=O)N(CCCc1cc(Cl)ccc1Oc1cc(F)c(S(=O)(=O)Nc2cscn2)cc1Cl)CC(=O)O ; O=C(O)CC1CCN(CCCc2cc(Cl)ccc2Oc2cc(F)c(S(=O)(=O)Nc3ncns3)cc2Cl)CC1 ; O=C(O)CCNCCCc1cc(Cl)ccc1Oc1cc(F)c(S(=O)(=O)Nc2nccs2)cc1Cl]

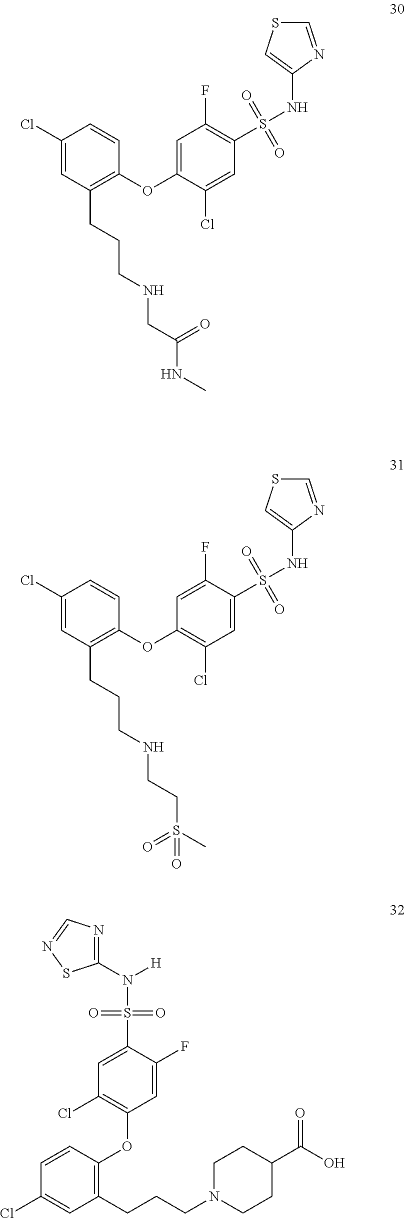 [SMILES: CNC(=O)CNCCCc1cc(Cl)ccc1Oc1cc(F)c(S(=O)(=O)Nc2cscn2)cc1Cl ; CS(=O)(=O)CCNCCCc1cc(Cl)ccc1Oc1cc(F)c(S(=O)(=O)Nc2cscn2)cc1Cl ; O=C(O)C1CCN(CCCc2cc(Cl)ccc2Oc2cc(F)c(S(=O)(=O)Nc3ncns3)cc2Cl)CC1]

### Example 12: 3-((3-(2-(4-(N-(1,2,4-thiadiazol-5-yl)sulfamoyl)-2-chloro-5-fluorophenoxy)-5-chlorophenyl)propyl)amino)propanoic acid

Compound 12 was synthesized according to the procedure described for the synthesis of compound 11 by replacing glycine methyl ester with beta alanine methyl ester in step 2, and replacing tert-butyl 5-chloro-2,4-difluorophenylsulfonyl(thiazol-4-yl)carbamate with 5-chloro-N-(2,4-dimethoxybenzyl)-2,4-difluoro-N-(1,2,4-thiadiazol-5-yl)benzenesulfonamide in step 4. LC-MS: m/z=549.6 (M+H). 1H-NMR (MeOD), δ 8.27 (s, 1H), 8.05 (d, J=7.2 Hz, 1H), 7.49 (d, J=2.4 Hz, 1H), 7.36 (dd, J=2.8, 8.8 Hz, 1H), 7.03 (d, J=8.8 Hz, 1H), 6.78 (d, J=6.4 Hz, 1H), 3.26 (t, J=6.4 Hz, 2H), 3.08 (t, J=7.6 Hz, 2H), 2.68-2.75 (m, 4H), 2.01-2.06 (m, 2H).

### Example 13: 2-((3-(5-chloro-2-(2-chloro-5-fluoro-4-(N-(thiazol-2-yl)sulfamoyl)phenoxy)phenyl)propyl)amino)acetic acid

Compound 13 was synthesized according to the procedure described for the synthesis of compound 11 by replacing tert-butyl 5-chloro-2,4-difluorophenylsulfonyl(thiazol-4-yl)carbamate with 5-chloro-N-(2,4-dimethoxybenzyl)-2,4-difluoro-N-(thiazol-2-yl)benzenesulfonamide in step 4. LC-MS: m/z=533.8 (M+H). 1H-NMR (MeOD), δ 7.94 (d, J=6.8 Hz, 1H), 7.52 (d, J=5.8, 1H), 7.35-7.38 (dd, J=2.4, 8.8 Hz, 1H), 7.33 (d, J=4.4 Hz, 1H), 7.11 (d, J=8.8 Hz, 1H), 6.91-6.94 (m, 2H), 3.60 (s, 2H), 2.80 (m, 2H), 2.56 (m, 2H), 1.99 (m, 2H).

### Example 14: 1-(3-(5-chloro-2-(2-chloro-5-fluoro-4-(N-(thiazol-4-yl)sulfamoyl)phenoxy)phenyl)propyl)piperidine-4-carboxylic acid

Compound 14 was synthesized according to the procedure described for the synthesis of compound 11 by replacing glycine methyl ester with methyl piperidine-4-carboxylate in step 2. LC-MS: m/z=589.8 (M+H).

### Example 15: 3-((3-(5-chloro-2-(2-chloro-5-fluoro-4-(N-(thiazol-4-yl)sulfamoyl)phenoxy)phenyl)propyl)amino)propanoic acid

Compound 15 was synthesized according to the procedure described for the synthesis of compound 11 by replacing glycine methyl ester with beta alanine methyl ester in step 2. LC-MS: m/z=547.8 (M+H). 1H-NMR (MeOD), δ 8.77 (d, J=2.0 Hz, 1H), 8.03 (d, J=10.8 Hz, 1H), 7.49 (d, J=2.4 Hz, 1H), 7.35-7.38 (m, 1H), 7.12 (d, J=2.8 Hz, 1H), 7.03 (d, J=8.4 Hz, 1H), 6.76 (d, J=10.4 Hz, 1H), 3.26 (br, 2H), 3.07 (br, 2H), 2.67-2.76 (m, 4H), 2.02 (br, 2H).

### Example 16: 4-amino-1-(3-(5-chloro-2-(2-chloro-5-fluoro-4-(N-(thiazol-4-yl)sulfamoyl)phenoxy)phenyl)propyl)piperidine-4-carboxylic acid

Compound 16 was synthesized according to the procedure described for the synthesis of compound 11 by replacing glycine methyl ester with methyl 4-((tert-butoxycarbonyl)amino)piperidine-4-carboxylate in step 2. LC-MS: m/z=602.8 (M+H). 1H-NMR (MeOD), δ 8.77 (d, J=2.0 Hz, 1H), 8.02 (d, J=7.2 Hz, 1H), 7.52 (d, J=2.8 Hz, 1H), 7.36-7.38 (dd, J=2.8, 8.8 Hz, 1H), 7.12 (d, J=2.0 Hz, 1H), 7.03 (d, J=8.4 Hz, 1H), 6.77 (d, J=10.4 Hz, 1H), 3.25-3.70 (m, 6H) 2.67-2.71 (m, 2H), 2.50 (br, 2H), 2.27 (br, 2H), 2.12 (br, 2H).

### Example 17: 2-amino-4-((3-(5-chloro-2-(2-chloro-5-fluoro-4-(N-(thiazol-4-yl)sulfamoyl)phenoxy)phenyl)propyl)amino)butanoic acid

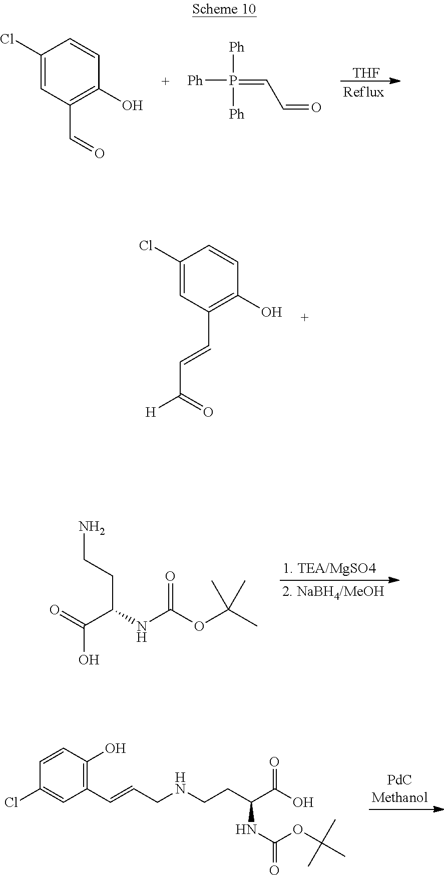 [SMILES: O=Cc1cc(Cl)ccc1O ; O=CC=P(c1ccccc1)(c1ccccc1)c1ccccc1 ; O=C/C=C/c1cc(Cl)ccc1O ; CC(C)(C)OC(=O)N[C@@H](CCN)C(=O)O ; "THF" ; CC(C)(C)OC(=O)N[C@@H](CCNC/C=C/c1cc(Cl)ccc1O)C(=O)O]

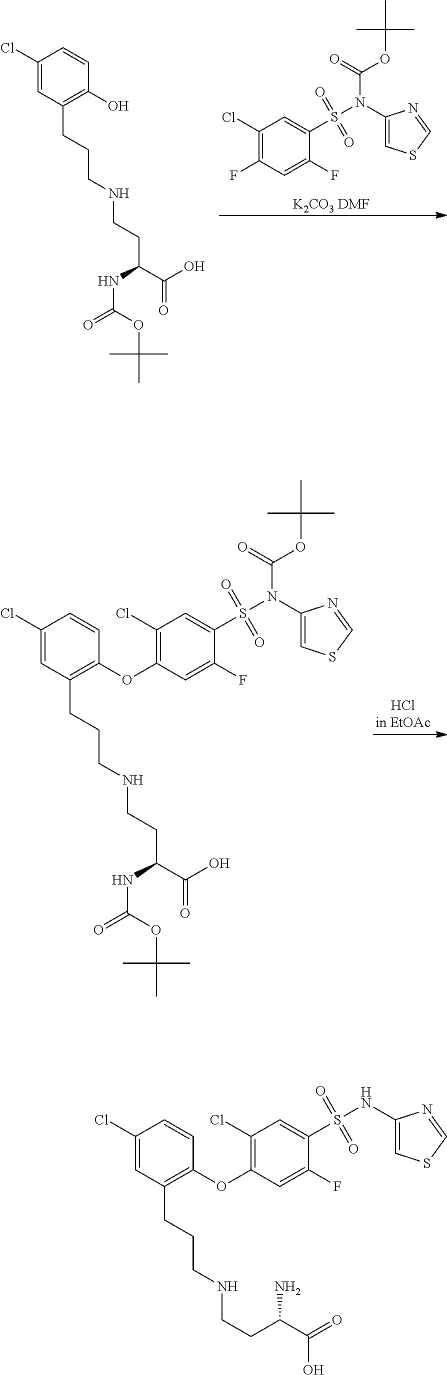

### Step 1: Preparation of (S)-4-amino-2-(tert-butoxycarbonylamino)butanoic acid

To a solution of (S)-5-amino-2-(tert-butoxycarbonylamino)-5-oxopentanoic acid (2 g, 8.1 mmol) in DMF: water (1:1,v/v,18 ml) was added pyridine (1.3 ml, 16.2 mmol). The resulting reaction mixture was stirred at room temperature for 5-10 minutes. Iodobenzene diacetate (3.92 g, 12.1 mmol) was added and further stirred for 4 hours. After completion of reaction D.M. water (100 ml) was added and the resulting mixture was extracted with ethyl acetate (3×100 ml). The combined organic extracts was washed with D.M. water (100 ml), brine (100 ml), dried over sodium sulphate and concentrated under vacuo to get the desired crude product. The crude product was purified by triturating with diethyl ether. Evaporation of the product fractions gave 1.1 g (yield, 62%) of desired compound as brown solid. LC-MS: m/z=219.1 (M+H).

### Step 2: Preparation of (E)-3-(5-chloro-2-hydroxyphenyl)acrylaldehyde

To a solution of 5-chloro-2-hydroxybenzaldehyde (20 g, 127 mmol) in THF (300 ml) was added (Formylmethylene)triphenylphosphorane (43 g, 140 mmol) at room temperature. The resulting reaction mixture was then refluxed at 100° C. for 20 hrs. After completion of reaction, the reaction mixture was allowed to cool to room temperature. D.M. water (200 ml) was added and the resulting mixture was extracted with ethyl acetate (3×250 ml). The combined organic extract was washed with D.M. water (200 ml), brine (200 ml), dried over sodium sulphate and concentrated under vacuo to get the desired crude product. The crude product was purified by column chromatography using normal phase silica gel. The desired product eluted at around 20-30% ethyl acetate in hexane. Evaporation of the product fractions gave 20 g (yield, 87%) of the desired compound as yellow solid. LC-MS: m/z=183.4 (M+H).

### Step 3: (S,E)-2-(tert-butoxycarbonylamino)-4-(3-(5-chloro-2-hydroxyphenyl)allylamino)butanoic acid

To a solution of 3-(5-chloro-2-hydroxyphenyl)acrylaldehyde (0.5 g, 3.2 mmol) and (S)-4-amino-2-(tert-butoxycarbonylamino)butanoic acid (0.769 g, 3.52 mmol) in dichloromethane (80 ml) was added magnesium sulphate (0.77 g, 6.4 mmol) and triethylamine (1.34 ml, 9.615 mmol) at room temperature. The above reaction mixture was stirred at room temperature for 12 hours. The resulting reaction mixture was then concentrated under vacuo. The concentrated mass thus obtained was dissolved in methanol (20 ml) and cooled to a temperature between 5-10° C. To the above mixture, sodium borohydride (0.36 g, 9.61 mmol) was added in small portions over a period of 10 minutes, during addition temperature of the reaction mixture was maintained between 10-20° C. After completion of addition, the resulting reaction mixture was allowed to stir at room temperature for 2 hours. After completion of reaction, the reaction mixture was concentrated under vacuo. D.M. water (40 ml) was added to the above crude mass and the resulting mixture was extracted with ethyl acetate (3×60 ml). The combined organic extract was washed with D.M. water (50 ml), brine (50 ml), dried over sodium sulphate and concentrated under vacuo to get the desired crude product. The crude product was purified by column chromatography using normal phase silica gel. The desired product eluted at around 1-5% methanol in dichloromethane. Evaporation of the product fractions gave 0.4 g (yield, 32.5%) of the desired compound as a brown liquid. LC-MS: m/z=385.2 (M+H).

### Step 4: (S)-2-(tert-butoxycarbonylamino)-4-(3-(5-chloro-2-hydroxyphenyl)propylamino)butanoic acid

To a solution of (S,E)-2-(tert-butoxycarbonylamino)-4-(3-(5-chloro-2-hydroxyphenyl)allylamino)butanoic acid (0.4 g, 13.6 mmol) in methanol (10 ml) was carefully added 10% Palladium on carbon with 50% moisture (0.120 g, 1.3 mmol). Hydrogen gas was then bubbled into the reaction mixture at room temperature for a period of 15-20 minutes. After completion of the reaction, the reaction mixture was filtered through celite hyflow. The celite bed was carefully washed with some amount of methanol. The filtrate thus obtained was concentrated under vacuo to afford 0.35 g (yield, 87.06%) of the desired compound as a colorless liquid. LC-MS: m/z=387.4 (M+H).

Note: For this particular step, we also observed occurrence of dechlorination, its proportion remained variable. This step was thus monitored cautiously and worked up soon upon completion.

### Step 5: (S)-4-(3-(2-(4-(N-(tert-butoxycarbonyl)-N-(thiazol-4-yl)sulfamoyl)-2-chloro-5-fluorophenoxy)-5-chlorophenyl)propylamino)-2-(tert-butoxycarbonylamino)butanoic acid

To a solution (S)-2-(tert-butoxycarbonylamino)-4-(3-(5-chloro-2-hydroxyphenyl)propylamino)butanoic acid (0.350 g, 2.7 mmol) in DMF (0.7 ml) was added K₂CO₃ (0.375 g, 2.7 mmol) in one portion under nitrogen atmosphere at room temperature. The resulting reaction mixture was then stirred at room temperature for 15 minutes. To the above mixture was added tert-butyl 5-chloro-2,4-difluorophenylsulfonyl(thiazol-4-yl) carbamate (0.408 g, 0.99 mmol) and the resulting reaction mixture was stirred at room temperature for 3 hours. After completion of reaction, D.M. water (20 ml) was added and the resulting mixture was extracted with ethyl acetate (3×30 ml). The combined organic extract was washed with ice cold water (100 ml), brine (50 ml), dried over sodium sulphate and concentrated under vacuo. The crude product was purified by column chromatography using normal phase silica gel. The desired product eluted at around 1 to 2% Methanol in DCM. Evaporation of the product fractions gave 0.4 g (yield, 56.8%) of the desired compound as a brown liquid. LC-MS: m/z=777.6 (M+H).

### Step 6: Preparation of (S)-2-amino-4-(3-(5-chloro-2-(2-chloro-5-fluoro-4-(N-thiazol-4-ylsulfamoyl)phenoxy) phenyl)propylamino)butanoic acid

To a solution of (S)-4-(3-(2-(4-(N-(tert-butoxycarbonyl)-N-(thiazol-4-yl)sulfamoyl)-2-chloro-5-fluorophenoxy)-5-chlorophenyl)propylamino)-2-(tert-butoxycarbonylamino)butanoic acid (0.4 g, 0.78 mmol) in dichloromethane (10 ml) was added drop-wise a 4N solution of hydrochloric acid in ethyl acetate (2 ml) at room temperature. The resulting reaction mixture was stirred room temperature for 2 hours. After completion of reaction, pentane (20 ml) was added in to the reaction mixture which resulted in precipitation of solid. The solvent layer was decanted off; the solid thus obtained was washed twice with pentane (15 ml) and dried under vacuo. The resulting crude material was further purified by Prep HPLC using 0.1% Formic acid in Water: Acetonitrile mobile phase. Evaporation of the pure product fractions obtained from Prep HPLC provided the desired product as HCl salt (0.0253 g, 8.6% yield). LC-MS: m/z=576.8 (M+H).

### Example 18: 2-((3-(5-chloro-2-(2,5-difluoro-4-(N-(thiazol-2-yl)sulfamoyl)phenoxy)phenyl)propyl)amino)acetic acid

Compound 18 was synthesized according to the procedure described for the synthesis of compound 11 by replacing tert-butyl 5-chloro-2,4-difluorophenylsulfonyl(thiazol-4-yl)carbamate with N-(2,4-dimethoxybenzyl)-2,4,5-trifluoro-N-(thiazol-2-yl)benzenesulfonamide in step 4. LC-MS: m/z=517.8 (M+H). 1H-NMR (MeOD), δ 7.81-7.85 (dd, J=6.4, 10.4 Hz, 1H), 7.46 (d, J=6.4, 1H), 7.31-7.34 (dd, J=2.8, 8.8 Hz, 1H), 7.17 (d, J=4.8 Hz, 1H), 6.99 (d, J=8.4 Hz, 1H), 6.86-6.90 (dd, J=6.4, 10.0 Hz, 1H), 6.81 (d, J=4.8 Hz, 1H), 3.92 (s, 2H), 3.08-3.12 (m, 2H), 2.75 (t, J=8.0 Hz, 2H), 2.03-2.08 (m, 2H).

### Example 19: 1-(3-(5-chloro-2-(2-chloro-5-fluoro-4-(N-(thiazol-4-yl)sulfamoyl)phenoxy)phenyl)propyl)piperidine-3-carboxylic acid

Compound 19 was synthesized according to the procedure described for the synthesis of compound 11 by replacing glycine methyl ester with methyl piperidine-3-carboxylate in step 2. LC-MS: m/z=589.8 (M+H).

### Example 20: 2-((3-(2-(4-(N-(1,2,4-thiadiazol-5-yl)sulfamoyl)-2-chloro-5-fluorophenoxy)phenyl)propyl)amino)acetic acid

Compound 20 was synthesized according to the procedure described for the synthesis of compound 11 by replacing 5-chloro-2-hydroxybenzaldehyde with 2-hydroxybenzaldehyde in step 1. LC-MS: m/z=500.8 (M+H). 1H-NMR (MeOD), δ 8.90 (s, 2H), 8.51 (s, 1H), 7.97 (d, J=7.2 Hz, 1H), 7.41-7.44 (dd, J=1.6, 7.2 Hz, 1H), 7.26-7.34 (m, 2H), 7.07 (dd, J=1.2, 8.0 Hz, 1H), 6.81 (d, J=10.8 Hz, 1H), 3.89 (s, 2H), 2.93 (br, 2H), 2.57-2.61 (m, 2H), 1.92 (br, 2H).

### Example 21: 2-((3-(5-chloro-2-(2,5-difluoro-4-(N-(thiazol-4-yl)sulfamoyl)phenoxy)phenyl)propyl)amino)acetic acid

Compound 21 was synthesized according to the procedure described for the synthesis of compound 11 by replacing tert-butyl 5-chloro-2,4-difluorophenylsulfonyl(thiazol-4-yl)carbamate with tert-butyl 2,4,5-trifluorophenylsulfonyl(thiazol-4-yl)carbamate in step 4. LC-MS: m/z=517.8 (M+H). 1H-NMR (MeOD), δ 8.77 (d, J=2.0 Hz, 1H), 7.79-7.83 (dd, J=6.4, 10.0 Hz, 1H), 7.47 (d, J=2.4 Hz, 1H), 7.32-7.35 (dd, J=2.4, 8.4 Hz, 1H), 7.11 (d, J=2.4 Hz, 1H), 7.02 (d, J=8.8 Hz, 1H), 6.85-6.89 (dd, J=6.4, 10.4 Hz, 1H), 3.92 (s, 2H), 3.09-3.16 (m, 2H), 2.73 (t, J=7.6 Hz, 2H), 1.99-2.07 (m, 2H).

### Example 22: 3-((3-(5-chloro-2-(2,5-difluoro-4-(N-(thiazol-4-yl)sulfamoyl)phenoxy)phenyl)propyl)amino)propanoic acid

Compound 22 was synthesized according to the procedure described for the synthesis of compound 11 by replacing glycine methyl ester with beta alanine methyl ester in step 2, and replacing tert-butyl 5-chloro-2,4-difluorophenylsulfonyl(thiazol-4-yl)carbamate with tert-butyl 2,4,5-trifluorophenylsulfonyl(thiazol-4-yl)carbamate in step 4. LC-MS: m/z=531.8 (M+H). 1H-NMR (MeOD), δ 8.78 (d, J=2.4 Hz, 1H), 7.79-7.83 (dd, J=6.4, 10.4 Hz, 1H), 7.47 (d, J=2.4 Hz, 1H), 7.32-7.35 (dd, J=2.4, 8.4 Hz, 1H), 7.11 (d, J=2.4 Hz, 1H), 7.01 (d, J=8.8 Hz, 1H), 6.85-6.90 (dd, J=6.4, 10.4 Hz, 1H), 3.27 (t, J=6.8 Hz, 2H), 3.07 (t, J=8.0 Hz, 2H), 2.71-2.78 (m, 4H), 1.97-2.05 (m, 2H).

### Example 23: 3-((3-(5-chloro-2-(2-cyano-4-(N-(thiazol-4-yl)sulfamoyl)phenoxy)phenyl)propyl)amino)propanoic acid

Compound 23 was synthesized according to the procedure described for the synthesis of compound 11 by replacing glycine methyl ester with beta alanine methyl ester in step 2, and replacing tert-butyl 5-chloro-2,4-difluorophenylsulfonyl(thiazol-4-yl)carbamate with tert-butyl (3-cyano-4-fluorophenyl)sulfonyl(thiazol-4-yl)carbamate in step 4. LC-MS: m/z=520.9 (M+H). 1H-NMR (MeOD), δ 8.77 (d, J=2.0 Hz, 1H), 8.30 (d, J=2.0 Hz, 1H), 8.03 (dd, J=2.4, 9.2 Hz, 1H), 7.52 (d, J=2.4 Hz, 1H), 7.39 (dd, J=2.8, 8.8 Hz, 1H), 7.16 (d, J=2.0 Hz, 1H), 7.14 (s, 1H), 6.96 (d, J=9.2 Hz, 1H), 3.09 (t, J=6.8 Hz, 2H), 3.09 (t, J=8.0 Hz, 2H), 2.76 (t, J=6.4 Hz, 2H), 2.69 (t, J=8.0 Hz, 2H), 1.99-2.07 (m, 2H).

### Example 24: methyl 2-((3-(5-chloro-2-(2-chloro-5-fluoro-4-(N-(thiazol-4-yl)sulfamoyl)phenoxy)phenyl)propyl)amino)acetate

Compound 24 was synthesized according to the procedure described for the synthesis of compound 11 without hydrolysis of methyl ester (step 5). LC-MS: m/z=548.4 (M+H). 1H-NMR (MeOD), δ 8.77 (d, J=2.4 Hz, 1H), 8.02 (d, J=6.8 Hz, 1H), 7.49 (d, J=2.4 Hz, 1H), 7.35-7.38 (dd, J=2.4, 8.4 Hz, 1H), 7.12 (d, J=2.4 Hz, 1H), 7.02 (d, J=8.8 Hz, 1H), 6.75 (d, J=10.4 Hz, 1H), 3.99 (s, 2H), 3.85 (s, 3H), 3.08-3.12 (m, 2H), 2.68 (t, J=7.6 Hz, 2H), 2.00-2.08 (m, 2H).

### Example 25: 3-((3-(2-(2-chloro-5-fluoro-4-(N-(thiazol-4-yl)sulfamoyl)phenoxy)-5-fluorophenyl)propyl)amino)propanoic acid

Compound 25 was synthesized according to the procedure described for the synthesis of compound 11 by replacing 5-chloro-2-hydroxybenzaldehyde with 5-fluoro-2-hydroxybenzaldehyde in step 1, and replacing glycine methyl ester with beta alanine methyl ester in step 2. LC-MS: m/z=531.9 (M+H). 1H-NMR (MeOD), δ 8.77 (d, J=2.4 Hz, 1H), 8.01 (d, J=6.8 Hz, 1H), 7.23 (dd, J=2.4, 8.8 Hz, 1H), 7.11-7.13 (m, 3H), 6.65 (d, J=10.8 Hz, 1H), 3.25 (t, J=6.8 Hz, 2H), 3.06 (t, J=8.0 Hz, 2H), 2.73 (t, J=6.4 Hz, 2H), 2.66 (t, J=7.6 Hz, 2H), 1.99-2.03 (m, 2H).

### Example 26: 3-((3-(5-chloro-2-(2-chloro-5-fluoro-4-(N-(thiazol-4-yl)sulfamoyl)phenoxy)phenyl)propyl)amino)propanamide

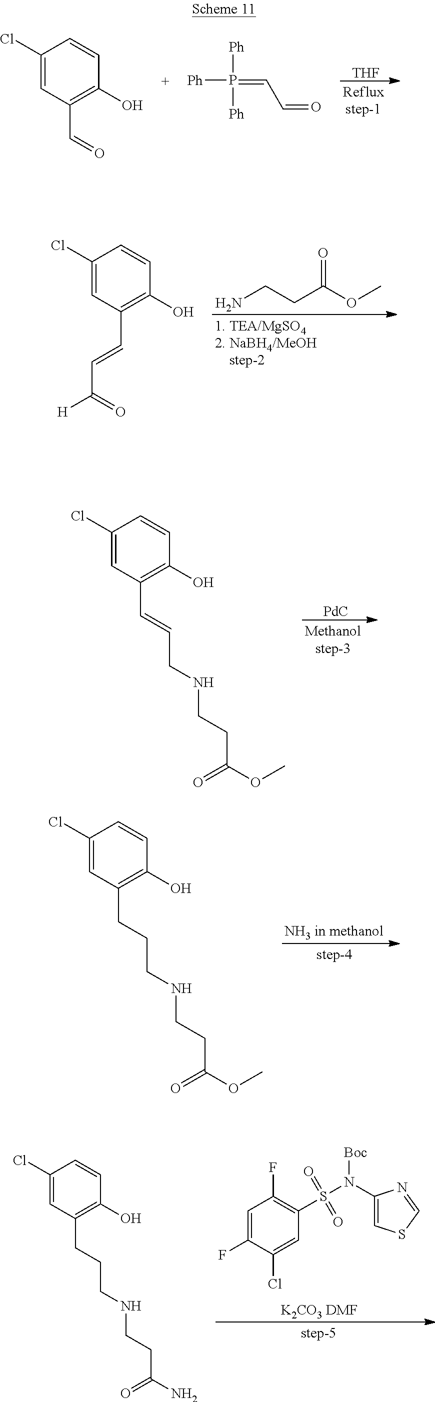

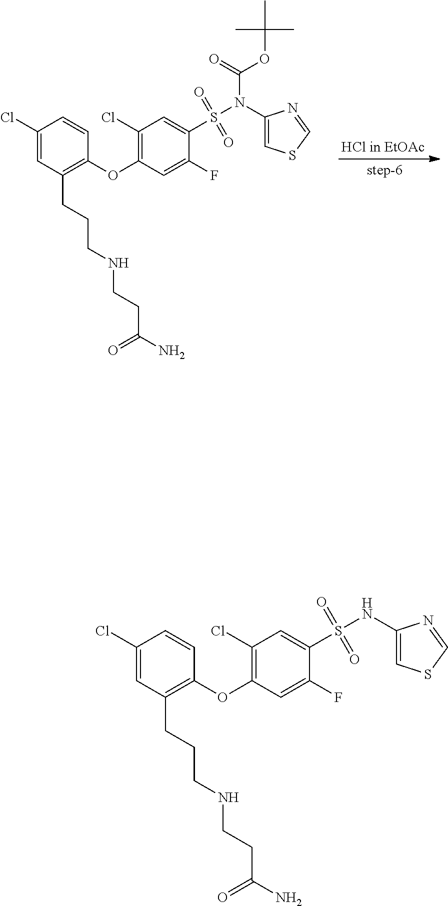 [SMILES: CC(C)(C)OC(=O)N(c1cscn1)S(=O)(=O)c1cc(Cl)c(Oc2ccc(Cl)cc2CCCNCCC(N)=O)cc1F ; NC(=O)CCNCCCc1cc(Cl)ccc1Oc1cc(F)c(S(=O)(=O)Nc2cscn2)cc1Cl]

### Step 1: Preparation of 3-(5-chloro-2-hydroxyphenyl)acrylaldehyde

To a solution of 5-chloro-2-hydroxybenzaldehyde (20 g, 127 mmol) in THF (300 ml) was added (formylmethylene)triphenylphosphorane (43 g, 140 mmol) at room temperature. The resulting reaction mixture was then refluxed at 100° C. for 20 hrs. After completion of reaction, the reaction mixture was allowed to cool to room temperature. Water (200 ml) was added and the resulting mixture was extracted with ethyl acetate (3×250 ml). The combined organic extract was washed with water (200 ml), brine (200 ml), dried over sodium sulphate and concentrated under vacuo to get the desired crude product. The crude product was purified by column chromatography using normal phase silica gel. The desired product eluted at around 20-30% ethyl acetate in hexane. Evaporation of the product fractions gave 20 g (yield, 87%) of desired compound as yellow solid. LC-MS: m/z=181.34 (M−H).

### Step 2: Preparation of methyl 3-[3-(5-chloro-2-hydroxyphenyl)allylamino]propanoate)

To a solution of 3-(5-chloro-2-hydroxyphenyl)acrylaldehyde (1.0 g, 5.47 mmol) and 13-Alanine methyl ester hydrochloride (0.917 g, 6.57 mmol) in DCM (20 ml) was added magnesium sulphate (1.317 g, 1.09 mmol) and TEA (2.3 ml, 16.41 mmol) at room temperature and the resulting reaction mixture was stirred at room temperature for 12 hours. The reaction mixture was then concentrated under vacuo. The concentrated mass thus obtained was dissolved in methanol (20 ml) and cooled to 5-10° C. To this cold reaction mixture, sodium borohydrate (0.620 g, 16.41 mmol) was then added in small portions over a period of 10-20 mins, during addition the temperature was maintained in between 10-20° C. After completion of addition the resulting reaction mixture was allowed to stir at room temperature for 2 hours. After completion of the reaction, it was concentrated under vacuo. To the resulting crude mass water (50 ml) was added and the mixture was extracted with EtOAc (3×25 ml). The combined organic extract was washed with water (20 ml), brine (20 ml), dried over sodium sulphate and concentrated under vacuo to get the desired crude product. The crude product was purified by column chromatography using normal phase silica gel. The desired product eluted at around 1-5% Methanol in DCM. Evaporation of the product fractions gave 0.9 g (yield, 61%) of desired compound as white solid. LC-MS: m/z=270.6 (M+H).

### Step 3: Preparation of methyl 3-[3-(5-chloro-2-hydroxyphenyl)propylamino] propanoate)

To a solution of 3-[3-(5-chloro-2-hydroxyphenyl)allylamino]propanoate) (0.35 g, 1.3 mmol) in methanol (20 ml) was carefully added 10% Palladium on carbon with 50% moisture (0.104 g, 0.065 mmol). Hydrogen gas was then bubbled into the reaction mixture at room temperature for a period of 30 mins. The reaction mixture was monitored on TLC using ethyl acetate as mobile phase. After completion of the reaction, the reaction mixture was filtered through celite. The celite bed was carefully washed with some amount of methanol. The filtrate thus obtained was concentrated under vacuo to afford 0.3 g (yield, 85%) of desired compound colorless liquid. m/z=272.6 (M+H).

### Step 4: Preparation of 3-[3-(5-chloro-2-hydroxyphenyl)propylamino]propanamide)

A solution of methyl 3-[3-(5-chloro-2-hydroxyphenyl)propylamino] propanoate) (0.3 g, 1.08 mmol) in methanolic ammonia (10 mL) was heated at 100° C. in sealed tube (35 mL) for a time period of 12 hours. After completion of reaction methanol was evaporated under vacuo. The crude product was purified by column chromatography using normal phase silica gel. The desired product eluted at around 30-40% ethyl acetate in hexane. Evaporation of the product fractions gave 0.16 g (yield, 33.9%) of the desired compound as a colorless liquid. m/z=257.2 (M+H).

### Step 5: Preparation of methyl 3-(3-(2-(4-(N-(tert-butoxycarbonyl)-N-(thiazol-4-yl) sulfamoyl)-2-chloro-5-fluorophenoxy)-5-chlorophenyl) propylamino) propanoate

To a solution 3-[3-(5-chloro-2-hydroxyphenyl)propylamino] propanoate) (0.09 g, 0.35 mmol) in DMF (2 ml) was added K₂CO₃ (0.145, 1.05 mmol) in one portion under nitrogen atmosphere at room temperature. The resulting reaction mixture was stirred at room temperature for 15 minutes. To the above mixture was added tert-butyl 5-chloro-2,4-difluorophenylsulfonyl(thiazol-4-yl)carbamate (0.143 g, 0.35 mmol) and the resulting mixture was stirred at room temperature for 3 hours. After completion of reaction, water (10 ml) was added and the resulting mixture was extracted with ethyl acetate (3×25 ml). The combined organic extract was washed with water (20 ml), brine (20 ml), dried over sodium sulphate and concentrated under vacuo. The crude product was purified by column chromatography using normal phase silica gel. The desired product eluted at around 20 to 25% ethyl acetate in hexane. Evaporation of the product fractions gave 0.15 g (yield, 66.2%) of desired compound as a solid. This material was used for the next step without any further purification and analysis. The material was used directly for the next step.

### Step 6: Preparation of 3-(3-(5-chloro-2(2-chloro-5-fluoro-4-(N-thiazol-4-ylsulfamoyl)phenoxy)phenyl)propylamino)propanamide fluorophenylsulfonyl(thiazol-4-yl)carbamate

To a solution of 3-(3-(2-(4-(N-(tert-butoxycarbonyl)-N-(thiazol-4-yl) sulfamoyl)-2-chloro-5-fluorophenoxy)-5-chlorophenyl) propylamino) propanoate (0.15 g, 0.23 mmol) in dichloromethane (5 ml) was added drop-wise a 4N solution of hydrochloric acid in ethyl acetate (0.5 ml) at room temperature. The resulting reaction mixture was stirred room temperature for 2 hours. After completion of reaction, pentane (20 ml) was added in to the reaction mixture which resulted in precipitation of solid. The solvent layer was decanted off; the solid thus obtained was washed twice with pentane (15 ml) and dried under vacuo. The resulting crude material was further purified by Prep HPLC using 0.1% Formic acid in Water:Acetonitrile mobile phase. Evaporation of the pure product fractions obtained from Prep HPLC provided the desired product as HCl salt. (0.009 g, 7.1% yield). LC-MS: m/z=548.8 (M+H). 1H-NMR (MeOD), δ 8.75 (d, J=2.4 Hz, 1H), 8.01 (d, J=7.2 Hz, 1H), 7.48 (d, J=2.4 Hz, 1H), 7.34-7.37 (dd, J=2.4, 8.8 Hz, 1H), 7.06 (d, J=2.4 Hz, 1H), 7.01 (d, J=8.4 Hz, 1H), 6.73 (d, J=10.4 Hz, 1H), 3.22 (t, J=6.4 Hz, 2H), 3.02-3.06 (m, 2H), 2.62-2.70 (m, 4H), 1.99-2.03 (m, 2H).

### Example 27: 2-(N-(3-(5-chloro-2-(2-chloro-5-fluoro-4-(N-(thiazol-4-yl)sulfamoyl)phenoxy)phenyl)propyl)acetamido)acetic acid

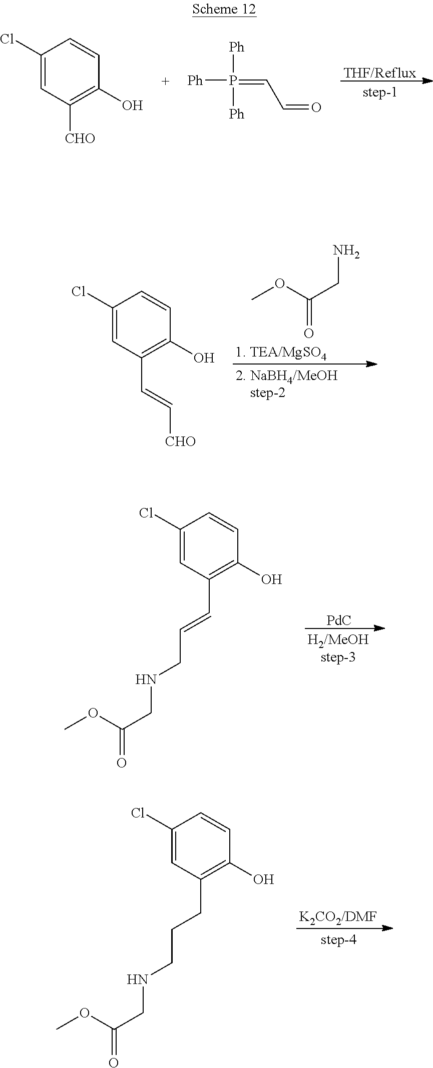

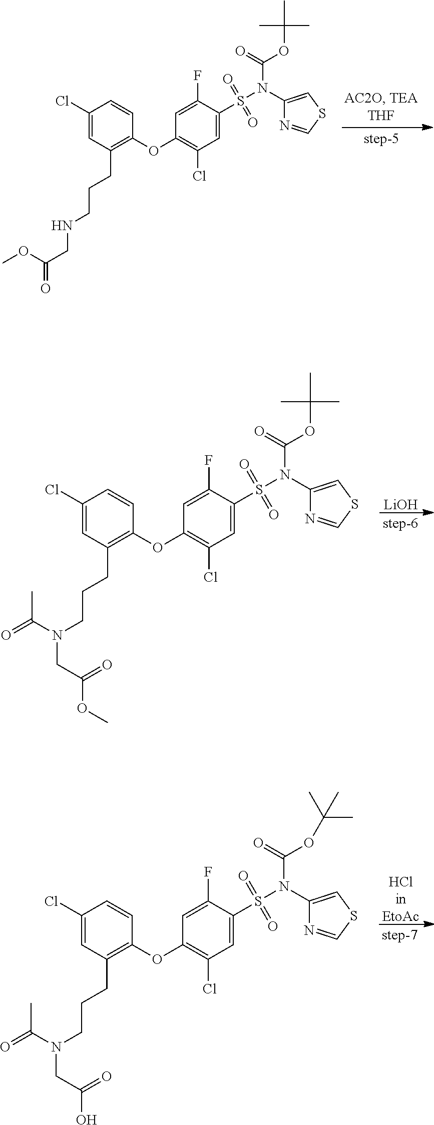

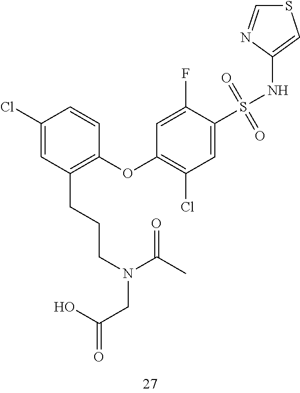 [SMILES: CC(=O)N(CCCc1cc(Cl)ccc1Oc1cc(F)c(S(=O)(=O)Nc2cscn2)cc1Cl)CC(=O)O]

### Step 1: Preparation of (E)-3-(5-chloro-2-hydroxyphenyl) acrylaldehyde

To a solution of 5-chloro-2-hydroxybenzaldehyde (20 g, 127 mmol) in THF (300 ml) was added (formylmethylene)triphenylphosphorane (43 g, 140 mmol) at room temperature. The resulting reaction mixture was then refluxed at 100° C. for 20 hrs. After completion of reaction, the reaction mixture was allowed to cool to room temperature. Water (200 ml) was added and the resulting mixture was extracted with ethyl acetate (3×250 ml). The combined organic extract was washed with water (200 ml), brine (200 ml), dried over sodium sulphate and concentrated under vacuo to get the desired crude product. The crude product was purified by column chromatography using normal phase silica gel. The desired product eluted at around 20-30% ethyl acetate in hexane. Evaporation of the product fractions gave 20 g (yield, 87%) of the desired compound as a yellow solid LC-MS: m/z=183.4 (M+H).

### Step 2: Preparation of (E)-methyl 2-(3-(5-chloro-2-hydroxyphenyl)allylamino)acetate

To a solution of (E)-3-(5-chloro-2-hydroxyphenyl)acrylaldehyde (1.0 g, 5.4 mmol) and glycine methyl ester hydrochloride (0.590 g, 6.55 mmol) in dichloromethane (20 ml) was added magnesium sulphate (1.5 g, 10.9 mmol) and triethylamine (2.28 ml, 16.38 mmol) at room temperature. The above reaction mixture was stirred at room temperature for 12 hours. The resulting reaction mixture was then concentrated under vacuo. The concentrated mass thus obtained was dissolved in methanol (20 ml) and cooled to a temperature between 5-10° C. To the above mixture, sodium borohydride (0.606 g, 16.38 mmol) was added in small portions over a period of 10 minutes; during addition temperature of the reaction mixture was maintained between 10-20° C. After completion of addition, the resulting reaction mixture was allowed to stir at room temperature for 2 hours. After completion of reaction, the reaction mixture was concentrated under vacuo. Water (40 ml) was added to the above crude mass and the resulting mixture was extracted with ethyl acetate (3×60 ml). The combined organic extract was washed with water (50 ml), brine (50 ml), dried over sodium sulphate and concentrated under vacuo to get the desired crude product. The crude product was purified by column chromatography using normal phase silica gel. The desired product eluted at around 2-3% methanol in dichloromethane. Evaporation of the product fractions gave 0.8 g (yield, 57.4%) of the desired compound as a brown liquid. LC-MS: m/z=256.07 (M+H).

### Step 3: Preparation of methyl 2-(3-(5-chloro-2-hydroxyphenyl)propylamino)acetate

To a solution of (E)-methyl 2-(3-(5-chloro-2-hydroxyphenyl)allylamino)acetate (0.8 g, 3.13 mmol) in methanol (50 ml) was carefully added Palladium hydroxide (0.199 g, 0.09 mmol). Hydrogen gas was then bubbled into the reaction mixture at room temperature for a period of 30 minutes. After completion of the reaction, the reaction mixture was filtered through celite. The celite bed was carefully washed with some amount of methanol. The filtrate thus obtained was concentrated under vacuo to afford 0.7 g (yield, 86.81%) of compound as colorless liquid. LC-MS: m/z=258.07 (M+H).

### Step 4: Preparation of methyl 2-(3-(2-(4-(N-(tert-butoxycarbonyl)-N-(thiazol-4-yl)sulfamoyl)-2-chloro-5-fluorophenoxy)-5-chlorophenyl)propylamino)acetate

To a solution of methyl 2-(3-(5-chloro-2-hydroxyphenyl)propylamino)acetate (0.7 g, 2.72 mmol) in DMF (7 ml) was added K₂CO₃ (1.12 g, 8.17 mmol) in one portion under nitrogen atmosphere at room temperature. The resulting reaction mixture was then stirred at room temperature for 15 minutes. To the above mixture was added tert-butyl 5-chloro-2,4-difluorophenylsulfonyl(thiazol-4-yl)carbamate (1.22 g, 2.996 mmol) and the resulting reaction mixture was stirred at room temperature for 3 hours. After completion of reaction, water (20 ml) was added and the resulting mixture was extracted with ethyl acetate (3×50 ml). The combined organic extract was washed with water (20 ml), brine (20 ml), dried over sodium sulphate and concentrated under vacuo to afford 0.54 g (yield, 30.64%) of the compound as a white solid. LC-MS: m/z=646.20 (M−H).

### Step 5: Preparation of methyl 2-(N-(3-(2-(4-(N-(tert-butoxycarbonyl)-N-(thiazol-4-yl)sulfamoyl)-2-chloro-5-fluorophenoxy)-5-chlorophenyl)propyl)acetamido)acetate

To a solution of methyl 2-(3-(2-(4-(N-(tert-butoxycarbonyl)-N-(thiazol-4-yl)sulfamoyl)-2-chloro-5-fluorophenoxy)-5-chlorophenyl)propylamino)acetate (0.35 g, 0.54 mmol) in THF (5 mL) was added triethyl amine (0.22 ml, 1.62 mmol). The resulting reaction mixture was stirred at 0° C. for 5-10 minutes. Acetic anhydride (0.102 ml, 1.08 mmol) was added at 0° C. The resulting reaction mixture was then refluxed at 80° C. for 12 hours. To the reaction mixture water (30 ml) was added and the resulting mixture was extracted with ethyl acetate (3×50 ml). The combined organic extracts was washed with water (30 ml), brine (30 ml), dried over sodium sulphate and concentrated under vacuo to get the desired crude product. The crude product was purified by triturating with diethyl ether. Evaporation of the product fractions gave 0.35 g (yield, 94.01%) of the desired compound as a brown solid. LC-MS: m/z=690.5 (M+H).

### Step 6: Preparation of 2-(N-(3-(2-(4-(N-(tert-butoxycarbonyl)-N-(thiazol-4-yl)sulfamoyl)-2-chloro-5-fluorophenoxy)-5-chlorophenyl)propyl)acetamido)acetic acid

To the solution of methyl 2-(N-(3-(2-(4-(N-(tert-butoxycarbonyl)-N-(thiazol-4-yl)sulfamoyl)-2-chloro-5-fluorophenoxy)-5-chlorophenyl)propyl)acetamido)acetate (0.35 g, 0.50 mmol) in THF (5 ml) was added a solution of lithium hydroxide monohydrate (0.212 g, 5.07 mmol) in water (0.5 ml) at room temperature. The resulting reaction mixture was stirred at room temperature for 3 hours. After completion of reaction ice cold water (15 ml) was added in to the reaction mixture, the resulting mixture was then acidified between 4-6 pH with aqueous 1N hydrochloric acid. The resulting acidic aqueous was extracted with ethyl acetate (3×25 ml). The combined organic extract was washed with water (20 ml), brine (20 ml), dried over sodium sulphate and concentrated under vacuo to afford 0.3 g (yield, 87.49%) of the compound as a white solid. This material was directly used for next step without any further purification and analysis. LC-MS: m/z=676.41 (M+H).

### Step 7: Preparation of 2-(N-(3-(5-chloro-2-(2-chloro-5-fluoro-4-(N-thiazol-4-ylsulfamoyl) phenoxy) phenyl) propyl)acetamido)acetic acid

To the solution of 2-(N-(3-(2-(4-(N-(tert-butoxycarbonyl)-N-(thiazol-4-yl)sulfamoyl)-2-chloro-5-fluorophenoxy)-5-chlorophenyl)propyl)acetamido)acetic acid (0.3 g, 0.44 mmol) in dichloromethane (4 ml) was added drop-wise a 4N solution of hydrochloric acid in ethyl acetate (1 ml) at room temperature. The resulting reaction mixture was stirred room temperature for 2 hours. After completion of reaction, pentane (20 ml) was added in to the reaction mixture which resulted in precipitation of solid. The solvent layer was decanted off; the solid thus obtained was washed twice with pentane (15 ml) and dried under vacuo. The resulting crude material was further purified by Prep HPLC using 0.1% Hydrochloric acid in water:acetonitrile mobile phase. Evaporation of the pure product fractions obtained from Prep HPLC provided the desired product as HCl salt (0.060 g, 23.47% yield). LC-MS: m/z=575.92 (M+H).

### Example 28: 2-(1-(3-(2-(4-(N-(1,2,4-thiadiazol-5-yl)sulfamoyl)-2-chloro-5-fluorophenoxy)-5-chlorophenyl)propyl)piperidin-4-yl)acetic acid

Compound 28 was synthesized according to the procedure described for the synthesis of compound 11 by replacing glycine methyl ester with methyl 2-(piperidin-4-yl)acetate in step 2, and replacing tert-butyl 5-chloro-2,4-difluorophenylsulfonyl(thiazol-4-yl)carbamate with 5-chloro-N-(2,4-dimethoxybenzyl)-2,4-difluoro-N-(1,2,4-thiadiazol-5-yl)benzenesulfonamide in step 4. LC-MS: m/z=601.2 (M+H).

### Example 29: 3-((3-(5-chloro-2-(2-chloro-5-fluoro-4-(N-(thiazol-2-yl)sulfamoyl)phenoxy)phenyl)propyl)amino)propanoic acid

Compound 29 was synthesized according to the procedure described for the synthesis of compound 11 by replacing glycine methyl ester with beta alanine methyl ester in step 2, and replacing tert-butyl 5-chloro-2,4-difluorophenylsulfonyl(thiazol-4-yl)carbamate with 5-chloro-N-(2,4-dimethoxybenzyl)-2,4-difluoro-N-(thiazol-2-yl)benzenesulfonamide in step 4. LC-MS: m/z=547.9 (M+H). 1H-NMR (MeOD), δ 8.05 (d, J=6.8 Hz, 1H), 7.49 (d, J=2.8 Hz, 1H), 7.34 (dd, J=2.4, 8.4 Hz, 1H), 7.17 (d, J=4.4 Hz, 1H), 7.02 (d, J=8.4 Hz, 1H), 6.80 (d, J=4.4 Hz, 1H), 6.75 (d, J=10.4 Hz, 1H), 3.14 (t, J=6.4 Hz, 2H), 3.04 (t, J=8.0 Hz, 2H), 2.71 (t, J=8.0 Hz, 2H), 2.49 (t, J=6.4 Hz, 2H), 2.00-2.03 (m, 2H).

### Example 30: 2-((3-(5-chloro-2-(2-chloro-5-fluoro-4-(N-(thiazol-4-yl)sulfamoyl)phenoxy)phenyl)propyl)amino)-N-methylacetamide

Compound 30 was synthesized according to the procedure described for the synthesis of compound 11 by replacing glycine methyl ester with 2-amino-N-methylacetamide in step 2. LC-MS: m/z=547.1 (M+H). 1H-NMR (MeOD), δ 8.77 (d, J=2.4 Hz, 1H), 8.01 (d, J=7.2 Hz, 1H), 7.48 (d, J=2.4 Hz, 1H), 7.35 (dd, J=2.4, 8.4 Hz, 1H), 7.10 (d, J=2.0 Hz, 1H), 7.02 (d, J=8.8 Hz, 1H), 6.73 (d, J=10.4 Hz, 1H), 3.70 (s, 2H), 2.97-3.02 (m, 2H), 2.80 (s, 3H), 2.65-2.69 (m, 2H), 1.96-2.06 (m, 2H).

### Example 31: 5-chloro-4-(4-chloro-2-(3-((2-(methylsulfonyl)ethyl)amino)propyl)phenoxy)-2-fluoro-N-(thiazol-4-yl)benzenesulfonamide

Compound 31 was synthesized according to the procedure described for the synthesis of compound 11 by replacing glycine methyl ester with 2-(methylsulfonyl)ethanamine in step 2. LC-MS: m/z=581.8 (M+H). 1H-NMR (MeOD), δ 8.77 (d, J=2.4 Hz, 1H), 8.02 (d, J=6.8 Hz, 1H), 7.48 (d, J=2.4 Hz, 1H), 7.36 (dd, J=2.8, 8.8 Hz, 1H), 7.10 (d, J=2.4 Hz, 1H), 7.02 (d, J=8.4 Hz, 1H), 6.73 (d, J=10.4 Hz, 1H), 3.33-3.50 (m, 4H), 3.03 (s, 3H), 2.99-3.01 (m, 2H), 2.65-2.68 (m, 2H), 1.95-2.03 (m, 2H).

### Example 32: 1-(3-(2-(4-(N-(1,2,4-thiadiazol-5-yl)sulfamoyl)-2-chloro-5-fluorophenoxy)-5-chlorophenyl)propyl)piperidine-4-carboxylic acid

Compound 32 was synthesized according to the procedure described for the synthesis of compound 11 by replacing glycine methyl ester with methyl piperidine-4-carboxylate in step 2, and replacing tert-butyl 5-chloro-2,4-difluorophenylsulfonyl(thiazol-4-yl)carbamate with 5-chloro-N-(2,4-dimethoxybenzyl)-2,4-difluoro-N-(1,2,4-thiadiazol-5-yl)benzenesulfonamide in step 4. LC-MS: m/z=589.6 (M+H).

### Example 33: 5-chloro-4-(4-chloro-2-(4,5,6,7-tetrahydropyrazolo[1,5-a]pyrimidin-3-yl)phenoxy)-2-fluoro-N-(thiazol-4-yl)benzenesulfonamide

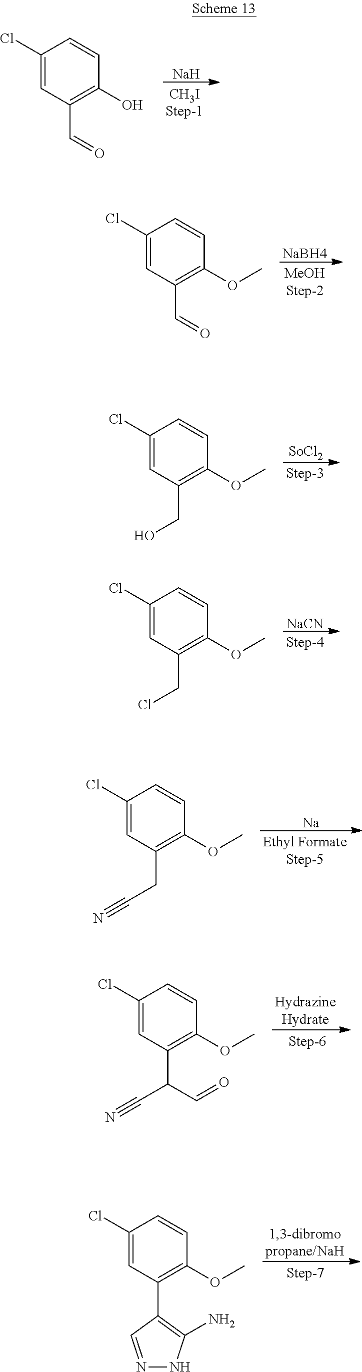

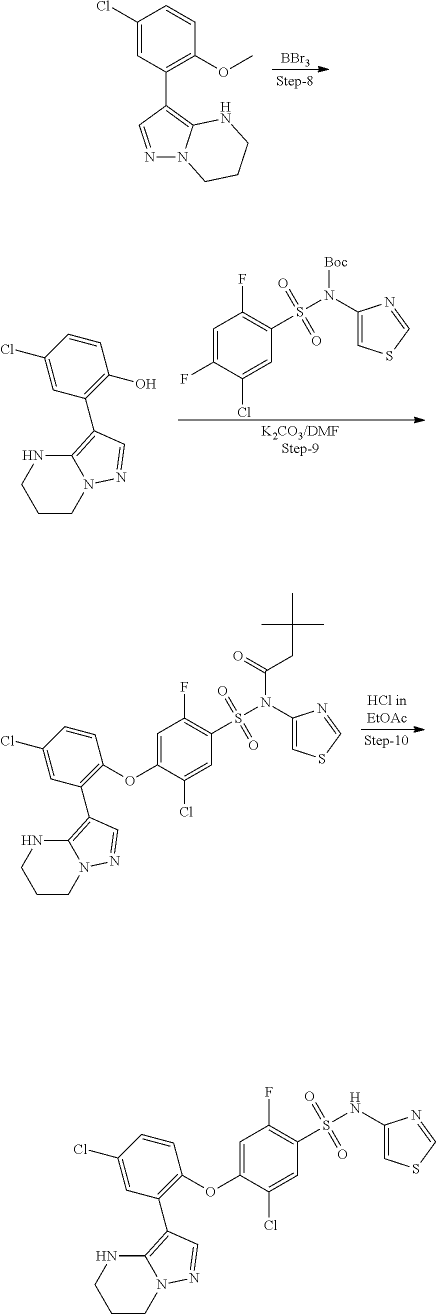

### Step 1: Preparation of 5-chloro-2-methoxybenzaldehyde

A solution of 5-chloro-2-hydroxybenzaldehyde (20 g, 128 mmol) in DMF (70 mL) was cooled to a temperature between 5-10° C. Sodium hydride (7.69 g, 192 mmol) was added to the above solution in small portions over a period of 20 minutes. Methyl iodide (23.8 ml, 384 mmol) was then added drop wise to the above reaction mixture whilst maintaining its temperature below 15° C. After completion of addition the reaction mixture was stirred at room temperature for 2 hours. Thereafter the reaction mixture was poured in to cold saturated ammonium chloride solution (250 mL) to get white precipitates. The precipitates thus formed were filtered off and dried under vacuo. The resulting solid was triturated with 100 ml of pentane:diethyl ether (4:1) to afford 18 g (yield, 82.58%) of the desired compound as a white solid. LC-MS: m/z=170.1 (M+H).

### Step 2: Preparation of (5-chloro-2-methoxyphenyl) methanol

A solution of 5-chloro-2-methoxybenzaldehyde (18 g, 105.8 mmol) in methanol (100 mL) was cooled to temperature in between 5-10° C. To the above solution sodium borohydride (11.8 g, 317 mmol) was added in portion over a period of 30 mins. After completion of addition the resulting reaction mixture was allowed to stir at room temperature for next ˜2 hours. The reaction was monitored on TLC using ethyl acetate:hexane (1:1) as mobile phase. After completion of the reaction, it was concentrated under vacuo. To the resulting crude mass, cold water (200 ml) was added to get white precipitate. The precipitate thus formed was filtered and dried to afford 16 g (yield, 87.8%) of desired compound as white solid. The material was used directly for the next step.

### Step 3: Preparation of 4-chloro-2-(chloromethyl)-1-methoxybenzene

A solution of 5-chloro-2-methoxyphenyl)methanol (16 g, 94 mmol) in DCM (100 ml) was cooled to a temperature between 5-10° C. To the above solution thionyl chloride (11 ml, 140 mmol) was added drop wise over a period of 30 minutes. After completion of addition the resulting reaction mixture was allowed to stir at room temperature for 4 hours. After completion of the reaction, it was concentrated under vacuo. To the resulting crude mass, cold water (150 ml) was added to get white precipitates. The precipitate thus formed was filtered off and dried under vacuo to afford 12 g (yield, 67.9%) of the desired compound as a white solid. The material was used directly for the next step.

### Step 4: Preparation of 2-(5-chloro-2-methoxyphenyl)acetonitrile

To a solution of 4-chloro-2-(chloromethyl)-1-methoxybenzene (12 g, 63.15 mmol) in DMSO (60 mL) was carefully added sodium cyanide (4.4 g, 95.6 mmol) at room temperature. Above reaction mixture was then heated at 100° C. for 3 hours. After cooling to room temperature, the reaction mixture was poured in to cold water (200 mL) to get precipitate. The precipitate thus formed were filtered off and dried under vacuo to afford 10 g (yield, 87.46%) of the desired compound as an off white solid. The material was used directly for the next step.

### Step 5: Preparation of 2-(5-chloro-2-methoxyphenyl)-3-oxopropanenitrile

To a solution of 2-(5-chloro-2-methoxyphenyl)acetonitrile (10 g, 47.84 mmol) in ethyl formate (50 mL) was added sodium metal (4.4 g, 95.6 mmol) at room temperature. The resulting reaction mixture was heated at 100° C. for 3 hours. After completion of the reaction, it was cooled to room temperature, water (100 ml) and dichloromethane (100 ml) were added to the reaction mixture and the solution was adjusted to pH-3 with the help of concentrated hydrochloric acid. The layers were separated and the aqueous layer was extracted with dichloromethane (2×100 ml). The combined organics were washed with saturated aqueous sodium chloride solution (150 ml), dried over sodium sulphate, filtered and evaporated under vacuo. The crude product was purified by column chromatography using normal phase silica gel. The desired product eluted at around 0.7 to 0.9% methanol in dichloromethane. Evaporation of the product fractions gave 9 g (yield, 77.94%) of the desired compound as a white solid. LC-MS: m/z=208.0 (M−H).

### Step 6: Preparation of 4-(5-chloro-2-methoxyphenyl)-1H-pyrazol-5-amine

To a solution of 2-(5-chloro-2-methoxyphenyl)-3-oxopropanenitrile (9 g, 43 mmol) in ethanol (90 mL) was added hydrazine hydrate (4.3 g, 86.12 mmol) and glacial acetic acid (2.7 mL, 51.6 mmol) at room temperature. The reaction mixture was then heated under reflux for 3 hours. After completion of the reaction, the reaction mixture was cooled to room temperature and quenched with aqueous sodium bicarbonate (150 ml). The resulting mixture was extracted with dichloromethane (3×100 ml). The combined organic layers were washed with brine, dried over sodium sulphate and concentrated under vacuo. The crude product was purified by column chromatography using normal phase silica gel. The desired product eluted at around 0.9 to 1.1% methanol in dichloromethane. Evaporation of the product fractions gave 7 g (yield, 72.8%) of the desired compound as a white solid. LC-MS: m/z=224.1 (M+H).

### Step 7: Preparation of 3-(5-chloro-2-methoxyphenyl)-4,5,6,7-tetrahydropyrazolo[1,5-a]pyrimidine

A solution of 4-(5-chloro-2-methoxyphenyl)-1H-pyrazol-5-amine (3 g, 13.45 mmol) in dry DMF (15 mL) was cooled to a temperature in between 5-10° C. Sodium hydride (0.806 g, 20.17 mmol) was added to the above solution in small portions over a period of 30 minutes. The resulting reaction mixture was stirred for 30 minutes at 5-10° C., thereafter 1, 3-dibromopropane (1.78 ml, 17.48 mmol) was added drop wise to the above mixture. The resulting reaction mixture was heated at 100° C. for a period of 4 hrs. After completion of reaction, the solution was diluted with cold water (100 mL) and the product was extracted with ethyl acetate (3×100). The combined organic layers were washed with brine, dried over sodium sulphate and concentrated under vacuo. The crude product was purified by column chromatography using normal phase silica gel. The desired product eluted at around 1.2 to 1.5% methanol in dichloromethane. Evaporation of the product fractions gave 0.65 g (yield, 18.36%) of the desired compound as a semisolid. LC-MS: m/z=264.2 (M+H).

### Step 8: Preparation of 4-chloro-2-(4,5,6,7-tetrahydropyrazolo[1,5-a]pyrimidin-3-yl)phenol

A solution of 3-(5-chloro-2-methoxyphenyl)-4,5,6,7-tetrahydropyrazolo[1,5-a]pyrimidine (0.65 g, 1.9 mmol) in dichloromethane (30 mL) was cooled to a temperature between 5-10° C. To the above solution, boron tribromide in dichloromethane (4.7 mL, 4.75 mmol) was added drop wise over a period of 30 minutes. After completion of addition, the resulting reaction mixture was stirred at room temperature for 4 hours. After completion of reaction, the solution was diluted with cold water (40 mL) and the product was extracted with ethyl acetate (3×30 mL). The combined organic layers were washed with brine, dried over sodium sulphate and concentrated under vacuo to afford 0.65 g (yield, 81.24%) of desired compound as white solid. LC-MS: m/z=250.2 (M+H).

### Step 9: Preparation of tert-butyl 5-chloro-4-(4-chloro-2-(4,5,6,7-tetrahydropyrazolo[1,5-a]pyrimidin-3-yl)phenoxy)-2-fluorophenylsulfonyl(thiazol-4-yl)carbamate

To a solution 4-chloro-2-(4,5,6,7-tetrahydropyrazolo[1,5-a]pyrimidin-3-yl)phenol (0.5 g, 2.008 mmol) in DMF (8 ml) was added K₂CO₃ (0.556 g, 4.016 mmol) in one portion under nitrogen atmosphere at room temperature. The resulting reaction mixture was stirred at room temperature for 15 minutes. To the above mixture was added tert-butyl 5-chloro-2,4-difluorophenylsulfonyl(thiazol-4-yl)carbamate (0.989 g, 2.409 mmol) and the resulting reaction mixture was stirred at room temperature for 3 hours. After completion of reaction, water (10 ml) was added and the resulting mixture was extracted with ethyl acetate (3×25 ml). The combined organic extract was washed with water (20 ml), brine (20 ml), dried over sodium sulphate and concentrated under vacuo. The crude product was purified by column chromatography using normal phase silica gel. The desired product eluted at around 40 to 50% ethyl acetate in hexane. Evaporation of the product fractions gave 0.4 g (yield, 31.18%) of the desired compound as a white solid. LC-MS: m/z=640.1 (M+H).

### Step 10: Preparation of 5-chloro-4-(4-chloro-2-(4,5,6,7-tetrahydropyrazolo[1,5-a]pyrimidin-3-yl)phenoxy)-2-fluoro-N-(thiazol-4-yl)benzenesulfonamide

To a solution of tert-butyl 5-chloro-4-(4-chloro-2-(4,5,6,7-tetrahydropyrazolo[1,5-a]pyrimidin-3-yl)phenoxy)-2-fluorophenylsulfonyl(thiazol-4-yl)carbamate (0.4 g, 0.626 mmol) in dichloromethane (15 ml) was added drop-wise a 4N solution of hydrochloric acid in ethyl acetate (0.8 ml) at room temperature. The resulting reaction mixture was stirred at room temperature for 2 hours. After completion of reaction, pentane (20 ml) was added in to the reaction mixture which resulted in precipitation of solid. The solvent layer was decanted off; the solid thus obtained was washed twice with pentane (15 ml) and dried under vacuo. The resulting crude material was further purified by Prep HPLC using 0.1% Hydrochloric acid in Water:Acetonitrile mobile phase. Evaporation of the pure product fractions obtained from Prep HPLC provided the desired product as HCl salt (0.130 g, 38.6% yield). LC-MS: m/z=539.78 (M+H). 1H NMR (400 MHz, Methanol-d4) δ 8.76 (d, J=2.4 Hz, 1H), 8.02 (s, 1H), 7.95 (d, J=7.2 Hz, 1H), 7.61 (d, J=2.4 Hz, 1H), 7.54 (dd, J=2.4, 8.4 Hz, 1H), 7.27 (d, J=8.4 Hz, 1H), 7.09 (d, J=2.0 Hz, 1H), 6.62 (d, J=10.8 Hz, 1H), 4.14 (t, J=6.0 Hz, 2H), 3.40 (t, J=5.6 Hz, 2H), 2.14 (p, J=6.0 Hz, 2H).

### Example 34: 2-((3-(5-chloro-2-(2-chloro-5-fluoro-4-(N-(thiazol-2-yl)sulfamoyl)phenoxy)phenyl)propyl)(ethoxycarbonyl)amino)acetic acid

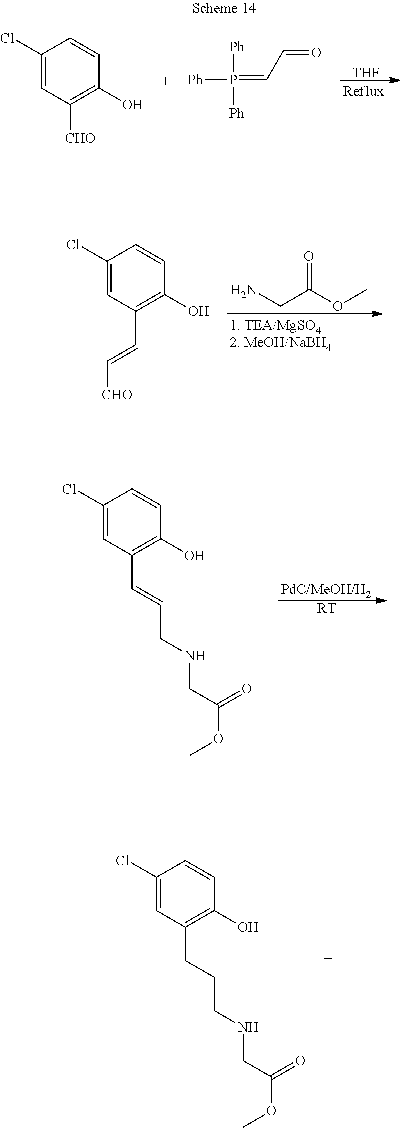

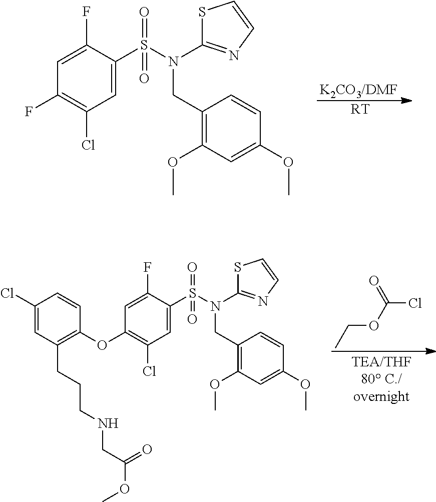

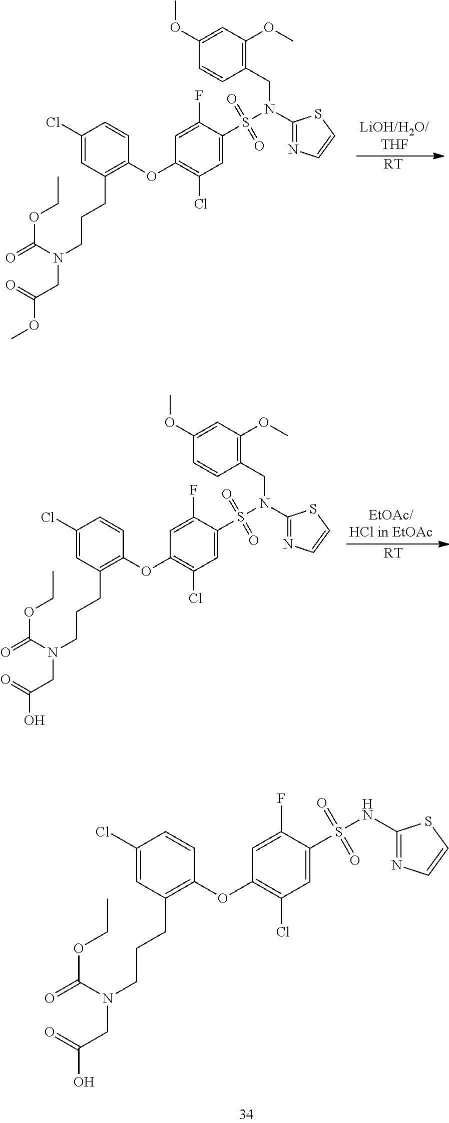 [SMILES: CCOC(=O)N(CCCc1cc(Cl)ccc1Oc1cc(F)c(S(=O)(=O)N(Cc2ccc(OC)cc2OC)c2nccs2)cc1Cl)CC(=O)OC ; "RT" ; CCOC(=O)N(CCCc1cc(Cl)ccc1Oc1cc(F)c(S(=O)(=O)N(Cc2ccc(OC)cc2OC)c2nccs2)cc1Cl)CC(=O)O ; "RT" ; CCOC(=O)N(CCCc1cc(Cl)ccc1Oc1cc(F)c(S(=O)(=O)Nc2nccs2)cc1Cl)CC(=O)O]

### Step 1: Preparation of 3-(5-chloro-2-hydroxyphenyl)acrylaldehyde

To a solution of 5-chloro-2-hydroxybenzaldehyde (20 g, 127 mmol) in THF (300 ml) was added (formylmethylene)triphenylphosphorane (43 g, 140 mmol) at room temperature. The resulting reaction mixture was refluxed at 100° C. for 20 hours. The reaction mixture was cooled to room temperature, and extracted with water (200 ml) and ethyl acetate (3×250 ml). The combined organic phase was washed with water (200 ml), brine (200 ml), dried over sodium sulphate and concentrated under vacuo to give the desired crude product. The crude product was purified by column chromatography using normal phase silica gel. The desired product eluted at around 20-30% ethyl acetate in hexane. Evaporation of the product fractions gave 20 g (yield, 87%) of desired compound as yellow solid. LC-MS: m/z=183.4 (M+H).

### Step 2: Preparation of methyl 2-(3-(5-chloro-2-hydroxyphenyl) allylamino) acetate

To a solution of 3-(5-chloro-2-hydroxyphenyl)acrylaldehyde (5 g, 27 mmol) and glycine methyl ester hydrochloride (4.1 g, 32 mmol) in dichloromethane (80 ml) was added magnesium sulphate (6 g, 50 mmol) and triethylamine (12 ml, 82 mmol) at room temperature. The above reaction mixture was stirred at room temperature for 18 hours. The resulting reaction mixture was then concentrated under vacuo. The concentrated mass thus obtained was dissolved in methanol (50 ml) and cooled to a temperature between 5-10° C. To the above mixture, sodium borohydride (3.0 g, 82 mmol) was added in small portions over a period of 20 minutes; during addition temperature of the reaction mixture was maintained between 10-20° C. The reaction mixture was allowed to stir at room temperature for 2 hours and concentrated under vacuo. Water (100 ml) was added to the above crude mass and the resulting mixture was extracted with ethyl acetate (3×100 ml). The combined organic extract was washed with water (50 ml), brine (50 ml), dried over sodium sulphate and concentrated under vacuo to get the desired crude product. The crude product was purified by column chromatography using normal phase silica gel. The desired product eluted at around 1-5% methanol in dichloromethane. Evaporation of the product fractions gave 4 g (yield, 58%) of desired compound as yellow solid. LC-MS: m/z=256.43 (M+H).

### Step-3: Preparation of methyl 2-(3-(5-chloro-2-hydroxyphenyl) propylamino) acetate

To a solution of methyl 2-(3-(5-chloro-2-hydroxyphenyl) allylamino) acetate (3.5 g, 13.6 mmol) in methanol (80 ml) was carefully added 10% Palladium on carbon with 50% moisture (0.145 g, 1.3 mmol). Hydrogen gas was then bubbled into the reaction mixture at room temperature for a period of 30 minutes. After completion of the reaction, the reaction mixture was filtered through celite. The celite bed was carefully washed with some amount of methanol. The filtrate thus obtained was concentrated under vacuo to afford 3 g (yield, 85%) of compound as colorless liquid and used as is in the next step. LC-MS: m/z=258.5 (M+H).

### Step-4: Preparation of methyl (3-(5-chloro-2-(2-chloro-4-(N-(2,4-dimethoxybenzyl)-N-(thiazol-2-yl) sulfamoyl)-5-fluorophenoxy)phenyl)propyl)glycinate

To a solution methyl 2-(3-(5-chloro-2-hydroxyphenyl) propylamino) acetate (1.1 g, 4.28 mmol) in DMF (12 ml) was added K₂CO₃ (1.77 g, 12.8 mmol) in one portion under nitrogen atmosphere at room temperature. The resulting reaction mixture was stirred at room temperature for 15 minutes. To the above mixture was added 5-chloro-N-(2,4-dimethoxybenzyl)-2,4-difluoro-N-(thiazol-2-yl)benzenesulfonamide (1.96 g, 4.28 mmol) and the resulting mixture was stirred at room temperature for 4 hours. After completion of reaction, D.M. water (100 ml) was added and the resulting mixture was extracted with ethyl acetate (3×50 ml). The combined organic extract was washed with D.M. water (50 ml), brine (50 ml) and concentrated under vacuo to afford 1.5 g (yield, 50.2%) of desired compound. The material was used directly for next step. LC-MS: m/z=698.5 (M+H).

### Step-5: Preparation of methyl N-(3-(5-chloro-2-(2-chloro-4-(N-(2,4-dimethoxybenzyl)-N-(thiazol-2-yl) sulfamoyl)-5-fluorophenoxy)phenyl)propyl)-N-(ethoxycarbonyl)glycinate

To a solution of methyl (3-(5-chloro-2-(2-chloro-4-(N-(2,4-dimethoxybenzyl)-N-(thiazol-2-yl)sulfamoyl)-5-fluorophenoxy)phenyl)propyl)glycinate (1.0 g, 1.43 mmol) in dichloromethane (50 mL) was added triethyl amine (0.596 ml, 4.29 mmol) at room temperature. The resulting reaction mixture was stirred at the same temperature for 10 minutes. Ethylchloroformate (0.407 ml, 4.29 mmol) was added to the reaction mixture at room temperature. The resulting reaction mixture was then refluxed at 80° C. for 12 hours. After completion of the reaction, the reaction mixture was cooled to room temperature and dumped in to D.M. water (50 ml). The resulting mixture was extracted with dichloromethane (3×50 ml). The combined organic extract was washed with D.M. water (50 ml), brine (50 ml), dried over sodium sulphate and concentrated under vacuo to get the desired crude product. The crude product was purified by triturating with diethyl ether which gave 0.860 g (yield, 78.09%) of the desired compound as a brown solid. The material was directly used for the next step. LC-MS: m/z=770.2 (M+H).

### Step-6: Preparation of N-(3-(5-chloro-2-(2-chloro-4-(N-(2,4-dimethoxybenzyl)-N-(thiazol-2-yl)sulfamoyl)-5-fluorophenoxy)phenyl)propyl)-N-(ethoxycarbonyl)glycine

To the solution of methyl N-(3-(5-chloro-2-(2-chloro-4-(N-(2,4-dimethoxybenzyl)-N-(thiazol-2-yl)sulfamoyl)-5-fluorophenoxy)phenyl)propyl)-N-(ethoxycarbonyl)glycinate (0.85 g, 1.10 mmol) in THF (10 ml) was added a solution of lithium hydroxide monohydrate (0.135 g, 5.62 mmol) in D.M. water (10 ml) at room temperature. The resulting reaction mixture was stirred at room temperature for 3 hours. After completion of reaction ice cold water (20 ml) was added in to the reaction mixture, the resulting mixture was then acidified between 4-6 pH with aqueous 1N hydrochloric acid. The resulting acidic aqueous was extracted with ethyl acetate (3×30 ml). The combined organic extract was washed with D.M. water (20 ml), brine (20 ml), dried over sodium sulphate and concentrated under vacuo to afford 0.55 g (yield, 66.13%) of the compound as a white solid. This material was directly used for next step.

### Step-7: Preparation of N-(3-(5-chloro-2-(2-chloro-5-fluoro-4-(N-(thiazol-2-yl) sulfamoyl) phenoxy) phenyl) propyl)-N-(ethoxycarbonyl)glycine

To a solution of N-(3-(5-chloro-2-(2-chloro-4-(N-(2,4-dimethoxybenzyl)-N-(thiazol-2-yl)sulfamoyl)-5-fluorophenoxy)phenyl)propyl)-N-(ethoxycarbonyl)glycine (0.5 g, 0.66 mmol) in dichloromethane (10 ml) was added drop-wise a 4N solution of hydrochloric acid in ethyl acetate (5 ml) at room temperature. The resulting reaction mixture was stirred room temperature for 4 hours. After completion of reaction, pentane (15 ml) was added in to the reaction mixture which resulted in precipitation of solid. The solvent layer was decanted off; the solid thus obtained was washed twice with pentane (10 ml) and dried under vacuo. The resulting crude material was further purified by Prep HPLC using 0.1% HCl in Water: Acetonitrile mobile phase (PREP HPLC Method A). Evaporation of the pure product fractions obtained from Prep HPLC provided the desired product (0.04 g, 10% yield). LC-MS: m/z=606.16 (M+H). 1H-NMR (MeOD), δ 8.00-8.06 (m, 1H), 7.45 (s, 1H), 7.28-7.35 (m, 1H), 7.16 (d, J=4.6 Hz, 1H), 6.97-7.06 (m, 1H), 6.80 (d, J=4.7 Hz, 1H), 6.61-6.71 (m, 1H), 4.01-4.12 (m, 1H), 3.95 (d, J=10.6 Hz, 2H), 2.52-2.63 (m, 2H), 1.79-1.90 (m, 2H), 1.14-1.25 (m, 3H).

### Example 35: ethyl 2-((3-(5-chloro-2-(2-chloro-5-fluoro-4-(N-(thiazol-2-yl)sulfamoyl)phenoxy)phenyl)propyl)amino)acetate

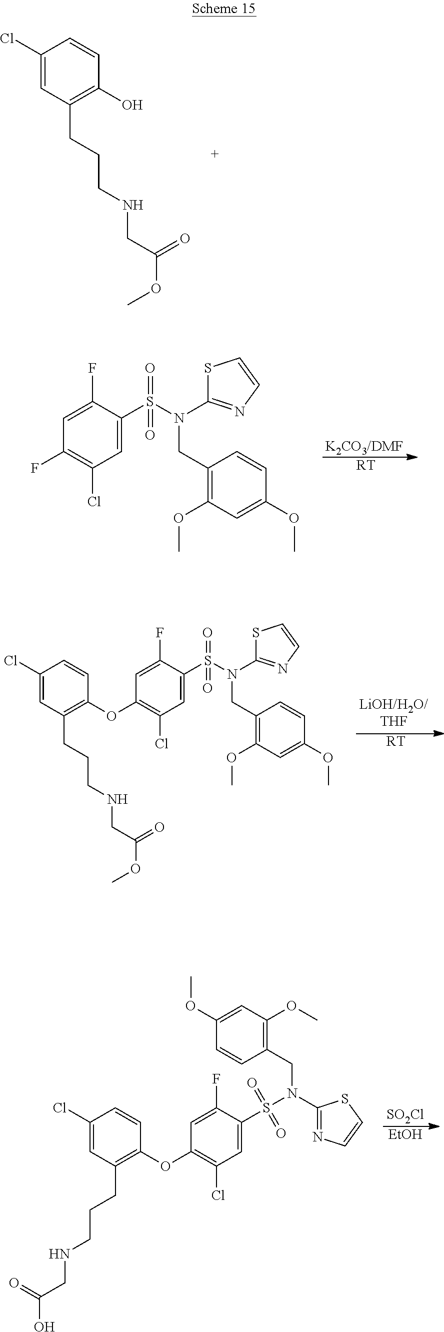

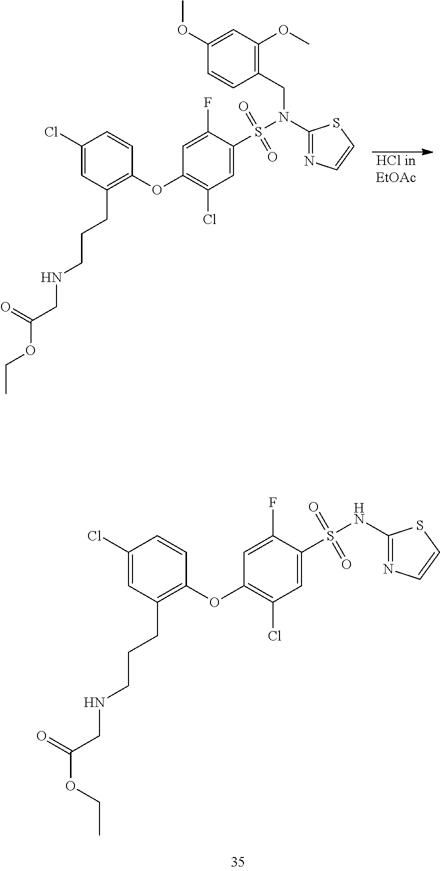 [SMILES: CCOC(=O)CNCCCc1cc(Cl)ccc1Oc1cc(F)c(S(=O)(=O)N(Cc2ccc(OC)cc2OC)c2nccs2)cc1Cl ; "HCl inEtOAc" ; CCOC(=O)CNCCCc1cc(Cl)ccc1Oc1cc(F)c(S(=O)(=O)Nc2nccs2)cc1Cl]

### Step-1: Preparation of methyl (3-(5-chloro-2-(2-chloro-4-(N-(2,4-dimethoxybenzyl)-N-(thiazol-2-yl) sulfamoyl)-5-fluorophenoxy)phenyl)propyl)glycinate

To a solution methyl 2-(3-(5-chloro-2-hydroxyphenyl) propylamino) acetate (1.6 g, 6.22 mmol), which was synthesized according to the procedure described for compound 11, in DMF (12 ml) was added K₂CO₃ (2.5 g, 18.6 mmol) in one portion under nitrogen atmosphere at room temperature. The resulting reaction mixture was stirred at room temperature for 15 minutes. To the above mixture was added 5-chloro-N-(2,4-dimethoxybenzyl)-2,4-difluoro-N-(thiazol-2-yl)benzenesulfonamide (3.14 g, 6.8 mmol) and the resulting mixture was stirred at room temperature for 4 hours. After completion of reaction, D.M. water (100 ml) was added and the resulting mixture was extracted with ethyl acetate (3×50 ml). The combined organic extract was washed with D.M. water (50 ml), brine (50 ml) and concentrated under vacuo to afford 1.0 g (yield, 23.2%) of desired compound as a solid. The material was used directly for next step.

### Step-2: Preparation of (3-(5-chloro-2-(2-chloro-4-(N-(2,4-dimethoxybenzyl)-N-(thiazol-2-yl)sulfamoyl)-5-fluorophenoxy)phenyl)propyl)glycine

To the solution of methyl (3-(5-chloro-2-(2-chloro-4-(N-(2,4-dimethoxybenzyl)-N-(thiazol-2-yl)sulfamoyl)-5-fluorophenoxy)phenyl)propyl)glycinate (1 g, 1.43 mmol) in THF (15 ml) was added a solution of lithium hydroxide monohydrate (0.3 g, 7.16 mmol) in D.M. water (15 ml) at room temperature. The resulting reaction mixture was stirred at room temperature for 3 hours. After completion of reaction ice cold water (20 ml) was added in to the reaction mixture, the resulting mixture was then acidified between 4-6 pH with aqueous 1N hydrochloric acid. The resulting acidic aqueous was extracted with ethyl acetate (3×30 ml). The combined organic extract was washed with D.M. water (20 ml), brine (20 ml), dried over sodium sulphate and concentrated under vacuo to afford 0.7 g (yield, 71.55%) of the compound as a white solid. This material was directly used for next step without any further purification and analysis. LC-MS: m/z=684.15 (M+H).

### Step-3: Preparation of ethyl (3-(5-chloro-2-(2-chloro-4-(N-(2,4-dimethoxybenzyl)-N-(thiazol-2-yl) sulfamoyl)-5-fluorophenoxy)phenyl)propyl)glycinate

To a solution of (3-(5-chloro-2-(2-chloro-4-(N-(2,4-dimethoxybenzyl)-N-(thiazol-2-yl)sulfamoyl)-5-fluorophenoxy)phenyl)propyl)glycine (0.5 g, 0.73 mmol) in ethanol (10 ml) was added thionyl chloride (0.53 ml, 7.3 mmol) at 0° C. The resulting reaction mixture was then refluxed for 12 hours. After completion of reaction the reaction mixture was evaporated under vacuo and D.M. water (30 ml) was added to the resulting residue. The resulting mixture was extracted with ethyl acetate (3×50 ml). The combined organic extracts was washed with D.M. water (20 ml), brine (20 ml), dried over sodium sulphate and concentrated under vacuo to get the desired crude product. The crude product was purified by triturating with diethyl ether which gave 0.4 g (yield, 76.9%) of the desired compound. The material was used directly for next step. LC-MS: m/z=712.4 (M+H).

### Step-4: Preparation of ethyl (3-(5-chloro-2-(2-chloro-5-fluoro-4-(N-(thiazol-2-yl) sulfamoyl) phenoxy) phenyl)propyl)glycinate

To a solution of ethyl (3-(5-chloro-2-(2-chloro-4-(N-(2,4-dimethoxybenzyl)-N-(thiazol-2-yl)sulfamoyl)-5-fluorophenoxy)phenyl)propyl)glycinate (0.35 g, 0.49 mmol) in dichloromethane (10 ml) was added drop-wise a 4N solution of hydrochloric acid in ethyl acetate (5 ml) at room temperature. The resulting reaction mixture was stirred room temperature for 4 hours. After completion of reaction, pentane (15 ml) was added in to the reaction mixture which resulted in precipitation of solid. The solvent layer was decanted off; the solid thus obtained was washed twice with pentane (10 ml) and dried under vacuo. The resulting crude material was further purified by Prep HPLC using 0.1% HCl in water: acetonitrile mobile phase (PREP HPLC Method A). Evaporation of the pure product fractions obtained from Prep HPLC provided the desired product (0.045 g, 16.32% yield). LC-MS: m/z=562.04 (M+H). ¹H-NMR (MeOD), δ 8.03-8.08 (m, 1H) 7.46-7.49 (m, 1H) 7.33-7.38 (m, 1H) 7.15-7.19 (m, 1H) 7.00-7.04 (m, 1H) 6.81-6.85 (m, 1H) 6.75-6.79 (m, 1H) 4.29-4.36 (m, 2H) 3.97 (s, 2H) 3.07-3.13 (m, 2H) 2.67-2.74 (m, 2H) 2.00-2.08 (m, 2H) 1.29-1.37 (m, 3H).

### Example 42: 4-(2-(3-((1H-pyrazol-4-yl)amino)propyl)-4-chlorophenoxy)-5-chloro-2-fluoro-N-(thiazol-2-yl)benzenesulfonamide

Compound 42 was synthesized according to the procedure described for the synthesis of compound 11 by replacing glycine methyl ester with 1H-pyrazol-4-amine in step 2, and omitting step 5. LC-MS: m/z=541.82 (M+H). 1H-NMR (MeOD), δ 8.78 (d, J=2.1 Hz, 1H), 8.02 (d, J=7.1 Hz, 1H), 7.89 (s, 2H), 7.48 (d, J=2.4 Hz, 1H), 7.32-7.40 (m, 1H), 7.12 (d, J=2.1 Hz, 1H), 7.01 (d, J=8.7 Hz, 1H), 6.74 (d, J=10.7 Hz, 1H), 3.36-3.44 (m, 2H), 2.70 (t, J=7.7 Hz, 2H), 2.06 (s, 2H).

### Example 43: 3-((3-(5-chloro-2-(2,5-difluoro-4-(N-(thiazol-2-yl)sulfamoyl)phenoxy) phenyl) propyl) amino) propanoic acid

Compound 43 was synthesized according to the procedure described for the synthesis of compound 11 by replacing glycine methyl ester with beta alanine methyl ester in step 2, and replacing tert-butyl 5-chloro-2,4-difluorophenylsulfonyl(thiazol-4-yl)carbamate with N-(2,4-dimethoxybenzyl)-2,4,5-trifluoro-N-(thiazol-2-yl)benzenesulfonamide in step 4. LC-MS: m/z=532.14 (M+H). 1H-NMR (MeOD), δ 7.78-7.85 (m, 1H) 7.44-7.51 (m, 1H) 7.27-7.36 (m, 1H) 7.13-7.19 (m, 1H) 6.96-7.03 (m, 1H) 6.83-6.91 (m, 1H) 6.79 (s, 1H) 3.11-3.18 (m, 2H) 3.01-3.08 (m, 2H) 2.71-2.80 (m, 2H) 2.43-2.52 (m, 2H) 1.97-2.08 (m, 2H).

### Example 44: 5-chloro-4-(4-chloro-2-(3-((2-(methylsulfonyl)ethyl)amino)propyl)phenoxy)-2-fluoro-N-(thiazol-4-yl)benzenesulfonamide

Compound 44 was synthesized according to the procedure described for the synthesis of compound 11 by replacing glycine methyl ester with 2-(methylsulfonyl)ethanamine in step 2, and omitting step 5. LC-MS: m/z=581.83 (M+H). 1H-NMR (MeOD), δ 8.77 (d, J=2.2 Hz, 1H), 7.99-8.07 (m, 1H), 7.46-7.53 (m, 1H), 7.31-7.41 (m, 1H), 7.10 (d, J=2.2 Hz, 1H), 7.04 (s, 1H), 6.69-6.77 (m, 1H), 3.46-3.52 (m, 2H), 3.39-3.45 (m, 2H), 3.09 (s, 3H), 2.97-3.04 (m, 2H), 2.63-2.71 (m, 2H), 1.94-2.04 (m, 2H).

### Example 45: 4-(2-(3-((1H-pyrazol-3-yl) amino) propyl)-4-chlorophenoxy)-5-chloro-2-fluoro-N-(thiazol-4-yl) benzenesulfonamide

Compound 45 was synthesized according to the procedure described for the synthesis of compound 11 by replacing glycine methyl ester with 1H-pyrazol-3-amine in step 2, and omitting step 5. LC-MS: m/z=541.99 (M+H). 1H-NMR (MeOD), δ 8.76 (d, J=2.2 Hz, 1H), 7.95 (d, J=7.2 Hz, 1H), 7.76-7.82 (m, 1H), 7.45 (d, J=2.6 Hz, 1H), 7.33 (dd, J=8.6, 2.6 Hz, 1H), 7.10 (d, J=2.2 Hz, 1H), 7.02 (d, J=8.7 Hz, 1H), 6.63 (d, J=10.8 Hz, 1H), 5.74 (d, J=2.9 Hz, 1H), 3.19 (t, J=6.7 Hz, 2H), 2.62-2.71 (m, 2H), 1.82-1.98 (m, 2H).

### Example 46: 2-((3-(5-chloro-2-(2-chloro-5-fluoro-4-(N-(thiazol-4-yl) sulfamoyl) phenoxy) phenyl) propyl) amino)-N-methylacetamide

Compound 46 was synthesized according to the procedure described for the synthesis of compound 11 by replacing glycine methyl ester with 2-amino-N-methylacetamide in step 2, and omitting step 5. LC-MS: m/z=546.88 (M+H). 1H-NMR (MeOD), δ 8.74-8.81 (m, 1H), 7.97-8.07 (m, 1H), 7.46-7.51 (m, 1H), 7.32-7.40 (m, 1H), 7.09-7.15 (m, 1H), 6.97-7.06 (m, 1H), 6.68-6.82 (m, 1H), 3.76 (s, 2H), 3.01-3.09 (m, 2H), 2.80 (s, 3H), 2.63-2.72 (m, 2H), 1.97-2.07 (m, 2H).

### Example 47: 2-((3-(5-chloro-2-(2-chloro-5-fluoro-4-(N-(thiazol-4-yl)sulfamoyl)phenoxy)phenyl)propyl)(methyl)amino)acetic acid

Compound 47 was synthesized according to the procedure described for the synthesis of compound 11 by replacing glycine methyl ester with sarcosine methyl ester in step 2. LC-MS: m/z=548.04 (M+H). 1H-NMR (MeOD), δ 8.73 (d, J=2.1 Hz, 1H), 8.01 (d, J=7.1 Hz, 1H), 7.49 (d, J=2.5 Hz, 1H), 7.34 (dd, J=8.7, 2.6 Hz, 1H), 6.99 (d, J=8.7 Hz, 1H), 6.94 (d, J=2.1 Hz, 1H), 6.74 (d, J=10.6 Hz, 1H), 3.61 (s, 2H), 3.09-3.19 (m, 2H), 2.84 (s, 3H), 2.67 (t, J=7.7 Hz, 2H), 2.06 (t, J=8.0 Hz, 2H).

### Example 48: 5-chloro-4-(4-chloro-2-(3-(6,7-dihydro-1H-pyrazolo[4,3-c]pyridin-5(4H)-yl)propyl)phenoxy)-2-fluoro-N-(thiazol-4-yl)benzenesulfonamide

Compound 48 was synthesized according to the procedure described for the synthesis of compound 11 by replacing glycine methyl ester with 4,5,6,7-tetrahydro-1H-pyrazolo[4,3-c]pyridine in step 2, and omitting step 5. LC-MS: m/z=581.90 (M+H). 1H-NMR (MeOD), δ 8.76 (br. s., 1H), 8.02 (br. s., 1H), 7.67 (br. s, 1H), 7.54 (br. s, 1H), 7.35 (br. s., 1H), 7.13 (br. s, 1H), 7.03 (d, J=6.7 Hz, 1H), 6.73-6.79 (m, 1H), 4.55-4.57 (m, 1H), 4.19-4.21 (m, 1H), 3.81 (br. s, 1H), 3.66 (br. s, 1H), 3.50 (br. s, 1H), 3.29 (s, 1H), 3.15 (br. s, 3H), 2.68-2.72 (m, 3H), 2.15 (br. s, 2H).

### Example 49: 2-((3-(5-chloro-2-(2-chloro-5-fluoro-4-(N-(thiazol-4-yl)sulfamoyl)phenoxy)phenyl) propyl)amino) acetamide

### Preparation of 2-((3-(5-chloro-2-hydroxyphenyl)propyl)amino)acetamide

Methyl (3-(5-chloro-2-hydroxyphenyl)propyl)glycinate was synthesized as described in the procedure for compound 11. A solution of methyl (3-(5-chloro-2-hydroxyphenyl)propyl)glycinate (6.0 g, 23.25 mmol) in methanol (200 ml) was cooled to −78° C. using acetone/dry ice bath. Ammonia gas was then purged in this cold reaction mixture for 1-3 hours. The reaction assembly was then tightly closed and the reaction mixture was allowed to warm to room temperature whereby it further stirred for next 18 hours. The reaction mixture was monitored on TLC using pure ethyl acetate as mobile phase. After completion of reaction, the reaction mixtures was mixed and evaporated under vacuo to get a crude material which was further co-evaporated two times with diethyl ether. This crude material was triturated with Diethyl ether (2×50 ml) and pentane (50 ml), the resulting solid filtered off under vacuo and was directly used for the next step without any further purification. LC-MS: m/z=243.08 (M+H).

### Preparation of 2-((3-(5-chloro-2-(2-chloro-5-fluoro-4-(N-(thiazol-4-yl)sulfamoyl)phenoxy)phenyl) propyl)amino)acetamide (49)

Compound 49 was synthesized according to the procedure described for the synthesis of compound 11 by replacing methyl 2-(3-(5-chloro-2-hydroxyphenyl) propylamino) acetate with 2-((3-(5-chloro-2-hydroxyphenyl)propyl)amino)acetamide in step 4, and omitting step 5. LC-MS: m/z=533. (M+H). 1H-NMR (MeOD), δ 8.77 (s, 1H), 8.03 (d, J=6.4 Hz, 1H), 7.49 (s, 1H), 7.37 (d, J=8.8 Hz, 1H), 7.12 (s, 1H), 7.03 (d, J=8.8 Hz, 1H), 6.76 (d, J=10.4 Hz, 1H), 3.80 (s, 2H), 3.06 (t, J=8 Hz, 2H), 2.68 (t, J=7.6 Hz, 2H), 2.03 (t, J=8 Hz, 2H).

### Example 50: isopentyl 2-((3-(5-chloro-2-(2-chloro-5-fluoro-4-(N-(thiazol-2-yl)sulfamoyl)phenoxy)phenyl)propyl)amino)acetate

Compound 50 was synthesized according to the procedure described for the synthesis of compound 35 by replacing ethanol with 3-methylbutan-1-ol in step 3. LC-MS: m/z=604.14 (M+H). 1H-NMR (DMSO), δ 7.91-8.00 (m, 1H), 7.50-7.56 (m, 1H), 7.32-7.40 (m, 2H), 7.06-7.14 (m, 1H), 6.90-6.99 (m, 2H), 4.16-4.25 (m, 2H), 3.98-4.04 (m, 2H), 2.90-2.97 (m, 2H), 2.57-2.64 (m, 2H), 1.87-1.96 (m, 2H), 1.61-1.72 (m, 1H), 1.45-1.55 (m, 2H), 0.90 (d, J=6.6 Hz, 6H).

### Example 51: isopropyl 2-((3-(5-chloro-2-(2-chloro-5-fluoro-4-(N-(thiazol-2-yl)sulfamoyl)phenoxy)phenyl)propyl)amino)acetate

Compound 51 was synthesized according to the procedure described for the synthesis of compound 35 by replacing ethanol with isopropanol in step 3. LC-MS: m/z=575.92 (M+H). 1H-NMR (MeOD), δ 1.31 (s, 3H) 1.32 (s, 3H) 2.01-2.09 (m, 2H) 2.71 (t, J=7.63 Hz, 2H) 3.07-3.15 (m, 2H) 3.95 (s, 2H) 5.11-5.19 (m, 1H) 6.76 (d, J=10.45 Hz, 1H) 6.82 (d, J=4.65 Hz, 1H) 7.01 (d, J=8.70 Hz, 1H) 7.18 (d, J=4.65 Hz, 1H) 7.35 (dd, J=8.70, 2.59 Hz, 1H) 7.49 (d, J=2.52 Hz, 1H) 8.05 (d, J=7.10 Hz, 1H).

### Example 52: methyl 2-((3-(5-chloro-2-(2-chloro-5-fluoro-4-(N-(thiazol-4-yl)sulfamoyl)phenoxy)phenyl)propyl)(methyl)amino)acetate

Compound 52 was synthesized according to the procedure described for the synthesis of compound 11 by replacing glycine methyl ester with sarcosine methyl ester in step 2, and omitting step 5. LC-MS: m/z=562.14 (M+H). 1H-NMR (MeOD), δ 8.76 (d, J=2.2 Hz, 1H), 8.00 (d, J=7.2 Hz, 1H), 7.45 (d, J=2.6 Hz, 1H), 7.29-7.36 (m, 1H), 6.97-7.10 (m, 2H), 6.66 (d, J=10.8 Hz, 1H), 3.68 (s, 3H), 3.24 (s, 2H), 2.56 (s, 2H), 2.48 (d, J=7.6 Hz, 2H), 2.28 (s, 3H), 1.71-1.81 (m, 2H).

### Example 53: 2-((3-(5-chloro-2-(2-chloro-5-fluoro-4-(N-(thiazol-2-yl)sulfamoyl)phenoxy)phenyl)propyl)((pentyloxy)carbonyl)amino)acetic acid

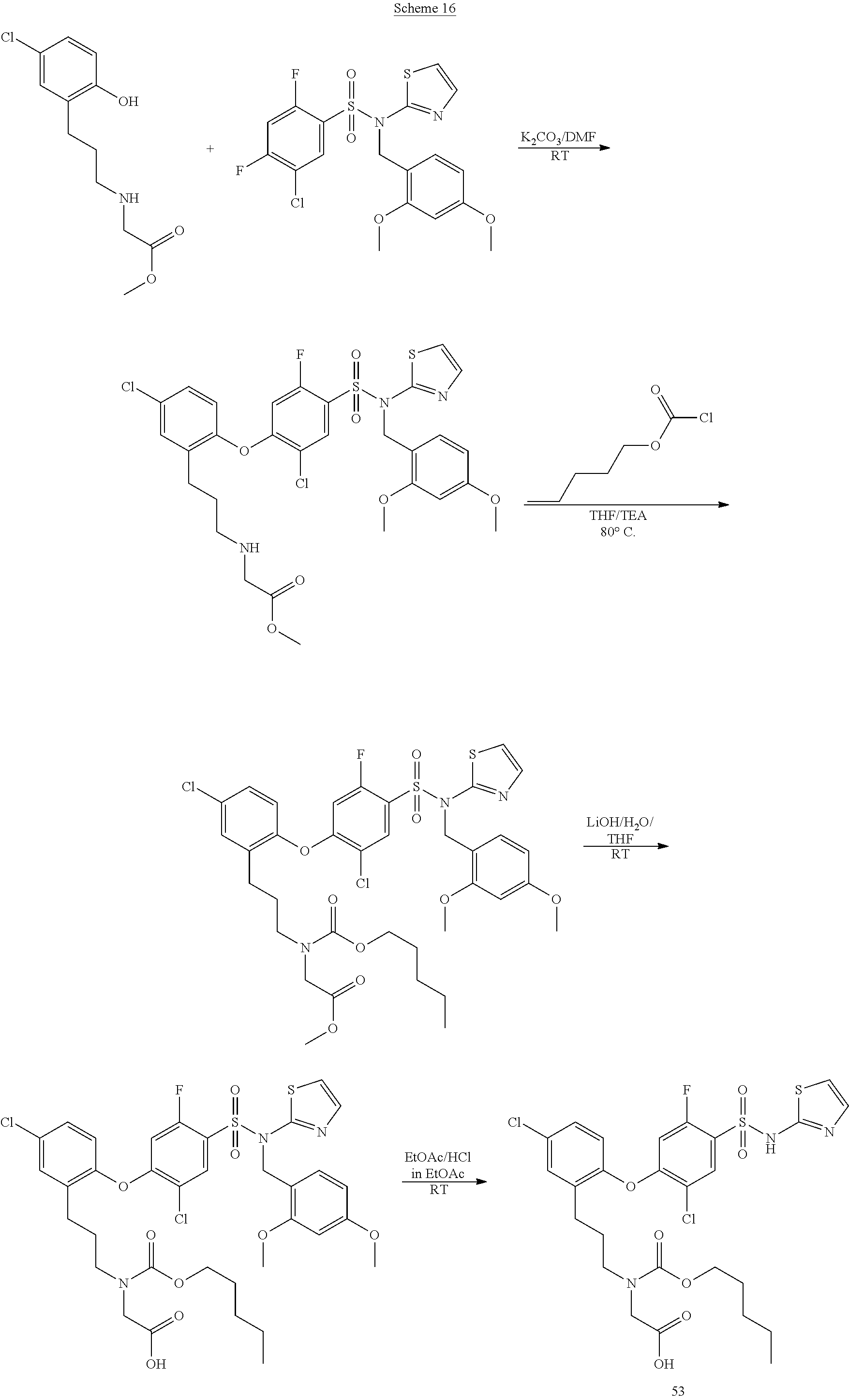

### Step-1: Preparation of methyl (3-(5-chloro-2-(2-chloro-4-(N-(2,4-dimethoxybenzyl)-N-(thiazol-2-yl) sulfamoyl)-5-fluorophenoxy)phenyl)propyl)glycinate

To a solution methyl 2-(3-(5-chloro-2-hydroxyphenyl) propylamino) acetate (0.6 g, 2.3 mmol), which was synthesized according to the procedure described for compound 11, in DMF (10 ml) was added K₂CO₃ (0.96 g, 6.9 mmol) in one portion under nitrogen atmosphere at room temperature. The resulting reaction mixture was stirred at room temperature for 15 minutes. To the above mixture was added 5-chloro-N-(2,4-dimethoxybenzyl)-2,4-difluoro-N-(thiazol-2-yl)benzenesulfonamide (1.17 g, 2.5 mmol) and the resulting mixture was stirred at room temperature for 4 hours. After completion of reaction, D.M. water (100 ml) was added and the resulting mixture was extracted with ethyl acetate (3×50 ml). The combined organic extract was washed with D.M. water (50 ml), brine (50 ml). The combined organic layers were washed with brine, dried over sodium sulphate and concentrated under vacuo to afford 0.6 g (yield, 37.36%) of the desired compound. The material was used directly for next step. LC-MS: m/z=698.1 (M+H).

### Step-2: Preparation of methyl N-(3-(5-chloro-2-(2-chloro-4-(N-(2,4-dimethoxybenzyl)-N-(thiazol-2-yl) sulfamoyl)-5-fluorophenoxy)phenyl)propyl)-N-((pentyloxy)carbonyl)glycinate

To a solution of methyl (3-(5-chloro-2-(2-chloro-4-(N-(2,4-dimethoxybenzyl)-N-(thiazol-2-yl)sulfamoyl)-5-fluorophenoxy)phenyl)propyl)glycinate (0.6 g, 0.85 mmol) in dichloromethane (30 ml) was added triethyl amine (0.36 ml, 2.57 mmol) at room temperature. The resulting reaction mixture was stirred at the same temperature for 10 minutes. Pentyl chloroformate (0.38 ml, 2.57 mmol) was added to the reaction mixture at room temperature. The resulting reaction mixture was then refluxed at 80° C. for 12 hours. After completion of the reaction, the reaction mixture was cooled to room temperature and dumped in to D.M. water (50 ml). The resulting mixture was extracted with dichloromethane (3×50 ml). The combined organic extract was washed with D.M. water (50 ml), brine (50 ml), dried over sodium sulphate and concentrated under vacuo to get the desired crude product. The crude product was purified by triturating with diethyl ether which gave 0.6 g (yield, 86.9%) of the desired compound as a brown solid. MS: m/z=812.21 (M+H).

### Step-3: Preparation of N-(3-(5-chloro-2-(2-chloro-4-(N-(2,4-dimethoxybenzyl)-N-(thiazol-2-yl)sulfamoyl)-5-fluorophenoxy)phenyl)propyl)-N-((pentyloxy)carbonyl)glycine

To the solution of methyl N-(3-(5-chloro-2-(2-chloro-4-(N-(2,4-dimethoxybenzyl)-N-(thiazol-2-yl)sulfamoyl)-5-fluorophenoxy)phenyl)propyl)-N-((pentyloxy)carbonyl)glycinate (0.6 g, 0.738 mmol) in THF (20 ml) was added a solution of lithium hydroxide monohydrate (0.1 g, 4.43 mmol) in D.M. water (10 ml) at room temperature. The resulting reaction mixture was stirred at room temperature for 3 hours. After completion of reaction ice cold water (20 ml) was added in to the reaction mixture, the resulting mixture was then acidified between 4-6 pH with aqueous 1N hydrochloric acid. The resulting acidic aqueous was extracted with ethyl acetate (3×30 ml). The combined organic extract was washed with D.M. water (20 ml), brine (20 ml), dried over sodium sulphate and concentrated under vacuo to afford 0.5 g (yield, 84.89%) of the compound as a white solid. This material was directly used for next step.

### Step-4: Preparation of N-(3-(5-chloro-2-(2-chloro-5-fluoro-4-(N-(thiazol-2-yl) sulfamoyl) phenoxy) phenyl) propyl)-N-((pentyloxy)carbonyl)glycine

To a solution of N-(3-(5-chloro-2-(2-chloro-4-(N-(2,4-dimethoxybenzyl)-N-(thiazol-2-yl)sulfamoyl)-5-fluorophenoxy)phenyl)propyl)-N-((pentyloxy)carbonyl)glycine (0.5 g, 0.626 mmol) in dichloromethane (15 ml) was added drop-wise a 4N solution of hydrochloric acid in ethyl acetate (5 ml) at room temperature. The resulting reaction mixture was stirred room temperature for 4 hours. After completion of reaction, pentane (10 ml) was added in to the reaction mixture which resulted in precipitation of solid. The solvent layer was decanted off; the solid thus obtained was washed twice with pentane (10 ml) and dried under vacuo. The resulting crude material was further purified by Prep HPLC using 0.1% HCl in water: acetonitrile mobile phase (PREP HPLC Method A). Evaporation of the pure product fractions obtained from Prep HPLC provided the desired product (0.05 g, 12.3% yield). LC-MS: m/z=648.14 (M+H). 1H-NMR (DMSO), δ 13.02 (br. s, 1H), 12.63 (br. s, 1H), 7.89-7.96 (m, 1H), 7.44-7.51 (m, 1H), 7.30-7.36 (m, 2H), 7.05-7.13 (m, 1H), 6.80-6.94 (m, 2H), 3.83-3.93 (m, 4H), 3.16-3.28 (m, 2H), 1.66-1.81 (m, 2H), 1.39-1.51 (m, 2H), 1.12-1.28 (m, 4H), 0.74-0.88 (m, 3H).

### Example 54: 2-((3-(5-chloro-2-(2-chloro-5-fluoro-4-(N-(thiazol-2-yl)sulfamoyl)phenoxy)phenyl)propyl)(prop-2-yn-1-yl)amino)acetic acid

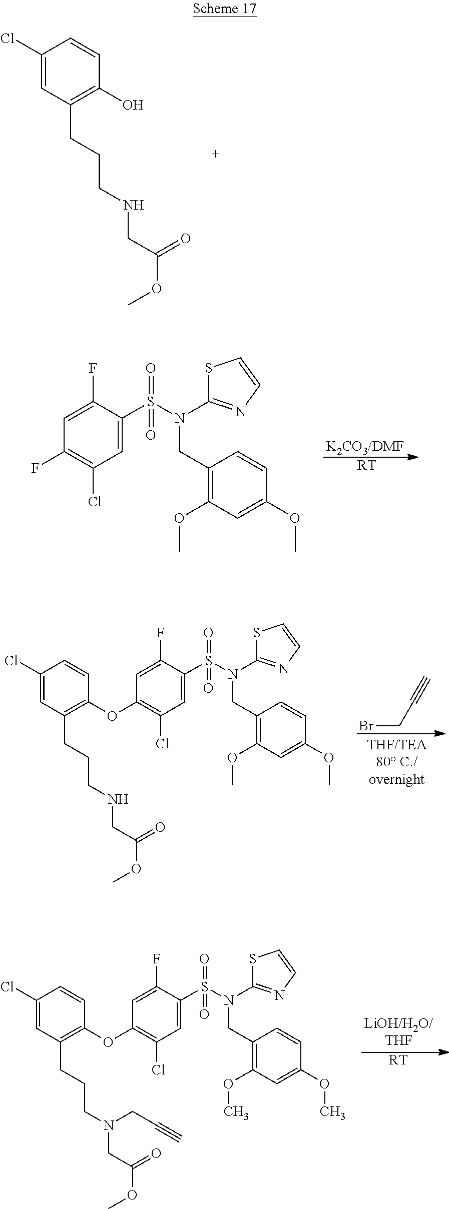

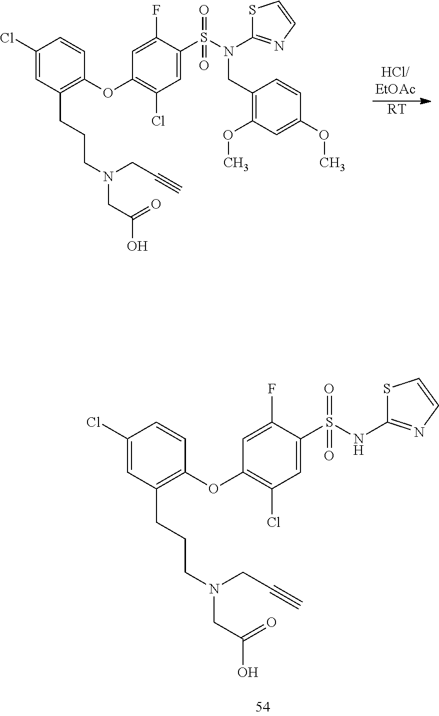 [SMILES: C#CCN(CCCc1cc(Cl)ccc1Oc1cc(F)c(S(=O)(=O)N(Cc2ccc(OC)cc2OC)c2nccs2)cc1Cl)CC(=O)O ; "RT" ; C#CCN(CCCc1cc(Cl)ccc1Oc1cc(F)c(S(=O)(=O)Nc2nccs2)cc1Cl)CC(=O)O]

### Step-1: Preparation of methyl (3-(5-chloro-2-(2-chloro-4-(N-(2,4-dimethoxybenzyl)-N-(thiazol-2-yl) sulfamoyl)-5-fluorophenoxy)phenyl)propyl)glycinate

To a solution methyl 2-(3-(5-chloro-2-hydroxyphenyl) propylamino) acetate (1.1 g, 4.28 mmol) in DMF (12 ml) was added K₂CO₃ (1.77 g, 12.8 mmol) in one portion under nitrogen atmosphere at room temperature. The resulting reaction mixture was stirred at room temperature for 15 minutes. To the above mixture was added 5-chloro-N-(2,4-dimethoxybenzyl)-2,4-difluoro-N-(thiazol-2-yl)benzenesulfonamide (1.96 g, 4.28 mmol) and the resulting mixture was stirred at room temperature for 4 hours. After completion of reaction, D.M. water (100 ml) was added and the resulting mixture was extracted with ethyl acetate (3×50 ml). The combined organic extract was washed with D.M. water (50 ml), brine (50 ml). The combined organic layers were washed with brine, dried over sodium sulphate and concentrated under vacuo to afford 1.5 g (yield, 50.2%) of desired compound as a solid. The material was used directly for next step. LC-MS: m/z=698.5 (M+H).

### Step-2: Preparation of methyl N-(3-(5-chloro-2-(2-chloro-4-(N-(2,4-dimethoxybenzyl)-N-(thiazol-2-yl) sulfamoyl)-5-fluorophenoxy)phenyl)propyl)-N-(prop-2-yn-1-yl)glycinate

To a solution of methyl (3-(5-chloro-2-(2-chloro-4-(N-(2,4-dimethoxybenzyl)-N-(thiazol-2-yl)sulfamoyl)-5-fluorophenoxy)phenyl)propyl)glycinate (0.9 g, 1.28 mmol) in dichloromethane (30 mL) was added triethyl amine (0.54 ml, 3.86 mmol) at room temperature. The resulting reaction mixture was stirred at the same temperature for 10 minutes. 3-Bromoprop-1-yne (0.346 ml, 3.86 mmol) was added to the reaction mixture at room temperature. The resulting reaction mixture was then refluxed at 80° C. for 12 hours. After completion of the reaction, the reaction mixture was cooled to room temperature and dumped in to D.M. water (50 ml). The resulting mixture was extracted with dichloromethane (3×50 ml). The combined organic extract was washed with D.M. water (50 ml), brine (50 ml), dried over sodium sulphate and concentrated under vacuo to get the desired crude product. The crude product was purified by triturating with diethyl ether which gave 0.780 g (yield, 77.8%) of the desired compound as a brown solid. LC-MS: m/z=736.15 (M+H).

### Step-3: Preparation of N-(3-(5-chloro-2-(2-chloro-4-(N-(2,4-dimethoxybenzyl)-N-(thiazol-2-yl)sulfamoyl)-5-fluorophenoxy)phenyl)propyl)-N-(prop-2-yn-1-yl)glycine

To the solution of methyl N-(3-(5-chloro-2-(2-chloro-4-(N-(2,4-dimethoxybenzyl)-N-(thiazol-2-yl)sulfamoyl)-5-fluorophenoxy)phenyl)propyl)-N-(prop-2-yn-1-yl)glycinate (0.7 g, 0.95 mmol) in THF (30 ml) was added a solution of lithium hydroxide monohydrate (0.2 g, 4.75 mmol) in D.M. water (10 ml) at room temperature. The resulting reaction mixture was stirred at room temperature for 3 hours. After completion of reaction ice cold water (20 ml) was added in to the reaction mixture, the resulting mixture was then acidified between 4-6 pH with aqueous 1N hydrochloric acid. The resulting acidic aqueous was extracted with ethyl acetate (3×30 ml). The combined organic extract was washed with D.M. water (20 ml), brine (20 ml), dried over sodium sulphate and concentrated under vacuo to afford 0.43 g (yield, 62.68%) of the compound as a white solid. This material was directly used for next step.

### Step-4: Preparation of N-(3-(5-chloro-2-(2-chloro-5-fluoro-4-(N-(thiazol-2-yl) sulfamoyl) phenoxy) phenyl) propyl)-N-(prop-2-yn-1-yl)glycine

To a solution of N-(3-(5-chloro-2-(2-chloro-4-(N-(2,4-dimethoxybenzyl)-N-(thiazol-2-yl)sulfamoyl)-5-fluorophenoxy)phenyl)propyl)-N-(prop-2-yn-1-yl)glycine (0.4 g, 0.554 mmol) in ethyl acetate (10 ml) was added drop-wise a 4N solution of hydrochloric acid in ethyl acetate (5 ml) at room temperature. The resulting reaction mixture was stirred room temperature for 4 hours. After completion of reaction, pentane (10 ml) was added in to the reaction mixture which resulted in precipitation of solid. The solvent layer was decanted off; the solid thus obtained was washed twice with pentane (10 ml) and dried under vacuo. The resulting crude material was further purified by Prep HPLC using 0.1% HCl in Water: Acetonitrile mobile phase (PREP HPLC Method A). Evaporation of the pure product fractions obtained from Prep HPLC provided the desired product (0.045 g (yield, 14.12%). LC-MS: m/z=572.09 (M+H). 1H-NMR (MeOD), δ 8.05 (d, J=7.1 Hz, 1H), 7.49 (d, J=2.6 Hz, 1H), 7.30-7.39 (m, 1H), 7.17 (d, J=4.7 Hz, 1H), 7.01 (d, J=8.7 Hz, 1H), 6.81 (d, J=4.7 Hz, 1H), 6.75 (d, J=10.5 Hz, 1H), 4.07 (d, J=2.3 Hz, 2H), 3.87 (s, 2H), 3.17-3.25 (m, 3H), 2.64-2.73 (m, 2H), 1.99-2.10 (m, 2H).

### Example 55: 5-chloro-4-(4-chloro-2-(3-(5,6-dihydroimidazo[1,2-a]pyrazin-7(8H)-yl) propyl)phenoxy)-2-fluoro-N-(thiazol-4-yl)benzenesulfonamide

Compound 55 was synthesized according to the procedure described for the synthesis of compound 11 by replacing glycine methyl ester with 5,6,7,8-tetrahydroimidazo[1,2-a]pyrazine in step 2, and omitting step 5. LC-MS: m/z=583 (M+H). 1H-NMR (MeOD), δ 8.72 (d, J=2.2 Hz, 1H), 7.93-8.03 (m, 1H), 7.45-7.52 (m, 1H), 7.29-7.42 (m, 1H), 7.00-7.10 (m, 2H), 6.95 (d, J=1.4 Hz, 1H), 6.61-6.69 (m, 1H), 3.94-4.05 (m, 2H), 3.63 (s, 2H), 2.82-2.87 (m, 2H), 2.72-2.77 (m, 2H), 2.53-2.65 (m, 4H), 1.79-1.92 (m, 2H).

### Example 56: 5-chloro-2-fluoro-4-(2-(4,5,6,7-tetrahydropyrazolo[1,5-a]pyrimidin-3-yl)phenoxy)-N-(thiazol-2-yl)benzenesulfonamide

Compound 56 was synthesized according to the procedure described for the synthesis of compound 33 by replacing 2-(5-chloro-2-methoxyphenyl)acetonitrile with 2-(2-methoxyphenyl)acetonitrile in step 5, and omitting steps 1 to 4. LC-MS: m/z=506.33 (M+H). 1H-NMR (DMSO), δ 7.86 (d, J=7.2 Hz, 1H), 7.55 (d, J=7.6 Hz, 1H), 7.22-7.36 (m, 4H), 7.14-7.19 (m, 1H), 6.85 (d, J=4.3 Hz, 1H), 6.44 (d, J=10.9 Hz, 1H), 6.03 (br. s., 1H), 3.91 (t, J=5.6 Hz, 2H), 3.16 (br. s., 2H), 1.93 (br. s., 2H).

### Example 57: 5-chloro-4-(4-chloro-2-(4,5,6,7-tetrahydropyrazolo[1,5-a]pyrimidin-3-yl)phenoxy)-2-fluoro-N-(thiazol-2-yl)benzenesulfonamide

Compound 57 was synthesized according to the procedure described for the synthesis of compound 33 by replacing tert-butyl 5-chloro-2,4-difluorophenylsulfonyl(thiazol-4-yl)carbamate with 5-chloro-N-(2,4-dimethoxybenzyl)-2,4-difluoro-N-(thiazol-2-yl)benzenesulfonamide in step 9. LC-MS: m/z=539.82 (M+H). 1H-NMR (DMSO), δ 7.89 (s, 1H), 7.56 (d, J=2.5 Hz, 1H), 7.48 (s, 1H), 7.32 (br. s., 1H), 7.22 (s, 1H), 6.88-6.94 (m, 1H), 6.65-6.70 (m, 1H), 3.95 (t, J=5.6 Hz, 2H), 3.18 (t, J=4.8 Hz, 2H), 1.90-2.00 (m, 2H).

### Example 58: 5-chloro-2-fluoro-4-(2-(4,5,6,7-tetrahydropyrazolo[1,5-a]pyrimidin-3-yl)phenoxy)-N-(thiazol-4-yl)benzenesulfonamide

Compound 58 was synthesized according to the procedure described for the synthesis of compound 33 by omitting steps 1 to 4, replacing 2-(5-chloro-2-methoxyphenyl)acetonitrile with 2-(2-methoxyphenyl)acetonitrile in step 5, and replacing tert-butyl 5-chloro-2,4-difluorophenylsulfonyl(thiazol-4-yl)carbamate with tert-butyl ((5-chloro-2,4-difluorophenyl)sulfonyl)(thiazol-4-yl)carbamate in step 9. LC-MS: m/z=505.87 (M+H). 1H-NMR (MeOD), δ 8.76 (d, J=2.2 Hz, 1H), 7.91-7.97 (m, 2H), 7.51-7.62 (m, 2H), 7.43-7.50 (m, 1H), 7.25-7.32 (m, 1H), 7.08 (d, J=2.2 Hz, 1H), 6.49 (d, J=10.8 Hz, 1H), 4.12 (s, 2H), 3.35-3.43 (m, 2H), 2.08-2.20 (m, 2H), 1.32 (s, 2H).

### Example 59: 5-chloro-4-(4-chloro-2-(3-((2-(methylsulfonyl)ethyl)amino)propyl)phenoxy)-2-fluoro-N-(thiazol-2-yl)benzenesulfonamide

Compound 59 was synthesized according to the procedure described for the synthesis of compound 11 by omitting step 5, replacing glycine methyl ester with 2-(methylsulfonyl)ethanamine in step 2 and replacing tert-butyl 5-chloro-2,4-difluorophenylsulfonyl(thiazol-4-yl)carbamate with 5-chloro-N-(2,4-dimethoxybenzyl)-2,4-difluoro-N-(thiazol-2-yl)benzenesulfonamide in step 4. LC-MS: m/z=584.44 (M+H). 1H-NMR (MeOD), δ 8.05 (d, J=7.1 Hz, 1H), 7.49 (d, J=2.5 Hz, 1H), 7.35 (dd, J=8.7, 2.6 Hz, 1H), 7.18 (d, J=4.7 Hz, 1H), 7.01 (d, J=8.7 Hz, 1H), 6.71-6.86 (m, 2H), 3.49-3.61 (m, 4H), 3.09-3.18 (m, 5H), 2.72 (t, J=7.7 Hz, 2H), 2.05 (d, J=1.8 Hz, 2H).

### Example 60: 2-((3-(2-(2-chloro-5-fluoro-4-(N-(thiazol-4-yl) sulfamoyl) phenoxy) phenyl) propyl)amino)acetamide

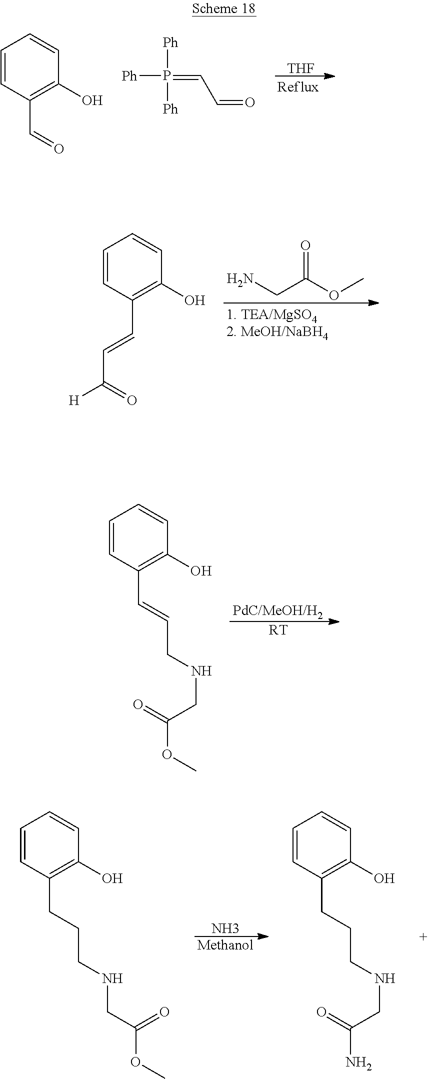

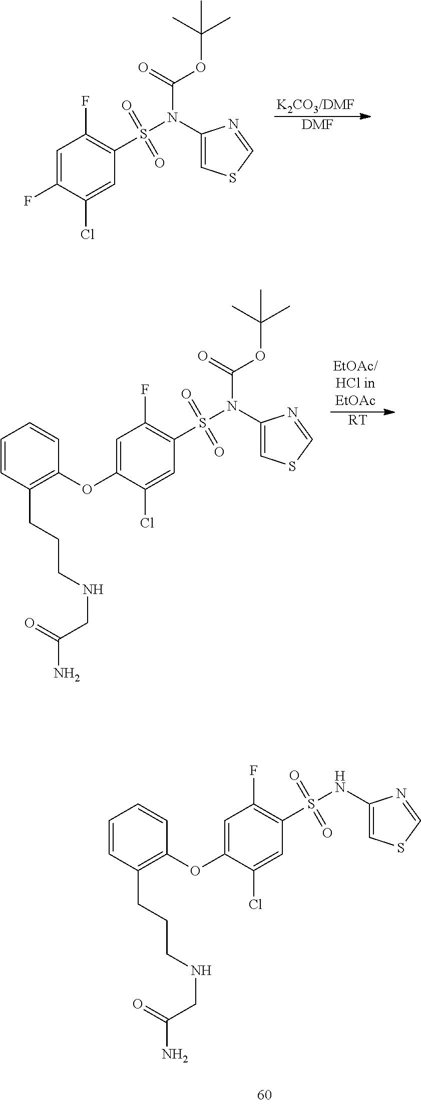

### Step 1: Preparation of 3-(2-hydroxyphenyl)acrylaldehyde

To a solution of 2-hydroxybenzaldehyde (10 g, 81.8 mmol) in THF (150 ml) was added (formylmethylene)triphenylphosphorane (24.89 g, 81.8 mmol) at room temperature. The resulting reaction mixture was refluxed at 100° C. for 20 hours. The reaction mixture was cooled to room temperature, and extracted with water (200 ml) and ethyl acetate (3×150 ml). The combined organic phase was washed with water (150 ml), brine (150 ml), dried over sodium sulphate and concentrated under vacuo to give the desired crude product. The crude product was purified by column chromatography using normal phase silica gel. The desired product eluted at around 20-30% ethyl acetate in hexane. Evaporation of the product fractions gave 8.7 g (yield, 71.86%) of desired compound as yellow solid. LC-MS: m/z=149.42 (M+H).

### Step 2: Preparation of methyl (3-(2-hydroxyphenyl)allyl)glycinate

To a solution of 3-(2-hydroxyphenyl)acrylaldehyde (8 g, 56.7 mmol) and glycine methyl ester hydrochloride (7.8 g, 62.4 mmol) in dichloromethane (100 ml) was added magnesium sulphate (10.21 g, 85.1 mmol) and triethylamine (16 ml, 113.4 mmol) at room temperature. The above reaction mixture was stirred at room temperature for 18 hours. The resulting reaction mixture was then concentrated under vacuo. The concentrated mass thus obtained was dissolved in methanol (50 ml) and cooled to a temperature between 5-10° C. To the above mixture, sodium borohydride (6.4 g, 170.2 mmol) was added in small portions over a period of 20 minutes; during addition temperature of the reaction mixture was maintained between 10-20° C. The reaction mixture was allowed to stir at room temperature for 2 hours and concentrated under vacuo. Water (100 ml) was added to the above crude mass and the resulting mixture was extracted with ethyl acetate (3×100 ml). The combined organic extract was washed with water (50 ml), brine (50 ml), dried over sodium sulphate and concentrated under vacuo to get the desired crude product. The crude product was purified by column chromatography using normal phase silica gel. The desired product eluted at around 1-5% methanol in dichloromethane. Evaporation of the product fractions gave 8 g (yield, 64.64%) of desired compound as yellow solid. LC-MS: m/z=222.33 (M+H).

### Step-3: Preparation of methyl (3-(2-hydroxyphenyl)propyl)glycinate

To a solution of methyl (3-(2-hydroxyphenyl)allyl)glycinate (7.0 g, 31.6 mmol) in methanol (70 ml) was carefully added 10% Palladium on carbon with 50% moisture (0.335 g, 3.1 mmol). Hydrogen gas was then bubbled into the reaction mixture at room temperature for a period of 30 minutes. After completion of the reaction, the reaction mixture was filtered through celite. The celite bed was carefully washed with some amount of methanol. The filtrate thus obtained was concentrated under vacuo to afford 6 g (yield, 85.14%) of compound as colorless liquid and used as is in the next step. LC-MS: m/z=224.33 (M+H).

### Step-4: Preparation of 2-((3-(2-hydroxyphenyl)propyl)amino)acetamide

A solution of methyl (3-(2-hydroxyphenyl)propyl)glycinate (2 g, 8.96 mmol) in methanol (60 ml) was cooled to −78° C. using acetone/dry ice bath. Ammonia gas was then purged in this cold reaction mixture for 1-2 hours. The reaction assembly was then tightly closed and the reaction mixture was allowed to warm to room temperature whereby it further stirred for next 18 hours. The reaction mixture was monitored on TLC using pure ethyl acetate as mobile phase. After completion of reaction, the reaction mixture is evaporated under vacuo and the obtained crude material is further co-evaporated two times with diethyl ether. This final crude material was directly used for the next step without purification. The above process gave 1.8 g (yield, 96.58%) of the desired compound. LC-MS: m/z=208.83 (M+H).

### Step-5: Preparation of tert-butyl 44-(2-(3-((2-amino-2-oxoethyl)amino)propyl)phenoxy)-5-chloro-2-fluorophenyl)sulfonyl)(thiazol-4-yl)carbamate

To a solution 2-((3-(2-hydroxyphenyl)propyl)amino)acetamide (0.1 g, 0.48 mmol) in DMF (3 ml) was added K₂CO₃ (0.13 g, 0.96 mmol) in one portion under nitrogen atmosphere at room temperature. The resulting reaction mixture was stirred at room temperature for 15 minutes. To the above reaction mixture was added tert-butyl ((5-chloro-2,4-difluorophenyl)sulfonyl)(thiazol-4-yl)carbamate (0.23 g, 0.576 mmol) and the resulting mixture was stirred at room temperature for 4-8 hours. After completion of reaction, D.M. water (20 ml) was added and the resulting mixture was extracted with ethyl acetate (2×30 ml). The combined organic extract was washed with D.M. water (20 ml), brine (20 ml), dried over sodium sulphate and concentrated under vacuo. The crude product was purified by column chromatography using normal phase silica gel. The desired product eluted at around 20 to 25% ethyl acetate in hexane. Evaporation of the product fractions gave 0.15 g (yield, 52.16%) of desired compound as a solid LC-MS: m/z=599.69 (M+H).

### Step-6: Preparation of 2-((3-(2-(2-chloro-5-fluoro-4-(N-(thiazol-4-yl) sulfamoyl) phenoxy) phenyl) propyl) amino)acetamide

To a solution of tert-butyl ((4-(2-(3-((2-amino-2-oxoethyl)amino)propyl)phenoxy)-5-chloro-2-fluorophenyl)sulfonyl)(thiazol-4-yl)carbamate (0.15 g, 0.25 mmol) in dichloromethane (10 ml) was added drop-wise a 4N solution of hydrochloric acid in ethyl acetate (5 ml) at room temperature. The resulting reaction mixture was stirred at room temperature for 4 hours. After completion of reaction, pentane (15 ml) was added in to the reaction mixture which resulted in precipitation of solid. The solvent layer was decanted off; the solid thus obtained was washed twice with pentane (15 ml) and dried under vacuo. The resulting crude material was further purified by Prep HPLC using 0.1% Formic acid in Water:Acetonitrile mobile phase (PREP HPLC Method B). Evaporation of the pure product fractions obtained from Prep HPLC provided the desired product (0.025 g, 20.04% yield). LC-MS: m/z=499.23 (M+H). 1H-NMR (MeOD), δ 8.71-8.88 (m, 1H), 7.96-8.08 (m, 1H), 7.42-7.53 (m, 1H), 7.25-7.40 (m, 2H), 7.09-7.13 (m, 1H), 7.00-7.07 (m, 1H), 6.48-6.68 (m, 1H), 3.73 (s, 2H), 2.95-3.05 (m, 2H), 2.62-2.72 (m, 2H), 1.93-2.06 (m, 2H).

### Example 61: 2-((3-(5-chloro-2-(2-chloro-5-fluoro-4-(N-(thiazol-4-yl)sulfamoyl)phenoxy)phenyl)propyl)(prop-2-yn-1-yl)amino)acetic acid

Compound 61 was synthesized according to the procedure described for the synthesis of compound 54 by replacing 5-chloro-N-(2,4-dimethoxybenzyl)-2,4-difluoro-N-(thiazol-2-yl)benzenesulfonamide with tert-butyl 5-chloro-2,4-difluorophenylsulfonyl(thiazol-4-yl)carbamate in step 1. LC-MS: m/z=572.20 (M+H). 1H-NMR (MeOD), δ 8.77 (d, J=2.1 Hz, 1H), 8.01 (d, J=7.1 Hz, 1H), 7.49 (d, J=2.4 Hz, 1H), 7.34 (dd, J=8.7, 2.5 Hz, 1H), 7.11 (d, J=2.2 Hz, 1H), 7.02 (d, J=8.7 Hz, 1H), 6.73 (d, J=10.8 Hz, 1H), 3.91 (br. s., 2H), 3.59 (br. s., 2H), 2.98-3.07 (m, 3H), 2.60-2.67 (m, 2H), 1.92-2.02 (m, 2H).

### Example 62: 2-(allyl(3-(5-chloro-2-(2-chloro-5-fluoro-4-(N-(thiazol-2-yl)sulfamoyl)phenoxy)phenyl)propyl)amino)acetic acid

Compound 62 was synthesized according to the procedure described for the synthesis of compound 54 by replacing 3-bromoprop-1-yne with allyl bromide in step 2. LC-MS: m/z=573.86 (M+H). 1H-NMR (DMSO), δ 12.66 (s, 1H), 7.93 (d, J=7.2 Hz, 1H), 7.50 (br. s., 1H), 7.32 (br. s., 2H), 7.09 (d, J=8.4 Hz, 1H), 6.82-6.93 (m, 2H), 5.66-5.78 (m, 1H), 5.04-5.19 (m, 2H), 3.22 (br. s., 4H), 2.60 (br. s., 2H), 2.52-2.57 (m, 2H), 1.63-1.75 (m, 2H).

### Example 63: 2-((3-(5-chloro-2-(2-chloro-5-fluoro-4-(N-(thiazol-2-yl) sulfamoyl) phenoxy) phenyl) propyl) amino) acetamide

Compound 63 was synthesized according to the procedure described for the synthesis of compound 60 by replacing 2-hydroxybenzaldehyde with 5-chloro-2-hydroxybenzaldehyde in step 1, and replacing tert-butyl 5-chloro-2,4-difluorophenylsulfonyl(thiazol-4-yl)carbamate with 5-chloro-N-(2,4-dimethoxybenzyl)-2,4-difluoro-N-(thiazol-2-yl)benzenesulfonamide in step 5. LC-MS: m/z=532.92 (M+H). 1H-NMR (MeOD), δ 7.97-8.10 (m, 1H), 7.44-7.51 (m, 1H), 7.29-7.41 (m, 1H), 7.13-7.22 (m, 1H), 6.98-7.08 (m, 1H), 6.78-6.84 (m, 1H), 6.71-6.77 (m, 1H), 3.73 (s, 2H), 2.95-3.08 (m, 2H), 2.64-2.78 (m, 2H), 1.92-2.10 (m, 2H).

### Example 64: 2-(but-2-yn-1-yl(3-(5-chloro-2-(2-chloro-5-fluoro-4-(N-(thiazol-2-yl)sulfamoyl)phenoxy)phenyl)propyl)amino)acetic acid

Compound 64 was synthesized according to the procedure described for the synthesis of compound 54 by replacing 3-bromoprop-1-yne with 1-bromo-2-butyne in step 2. LC-MS: m/z=585.95 (M+H). 1H-NMR (MeOD), δ 7.98-8.11 (m, 1H), 7.46-7.54 (m, 1H), 7.32-7.43 (m, 1H), 7.13-7.25 (m, 1H), 6.98-7.05 (m, 1H), 6.72-6.85 (m, 2H), 3.86-4.09 (m, 2H), 3.68-3.80 (m, 2H), 3.17-3.28 (m, 2H), 2.60-2.75 (m, 2H), 1.99-2.12 (m, 2H), 1.87 (s, 3H).

### Example 65: 2-((3-(5-chloro-2-(2-chloro-5-fluoro-4-(N-(thiazol-2-yl)sulfamoyl)phenoxy)phenyl)propyl)(propyl)amino)acetic acid

Compound 65 was synthesized according to the procedure described for the synthesis of compound 54 by replacing 3-bromoprop-1-yne with 1-bromopropane in step 2. LC-MS: m/z=575.90 (M+H). 1H-NMR (MeOD), δ 8.03-8.08 (m, 1H), 7.48-7.52 (m, 1H), 7.30-7.37 (m, 1H), 7.17 (d, J=4.7 Hz, 1H), 7.02 (d, J=8.8 Hz, 1H), 6.81 (d, J=4.6 Hz, 2H), 3.65 (s, 2H), 3.16-3.23 (m, 2H), 3.05-3.13 (m, 2H), 2.63-2.71 (m, 2H), 2.01-2.11 (m, 2H), 1.66-1.78 (m, 2H), 0.98 (t, J=7.4 Hz, 3H).

### Example 66: 3-((3-(5-chloro-2-(2-chloro-5-fluoro-4-(N-(thiazol-2-yl) sulfamoyl) phenoxy) phenyl) propyl) (prop-2-yn-1-yl)amino)propanoic acid

Compound 66 was synthesized according to the procedure described for the synthesis of compound 54 by replacing methyl 2-(3-(5-chloro-2-hydroxyphenyl) propylamino) acetate with methyl 3-[3-(5-chloro-2-hydroxyphenyl)propylamino] propanoate in step 1. LC-MS: m/z=585.88 (M+H). 1H-NMR (DMSO), δ 12.26-12.67 (m, 1H), 7.93 (d, J=7.2 Hz, 1H), 7.50 (d, J=2.6 Hz, 1H), 7.29-7.36 (m, 2H), 7.09 (d, J=8.7 Hz, 1H), 6.85-6.93 (m, 2H), 3.05 (s, 1H), 2.61-2.68 (m, 2H), 2.38-2.44 (m, 2H), 2.27-2.36 (m, 2H), 1.58-1.71 (m, 2H).

### Example 67: 2-((3-(5-chloro-2-(2,5-difluoro-4-(N-(thiazol-2-yl)sulfamoyl)phenoxy)phenyl)propyl)(prop-2-yn-1-yl)amino)acetic acid

Compound 67 was synthesized according to the procedure described for the synthesis of compound 54 by replacing 5-chloro-N-(2,4-dimethoxybenzyl)-2,4-difluoro-N-(thiazol-2-yl)benzenesulfonamide with N-(2,4-dimethoxybenzyl)-2,4,5-trifluoro-N-(thiazol-2-yl)benzenesulfonamide step 1. LC-MS: m/z=555.93 (M+H). 1H-NMR (MeOD), δ 7.77-7.86 (m, 1H), 7.46 (d, J=2.6 Hz, 1H), 7.27-7.34 (m, 1H), 7.17 (d, J=4.7 Hz, 1H), 6.99 (d, J=8.7 Hz, 1H), 6.78-6.89 (m, 2H), 3.93 (s, 2H), 3.59 (s, 2H), 2.99-3.10 (m, 3H), 2.66-2.76 (m, 2H), 1.92-2.03 (m, 2H).

### Example 68: ethyl 2-((3-(5-chloro-2-(2-chloro-5-fluoro-4-(N-(thiazol-2-yl)sulfamoyl)phenoxy)phenyl)propyl)(methyl)amino)acetate

Compound 68 was synthesized according to the procedure described for the synthesis of compound 11 by replacing glycine methyl ester with sarcosine ethyl ester in step 2, and replacing tert-butyl 5-chloro-2,4-difluorophenylsulfonyl(thiazol-4-yl)carbamate with N-(2,4-dimethoxybenzyl)-2,4,5-trifluoro-N-(thiazol-2-yl)benzenesulfonamide in step 4, and omitting step 5. LC-MS: m/z=575.85 (M+H). 1H-NMR (MeOD), δ 8.05 (d, J=7.0 Hz, 1H), 7.50 (d, J=2.5 Hz, 1H), 7.33-7.39 (m, 1H), 7.18 (d, J=4.7 Hz, 1H), 7.01 (d, J=8.6 Hz, 1H), 6.81 (d, J=4.7 Hz, 1H), 6.77 (d, J=10.5 Hz, 1H), 4.32 (d, J=7.2 Hz, 2H), 4.07-4.22 (m, 2H), 3.14-3.24 (m, 2H), 2.96 (s, 3H), 2.70 (s, 2H), 2.05-2.15 (m, 2H), 1.32 (t, J=7.1 Hz, 3H).

### Example 69: 2-((3-(5-chloro-2-(2,5-difluoro-4-(N-(thiazol-4-yl)sulfamoyl)phenoxy)phenyl)propyl)amino)acetamide

Compound 69 was synthesized according to the procedure described for the synthesis of compound 49 by replacing tert-butyl 5-chloro-2,4-difluorophenylsulfonyl(thiazol-4-yl)carbamate with tert-butyl thiazol-4-yl((2,4,5-trifluorophenyl)sulfonyl)carbamate in step 4. LC-MS: m/z=516.8 (M+H). 1H-NMR (DMSO-d6), d 8.94 (d, J=2.0 Hz, 1H), 8.90 (br, 2H), 7.84-7.88 (m, 2H), 7.58 (s, 1H), 7.50 (d, J=2.4 Hz, 1H), 7.33-7.37 (dd, J=2.8, 8.8 Hz, 1H), 7.09-7.13 (m, 3H), 3.66 (s, 2H), 2.90 (br, 2H), 2.62 (t, J=7.6 Hz, 2H), 1.88-1.92 (m, 2H).

The embodiments described herein are intended to be merely exemplary, and those skilled in the art will recognize, or be able to ascertain using no more than routine experimentation, numerous equivalents to the specific procedures described herein. All such equivalents are considered to be within the scope of the present invention and are covered by the following embodiments.

All references (including patent applications, patents, and publications) cited herein are incorporated herein by reference in their entirety and for all purposes to the same extent as if each individual publication or patent or patent application was specifically and individually indicated to be incorporated by reference in its entirety for all purposes.

What is claimed is:

1. A method for treating or preventing prediabetes comprising administering to a subject in need thereof a therapeutically effective amount of a compound selectively inhibiting NaV1.7, wherein compound is a compound of Formula (I′): 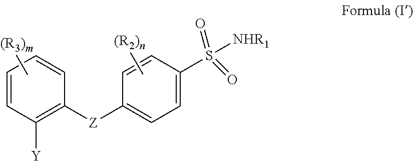
or a pharmaceutically acceptable salt, or a stereoisomeric or tautomeric form thereof,
wherein:
Z is —O— or —S—;
Y is —X—C(═O)NR₄R₅, —(CH₂)₃—NR₉R₁₀, or 4,5,6,7-tetrahydropyrazolo[1,5-a]pyrimidine-(2-yl or 3-yl);
X is (C₆-C₁₀)aryl or 5- or 6-membered heteroaryl;
R₁ is a partially unsaturated or aromatic 5- or 6-membered heterocycle;
R₂ is independently at each occurrence —F, —Cl, —Br, —CH₃ or —CN,
R₃ is independently at each occurrence —H, —F, —Cl, —Br, —CF₃—OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
R₄ and R₅ are not both H; and
at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
R₆ is (C₁-C₁₂)alkyl;
R₇ and R₈ are each independently H, (C₁-C₁₂)alkyl, or R₇ and R₈ together form a 4- to 7-membered heterocycloalkyl ring;
R₉ is (C₁-C₆)alkyl, (C₃-C₈)cycloalkyl, pyrazolyl or pyridinyl; wherein R₉ is optionally further substituted with 1 or 2 substituents selected from the group consisting of —COOH, —COOR₁₁, —CONR₁₁R₁₂, —SO₂R₁₁, —SO₂NR₁₁R₁₂, —OH, —CN, —OR₁₁, and —NR₁₁R₁₂; wherein R₁₁ and R₁₂ may form a 6 membered heterocycloalkyl ring
R₁₀ is R₁₁, (C₃-C₆)alkynyl, (C₃-C₆)alkenyl, —COR₁₁, —COOR₁₁, —SO₂R₁₁, 5-methyl-2-oxo-1,3-dioxol-4-yl, 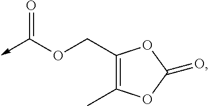
—COO—CH(CH₃)OCOCH(CH₃)₂ or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy; or R₉ and R₁₀ together form a unsubstituted 4- to 8-membered heterocycloalkyl ring, wherein said heterocycloalkyl ring is fused with a 5-membered heteroaryl; and
R₁₁ and R₁₂ are independently H or (C₁-C₆)alkyl, optionally substituted with 4- to 8-membered heterocycloalkyl ring; and
m and n are each independently 1, 2, 3, or 4.

2. A method for treating or preventing diabetes comprising administering to a subject in need thereof a therapeutically effective amount of a compound selectively inhibiting NaV1.7, wherein compound is a compound of Formula (I′): 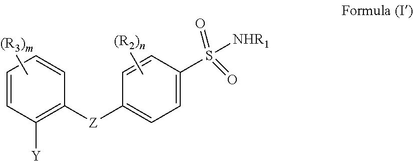
or a pharmaceutically acceptable salt, or a stereoisomeric or tautomeric form thereof,
wherein:
Z is —O— or —S—;
Y is —X—C(═O)NR₄R₅, —(CH₂)₃—NR₉R₁₀, or 4,5,6,7-tetrahydropyrazolo[1,5-a]pyrimidine-(2-yl or 3-yl);
X is (C₆-C₁₀)aryl or 5- or 6-membered heteroaryl;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
R₄ and R₅ are not both H; and
at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
R₆ is (C₁-C₁₂)alkyl;
R₇ and R₈ are each independently H, (C₁-C₁₂)alkyl, or R₇ and R₈ together form a 4- to 7-membered heterocycloalkyl ring;
R₉ is (C₁-C₆)alkyl, (C₃-C₈)cycloalkyl, pyrazolyl or pyridinyl; wherein R₉ is optionally further substituted with 1 or 2 substituents selected from the group consisting of —COOH, —COOR₁₁, —CONR₁₁R₁₂, —SO₂R₁₁, —SO₂NR₁₁R₁₂, —OH, —CN, —OR₁₁, and —NR₁₁R₁₂; wherein R₁₁ and R₁₂ may form a 6 membered heterocycloalkyl ring
R₁₀ is R₁₁, (C₃-C₆)alkynyl, (C₃-C₆)alkenyl, —COR₁₁, —COOR₁₁, —SO₂R₁₁, 5-methyl-2-oxo-1,3-dioxol-4-yl, 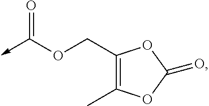
—COO—CH(CH₃)OCOCH(CH₃)₂; or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy; or R₉ and R₁₀ together form a unsubstituted 4- to 8-membered heterocycloalkyl ring, wherein said heterocycloalkyl ring is fused with a 5-membered heteroaryl; and
R₁₁ and R₁₂ are independently H or (C₁-C₆)alkyl, optionally substituted with 4- to 8-membered heterocycloalkyl ring; and
m and n are each independently 1, 2, 3, or 4.

3. A method for maintaining or lowering blood or plasma glucose levels in a subject in need thereof comprising administering to the subject, a therapeutically effective amount of a compound selectively inhibiting NaV1.7, wherein compound is a compound of Formula (I′): 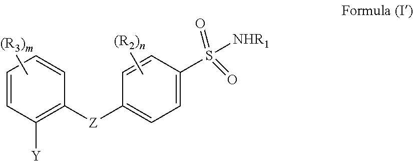
or a pharmaceutically acceptable salt, or a stereoisomeric or tautomeric form thereof,
wherein:
Z is —O— or —S—;
Y is —X—C(═O)NR₄R₅, —(CH₂)₃—NR₉R₁₀, or 4,5,6,7-tetrahydropyrazolo[1,5-a]pyrimidine-(2-yl or 3-yl);
X is (C₆-C₁₀)aryl or 5- or 6-membered heteroaryl;
R₁ is a partially unsaturated or aromatic 5- or 6-membered heterocycle;
R₂ is independently at each occurrence —F, —Cl, —Br, —CH₃ or —CN;
R₃ is independently at each occurrence —H, —F, —Cl, —Br, —CF₃, —OCF₃, —CN, (C₁-C₁₂)alkyl, or (C₁-C₁₂)alkoxy;
R₄ and R₅ are each independently H, (C₁-C₉)alkyl, (C₄-C₁₂)cycloalkyl, or R₄ and R₅ together form a 5- to 7-membered heterocycloalkyl ring; with the proviso that:
R₄ and R₅ are not both H; and
at least one of R₄ and R₅ independently or said heterocycloalkyl ring formed by R₄ and R₅ together is substituted with 1 or 2 substituents selected from the group consisting of —CO₂H, —CO₂R₆, —CN, —OH, —CONR₇R₈, and —NR₇R₈; wherein:
R₆ is (C₁-C₁₂)alkyl;
R₇ and R₈ are each independently H, (C₁-C₁₂)alkyl, or R₇ and R₈ together form a 4- to 7-membered heterocycloalkyl ring;
R₉ is (C₁-C₆)alkyl, (C₃-C₈)cycloalkyl, pyrazolyl or pyridinyl; wherein R₉ is optionally further substituted with 1 or 2 substituents selected from the group consisting of —COOH, —COOR₁₁, —CONR₁₁R₁₂, —SO₂R₁₁, —SO₂NR₁₁R₁₂, —OH, —CN, —OR₁₁, and —NR₁₁R₁₂; wherein R₁₁ and R₁₂ may form a 6 membered heterocycloalkyl ring
R₁₀ is R₁₁, (C₃-C₆)alkynyl, (C₃-C₆)alkenyl, —COR₁₁, —COOR₁₁, —SO₂R₁₁, 5-methyl-2-oxo-1,3-dioxol-4-yl, 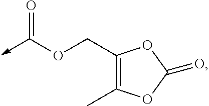
—COO—CH(CH₃)OCOCH(CH₃)₂ or R₉ and R₁₀ together form a piperazinone or a 4- to 8-membered heterocycloalkyl ring, wherein said heterocycloalkyl ring is substituted with 1 or 2 substituents selected from the group consisting of —COOH, —COOR₁₁, —CH₂—COOR₁₁, —OH, —NH₂, —CN, and (C₁-C₈)alkoxy; or R₉ and R₁₀ together form a unsubstituted 4- to 8-membered heterocycloalkyl ring, wherein said heterocycloalkyl ring is fused with a 5-membered heteroaryl; and
R₁₁ and R₁₂ are independently H or (C₁-C₆)alkyl, optionally substituted with 4- to 8-membered heterocycloalkyl ring; and
m and n are each independently 1, 2, 3, or 4.

4-6. (canceled)

7. The method of claim 2, wherein Y is —(CH₂)₃—NR₉R₁₀.

8. The method of claim 7, wherein R₁ is an aromatic 5- or 6-membered heterocycle, with 1-3 heteroatoms independently selected from the group consisting of N, O, and S.

9-14. (canceled)

15. The method of claim 7, wherein R₂ is independently at each occurrence —F or —Cl.

16. The method of claim 7, wherein n is 1, 2, or 3.

17. (canceled)

18. The method of claim 7, wherein Z is —O—.

19. The method of claim 7, wherein R₃ is independently at each occurrence —H, —F, —Cl, or —Br.

20-21. (canceled)

22. The method of claim 7, wherein m is 1, 2, or 3.

23. (canceled)

24. The method of claim 7, wherein R₉ is (C₁-C₆)alkyl; wherein R₉ is optionally further substituted with 1 or 2 substituents selected from the group consisting of —COOH, —COOMe, —CONH₂, and —NH₂.

25. (canceled)

26. The method of claim 7, wherein R₉ is further substituted with —COOH.

27. The method of claim 7, wherein R₁₀ is —H.

28-29. (canceled)

30. The method of claim 7, wherein R₁₀ is H and R₉ is (C₁-C₆)alkyl, wherein R₉ is further substituted with —COR₁₁R₁₂, and wherein R₁₁ and R₁₂ are independently H or (C₁-C₆)alkyl.

31. The method of claim 30, wherein the R₉ is methyl.

32. The method of claim 31, wherein the R₉ is further substituted with —CONH₂.

33. The method of claim 7, wherein R₉ and R₁₀ together form a 4 to 8 membered heterocycloalkyl ring, wherein said heterocycloalkyl ring is substituted with 1 or 2 groups selected from the group consisting of —COOH, —COOMe, —COOEt, —CH₂—COOH, and —NH₂, wherein R₉ and Rio together form a 4 to 8 membered heterocycloalkyl ring, wherein said heterocycloalkyl ring is substituted with 1 or 2 groups selected from the group consisting of —COOH, —CH₂—COOH, and —NH₂; or wherein R₉ and Rio together form a piperidine substituted with 1 or 2 groups selected from the group consisting of —COOH, —COOMe, —COOEt, —CH₂—COOH, —CH₂—COOMe, —CH₂—COOEt, and —NH₂, or wherein R₉ and R₁₀ together form a piperidine substituted with 1 or 2 groups selected from the group consisting of —COOH, —COOMe, —COOEt, —CH₂—COOH, —CH₂—COOMe, —CH₂—COOEt, and —NH₂.

34-81. (canceled)

82. The method of claim 2, wherein diabetes is gestational diabetes, type-1 diabetes, type-2 diabetes, or latent autoimmune diabetes of adults.

83-93. (canceled)

94. The method of claim 2, wherein the compound is selected from the group consisting of: 2-((3-(5-chloro-2-(2-chloro-5-fluoro-4-(N-(thiazol-2-yl)sulfamoyl)phenoxy)phenyl)propyl)(propyl)amino)acetic acid, 2-(allyl(3-(5-chloro-2-(2-chloro-5-fluoro-4-(N-(thiazol-2-yl)sulfamoyl)phenoxy)phenyl)propyl)amino)acetic acid, 3-((3-(5-chloro-2-(2-chloro-5-fluoro-4-(N-(thiazol-2-yl)sulfamoyl)phenoxy)phenyl)propyl)(prop-2-yn-1-yl)amino)propanoic acid, and 2-((3-(5-chloro-2-(2-chloro-5-fluoro-4-(N-(thiazol-4-yl)sulfamoyl)phenoxy)phenyl)propyl)amino)acetamide; or a pharmaceutically acceptable salt, or a tautomeric form thereof.
